(19) United States
(12) Patent Application Publication
(10) Pub. No.: US 2023/0355674 A1
(43) Pub. Date: Nov. 9, 2023

(54) DONOR T-CELLS WITH KILL SWITCH

(71) Applicant: CSL Behring LLC, King of Prussia, PA (US)

(72) Inventors: Chao-Guang Chen, Parkville (AU); Walid Jhan Azar, Elsternwick (AU); Arthur Lian-Chi Hsu, Balwyn (AU); Christopher Walter Alma, Castle Hill (AU)

(21) Appl. No.: 18/078,977

(22) Filed: Dec. 11, 2022

Related U.S. Application Data
(63) Continuation of application No. PCT/US21/39022, filed on Jun. 25, 2021.
(60) Provisional application No. 63/044,697, filed on Jun. 26, 2020.

(51) Int. Cl. A61K 35/17; C12N 9/22; C12N 15/11; C12N 15/90; A61K 31/519; A61P 37/06; A61K 35/28

(52) U.S. Cl. CPC A61K 35/17; C12N 9/22; C12N 15/111; C12N 15/907; A61K 31/519; A61P 37/06; A61K 35/28; C12N 2310/20

(57) ABSTRACT

The disclosed methods are generally directed to preventing, treating, suppressing, controlling or otherwise mitigating side effects of T-cell therapy, the T-cell therapy designed to accelerate immune reconstitution, induce a graft-versus-malignancy effect, and/or target tumor cells. In some embodiments, the present disclosure provides delivery vehicles including components adapted to knockout HPRT. In some embodiments, the delivery vehicles include a gRNA molecule and an endonuclease, e.g. a Cas protein. In some embodiments, the gRNA molecules target locations within Exons 2, 3, or 8 of the HPRT 1 gene.

### CROSS REFERENCE TO RELATED APPLICATIONS

The present application is a continuation of International Application No. PCT/US21/39022 filed on Jun. 25, 2021, which application claims the benefit of the filing date of U.S. Provisional Patent Application No. 63/044,697 filed on Jun. 26, 2020, the disclosure of which is hereby incorporated by reference herein in its entirety.

### FIELD OF DISCLOSURE

The present disclosure generally relates to gene therapy and, in particular, hematopoietic stem cells and/or lymphocytes, such as T-cells transduced with expression vectors. The present disclosure also relates to gene editing, such as through the CRISPR-Cas system.

### BACKGROUND OF THE DISCLOSURE

Allogeneic hematopoietic stem-cell transplantation (allo-HSCT) is a curative therapy for hematological malignancies and inherited disorders of blood cells, such as sickle cell disease. Challenges associated with allo-HSCT include identification of an appropriate source of donor cells. While matched-related donors (MRD) and matched unrelated donors (MUD) provide a source of HSC with lower associated risks, the availability of these donors is reduced significantly compared to the availability of donors that are haplo-identical, of which almost everyone has an immediate donor (typically a parent or sibling). There are, however, complications associated with the use of haplo-identical donors for allo-HSCT, the most significant being the potential for development of graft-versus-host disease (GvHD), which remains an obstacle for successful allo-HSCT. It is believed that approximately half of the patients undergoing allo-HSCT develop GvHD requiring treatment, and more than 10% of the patients may die because of it. GvHD presents with heterogeneous symptoms involving multiple organ systems including gastrointestinal tract, skin, mucosa, liver and lungs. Immunosuppressive drugs have served as a central strategy to prevent and reduce GvHD. Currently, the standard treatment with corticosteroids for GvHD with corticosteroids has very limited success, as many patients develop steroid-refractory disease. There is no clear consensus on what comprises the best second- and third-line approach in the treatment of acute and chronic GvHD (see Jamil, M. O. & Mineishi, S. Int J Hematol (2015) 101: 452).

In order to reduce the risk of GvHD development, haplo-identical transplants are often T-cell depleted. However, a lack of donor T-cells leaves transplant recipients immunocompromised and can result in increased rates of deadly infections in the transplanted patient. In addition, more recent work has shown that the presence of donor T-cells significantly improves donor cell engraftment thereby reducing the potential need for repeat HSCT, in addition to providing T-cell immunity for the extended period of time required for CD4+ and CD8+ T-cell engraftment (up to 2 years).

In an allo-HSCT malignant setting, the benefits afforded by the presence of donor T-cells include anti-tumor activity, or graft-versus-tumor (GVT) effect (also known as graft-vs-leukemia—GVL). The first report of donor lymphocyte infusions (DLI) leading to remission of disease following relapse after HSCT was in a patient with chronic myeloid leukemia (CML) in 1990. Prior to DLI, patients relapsing following HSCT would have likely succumbed to their disease and few patients would have received a second transplant. Following success in CML, DLI was then utilized for other hematological malignancies such as acute leukemia and myeloma. A significant challenge therefore relates to the appropriate balance of the GVT effect, which is responsible for enabling sustained remission, but which is also responsible for the toxicity associated with GvHD.

### BRIEF SUMMARY OF THE DISCLOSURE

Gene therapy strategies to modify human stem cells hold great promise for curing many human diseases. It is believed that the full therapeutic potential of allo-HSCT will not be realized until approaches are developed which minimize GvHD while concomitantly maintaining the positive contributions of donor T-cells.

In a first aspect of the present disclosure is a method of providing the benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA molecule targeting a sequence within one of Exon 3 or Exon 8 of the HPRT 1 gene; (b) positively selecting for the population of substantially HPRT deficient lymphocytes ex vivo to provide a population of modified lymphocytes; and (c) administering a therapeutically effective amount of the population of modified lymphocytes to the patient. In some embodiments, the lymphocytes are T-cells, preferably human primary T-cells. In some embodiments, the method further comprises administering an HSC graft to the patient. In some embodiments, the HSC graft is administered prior to, contemporaneously with, or following the administration of the population of modified lymphocytes.

In some embodiments, the guide RNA molecule targets a sequence within Exon 3 of the HPRT1 gene. In some embodiments, the guide RNA molecule targets a sequence within Exon 8 of the HPRT1 gene. In some embodiments, the guide RNA molecule targeting the sequence within the one of Exon 3 or Exon 8 of the HPRT1 gene has at least 90% sequence identity to any one of SEQ ID NOS: 40-44 and 46-56. In some embodiments, the guide RNA molecule targeting the sequence within the one of Exon 3 or Exon 8 of the HPRT1 gene has at least 91% sequence identity to any one of SEQ ID NOS: 40-44 and 46-56. In some embodiments, the guide RNA molecule targeting the sequence within the one of Exon 3 or Exon 8 of the HPRT1 gene has at least 92% sequence identity to any one of SEQ ID NOS: 40-44 and 46-56. In some embodiments, the guide RNA molecule targeting the sequence within the one of Exon 3 or Exon 8 of the HPRT1 gene has at least 93% sequence identity to any one of SEQ ID NOS: 40-44 and 46-56. In some embodiments, the guide RNA molecule targeting the sequence within the one of Exon 3 or Exon 8 of the HPRT1 gene has at least 94% sequence identity to any one of SEQ ID NOS: 40-44 and 46-56.

In some embodiments, the guide RNA molecule targeting the sequence within the one of Exon 3 or Exon 8 of the HPRT1 gene has at least 95% sequence identity to any one of SEQ ID NOS: 40-44 and 46-56. In some embodiments, the guide RNA molecule targeting the sequence within the one of Exon 3 or Exon 8 of the HPRT1 gene has at least 96% sequence identity to any one of SEQ ID NOS: 40-44 and 46-56. In some embodiments, the guide RNA molecule targeting the sequence within the one of Exon 3 or Exon 8 of the HPRT1 gene has at least 97% sequence identity to any one of SEQ ID NOS: 40-44 and 46-56. In some embodiments, the guide RNA molecule targeting the sequence within the one of Exon 3 or Exon 8 of the HPRT1 gene has at least 98% sequence identity to any one of SEQ ID NOS: 40-44 and 46-56. In some embodiments, the guide RNA molecule targeting the sequence within the one of Exon 3 or Exon 8 of the HPRT1 gene has at least 99% sequence identity to any one of SEQ ID NOS: 40-44 and 46-56. In some embodiments, the guide RNA molecule targeting the sequence within the one of Exon 3 or Exon 8 of the HPRT1 gene comprises any one of SEQ ID NOS: 40-44 and 46-56.

In some embodiments, the endonuclease comprises a Cas protein. In some embodiments, the Cas protein comprises a Cas9 protein. In some embodiments, the Cas protein comprises a Cas12 protein. In some embodiments, the Cas12 protein is a Cas12a protein. In some embodiments, the Cas12 protein is a Cas12b protein.

In some embodiments, the lymphocytes obtained from the donor sample are transfected or transduced with a viral delivery vehicle, a non-viral delivery vehicle, and/or through a physical method. In some embodiments, the physical method is selected from microinjection and electroporation.

In some embodiments, the non-viral delivery vehicle is a nanocapsule. In some embodiments, the nanocapsule optionally comprises at least one targeting moiety. In some embodiments, the nanocapsule comprises at least one targeting moiety. In some embodiments, the at least one targeting moiety targets any one of a human mesenchymal stem cell CD marker, including CD29, CD44, CD90, CD49a-f, CD51, CD73 (SH3), CD105 (SH2), CD106, CD166. and. Stro-1 markers. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127, FoxP3 and CD44. In some embodiments, the T-cell marker is CD3. In some embodiments, the T-cell marker is CD28.

In some embodiments, co-stimulation with one or more co-stimulating moieties may be used to activate target cells, including T-cells. In some embodiments, co-stimulation may be achieved by activating one or more cell surface markers, including but not limited to CD28, ICOS, CTLA4, PD1, PD1H, and BTLA. In some embodiments, the co-stimulating moieties are antibodies.

In some embodiments, the viral delivery vehicle is an expression vector, and wherein the expression vector includes a first nucleic acid sequence encoding for the endonuclease and a second nucleic acid encoding for the guide RNA molecule. In some embodiments, the expression vector is a lentiviral expression vector.

In some embodiments, a level of HPRT1 gene expression within the population of substantially HPRT deficient lymphocytes is reduced by at least about 70% as compared with the donor lymphocytes which have not been transfected. In some embodiments, a level of HPRT1 gene expression within the population of substantially HPRT deficient lymphocytes is reduced by at least about 75% as compared with the donor lymphocytes which have not been transfected. In some embodiments, a level of HPRT1 gene expression within the population of substantially HPRT deficient lymphocytes is reduced by at least about 80% as compared with the donor lymphocytes which have not been transfected. In some embodiments, a level of HPRT1 gene expression within the population of substantially HPRT deficient lymphocytes is reduced by at least about 85% as compared with the donor lymphocytes which have not been transfected. In some embodiments, a level of HPRT1 gene expression within the population of substantially HPRT deficient lymphocytes is reduced by at least about 90% as compared with the donor lymphocytes which have not been transfected. In some embodiments, the lymphocytes are T-cells, preferably human primary T-cells. In some embodiments, a level of HPRT1 gene expression within the population of substantially HPRT deficient lymphocytes is reduced by at least about 95% as compared with the donor lymphocytes which have not been transfected.

In some embodiments, the positive selection comprises contacting the generated population of substantially HPRT deficient lymphocytes with a purine analog. In some embodiments, the purine analog is 6-TG. In some embodiments, the purine analog is 6-mercaptopurine (6-MP). In some embodiments, an amount of the purine analog ranges from between about 1 to about 15 μg/mL. In some embodiments, the positive selection comprises contacting the generated population of substantially HPRT deficient lymphocytes with both a purine analog (e.g., in an amount ranging from between about 1 to about 15 μg/mL) and allopurinol.

In some embodiments, at least about 70% of the population of modified lymphocytes are sensitive to a dihydrofolate reductase inhibitor. In some embodiments, at least about 80% of the population of modified lymphocytes are sensitive to a dihydrofolate reductase inhibitor. In some embodiments, the method further comprises administering to the patient one or more doses of a dihydrofolate reductase inhibitor (e.g. two or more doses, three or more doses, four or more doses, etc.). In some embodiments, the dihydrofolate reductase inhibitor is selected from the group consisting of MTX or MPA.

In some embodiments, the population of modified lymphocytes are administered as a single bolus. In some embodiments, multiple doses of the population of modified lymphocytes are administered to the patient (e.g., two or more doses, three or more doses, four or more doses, etc.). In some embodiments, each dose of the multiple doses comprises between about 0.1×10⁶ cells/kg to about 240×10⁶ cells/kg. In some embodiments, a total dosage comprises between about 0.1×10⁶ cells/kg to about 730×10⁶ cells/kg.

In a second aspect of the present disclosure is a method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA molecule targeting a sequence within Chromosome X located between about 134475181 to about 134475364 (based on genome build GRCh38 or the equivalent position in a genome build other than GRCh38) or between about 134498608 to about 134498684 (based on genome build GRCh38 or the equivalent position in a genome build other than GRCh38); (b) positively selecting for the population of substantially HPRT deficient lymphocytes ex vivo to provide a population of modified lymphocytes; and (c) administering a therapeutically effective amount of the population of modified lymphocytes to the patient. In some embodiments, the lymphocytes are T-cells, preferably human primary T-cells. In some embodiments, the method further comprises administering an HSC graft to the patient. In some embodiments, the HSC graft is administered prior to, contemporaneously with, or following the administration of the population of modified lymphocytes.

In some embodiments, the guide RNA molecules targets a sequence within Chromosome X located between about 134475181 to about 134475364 based on genome build GRCh38 or the equivalent position in a genome build other than GRCh38. In some embodiments, the guide RNA molecule is at least about 85% complementary to a sequence within Chromosome X located between about 134475181 to about 134475364 based on genome build GRCh38 or the equivalent position in a genome build other than GRCh38. In some embodiments, the sequence targeted has a length ranging from between about 14 nucleotides to about 30 nucleotides. In some embodiments, the sequence targeted has a length ranging from between about 16 nucleotides to about 28 nucleotides. In some embodiments, the sequence targeted has a length ranging from between about 18 nucleotides to about 26 nucleotides. In some embodiments, the sequence targeted has a length ranging from between about 21 nucleotides to about 25 nucleotides. In some embodiments, the sequence targeted has a length of about 21 nucleotides. In some embodiments, the sequence targeted has a length of about 22 nucleotides. In some embodiments, the sequence targeted has a length of about 23 nucleotides. In some embodiments, the sequence targeted has a length of about 24 nucleotides. In some embodiments, the sequence targeted has a length of about 25 nucleotides.

In some embodiments, the guide RNA molecules targets a sequence within Chromosome X located between about 134498608 to about 134498684 based on GRCh38 or the equivalent position in a genome build other than GRCh38. In some embodiments, the guide RNA molecule is at least about 85% complementary to a sequence within Chromosome X located between about 134498608 to about 134498684 based on GRCh38 or the equivalent position in a genome build other than GRCh38. In some embodiments, the sequence targeted has a length ranging from between about 14 nucleotides to about 30 nucleotides. In some embodiments, the sequence targeted has a length ranging from between about 16 nucleotides to about 28 nucleotides. In some embodiments, the sequence targeted has a length ranging from between about 18 nucleotides to about 26 nucleotides. In some embodiments, the sequence targeted has a length ranging from between about 21 nucleotides to about 25 nucleotides. In some embodiments, the sequence targeted has a length of about 21 nucleotides. In some embodiments, the sequence targeted has a length of about 22 nucleotides. In some embodiments, the sequence targeted has a length of about 23 nucleotides. In some embodiments, the sequence targeted has a length of about 24 nucleotides. In some embodiments, the sequence targeted has a length of about 25 nucleotides.

In some embodiments, the guide RNA molecule has at least 90% sequence identity to any one of SEQ ID NOS: 40-44 and 46-56. In some embodiments, the guide RNA molecule has at least 91% sequence identity to any one of SEQ ID NOS: 40-44 and 46-56. In some embodiments, the guide RNA molecule has at least 92% sequence identity to any one of SEQ ID NOS: 40-44 and 46-56. In some embodiments, the guide RNA molecule has at least 93% sequence identity to any one of SEQ ID NOS: 40-44 and 46-56. In some embodiments, the guide RNA molecule has at least 94% sequence identity to any one of SEQ ID NOS: 40-44 and 46-56. In some embodiments, the guide RNA molecule gene has at least 95% sequence identity to any one of SEQ ID NOS: 40-44 and 46-56. In some embodiments, the guide RNA molecule has at least 96% sequence identity to any one of SEQ ID NOS: 40-44 and 46-56. In some embodiments, guide RNA molecule has at least 97% sequence identity to any one of SEQ ID NOS: 40-44 and 46-56. In some embodiments, guide RNA molecule has at least 98% sequence identity to any one of SEQ ID NOS: 40-44 and 46-56. In some embodiments, guide RNA molecule has at least 99% sequence identity to any one of SEQ ID NOS: 40-44 and 46-56. In some embodiments, the guide RNA molecule comprises any one of SEQ ID NOS: 40-44 and 46-56.

In some embodiments, the endonuclease comprises a Cas protein. In some embodiments, the Cas protein comprises a Cas9 protein. In some embodiments, the Cas protein comprises a Cas12 protein. In some embodiments, the Cas12 protein is a Cas12a protein. In some embodiments, the Cas12 protein is a Cas12b protein.

In some embodiments, the lymphocytes obtained from the donor sample are transfected or transduced with a viral delivery vehicle, a non-viral delivery vehicle, and/or through a physical method. In some embodiments, the physical method is selected from microinjection and electroporation.

In some embodiments, the non-viral delivery vehicle is a nanocapsule. In some embodiments, the nanocapsule comprises at least one targeting moiety. In some embodiments, the at least one targeting moiety targets any one of a human mesenchymal stem cell CD marker, including the CD29, CD44, CD90, CD49a-f, CD51, CD73 (SH3), CD105 (SH2), CD106, CD166, and Stro-1 markers. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127, FoxP3 and CD44. In some embodiments, the T-cell marker is CD3. In some embodiments, the T-cell marker is CD28.

In some embodiments, co-stimulation with one or more co-stimulating moieties may be used to activate target cells, including T-cells. In some embodiments, co-stimulation may be achieved by activating one or more cell surface markers, including but not limited to CD28, ICOS, CTLA4, PD1, PD1H, and BTLA. In some embodiments, the co-stimulating moieties are antibodies.

In some embodiments, the viral delivery vehicle is an expression vector, and wherein the expression vector includes a first nucleic acid sequence encoding for the endonuclease and a second nucleic acid encoding for the guide RNA molecule. In some embodiments, the expression vector is a lentiviral expression vector.

In some embodiments, a level of HPRT1 gene expression within the population of substantially HPRT deficient lymphocytes is reduced by at least about 70% as compared with the donor lymphocytes which have not been transfected. In some embodiments, a level of HPRT1 gene expression within the population of substantially HPRT deficient lymphocytes is reduced by at least about 75% as compared with the donor lymphocytes which have not been transfected. In some embodiments, a level of HPRT1 gene expression within the population of substantially HPRT deficient lymphocytes is reduced by at least about 80% as compared with the donor lymphocytes which have not been transfected. In some embodiments, a level of HPRT1 gene expression within the population of substantially HPRT deficient lymphocytes is reduced by at least about 85% as compared with the donor lymphocytes which have not been transfected. In some embodiments, a level of HPRT 1 gene expression within the population of substantially HPRT deficient lymphocytes is reduced by at least about 90% as compared with the donor lymphocytes which have not been transfected.

In some embodiments, the positive selection comprises contacting the generated population of substantially HPRT deficient lymphocytes with a purine analog. In some embodiments, the purine analog is 6-TG. In some embodiments, the purine analog is 6-MP. In some embodiments, an amount of the purine analog ranges from between about 1 to about 15 μg/mL. In some embodiments, the positive selection comprises contacting the generated population of substantially HPRT deficient lymphocytes with both a purine analog and allopurinol.

In some embodiments, at least about 70% of the modified lymphocytes are sensitive to a dihydrofolate reductase inhibitor. In some embodiments, the method further comprises administering to the patient one or more doses of a dihydrofolate reductase inhibitor. In some embodiments, the dihydrofolate reductase inhibitor is selected from the group consisting of MTX or MPA.

In some embodiments, the modified lymphocytes are administered as a single bolus. In some embodiments, multiple doses of the modified lymphocytes are administered to the patient. In some embodiments, each dose of the multiple doses comprises between about 0.1×10⁶ cells/kg to about 240×10⁶ cells/kg. In some embodiments, a total dosage comprises between about 0.1×10⁶ cells/kg to about 730×10⁶ cells/kg.

In a third aspect of the present disclosure is a method of treating a hematological cancer in a patient in need of treatment thereof comprising: (a) generating a population of substantially HPRT deficient lymphocytes by transfecting or transducing lymphocytes obtained from a donor sample with (i) an endonuclease, and (ii) a guide RNA molecule targeting a sequence within one of Exon 3 or Exon 8 of the HPRT1 gene; (b) positively selecting for the population of substantially HPRT deficient lymphocytes ex vivo to provide a population of modified lymphocytes; (c) inducing at least a partial graft versus malignancy effect by administering an HSC graft to the patient; and (d) administering a therapeutically effective amount of the population of modified lymphocytes to the patient following the detection of residual disease or disease recurrence. In some embodiments, the lymphocytes are T-cells, preferably human primary T-cells.

In some embodiments, the guide RNA molecule targets a sequence within Chromosome X located between about 134475181 to about 134475364 based on GRCh38 or the equivalent position in a genome build other than GRCh38. In some embodiments, guide RNA molecule is at least about 85% complementary to the sequence within Chromosome X located between about 134475181 to about 134475364 based on GRCh38 or the equivalent position in a genome build other than GRCh38. In some embodiments, sequence targeted has a length ranging from between about 14 nucleotides to about 30 nucleotides. In some embodiments, the sequence targeted has a length ranging from between about 16 nucleotides to about 28 nucleotides. In some embodiments, the sequence targeted has a length ranging from between about 18 nucleotides to about 26 nucleotides. In some embodiments, the sequence targeted has a length ranging from between about 21 nucleotides to about 25 nucleotides. In some embodiments, the sequence targeted has a length of about 21 nucleotides. In some embodiments, the sequence targeted has a length of about 22 nucleotides. In some embodiments, the sequence targeted has a length of about 23 nucleotides. In some embodiments, the sequence targeted has a length of about 24 nucleotides. In some embodiments, the sequence targeted has a length of about 25 nucleotides.

In some embodiments, the guide RNA molecules target a sequence within Chromosome X located between about 134498608 to about 134498684 based on GRCh38 or the equivalent position in a genome build other than GRCh38. In some embodiments, the guide RNA molecule is at least about 85% complementary to the sequence within Chromosome X located between about 134498608 to about 134498684 based on GRCh38 or the equivalent position in a genome build other than GRCh38. In some embodiments, the sequence targeted has a length ranging from between about 14 nucleotides to about 30 nucleotides. In some embodiments, the sequence targeted has a length ranging from between about 16 nucleotides to about 28 nucleotides. In some embodiments, sequence targeted has a length ranging from between about 18 nucleotides to about 26 nucleotides. In some embodiments, the sequence targeted has a length ranging from between about 21 nucleotides to about 25 nucleotides. In some embodiments, the sequence targeted has a length of about 21 nucleotides. In some embodiments, the sequence targeted has a length of about 22 nucleotides. In some embodiments, the sequence targeted has a length of about 23 nucleotides. In some embodiments, the sequence targeted has a length of about 24 nucleotides. In some embodiments, the sequence targeted has a length of about 25 nucleotides.

In some embodiments, the guide RNA molecule has at least 90% sequence identity to any one of SEQ ID NOS: 40-44 and 46-56. In some embodiments, the guide RNA molecule has at least 91% sequence identity to any one of SEQ ID NOS: 40-44 and 46-56. In some embodiments, the guide RNA molecule has at least 92% sequence identity to any one of SEQ ID NOS: 40-44 and 46-56. In some embodiments, the guide RNA molecule has at least 93% sequence identity to any one of SEQ ID NOS: 40-44 and 46-56. In some embodiments, the guide RNA molecule has at least 94% sequence identity to any one of SEQ ID NOS: 40-44 and 46-56. In some embodiments, the guide RNA molecule gene has at least 95% sequence identity to any one of SEQ ID NOS: 40-44 and 46-56. In some embodiments, the guide RNA molecule has at least 96% sequence identity to any one of SEQ ID NOS: 40-44 and 46-56. In some embodiments, guide RNA molecule has at least 97% sequence identity to any one of SEQ ID NOS: 40-44 and 46-56. In some embodiments, guide RNA molecule has at least 98% sequence identity to any one of SEQ ID NOS: 40-44 and 46-56. In some embodiments, guide RNA molecule has at least 99% sequence identity to any one of SEQ ID NOS: 40-44 and 46-56. In some embodiments, the guide RNA molecule comprises any one of SEQ ID NOS: 40-44 and 46-56.

In some embodiments, the Cas protein comprises a Cas9 protein. In some embodiments, the Cas protein comprises a Cas12 protein. In some embodiments, the Cas12 protein is a Cas12a protein. In some embodiments, the Cas12 protein is a Cas12b protein.

In some embodiments, the lymphocytes obtained from the donor sample are transfected or transduced with a viral delivery vehicle, a non-viral delivery vehicle, or through a physical method. In some embodiments, the physical method is selected from microinjection and electroporation.

In some embodiments, the non-viral delivery vehicle is a nanocapsule. In some embodiments, the nanocapsule comprises at least one targeting moiety. In some embodiments, the at least one targeting moiety targets any one of a human mesenchymal stem cell CD marker, including the CD29, CD44, CD90, CD49a-f, CD51, CD73 (SH3), CD105 (SH2), CD106, CD166, and Stro-1 markers. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127, FoxP3 and CD44. In some embodiments, the T-cell marker is CD3. In some embodiments, the T-cell marker is CD28.

In some embodiments, co-stimulation with one or more co-stimulating moieties may be used to activate target cells, including T-cells. In some embodiments, co-stimulation may be achieved by activating one or more cell surface markers, including but not limited to CD28, ICOS, CTLA4, PD1, PD1H, and BTLA. In some embodiments, the co-stimulating moieties are antibodies.

In some embodiments, the viral delivery vehicle is an expression vector, and wherein the expression vector includes a first nucleic acid sequence encoding for the endonuclease and a second nucleic acid encoding for the guide RNA molecule. In some embodiments, the expression vector is a lentiviral expression vector.

In some embodiments, a level of HPRT 1 gene expression within the population of substantially HPRT deficient lymphocytes is reduced by at least about 70% as compared with the donor lymphocytes which have not been transfected. In some embodiments, a level of HPRT 1 gene expression within the population of substantially HPRT deficient lymphocytes is reduced by at least about 80% as compared with the donor lymphocytes which have not been transfected. In some embodiments, a level of HPRT 1 gene expression within the population of substantially HPRT deficient lymphocytes is reduced by at least about 90% as compared with the donor lymphocytes which have not been transfected.

In some embodiments, the positive selection comprises contacting the generated population of substantially HPRT deficient lymphocytes with a purine analog. In some embodiments, the purine analog is 6-TG. In some embodiments, the purine analog is 6-MP. In some embodiments, an amount of the purine analog ranges from between about 1 to about 15 μg/mL. In some embodiments, the positive selection comprises contacting the generated population of substantially HPRT deficient lymphocytes with both a purine analog and allopurinol.

In some embodiments, at least about 70% of the modified lymphocytes are sensitive to a dihydrofolate reductase inhibitor. In some embodiments, the method further comprises administering to the patient one or more doses of a dihydrofolate reductase inhibitor. In some embodiments, the dihydrofolate reductase inhibitor is selected from the group consisting of MTX or MPA.

In some embodiments, the modified lymphocytes are administered as a single bolus. In some embodiments, multiple doses of the modified lymphocytes are administered to the patient. In some embodiments, each dose of the multiple doses comprises between about 0.1×10⁶ cells/kg to about 240×10⁶ cells/kg. In some embodiments, a total dosage comprises between about 0.1×10⁶ cells/kg to about 730×10⁶ cells/kg.

In a fourth aspect of the present disclosure is a method of treating a patient with HPRT deficient lymphocytes including the steps of: (a) isolating lymphocytes from a donor subject; (b) contacting the isolated lymphocytes with (i) an endonuclease, and (ii) a guide RNA molecule targeting a sequence within one of Exon 3 or Exon 8 of the HPRT 1 gene; (c) exposing the population of HPRT deficient lymphocytes to an agent which positively selects for HPRT deficient lymphocytes to provide a preparation of modified lymphocytes; (d) administering a therapeutically effective amount of the preparation of the modified lymphocytes to the patient following hematopoietic stem-cell transplantation; and (e) optionally administering a dihydrofolate reductase inhibitor following the development of graft-versus-host disease (GvHD) in the patient. In some embodiments, the lymphocytes are T-cells, preferably human primary T-cells.

In some embodiments, dihydrofolate reductase inhibitor is selected from the group consisting of MTX or MPA. In some embodiments, the agent which positively selects for the HPRT deficient lymphocytes comprises a purine analog. In some embodiments, the purine analog is 6-TG. In some embodiments, an amount of 6-TG ranges from between about 1 to about 15 μg/mL.

In some embodiments, the guide RNA molecule targets a sequence within Chromosome X located between about 134475181 to about 134475364 based on GRCh38 or the equivalent position in a genome build other than GRCh38. In some embodiments, the guide RNA molecule is at least about 85% complementary to the sequence within Chromosome X located between about 134475181 to about 134475364 based on GRCh38 or the equivalent position in a genome build other than GRCh38. In some embodiments, the sequence targeted has a length ranging from between about 14 nucleotides to about 30 nucleotides. In some embodiments, the sequence targeted has a length ranging from between about 16 nucleotides to about 28 nucleotides. In some embodiments, the sequence targeted has a length ranging from between about 18 nucleotides to about 26 nucleotides. In some embodiments, the sequence targeted has a length ranging from between about 21 nucleotides to about 25 nucleotides. In some embodiments, the sequence targeted has a length of about 21 nucleotides. In some embodiments, the sequence targeted has a length of about 22 nucleotides. In some embodiments, the sequence targeted has a length of about 23 nucleotides. In some embodiments, the sequence targeted has a length of about 24 nucleotides. In some embodiments, the sequence targeted has a length of about 25 nucleotides.

In some embodiments, the guide RNA molecules targets a sequence within Chromosome X located between about 134498608 to about 134498684 based on GRCh38 or the equivalent position in a genome build other than GRCh38. some embodiments, the guide RNA molecule is at least about 85% complementary to the sequence within Chromosome X located between about 134498608 to about 134498684 based on GRCh38 or the equivalent position in a genome build other than GRCh38. In some embodiments, the sequence targeted has a length ranging from between about 14 nucleotides to about 30 nucleotides. In some embodiments, the sequence targeted has a length ranging from between about 16 nucleotides to about 28 nucleotides. In some embodiments, the sequence targeted has a length ranging from between about 18 nucleotides to about 26 nucleotides. In some embodiments, the sequence targeted has a length ranging from between about 21 nucleotides to about 25 nucleotides. In some embodiments, the sequence targeted has a length of about 21 nucleotides. In some embodiments, the sequence targeted has a length of about 22 nucleotides. In some embodiments, the sequence targeted has a length of about 23 nucleotides. In some embodiments, the sequence targeted has a length of about 24 nucleotides. In some embodiments, the sequence targeted has a length of about 25 nucleotides.

In some embodiments, the guide RNA molecule has at least 90% sequence identity to any one of SEQ ID NOS: 40-44 and 46-56. In some embodiments, the guide RNA molecule has at least 91% sequence identity to any one of SEQ ID NOS: 40-44 and 46-56. In some embodiments, the guide RNA molecule has at least 92% sequence identity to any one of SEQ ID NOS: 40-44 and 46-56. In some embodiments, the guide RNA molecule has at least 93% sequence identity to any one of SEQ ID NOS: 40-44 and 46-56. In some embodiments, the guide RNA molecule has at least 94% sequence identity to any one of SEQ ID NOS: 40-44 and 46-56. In some embodiments, the guide RNA molecule gene has at least 95% sequence identity to any one of SEQ ID NOS: 40-44 and 46-56. In some embodiments, the guide RNA molecule has at least 96% sequence identity to any one of SEQ ID NOS: 40-44 and 46-56. In some embodiments, guide RNA molecule has at least 97% sequence identity to any one of SEQ ID NOS: 40-44 and 46-56. In some embodiments, guide RNA molecule has at least 98% sequence identity to any one of SEQ ID NOS: 40-44 and 46-56. In some embodiments, guide RNA molecule has at least 99% sequence identity to any one of SEQ ID NOS: 40-44 and 46-56. In some embodiments, the guide RNA molecule comprises any one of SEQ ID NOS: 40-44 and 46-56.

In some embodiments, the Cas protein comprises a Cas9 protein. In some embodiments, the Cas protein comprises a Cas12 protein. In some embodiments, the Cas12 protein is a Cas12a protein. In some embodiments, the Cas12 protein is a Cas12b protein.

In some embodiments, the lymphocytes obtained from the donor sample are transfected or transduced with a viral delivery vehicle, a non-viral delivery vehicle, or through a physical method. In some embodiments, the physical method is selected from microinjection and electroporation.

In some embodiments, the non-viral delivery vehicle is a nanocapsule. In some embodiments, the nanocapsule comprises at least one targeting moiety. In some embodiments, the at least one targeting moiety targets any one of a human mesenchymal stem cell CD marker, including the CD29, CD44, CD90, CD49a-f, CD51, CD73 (SH3), CD105 (SH2), CD106, CD166, and Stro-1 markers. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127, FoxP3 and CD44. In some embodiments, the T-cell marker is CD3. In some embodiments, the T-cell marker is CD28.

In some embodiments, co-stimulation with one or more co-stimulating moieties may be used to activate target cells, including T-cells. In some embodiments, co-stimulation may be achieved by activating one or more cell surface markers, including but not limited to CD28, ICOS, CTLA4, PD1, PD1H, and BTLA. In some embodiments, the co-stimulating moieties are antibodies.

In some embodiments, the viral delivery vehicle is an expression vector, and wherein the expression vector includes a first nucleic acid sequence encoding for the endonuclease and a second nucleic acid encoding for the guide RNA molecule. In some embodiments, the expression vector is a lentiviral expression vector.

In some embodiments, the preparation is administered as a single bolus. In some embodiments, multiple doses of the preparation are administered to the patient. In some embodiments, each dose of the preparation comprises between about 0.1×10⁶ cells/kg to about 240×10⁶ cells/kg. In some embodiments, a total dosage of preparation comprises between about 0.1×10⁶ cells/kg to about 730×10⁶ cells/kg.

In a fifth aspect of the present disclosure is a use of a preparation including modified lymphocytes for providing the benefits of a lymphocyte infusion to a subject in need of treatment thereof, wherein the preparation including modified lymphocytes are generated by: (a) isolating lymphocytes from a donor subject; (b) contacting the isolated lymphocytes with (i) an endonuclease, and (ii) a guide RNA molecule targeting a sequence within one of Exon 3 or Exon 8 of the HPRT 1 gene to provide a population of substantially HPRT deficient lymphocytes; and (c) exposing the population of HPRT deficient lymphocytes to an agent which positively selects for HPRT deficient lymphocytes to provide a preparation of modified lymphocytes. In some embodiments, the lymphocytes are T-cells, preferably human primary T-cells. In some embodiments, the subject is in need of treatment following hematopoietic stem cell transplantation.

In some embodiments, the guide RNA molecule targets a sequence within Chromosome X located between about 134475181 to about 134475364 based on GRCh38 or the equivalent position in a genome build other than GRCh38. In some embodiments, the guide RNA molecule is at least about 85% complementary to the sequence within Chromosome X located between about 134475181 to about 134475364 based on GRCh38 or the equivalent position in a genome build other than GRCh38. In some embodiments, the sequence targeted has a length ranging from between about 14 nucleotides to about 30 nucleotides. In some embodiments, the sequence targeted has a length ranging from between about 16 nucleotides to about 28 nucleotides. In some embodiments, the sequence targeted has a length ranging from between about 18 nucleotides to about 26 nucleotides. In some embodiments, the sequence targeted has a length ranging from between about 21 nucleotides to about 25 nucleotides. In some embodiments, the sequence targeted has a length of about 21 nucleotides. In some embodiments, the sequence targeted has a length of about 22 nucleotides. In some embodiments, the sequence targeted has a length of about 23 nucleotides. In some embodiments, the sequence targeted has a length of about 24 nucleotides. In some embodiments, the sequence targeted has a length of about 25 nucleotides.

In some embodiments, the guide RNA molecules targets a sequence within Chromosome X located between about 134498608 to about 134498684 based on genome build GRCh38 or the equivalent position in a genome build other than GRCh38. In some embodiments, the guide RNA molecule is at least about 85% complementary to the sequence within Chromosome X located between about 134498608 to about 134498684 based on GRCh38 or the equivalent position in a genome build other than GRCh38. In some embodiments, the sequence targeted has a length ranging from between about 14 nucleotides to about 30 nucleotides. In some embodiments, the sequence targeted has a length ranging from between about 16 nucleotides to about 28 nucleotides. In some embodiments, the sequence targeted has a length ranging from between about 18 nucleotides to about 26 nucleotides. In some embodiments, the sequence targeted has a length ranging from between about 21 nucleotides to about 25 nucleotides. In some embodiments, the sequence targeted has a length of about 21 nucleotides. In some embodiments, the sequence targeted has a length of about 22 nucleotides. In some embodiments, the sequence targeted has a length of about 23 nucleotides. In some embodiments, the sequence targeted has a length of about 24 nucleotides. In some embodiments, the sequence targeted has a length of about 25 nucleotides.

In some embodiments, the guide RNA molecule has at least 90% sequence identity to any one of SEQ ID NOS: 40-44 and 46-56. In some embodiments, the guide RNA molecule has at least 91% sequence identity to any one of SEQ ID NOS: 40-44 and 46-56. In some embodiments, the guide RNA molecule has at least 92% sequence identity to any one of SEQ ID NOS: 40-44 and 46-56. In some embodiments, the guide RNA molecule has at least 93% sequence identity to any one of SEQ ID NOS: 40-44 and 46-56. In some embodiments, the guide RNA molecule has at least 94% sequence identity to any one of SEQ ID NOS: 40-44 and 46-56. In some embodiments, the guide RNA molecule gene has at least 95% sequence identity to any one of SEQ ID NOS: 40-44 and 46-56. In some embodiments, the guide RNA molecule has at least 96% sequence identity to any one of SEQ ID NOS: 40-44 and 46-56. In some embodiments, guide RNA molecule has at least 97% sequence identity to any one of SEQ ID NOS: 40-44 and 46-56. In some embodiments, guide RNA molecule has at least 98% sequence identity to any one of SEQ ID NOS: 40-44 and 46-56. In some embodiments, guide RNA molecule has at least 99% sequence identity to any one of SEQ ID NOS: 40-44 and 46-56. In some embodiments, the guide RNA molecule comprises any one of SEQ ID NOS: 40-44 and 46-56.

In a sixth aspect of the present disclosure is a kit comprising: (i) a guide RNA molecule having at least 90% sequence identity to any one of SEQ ID NOS: 40-61; and (ii) a Cas protein. In some embodiments, the Cas protein is selected from the group consisting of a Cas9 protein and a Cas12 protein. In some embodiments, the Cas12 protein is a Cas12a protein. In some embodiments, the Cas12 protein is a Cas12b protein. In some embodiments, the guide RNA molecule has at least 90% sequence identity to any one of SEQ ID NOS: 40-61. In some embodiments, the guide RNA molecule has at least 91% sequence identity to any one of SEQ ID NOS: 40-61. In some embodiments, the guide RNA molecule has at least 92% sequence identity to any one of SEQ ID NOS: 40-61. In some embodiments, the guide RNA molecule has at least 93% sequence identity to any one of SEQ ID NOS: 40-61. In some embodiments, the guide RNA molecule has at least 94% sequence identity to any one of SEQ ID NOS: 40-61. In some embodiments, the guide RNA molecule has at least 95% sequence identity to any one of SEQ ID NOS: 40-61. In some embodiments, the guide RNA molecule has at least 96% sequence identity to any one of SEQ ID NOS: 40-61. In some embodiments, the guide RNA molecule has at least 97% sequence identity to any one of SEQ ID NOS: 40-61. In some embodiments, the guide RNA molecule has at least 98% sequence identity to any one of SEQ ID NOS: 40-61. In some embodiments, the guide RNA molecule has at least 99% sequence identity to any one of SEQ ID NOS: 40-61. In some embodiments, the guide RNA molecule comprises any one of SEQ ID NOS: 40-61.

In a seventh aspect of the present disclosure is a kit comprising: (i) a guide RNA molecule which targets a sequence within Chromosome X located between about 134475181 to about 134475364 based on GRCh38 or the equivalent position in a genome build other than GRCh38, and (ii) a Cas protein. In some embodiments, the Cas protein is selected from the group consisting of a Cas9 protein and a Cas12 protein. In some embodiments, the Cas12 protein is a Cas12a protein. In some embodiments, the Cas12 protein is a Cas12b protein. In some embodiments, the guide RNA molecule is at least about 85% complementary to the sequence within Chromosome X located between about 134475181 to about 134475364 based on GRCh38 or the equivalent position in a genome build other than GRCh38. In some embodiments, the guide RNA molecule is at least about 90% complementary to the sequence within Chromosome X located between about 134475181 to about 134475364 based on GRCh38 or the equivalent position in a genome build other than GRCh38. In some embodiments, the guide RNA molecule is at least about 95% complementary to the sequence within Chromosome X located between about 134475181 to about 134475364 based on GRCh38 or the equivalent position in a genome build other than GRCh38. In some embodiments, the sequence targeted has a length ranging from between about 16 nucleotides to about 28 nucleotides. In some embodiments, the sequence targeted has a length ranging from between about 18 nucleotides to about 26 nucleotides. In some embodiments, the sequence targeted has a length ranging from between about 21 nucleotides to about 25 nucleotides. In some embodiments, the sequence targeted has a length of about 21 nucleotides. In some embodiments, the sequence targeted has a length of about 22 nucleotides. In some embodiments, the sequence targeted has a length of about 23 nucleotides. In some embodiments, the sequence targeted has a length of about 24 nucleotides. In some embodiments, the sequence targeted has a length of about 25 nucleotides.

In an eighth aspect of the present disclosure is a kit comprising: (i) a guide RNA molecule which targets a sequence within Chromosome X located between about 134498608 to about 134498684 based on GRCh38 or the equivalent position in a genome build other than GRCh38, and (ii) a Cas protein. In some embodiments, the Cas protein is selected from the group consisting of a Cas9 protein and a Cas12 protein. In some embodiments, the guide RNA molecule is at least about 85% complementary to the sequence within Chromosome X located between about 134498608 to about 134498684 based on GRCh38 or the equivalent position in a genome build other than GRCh38. In some embodiments, the guide RNA molecule is at least about 90% complementary to the sequence within Chromosome X located between about 134498608 to about 134498684 based on GRCh38 or the equivalent position in a genome build other than GRCh38. In some embodiments, the guide RNA molecule is at least about 95% complementary to the sequence within Chromosome X located between about 134498608 to about 134498684 based on GRCh38 or the equivalent position in a genome build other than GRCh38. In some embodiments, the sequence targeted has a length ranging from between about 18 nucleotides to about 26 nucleotides. In some embodiments, the sequence targeted has a length ranging from between about 16 nucleotides to about 28 nucleotides. In some embodiments, the sequence targeted has a length ranging from between about 21 nucleotides to about 25 nucleotides. In some embodiments, the sequence targeted has a length of about 21 nucleotides. In some embodiments, the sequence targeted has a length of about 22 nucleotides. In some embodiments, the sequence targeted has a length of about 23 nucleotides. In some embodiments, the sequence targeted has a length of about 24 nucleotides. In some embodiments, the sequence targeted has a length of about 25 nucleotides.

In a ninth aspect of the present disclosure is a nanocapsule comprising (i) a guide RNA molecule having at least 90% sequence identity to any one of SEQ ID NOS: 40-61; and (ii) a Cas protein. In some embodiments, the Cas protein is selected from the group consisting of a Cas9 protein and a Cas12 protein. In some embodiments, the Cas12 protein is a Cas12a protein. In some embodiments, the Cas12 protein is a Cas12b protein.

In some embodiments, the guide-RNA has at least 91% sequence identity to any one of SEQ ID NOS: 40-61. In some embodiments, the guide-RNA has at least 92% sequence identity to any one of SEQ ID NOS: 40-61. In some embodiments, the guide-RNA has at least 93% sequence identity to any one of SEQ ID NOS: 40-61. In some embodiments, the guide-RNA has at least 94% sequence identity to any one of SEQ ID NOS: 40-61. In some embodiments, the guide-RNA has at least 95% sequence identity to any one of SEQ ID NOS: 40-61. In some embodiments, the guide-RNA has at least 96% sequence identity to any one of SEQ ID NOS: 40-61. In some embodiments, the guide-RNA has at least 97% sequence identity to any one of SEQ ID NOS: 40-61. In some embodiments, the guide-RNA has at least 98% sequence identity to any one of SEQ ID NOS: 40-61. In some embodiments, the guide-RNA has at least 99% sequence identity to any one of SEQ ID NOS: 40-61. In some embodiments, the guide-RNA has the sequence of any one of SEQ ID NOS: 40-61.

In some embodiments, the nanocapsules comprise at least one targeting moiety. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127 or FoxP3 and CD44. In some embodiments, the T-cell marker is CD3. In some embodiments, the T-cell marker is CD28.

In some embodiments, the nanocapsule comprises a polymeric shell. In some embodiments, polymeric nanocapsules are comprised of two different positively charged monomers, at least one neutral monomer, and a cross-linker.

In a tenth aspect of the present disclosure is a host cell transfected with a nanocapsule comprising (i) a guide RNA molecule having at least 90% sequence identity to any one of SEQ ID NOS: 40-61; and (ii) a Cas protein. In some embodiments, the Cas protein is selected from the group consisting of a Cas9 protein and a Cas12 protein. In some embodiments, the Cas12 protein is a Cas12a protein. In some embodiments, the Cas12 protein is a Cas12b protein.

In some embodiments, the guide-RNA has at least 91% sequence identity to any one of SEQ ID NOS: 40-61. In some embodiments, the guide-RNA has at least 92% sequence identity to any one of SEQ ID NOS: 40-61. In some embodiments, the guide-RNA has at least 93% sequence identity to any one of SEQ ID NOS: 40-61. In some embodiments, the guide-RNA has at least 94% sequence identity to any one of SEQ ID NOS: 40-61. In some embodiments, the guide-RNA has at least 95% sequence identity to any one of SEQ ID NOS: 40-61. In some embodiments, the guide-RNA has at least 96% sequence identity to any one of SEQ ID NOS: 40-61. In some embodiments, the guide-RNA has at least 97% sequence identity to any one of SEQ ID NOS: 40-61. In some embodiments, the guide-RNA has at least 98% sequence identity to any one of SEQ ID NOS: 40-61. In some embodiments, the guide-RNA has at least 99% sequence identity to any one of SEQ ID NOS: 40-61. In some embodiments, the guide-RNA has any one of SEQ ID NOS: 40-61. In some embodiments, the nanocapsules comprise at least one targeting moiety. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127, FoxP3 and CD44. In some embodiments, the T-cell marker is CD3. In some embodiments, the T-cell marker is CD28.

In some embodiments, the nanocapsule comprises a polymeric shell. In some embodiments, polymeric nanocapsules are comprised of two different positively charged monomers, at least one neutral monomer, and a cross-linker. In some embodiments, the host cell is a primary T-lymphocyte. In some embodiments, the host cell is a CEM cell.

In an eleventh aspect of the present disclosure is a use of a preparation of modified lymphocytes for providing the benefits of a lymphocyte infusion to a subject in need of treatment thereof following hematopoietic stem-cell transplantation, wherein the preparation of the modified lymphocytes are generated by: (a) isolating lymphocytes from a donor subject; (b) contacting the isolated lymphocytes with a nanocapsule comprising (i) a guide RNA molecule having at least 90% sequence identity to any one of SEQ ID NOS: 40-61; and (ii) a Cas protein; and (c) exposing the population of HPRT deficient lymphocytes to an agent which positively selects for HPRT deficient lymphocytes to provide a preparation of modified lymphocytes. In some embodiments, the Cas protein is selected from the group consisting of a Cas9 protein and a Cas12 protein. In some embodiments, the Cas12 protein is a Cas12a protein. In some embodiments, the Cas12 protein is a Cas12b protein.

In some embodiments, the guide-RNA has at least 91% sequence identity to any one of SEQ ID NOS: 40-61. In some embodiments, the guide-RNA has at least 92% sequence identity to any one of SEQ ID NOS: 40-61. In some embodiments, the guide-RNA has at least 93% sequence identity to any one of SEQ ID NOS: 40-61. In some embodiments, the guide-RNA has at least 94% sequence identity to any one of SEQ ID NOS: 40-61. In some embodiments, the guide-RNA has at least 95% sequence identity to any one of SEQ ID NOS: 40-61. In some embodiments, the guide-RNA has at least 97% sequence identity to any one of SEQ ID NOS: 40-61. In some embodiments, the guide-RNA has at least 98% sequence identity to any one of SEQ ID NOS: 40-61. In some embodiments, the guide-RNA has at least 99% sequence identity to any one of SEQ ID NOS: 40-61. In some embodiments, the guide-RNA has any one of SEQ ID NOS: 40-61. In some embodiments, the nanocapsules comprise at least one targeting moiety. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127, FoxP3 and CD44. In some embodiments, the T-cell marker is CD3. In some embodiments, the T-cell marker is CD28. In some embodiments, the lymphocytes are T-cells, preferably human primary T-cells.

In some embodiments, the nanocapsule comprises a polymeric shell. In some embodiments, polymeric nanocapsules are comprised of two different positively charged monomers, at least one neutral monomer, and a cross-linker.

In a twelfth aspect of the present disclosure is a kit comprising: (a) a nanocapsule comprising (i) a guide RNA molecule having at least 90% sequence identity to any one of SEQ ID NOS: 40-61; and (ii) a Cas protein, and (b) a dihydrofolate reductase inhibitor. In some embodiments, the dihydrofolate reductase inhibitor is MTX or MPA. In some embodiments, the guide-RNA has at least 91% sequence identity to any one of SEQ ID NOS: 40-61. In some embodiments, the guide-RNA has at least 92% sequence identity to any one of SEQ ID NOS: 40-61. In some embodiments, the guide-RNA has at least 93% sequence identity to any one of SEQ ID NOS: 40-61. In some embodiments, the guide-RNA has at least 94% sequence identity to any one of SEQ ID NOS: 40-61. In some embodiments, the guide-RNA has at least 95% sequence identity to any one of SEQ ID NOS: 40-61. In some embodiments, the guide-RNA has at least 97% sequence identity to any one of SEQ ID NOS: 40-61. In some embodiments, the guide-RNA has at least 98% sequence identity to any one of SEQ ID NOS: 40-61. In some embodiments, the guide-RNA has at least 99% sequence identity to any one of SEQ ID NOS: 40-61. In some embodiments, the guide-RNA has any one of SEQ ID NOS: 40-61.

In a thirteenth aspect of the present disclosure is a method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA having at least 90% sequence identity to any one of SEQ ID NOS: 40-61; (b) positively selecting for the population of substantially HPRT deficient lymphocytes ex vivo to provide a population of modified lymphocytes; and (c) administering a therapeutically effective amount of the population of modified lymphocytes to the patient following the administration of the HSC graft. In some embodiments, the guide RNA has at least 91% sequence identity to any one of SEQ ID NOS: 40-61. In some embodiments, the guide RNA has at least 92% sequence identity to any one of SEQ ID NOS: 40-61. In some embodiments, the guide RNA has at least 93% sequence identity to any one of SEQ ID NOS: 40-61. In some embodiments, the guide RNA has at least 94% sequence identity to any one of SEQ ID NOS: 40-61. In some embodiments, the guide RNA has at least 95% sequence identity to any one of SEQ ID NOS: 40-61. In some embodiments, the guide RNA has at least 97% sequence identity to any one of SEQ ID NOS: 40-61. In some embodiments, the guide RNA has at least 99% sequence identity to any one of SEQ ID NOS: 40-61. In some embodiments, the guide RNA comprises any one of SEQ ID NOS: 40-61. In some embodiments, the lymphocytes are T-cells, preferably human primary T-cells. In some embodiments, the method further comprises administering an HSC graft to the patient. In some embodiments, the HSC graft is administered prior to, contemporaneously with, or following the administration of the population of modified lymphocytes.

In a fourteenth aspect of the present disclosure is a method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA molecule targeting a sequence within one of Exon 2, Exon 3 or Exon 8 of the HPRT 1 gene; (b) positively selecting for the population of substantially HPRT deficient lymphocytes ex vivo to provide a population of modified lymphocytes; and (c) administering a therapeutically effective amount of the population of modified lymphocytes to the patient. In some embodiments, the lymphocytes are T-cells, preferably human primary T-cells. In some embodiments, the method further comprises administering an HSC graft to the patient. In some embodiments, the HSC graft is administered prior to, contemporaneously with, or following the administration of the population of modified lymphocytes. In some embodiments, the guide RNA has at least 90% sequence identity to any one of SEQ ID NOS: 40-61. In some embodiments, the guide RNA has at least 95% sequence identity to any one of SEQ ID NOS: 40-61.

In some embodiments, the guide RNA targets a sequence with Exon 2. In some embodiments, the guide RNA has at least 90% sequence identity to any one of SEQ ID NOS: 45 and 57-61. In some embodiments, the guide RNA has at least 95% sequence identity to any one of SEQ ID NOS: 45 and 57-61. In some embodiments, the guide RNA targets a sequence with Exon 3. In some embodiments, the guide RNA has at least 90% sequence identity to any one of SEQ ID NOS: 50-54. In some embodiments, the guide RNA has at least 95% sequence identity to any one of SEQ ID NOS: 50-54. In some embodiments, the guide RNA targets a sequence with Exon 8. In some embodiments, the guide RNA has at least 90% sequence identity to any one of SEQ ID NOS: 55-56. In some embodiments, the guide RNA has at least 95% sequence identity to any one of SEQ ID NOS: 55-56.

In a fifteenth aspect of the present disclosure is a method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA molecule targeting a sequence within Exon 2 of the HPRT 1 gene; (b) positively selecting for the population of substantially HPRT deficient lymphocytes ex vivo to provide a population of modified lymphocytes; and (c) administering an HSC graft to the patient; (d) administering a therapeutically effective amount of the population of modified lymphocytes to the patient following the administration of the HSC graft. In some embodiments, the guide RNA molecule targeting Exon 2 of the HPRT 1 gene has at least 90% sequence identity to any one of SEQ ID NOS: 45 and 57-61. In some embodiments, the guide RNA molecule targeting Exon 2 of the HPRT 1 gene has at least 91% sequence identity to any one of SEQ ID NOS: 45 and 57-61. In some embodiments, the guide RNA molecule targeting Exon 2 of the HPRT 1 gene has at least 92% sequence identity to any one of SEQ ID NOS: 45 and 57-61. In some embodiments, the guide RNA molecule targeting Exon 2 of the HPRT 1 gene has at least 93% sequence identity to any one of SEQ ID NOS: 45 and 57-61. In some embodiments, the guide RNA molecule targeting Exon 2 of the HPRT 1 gene has at least 94% sequence identity to any one of SEQ ID NOS: 45 and 57-61. In some embodiments, the guide RNA molecule targeting Exon 2 of the HPRT 1 gene has at least 95% sequence identity to any one of SEQ ID NOS: 45 and 57-61. In some embodiments, the guide RNA molecule targeting Exon 2 of the HPRT 1 gene has at least 97% sequence identity to any one of SEQ ID NOS: 45 and 57-61. In some embodiments, the guide RNA molecule targeting Exon 2 of the HPRT 1 gene has at least 98% sequence identity to any one of SEQ ID NOS: 45 and 57-61. In some embodiments, the guide RNA molecule targeting Exon 2 of the HPRT 1 gene has at least 99% sequence identity to any one of SEQ ID NOS: 45 and 57-61.

In some embodiments, the guide RNA molecule targeting Exon 2 of the HPRT 1 gene comprises SEQ ID NO: 45. In some embodiments, the guide RNA molecule targeting Exon 2 of the HPRT 1 gene comprises SEQ ID NO: 57. In some embodiments, the guide RNA molecule targeting Exon 2 of the HPRT 1 gene comprises SEQ ID NO: 58. In some embodiments, the guide RNA molecule targeting Exon 2 of the HPRT 1 gene comprises SEQ ID NO: 59. In some embodiments, the guide RNA molecule targeting Exon 2 of the HPRT 1 gene comprises SEQ ID NO: 60. In some embodiments, the guide RNA molecule targeting Exon 2 of the HPRT 1 gene comprises SEQ ID NO: 61. In some embodiments, the lymphocytes are T-cells, preferably human primary T-cells.

In some embodiments, the lymphocytes obtained from the donor sample are transfected or transduced with a viral delivery vehicle, a non-viral delivery vehicle, and/or through a physical method. In some embodiments, the physical method is selected from microinjection and electroporation.

In some embodiments, the non-viral delivery vehicle is a nanocapsule. In some embodiments, the nanocapsule optionally comprises at least one targeting moiety. In some embodiments, the nanocapsule comprises at least one targeting moiety. In some embodiments, the at least one targeting moiety targets any one of a human mesenchymal stem cell CD marker, including CD29, CD44, CD90, CD49a-f, CD51, CD73 (SH3), CD105 (SH2), CD106, CD166, and Stro-1 markers. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127, FoxP3 and CD44. In some embodiments, the T-cell marker is CD3. In some embodiments, the T-cell marker is CD28.

In some embodiments, co-stimulation with one or more co-stimulating moieties may be used to activate target cells, including T-cells. In some embodiments, co-stimulation may be achieved by activating one or more cell surface markers, including but not limited to CD28, ICOS, CTLA4, PD1, PD1H, and BTLA. In some embodiments, the co-stimulating moieties are antibodies.

In some embodiments, the viral delivery vehicle is an expression vector, and wherein the expression vector includes a first nucleic acid sequence encoding for the endonuclease and a second nucleic acid encoding for the guide RNA molecule. In some embodiments, the expression vector is a lentiviral expression vector.

In some embodiments, a level of HPRT1 gene expression within the population of substantially HPRT deficient lymphocytes is reduced by at least about 70% as compared with the donor lymphocytes which have not been transfected. In some embodiments, a level of HPRT1 gene expression within the population of substantially HPRT deficient lymphocytes is reduced by at least about 75% as compared with the donor lymphocytes which have not been transfected. In some embodiments, a level of HPRT1 gene expression within the population of substantially HPRT deficient lymphocytes is reduced by at least about 80% as compared with the donor lymphocytes which have not been transfected. In some embodiments, a level of HPRT1 gene expression within the population of substantially HPRT deficient lymphocytes is reduced by at least about 85% as compared with the donor lymphocytes which have not been transfected. In some embodiments, a level of HPRT1 gene expression within the population of substantially HPRT deficient lymphocytes is reduced by at least about 90% as compared with the donor lymphocytes which have not been transfected. In some embodiments, the lymphocytes are T-cells, preferably human primary T-cells. In some embodiments, a level of HPRT1 gene expression within the population of substantially HPRT deficient lymphocytes is reduced by at least about 95% as compared with the donor lymphocytes which have not been transfected.

In some embodiments, the positive selection comprises contacting the generated population of substantially HPRT deficient lymphocytes with a purine analog. In some embodiments, the purine analog is 6-TG. In some embodiments, the purine analog is 6-mercaptopurine (6-MP). In some embodiments, an amount of the purine analog ranges from between about 1 to about 15 μg/mL. In some embodiments, the positive selection comprises contacting the generated population of substantially HPRT deficient lymphocytes with both a purine analog (e.g., in an amount ranging from between about 1 to about 15 μg/mL) and allopurinol.

In some embodiments, at least about 70% of the population of modified lymphocytes are sensitive to a dihydrofolate reductase inhibitor. In some embodiments, at least about 80% of the population of modified lymphocytes are sensitive to a dihydrofolate reductase inhibitor. In some embodiments, the method further comprises administering to the patient one or more doses of a dihydrofolate reductase inhibitor (e.g. two or more doses, three or more doses, four or more doses, etc.). In some embodiments, the dihydrofolate reductase inhibitor is selected from the group consisting of MTX or MPA.

In some embodiments, the population of modified lymphocytes are administered as a single bolus. In some embodiments, multiple doses of the population of modified lymphocytes are administered to the patient (e.g., two or more doses, three or more doses, four or more doses, etc.). In some embodiments, each dose of the multiple doses comprises between about 0.1×10⁶ cells/kg to about 240×10⁶ cells/kg. In some embodiments, a total dosage comprises between about 0.1×10⁶ cells/kg to about 730×10⁶ cells/kg.

In a sixteenth aspect of the present disclosure is a method of treating a hematological cancer in a patient in need of treatment thereof comprising: (a) generating a population of substantially HPRT deficient lymphocytes by transfecting or transducing lymphocytes obtained from a donor sample with (i) an endonuclease, and (ii) a guide RNA molecule targeting a sequence within Exon 2 of the HPRT 1 gene; (b) positively selecting for the population of substantially HPRT deficient lymphocytes ex vivo to provide a population of modified lymphocytes; (c) inducing at least a partial graft versus malignancy effect by administering an HSC graft to the patient; and (d) administering a therapeutically effective amount of the population of modified lymphocytes to the patient following the detection of residual disease or disease recurrence. In some embodiments, the lymphocytes are T-cells, preferably human primary T-cells. In some embodiments, the endonuclease is a Cas9 protein. In some embodiments, the endonuclease is a Cas12 protein. In some embodiments, the Cas12 protein is a Cas12a protein. In some embodiments, the Cas12 protein is a Cas12b protein.

In some embodiments, the guide RNA molecule targeting Exon 2 of the HPRT 1 gene has at least 90% sequence identity to any one of SEQ ID NOS: 45 and 57-61. In some embodiments, the guide RNA molecule targeting Exon 2 of the HPRT 1 gene has at least 91% sequence identity to any one of SEQ ID NOS: 45 and 57-61. In some embodiments, the guide RNA molecule targeting Exon 2 of the HPRT 1 gene has at least 92% sequence identity to any one of SEQ ID NOS: 45 and 57-61. In some embodiments, the guide RNA molecule targeting Exon 2 of the HPRT 1 gene has at least 93% sequence identity to any one of SEQ ID NOS: 45 and 57-61. In some embodiments, the guide RNA molecule targeting Exon 2 of the HPRT 1 gene has at least 94% sequence identity to any one of SEQ ID NOS: 45 and 57-61. In some embodiments, the guide RNA molecule targeting Exon 2 of the HPRT 1 gene has at least 95% sequence identity to any one of SEQ ID NOS: 45 and 57-61. In some embodiments, the guide RNA molecule targeting Exon 2 of the HPRT 1 gene has at least 97% sequence identity to any one of SEQ ID NOS: 45 and 57-61. In some embodiments, the guide RNA molecule targeting Exon 2 of the HPRT 1 gene has at least 98% sequence identity to any one of SEQ ID NOS: 45 and 57-61. In some embodiments, the guide RNA molecule targeting Exon 2 of the HPRT 1 gene has at least 99% sequence identity to any one of SEQ ID NOS: 45 and 57-61.

In some embodiments, the guide RNA molecule targeting Exon 2 of the HPRT 1 gene comprises SEQ ID NO: 45. In some embodiments, the guide RNA molecule targeting Exon 2 of the HPRT 1 gene comprises SEQ ID NO: 57. In some embodiments, the guide RNA molecule targeting Exon 2 of the HPRT 1 gene comprises SEQ ID NO: 58. In some embodiments, the guide RNA molecule targeting Exon 2 of the HPRT 1 gene comprises SEQ ID NO: 59. In some embodiments, the guide RNA molecule targeting Exon 2 of the HPRT 1 gene comprises SEQ ID NO: 60. In some embodiments, the guide RNA molecule targeting Exon 2 of the HPRT 1 gene comprises SEQ ID NO: 61. In some embodiments, the lymphocytes are T-cells, preferably human primary T-cells.

In a seventeenth aspect of the present disclosure is a method of treating a patient with HPRT deficient lymphocytes including the steps of: (a) isolating lymphocytes from a donor subject; (b) contacting the isolated lymphocytes with (i) an endonuclease, and (ii) a guide RNA molecule targeting a sequence within Exon 2 of the HPRT 1 gene; (c) exposing the population of HPRT deficient lymphocytes to an agent which positively selects for HPRT deficient lymphocytes to provide a preparation of modified lymphocytes; (d) administering a therapeutically effective amount of the preparation of the modified lymphocytes to the patient following hematopoietic stem-cell transplantation; and (e) optionally administering a dihydrofolate reductase inhibitor following the development of graft-versus-host disease (GvHD) in the patient. In some embodiments, the lymphocytes are T-cells, preferably human primary T-cells. In some embodiments, the endonuclease is a Cas9 protein. In some embodiments, the endonuclease is a Cas12 protein. In some embodiments, the Cas12 protein is a Cas12a protein. In some embodiments, the Cas12 protein is a Cas12b protein.

In some embodiments, the guide RNA molecule targeting Exon 2 of the HPRT 1 gene has at least 90% sequence identity to any one of SEQ ID NOS: 45 and 57-61. In some embodiments, the guide RNA molecule targeting Exon 2 of the HPRT 1 gene has at least 91% sequence identity to any one of SEQ ID NOS: 45 and 57-61. In some embodiments, the guide RNA molecule targeting Exon 2 of the HPRT 1 gene has at least 92% sequence identity to any one of SEQ ID NOS: 45 and 57-61. In some embodiments, the guide RNA molecule targeting Exon 2 of the HPRT 1 gene has at least 93% sequence identity to any one of SEQ ID NOS: 45 and 57-61. In some embodiments, the guide RNA molecule targeting Exon 2 of the HPRT 1 gene has at least 94% sequence identity to any one of SEQ ID NOS: 45 and 57-61. In some embodiments, the guide RNA molecule targeting Exon 2 of the HPRT 1 gene has at least 95% sequence identity to any one of SEQ ID NOS: 45 and 57-61. In some embodiments, the guide RNA molecule targeting Exon 2 of the HPRT 1 gene has at least 97% sequence identity to any one of SEQ ID NOS: 45 and 57-61. In some embodiments, the guide RNA molecule targeting Exon 2 of the HPRT 1 gene has at least 98% sequence identity to any one of SEQ ID NOS: 45 and 57-61. In some embodiments, the guide RNA molecule targeting Exon 2 of the HPRT 1 gene has at least 99% sequence identity to any one of SEQ ID NOS: 45 and 57-61.

In some embodiments, the guide RNA molecule targeting Exon 2 of the HPRT 1 gene comprises SEQ ID NO: 45. In some embodiments, the guide RNA molecule targeting Exon 2 of the HPRT 1 gene comprises SEQ ID NO: 57. In some embodiments, the guide RNA molecule targeting Exon 2 of the HPRT 1 gene comprises SEQ ID NO: 58. In some embodiments, the guide RNA molecule targeting Exon 2 of the HPRT 1 gene comprises SEQ ID NO: 59. In some embodiments, the guide RNA molecule targeting Exon 2 of the HPRT 1 gene comprises SEQ ID NO: 60. In some embodiments, the guide RNA molecule targeting Exon 2 of the HPRT 1 gene comprises SEQ ID NO: 61. In some embodiments, the lymphocytes are T-cells, preferably human primary T-cells.

In an eighteenth aspect of the present disclosure is a use of a preparation of modified lymphocytes for providing the benefits of a lymphocyte infusion to a subject in need of treatment thereof following hematopoietic stem-cell transplantation, wherein the preparation of the modified lymphocytes are generated by: (a) isolating lymphocytes from a donor subject; (b) contacting the isolated lymphocytes with (i) an endonuclease, and (ii) a guide RNA molecule targeting a sequence within Exon 2 of the HPRT 1 gene to provide a population of substantially HPRT deficient lymphocytes; and (c) exposing the population of HPRT deficient lymphocytes to an agent which positively selects for HPRT deficient lymphocytes to provide a preparation of modified lymphocytes. In some embodiments, the lymphocytes are T-cells, preferably human primary T-cells. In some embodiments, the endonuclease is a Cas9 protein. In some embodiments, the endonuclease is a Cas12 protein. In some embodiments, the Cas12 protein is a Cas12a protein. In some embodiments, the Cas12 protein is a Cas12b protein.

In some embodiments, the guide RNA molecule targeting Exon 2 of the HPRT 1 gene has at least 90% sequence identity to any one of SEQ ID NOS: 45 and 57-61. In some embodiments, the guide RNA molecule targeting Exon 2 of the HPRT 1 gene has at least 91% sequence identity to any one of SEQ ID NOS: 45 and 57-61. In some embodiments, the guide RNA molecule targeting Exon 2 of the HPRT 1 gene has at least 92% sequence identity to any one of SEQ ID NOS: 45 and 57-61. In some embodiments, the guide RNA molecule targeting Exon 2 of the HPRT 1 gene has at least 93% sequence identity to any one of SEQ ID NOS: 45 and 57-61. In some embodiments, the guide RNA molecule targeting Exon 2 of the HPRT 1 gene has at least 94% sequence identity to any one of SEQ ID NOS: 45 and 57-61. In some embodiments, the guide RNA molecule targeting Exon 2 of the HPRT 1 gene has at least 95% sequence identity to any one of SEQ ID NOS: 45 and 57-61. In some embodiments, the guide RNA molecule targeting Exon 2 of the HPRT 1 gene has at least 97% sequence identity to any one of SEQ ID NOS: 45 and 57-61. In some embodiments, the guide RNA molecule targeting Exon 2 of the HPRT 1 gene has at least 98% sequence identity to any one of SEQ ID NOS: 45 and 57-61. In some embodiments, the guide RNA molecule targeting Exon 2 of the HPRT 1 gene has at least 99% sequence identity to any one of SEQ ID NOS: 45 and 57-61.

In some embodiments, the guide RNA molecule targeting Exon 2 of the HPRT 1 gene comprises SEQ ID NO: 45. In some embodiments, the guide RNA molecule targeting Exon 2 of the HPRT 1 gene comprises SEQ ID NO: 57. In some embodiments, the guide RNA molecule targeting Exon 2 of the HPRT 1 gene comprises SEQ ID NO: 58. In some embodiments, the guide RNA molecule targeting Exon 2 of the HPRT 1 gene comprises SEQ ID NO: 59. In some embodiments, the guide RNA molecule targeting Exon 2 of the HPRT 1 gene comprises SEQ ID NO: 60. In some embodiments, the guide RNA molecule targeting Exon 2 of the HPRT 1 gene comprises SEQ ID NO: 61. In some embodiments, the lymphocytes are T-cells, preferably human primary T-cells.

A nineteenth aspect of the present disclosure is a method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA molecule targeting a sequence within Chromosome X located between about 134473409 to about 134473460 based on genome build GRCh38 or the equivalent positions in a genome build other than GRCh38; (b) positively selecting for the population of substantially HPRT deficient lymphocytes ex vivo to provide a population of modified lymphocytes; (c) administering a therapeutically effective amount of the population of modified lymphocytes to the patient. In some embodiments, the method further comprises administering an HSC graft to the patient. In some embodiments, the HSC graft is administered prior to, contemporaneously with, or following the administration of the population of modified lymphocytes. In some embodiments, the guide RNA molecule is at least about 85% complementary to the sequence within Chromosome X located between about 134473409 to about 134473460 based on genome build GRCh38 or the equivalent position in a genome build other than GRCh38. In some embodiments, the sequence targeted has a length ranging from between about 14 nucleotides to about 30 nucleotides. In some embodiments, the sequence targeted has a length ranging from between about 18 nucleotides to about 26 nucleotides. In some embodiments, the sequence targeted has a length ranging from between about 21 nucleotides to about 25 nucleotides.

In a twentieth aspect of the present disclosure is a kit comprising: (i) a guide RNA molecule which targets a sequence within Chromosome X located between about 134473409 to about 134473460 based on GRCh38 or the equivalent position in a genome build other than GRCh38, and (ii) a Cas protein. In some embodiments, the Cas protein is selected from the group consisting of a Cas9 protein and a Cas12 protein. In some embodiments, the Cas12 protein is a Cas12a protein. In some embodiments, the Cas12 protein is a Cas12b protein. In some embodiments, the guide RNA molecule is at least about 85% complementary to the sequence within Chromosome X located between about 134473409 to about 134473460 based on GRCh38 or the equivalent position in a genome build other than GRCh38. In some embodiments, the guide RNA molecule is at least about 90% complementary to the sequence within Chromosome X located between about 134473409 to about 134473460 based on GRCh38 or the equivalent position in a genome build other than GRCh38. In some embodiments, the guide RNA molecule is at least about 95% complementary to the sequence within Chromosome X located between about 134473409 to about 134473460 based on GRCh38 or the equivalent position in a genome build other than GRCh38. In some embodiments, the sequence targeted has a length ranging from between about 16 nucleotides to about 28 nucleotides. In some embodiments, the sequence targeted has a length ranging from between about 18 nucleotides to about 26 nucleotides. In some embodiments, the sequence targeted has a length ranging from between about 21 nucleotides to about 25 nucleotides. In some embodiments, the sequence targeted has a length of about 21 nucleotides. In some embodiments, the sequence targeted has a length of about 22 nucleotides. In some embodiments, the sequence targeted has a length of about 23 nucleotides. In some embodiments, the sequence targeted has a length of about 24 nucleotides. In some embodiments, the sequence targeted has a length of about 25 nucleotides.

In a twenty-first aspect of the present disclosure is a method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA molecule targeting a sequence within one of Exon 2, Exon 3 or Exon 8 of the HPRT 1 gene; (b) positively selecting for the population of substantially HPRT deficient lymphocytes ex vivo to provide a population of modified lymphocytes; and (c) administering a therapeutically effective amount of the population of modified lymphocytes to the patient.

In some embodiments, the method further comprises administering an HSC graft to the patient. In some embodiments, the HSC graft is administered prior to, contemporaneously with, or following the administration of the population of modified lymphocytes.

In some embodiments, the guide RNA molecule targets a sequence within Exon 2 of the HPRT 1 gene. In some embodiments, the guide RNA molecule targets a sequence within Exon 3 of the HPRT 1 gene. In some embodiments, the guide RNA molecule targets a sequence within Exon 8 of the HPRT 1 gene.

In some embodiments, the guide RNA molecule targeting Exon 2 of the HPRT 1 gene has at least 95% sequence identity to any one of SEQ ID NOS: 45 and 57-61. In some embodiments, the guide RNA molecule targeting Exon 2 of the HPRT 1 gene has at least 99% sequence identity to any one of SEQ ID NOS: 45 and 57-61. In some embodiments, the guide RNA molecule targeting Exon 2 of the HPRT 1 gene comprises any one of SEQ ID NOS: 45 and 57-61.

In some embodiments, the guide RNA molecule targeting the sequence within the one of Exon 3 of the HPRT 1 gene has at least 95% sequence identity to any one of SEQ ID NOS: 41-44, 46 and 50-51. In some embodiments, the guide RNA molecule targeting the sequence within the one of Exon 3 of the HPRT 1 gene has at least 99% sequence identity to any one of SEQ ID NOS: 41-44, 46 and 50-51. In some embodiments, the guide RNA molecule targeting the sequence within the one of Exon 3 of the HPRT 1 gene comprises any one of SEQ IDS: 41-44, 46 and 50-51.

In some embodiments, the guide RNA molecule targeting the sequence within the one of Exon 8 of the HPRT 1 gene has at least 95% sequence identity to any one of SEQ ID NOS: 47-49, 46, 55 and 56. In some embodiments, the guide RNA molecule targeting the sequence within the one of Exon 8 of the HPRT 1 gene has at least 99% sequence identity to any one of SEQ ID NOS: 47-49, 46, 55 and 56. In some embodiments, the guide RNA molecule targeting the sequence within the one of Exon 8 of the HPRT 1 gene comprises any one of SEQ IDS: 47-49, 46, 55 and 56.

In some embodiments, the guide RNA molecule is at least about 85% complementary to the sequence within Chromosome X located between about 134475181 to about 134475364 based on genome build GRCh38 or the equivalent position in a genome build other than GRCh38. In some embodiments, the guide RNA molecules targets the sequence within Chromosome X located between about 134475181 to about 134475364 based on genome build GRCh38 or the equivalent position in a genome build other than GRCh38.

In some embodiments, the endonuclease comprises a Cas protein. In some embodiments, the Cas protein comprises a Cas9 protein. In some embodiments, the Cas protein comprises a Cas12 protein. In some embodiments, the Cas12 protein is a Cas12a protein. In some embodiments, the Cas12 protein is a Cas12b protein.

In some embodiments, the lymphocytes obtained from the donor sample are transfected or transduced with a viral delivery vehicle, a non-viral delivery vehicle, or through a physical method. In some embodiments, the physical method is selected from microinjection and electroporation. In some embodiments, the non-viral delivery vehicle is a nanocapsule, optionally wherein the nanocapsule comprises at least one targeting moiety. In some embodiments, the viral delivery vehicle is an expression vector, and wherein the expression vector includes a first nucleic acid sequence encoding for the endonuclease and a second nucleic acid encoding for the guide RNA molecule. In some embodiments, the expression vector is a lentiviral expression vector.

In some embodiments, a level of HPRT1 gene expression within the population of substantially HPRT deficient lymphocytes is reduced by at least about 70%, preferably reduced by at least about 80%, more preferably reduced by at least about 90% as compared with the donor lymphocytes which have not been transfected.

In some embodiments, the positive selection comprises contacting the generated population of substantially HPRT deficient lymphocytes with a purine analog, preferably wherein the purine analog is selected from the group consisting of 6-TG and 6-MP. In some embodiments, an amount of the purine analog ranges from between about 1 to about 15 μg/mL. In some embodiments, the positive selection comprises contacting the generated population of substantially HPRT deficient lymphocytes with both a purine analog and allopurinol.

In some embodiments, the method further comprises administering to the patient one or more doses of a dihydrofolate reductase inhibitor, preferably wherein the dihydrofolate reductase inhibitor is selected from the group consisting of MTX or MPA.

In some embodiments, the population modified lymphocytes are administered as a single bolus or as multiple doses. In some embodiments, each dose of the multiple doses comprises between about 0.1×106 cells/kg to about 240×10⁶ cells/kg. In some embodiments, a total dosage comprises between about 0.1×106 cells/kg to about 730×106 cells/kg.

In a twenty-second aspect of the present disclosure is a method of treating a patient with HPRT deficient lymphocytes including the steps of: (a) isolating lymphocytes from a donor subject; (b) contacting the isolated lymphocytes with (i) an endonuclease, and (ii) a guide RNA molecule targeting a sequence within one of Exon2, Exon 3 or Exon 8 of the HPRT 1 gene; (c) exposing the population of HPRT deficient lymphocytes to an agent which positively selects for HPRT deficient lymphocytes to provide a preparation of modified lymphocytes; (d) administering a therapeutically effective amount of the preparation of the modified lymphocytes to the patient following hematopoietic stem-cell transplantation; and (e) optionally administering a dihydrofolate reductase inhibitor following the development of graft-versus-host disease (GvHD) in the patient. In some embodiments, the dihydrofolate reductase inhibitor is selected from the group consisting of MTX or MPA.

In a twenty-third aspect of the present disclosure is a use of a preparation of modified lymphocytes for providing the benefits of a lymphocyte infusion to a subject in need of treatment thereof, wherein the preparation of the modified lymphocytes are generated by: (a) isolating lymphocytes from a donor subject; (b) contacting the isolated lymphocytes with comprising (i) an endonuclease, and (ii) a guide RNA molecule targeting a sequence within one of Exon 2, Exon 3 or Exon 8 of the HPRT 1 gene to provide a population of substantially HPRT deficient lymphocytes; and (c) exposing the population of HPRT deficient lymphocytes to an agent which positively selects for HPRT deficient lymphocytes to provide a preparation of modified lymphocytes. In some embodiments, the subject is in need of treatment following hematopoietic stem cell transplantation. In some embodiments, the guide RNA molecule targets a sequence within Chromosome X located between about 134475181 to about 134475364 based on genome build GRCh38 or an equivalent position in a genome build other than GRCh38. In some embodiments, the guide RNA molecule targeting Exon 2 of the HPRT 1 gene has at least 95% sequence identity to any one of SEQ ID NOS: 45 and 57-61. In some embodiments, the guide RNA molecule targeting Exon 2 of the HPRT 1 gene comprises any one of SEQ ID NOS: 45 and 57-61. In some embodiments, the guide RNA molecule targeting Exon 3 of the HPRT 1 gene has at least 95% sequence identity to any one of SEQ ID NOS: 41-44, 46 and 50-51. In some embodiments, the guide RNA molecule targeting Exon 3 of the HPRT 1 gene comprises any one of SEQ ID NOS: 41-44, 46 and 50-51. In some embodiments, the guide RNA molecule targeting Exon 8 of the HPRT 1 gene has at least 95% sequence identity to any one of SEQ ID NOS: 47-49, 55 and 56. In some embodiments, the guide RNA molecule targeting Exon 8 of the HPRT 1 gene comprises any one of SEQ ID NOS: 47-49, 55 and 56.

In a twenty-fourth aspect of the present disclosure is a method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA having at least 90% sequence identity to any one of SEQ ID NOS: 40-61; (b) positively selecting for the population of substantially HPRT deficient lymphocytes ex vivo to provide a population of modified lymphocytes; and (c) administering a therapeutically effective amount of the population of modified lymphocytes to the patient. In some embodiments, the method further comprises administering an HSC graft to the patient. In some embodiments, the guide RNA has at least 95% sequence identity to any one of SEQ ID NOS: 40-61. In some embodiments, the guide RNA comprises any one of SEQ ID NOS: 40-61.

In a twenty-fifth aspect of the present disclosure is a kit comprising: (i) a guide RNA molecule having at least 95% sequence identity to any one of SEQ ID NOS: 40-61; and (ii) a Cas protein. In some embodiments, the Cas protein is selected from the group consisting of a Cas9 protein and a Cas12 protein.

In a twenty-sixth aspect of the present disclosure is a kit comprising: (i) a guide RNA molecule which targets a sequence within Chromosome X located between about 134475181 to about 134475364 based on genome build GRCh38 or an equivalent position in a genome build other than GRCh38, and (ii) a Cas protein. In some embodiments, the Cas protein is selected from the group consisting of a Cas9 protein and a Cas12 protein.

In comparison to other “off switch” methods, hematopoietic cells, (including T-cells), treated according to the disclosed methods do not need to express a “suicide gene.” Rather, the disclosed method provides for knockdown or knockout of an endogenous gene that causes no undesirable effects in hematological cells and, overall, superior results. Applicant submits that given ex vivo 6-TG chemoselection of gene-modified cells according to the methods described herein, a population of HSCs (or lymphocytes) may be provided for administration to a subject that permits the quantitative elimination of cells in vivo via dosing with a dihydrofolate reductase inhibitor (e.g. methotrexate (MTX)). In addition, treatment according to the disclosed methods provides for potentially higher doses and a more aggressive therapy of donor T-cells than therapy where a “kill switch” is not incorporated. Further, the use of a dihydrofolate reductase inhibitor to regulate the number of modified T-cells is clinically compatible with existing methods of treating GvHD, i.e. where MTX is used to help alleviate GvHD symptoms in patients not receiving the disclosed modified T-cells.

Applicant further submits that in comparison to donor lymphocytes transduced with the herpes simplex thymidine kinase gene, treatment according to the disclosed methods mitigates limitations including immunogenicity resulting in the elimination of the cells and precluding the possibility of future infusions (see Zhou X, Brenner M K, “Improving the safety of T-Cell therapies using an inducible caspase-9 gene,” Exp Hematol. 2016 November; 44(11):1013-1019, the disclosure of which is hereby incorporated by reference herein in its entirety). Also, Applicant submits that the present methods allow for use of ganciclovir for concurrent clinical conditions other than GvHD without resulting in undesired clearance of HSV-tk donor lymphocytes (e.g. ganciclovir would not be precluded from being administered to control CMV infections, which are common in the allo-HSCT setting, when the currently described methods are utilized).

### BRIEF DESCRIPTION OF THE FIGURES

FIG. 1 illustrates a general method of contacting T-cells with either an expression vector adapted to knockdown HPRT or with a nanocapsule including a payload (e.g. a Cas protein and a gRNA) configured to knockout HPRT. The figures further illustrates that a kill switch may be activated, such as in the event that side effects of treatment with modified T-cells is observed.

FIG. 2 illustrates the secondary structure and theoretical primary DICER cleavage sites (arrows) of sh734 (see also SEQ ID NO: 1). The secondary structure has an MFE value of about −30.9 kcal/mol.

FIG. 3 illustrates the secondary RNA structure and minimum free energy (dG) for sh616 (see also SEQ ID NO: 5).

FIG. 4 illustrates the secondary RNA structure and minimum free energy (dG) for sh211 (see also SEQ ID NO: 6).

FIG. 5 illustrates a modified version of sh734 (sh734.1) (see also SEQ ID NO: 7). The secondary structure has an MFE value of −36.16 kcal/mol.

FIG. 6 illustrates the de novo design of an artificial miRNA734 (111 nt). 5′ and 3′ DROSHA target sites and 5′ and 3′ DICER cut sites are indicated by arrows in the secondary structure (see also SEQ ID NO: 8).

FIG. 7 illustrates the de novo design of an artificial miRNA211 (111 nt) (see also SEQ ID NO: 9).

FIG. 8 illustrates a sh734 embedded in the miRNA-3G backbone, a third generation miRNA scaffold derived from the native miRNA 16-2 structure (see also SEQ ID NO: 11).

FIG. 9 illustrates the sh211 embedded in the miRNA-3G backbone, a 3rd generation miRNA scaffold derived from the native miRNA 16-2 structure (see also SEQ ID NO: 10).

FIG. 10 illustrate human 7sk promoter mutations. Mutations (arrows) and deletions introduced into the cis-distal sequence enhancer (DSE) and proximal sequence enhancer (PSE) elements (long, wide boxes) in the 7sk promoter relative to the TATA box (tall, thin boxes) are illustrated. These mutations and others are described by Boyd, D. C., Turner, P. C., Watkins, N.J., Gerster, T. & Murphy, S. Functional Redundancy of Promoter Elements Ensures Efficient Transcription of the Human 7SK Gene in vivo, Journal of Molecular Biology 253, 677-690 (1995), the disclosure of which is hereby incorporated by reference herein in its entirety.

FIG. 11 is a flowchart illustrating the steps of preparing modified T-cells and administering those modified T-cells to a patient in need thereof.

FIGS. 12A and 12B depict successful ex vivo selection and expansion of modified cells (HPRT knockdown via LV transduction or knockout via CRISPR/Cas9 nanocapsules) with 6-TG. These initial preliminary experiments were performed in K562 cells (human immortalized myelogenous leukemia line) (rsh7-GFP=short hairpin to HPRT/GFP lentiviral vector to knockdown HPRT; nanoRNP-HPRT=CRISPR/Cas9 ribonucleoprotein nanocapsules to knockout HPRT). FIG. 12A illustrates that K562 cells transduced with shHPRT-GFP vector at MOI=5 (multiplicity of infection)=5 can be ex-vivo selected with 6-TG to reach a state of more than 95% cells carrying shHPRT in 10 days. FIG. 12B illustrates that HPRT knockout cells, via CRISPR RNP nanocapsules, can also reach higher than 95% in total population in 10 days under 600 nM or 900 nM of 6-TG. These data suggest a feasibility of producing a high content of gene-modified cells through 6-TG chemoselection.

FIG. 13A illustrates the effect of positive selection with 6-TG (ex vivo) on CEM cells.

FIG. 13B illustrates that HPRT knockout population of CEM cells increased from day 3 to day 17 under treatment of 6-TG.

FIGS. 14A and 14B illustrate the effect of negative selection with MTX on K562 cells.

FIGS. 15A and 15B illustrate the effect of negative selection with MTX or MPA on CEM cells.

FIG. 16 illustrates the effect of negative selection with MTX on K562 cells.

FIG. 17 illustrates the de novo path for the synthesis of deoxythymidine triphosphate (dTTP).

FIG. 18 illustrates the selection of HPRT-deficient cells in the presence of 6-TG.

FIG. 19A is a flowchart illustrating the steps of preparing modified T-cells and administering those modified T-cells to a patient following a stem cell graft, such that the patient's immune system may be at least partially reconstituted.

FIG. 19B is a flowchart illustrating the steps of preparing modified T-cells and administering those modified T-cells.

FIG. 20 is a flowchart illustrating the steps of preparing modified T-cells and administering those modified T-cells to a patient following a stem cell graft, such that the modified T-cells assist in inducing the GVM effect.

FIG. 21 is a flowchart illustrating the steps of preparing modified T-cells (CAR-T cells that are HRPT-deficient) and administering those modified T-cells to a patient in need thereof.

FIG. 22 illustrates the relative expression of levels of HPRT and further illustrates a cutoff at which point HPRT deficient cells may be selected for with a purine analog.

FIG. 23 sets forth a table illustrating various guide RNAs examined for on target and off target effects.

FIG. 24 provides a graph depicting luminescence versus 6-TG concentration in HPRT knockout Jurkat cells.

FIG. 25 provides western blots of HPRT knockout and wild-type Jurkat cells, where actin was used as a protein control.

FIG. 26 sets forth a graph of green fluorescent protein (GFP) versus HPRT knock out survival advantage, where the graph provides for the percentage of live cells versus time.

FIG. 27 provides data from fluorescence-activated cell sorting (FACS) of GFP versus HPRT knockout cells.

FIG. 28 provides a graph setting forth a determination of methotrexate (MTX) dose response for wild-type Jurkat cells, where the graph shows the percentage of viable cells.

FIG. 29 provides a graph which illustrates a determination of methotrexate dose response for HPRT knockout and wild-type Jurkat cells, where the graphs illustrate the change in dose response versus methotrexate concentration.

FIG. 30A provides FACS data corresponding to HPRT Knockdown Jurkat T cells transducer with the lentiviral vector TL20cw-7SK/sh734-UbC/GFP.

FIG. 30B provides FACS data corresponding to HPRT Knockdown Jurkat T cells traduced with the lentiviral vector TL20cw-UbC/GFP-7SK/sh734.

FIG. 31 provides graphs illustrating 6-TG selection with HPRT knockdown CEM cells transducer with the lentiviral vectors TL20cw-7SK/sh734-UbC/GFP or TL20cw-UbC/GFP-7SK/sh734.

FIG. 32 illustrates the elements included within lentiviral vectors in accordance with some embodiments of the present disclosure. The figure further illustrates the relative orientations of certain elements relative to others. For example, the 7sk driven sh734 element may be oriented in the same direction or in opposite directions as compared with the UbC driven GFP. In addition, the figure illustrates that the 7sk driven sh734 element may be located either upstream or downstream of other vector elements, e.g. upstream or downstream of the UbC driven GFP.

FIG. 33 provides graphs of the percentage of cells expressing GFP after transduction with one of four vectors.

FIG. 34 illustrates the exons targeted by two sets of gRNAs (such as those having SEQ ID NOS: 25-39 (“round 1”) to those having SEQ ID NOS: 40-49 (“round 2”).

FIG. 35A illustrates Inference of CRISPR Edits (ICE) scores (namely, CRISPER editing efficiency) for the gRNAs having SEQ ID NOS: 40-49.

FIG. 35B illustrates ICE scores (namely, CRISPER editing efficiency) for the gRNAs having SEQ ID NOS: 40-49.

FIG. 36 illustrates the viability of CEM cells transfected with eight different gRNAs in different concentrations of 6-TG.

FIG. 37 sets forth a workflow illustrating the steps of modifying CEM cells.

FIG. 38 illustrates 6-TG dose responses for CEM cells transfected with one of eight different gRNAs 72-hours following electroporation. Guide RNAs generally showed increased resistance to 6-TG when compared to wild-type (“WT”).

FIG. 39 sets forth Western Blot results 72-hours following electroporation of CEM cells transfected with one of eight different gRNAs. Western Blot results correlated well with ICE scoring. The bottom panel of the Western Blot provided anti-beta ACTIN control.

FIG. 40A illustrates the selection of modified CEM cells after being exposed to 6-TG in accordance with the methods described herein. CEM cells 1-week after 10 micromolar 6-TG selection. Cells with lower editing scores were still successfully selected by 6-TG.

FIG. 40B illustrates 6-TG dose response in 6-TG selected CEM cells in accordance with the methods described herein. All modified cells showed resistance to high doses of 6-TG.

FIG. 41 sets forth Western Blot results 72-hours of CEM cells positively selected with 6-TG in accordance with the methods described herein. The HPRT knockout population was successfully selected with 6-TG.

FIGS. 42 and 43 illustrate MTX dose response in 6-TG selected CEM cells in accordance with the methods described herein.

FIG. 44 sets forth a workflow illustrating the steps of modifying primary T-cells.

FIG. 45A depicts 6-TG dose response in T-cells modified using a gRNA targeting Exon 3 of the HPRT 1 gene, e.g. T-cells electroporated in the presence of an RNP including a gRNA targeting Exon 3 of the HPRT1 gene.

FIG. 45B depicts 6-TG dose response in T-cells modified using a gRNA targeting Exon 8 of the HPRT 1 gene, e.g. T-cells electroporated in the presence of an RNP including a gRNA targeting Exon 8 of the HPRT 1 gene.

FIG. 45C depicts primary T-cells electroporated without an RNP.

FIG. 46 illustrate the results of Western Blotting 72-hours after electroporation of T-cells edited with an RNP targeting either Exon 3 or Exon 8 of the HPRT1 gene.

FIG. 47A illustrates the selection of modified primary T-cells (e.g. those modified with a RNP including a gRNA targeting Exon 3 of HPRT1) with 6-TG in accordance with the methods described herein. FIG. 47A illustrates that the successful selection of modified cells.

FIG. 47B illustrates the selection of modified primary T-cells (e.g. those modified with a RNP including a gRNA targeting Exon 8 of HPRT 1) with 6-TG in accordance with the methods described herein. FIG. 47B illustrates that the successful selection of modified cells.

FIG. 48 illustrates the results of Western Blotting after 6-TG selection, e.g. using the selected T-cells of FIGS. 47A and 47B.

FIG. 49A illustrates MTX dose response in 6-TG selected primary T-cells, e.g. those primary T-cells modified with a RNP including a gRNA targeting Exon 3 of the HPRT1 gene).

FIG. 49B illustrates MTX dose response in 6-TG selected primary T-cells, e.g. those primary T-cells modified with a RNP including a gRNA targeting Exon 3 of the HPRT1 gene).

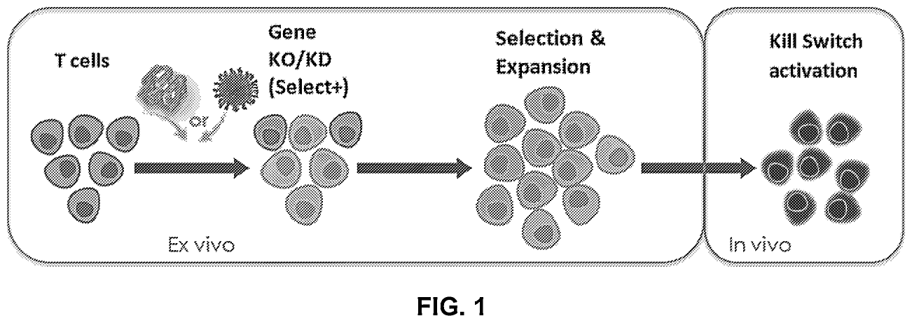

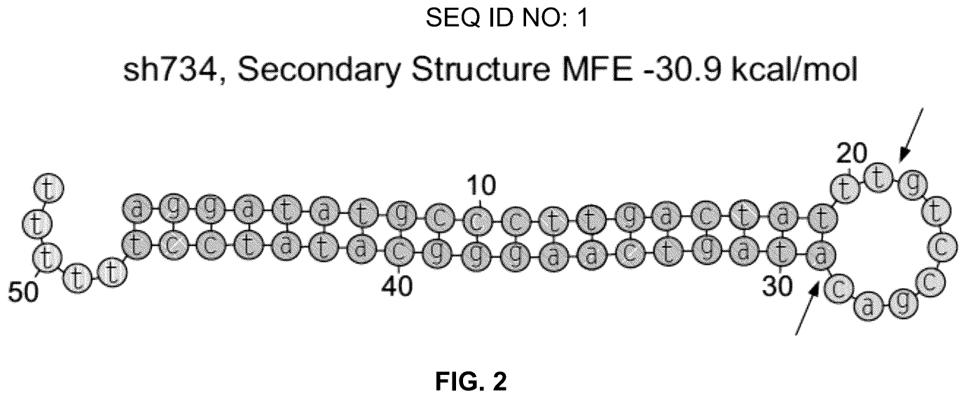

### SEQUENCE LISTING

The contents of the electronic sequence listing (CSL-097W0.xml; Size: 89,428 bytes; and Date of Creation: Nov. 25, 2022) is herein incorporated by reference in its entirety.

### DETAILED DESCRIPTION

### Definitions

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless the context clearly indicates otherwise. Similarly, the word “or” is intended to include “and” unless the context clearly indicates otherwise.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the terms “comprising,” “including,” “having,” and the like are used interchangeably and have the same meaning. Similarly, “comprises,” “includes,” “has,” and the like are used interchangeably and have the same meaning. Specifically, each of the terms is to be interpreted to be an open term meaning “at least the following,” and is also interpreted not to exclude additional features, limitations, aspects, etc. Thus, for example, “a device having components a, b, and c” means that the device includes at least components a, b and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

As used herein, the terms “administer” or “administering” mean providing a composition, formulation, or specific agent to a subject (e.g. a human patient) in need of treatment, including those described herein.

As used herein, the term “Cas protein” refers an RNA-guided nuclease comprising a Cas protein, or a fragment thereof. A Cas protein may also be referred to as a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). Cas proteins include, but are not limited to, Cas9 proteins, Cas9-like proteins encoded by Cas9 orthologs, Cas9-like synthetic proteins, Cpf1 proteins, proteins encoded by Cpf1 orthologs, Cpf1-like synthetic proteins, C2c1 proteins, C2c2 proteins, C2c3 proteins, and variants and modifications thereof. Further examples of Cas proteins include, but are not limited to, Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c.

In some embodiments, a Cas protein is a Class 2 CRISPR-associated protein. “Class 2 type CRISPR-Cas systems” as defined herein refer to CRISPR-Cas systems functioning with a single protein as effector complex (such as Cas9). As defined herein, “class 2 type II CRISPR-Cas system” refers to CRISPR-Cas systems comprising the Cas9 gene among its cas genes. A “class 2 type II— A CRISPR-Cas system” refers to CRISPR-Cas systems comprising cas9 and Csn2 genes. A “class 2 type 11-B CRISPR-Cas system” refers to CRISPR-Cas systems comprising the cas9 and cas4 genes. A “class 2 type 11-C CRISPR-Cas system” refers to CRISPR-Cas systems comprising the Cas9 gene but neither the Csn2 nor the Cas4 gene. A “class 2 type V CRISPR-Cas system” refers to CRISPR-Cas systems comprising the cas12 gene (Cas12a, 12b or 12c gene) in its cas genes. A “class 2 type VI CRISPR-Cas system” refers to CRISPR-Cas systems comprising the Cas13 gene (Cas13a, 13b or 13c gene) in its Cas genes. Each wild-type Cas protein interacts with one or more cognate polynucleotide (most typically RNA) to form a nucleoprotein complex (most typically a ribonucleoprotein complex). Additional Cas proteins are described by Haft et. al., “A Guild of 45 CRISPR-Associated (Cas) Protein Families and Multiple CRISPR/Cas Subtypes Exist in Prokaryotic Genomes, PLoS Comput. Biol., 2005, November; 1(6): e60. In some embodiments, the Cas protein is a modified Cas protein, e.g. a modified variant of any of the Cas proteins identified herein.

As used herein, the terms “Cas9” or “Cas9 protein” refer to an enzyme (wild-type or recombinant) that can exhibit least endonuclease activity (e.g. cleaving the phosphodiester bond within a polynucleotide) guided by a CRISPR RNA (crRNA) bearing complementary sequence to a target polynucleotide. Cas9 polypeptides are known in the art and include Cas9 polypeptides from any of a variety of biological sources, including, e.g., prokaryotic sources such as bacteria and archaea. Bacterial Cas9 includes, Actinobacteria (e.g., Actinomyces naeslundii) Cas9, Aquificae Cas9, Bacteroidetes Cas 9, Chlamydiae Cas9, Chloroflexi Cas9, Cyanobacteria Cas9, Elusimicrobia Cas9, Fibrobacteres Cas9, Firmicutes Cas9 (e.g., Streptococcus pyogenes Cas9, Streptococcus thermophilus Cas9, Listeria innocua Cas9, Streptococcus agalactiae Cas9, Streptococcus mutans Cas9, and Enterococcus faecium Cas9), Fusobacteria Cas9, Proteobacteria (e.g., Neisseria meningitides, Campylobacter jejuni and lari) Cas9, Spirochaetes (e.g., Treponema denticola) Cas9, and the like. Archaea Cas 9 includes Euryarchaeota Cas9 (e.g., Methanococcus maripaludis Cas9) and the like. A variety of Cas9 and related polypeptides are known, and are reviewed in, e.g., Makarova et al. (2011) Nature Reviews Microbiology 9:467-477, Makarova et al. (2011) Biology Direct 6:38, Haft et al. (2005) PLOS Computational Biology I:e60 and Chylinski et al. (2013) RNA Biology 10:726-737; K. Makarova et al., An updated evolutionary classification of CRISPR-Cas systems. (2015) Nat. Rev. Microbio. 13:722-736; and B. Zetsche et al. Cpf1 is a single RNA-guided endonuclease of a class 2 CRISPR-Cas system. (2015) Cell. 163(3):759-771.

Other Cas9 polypeptides include Francisella tularensis subsp. novicida Cas9, Pasteurella multocida Cas9, Mycoplasma gallisepticum str. F Cas9, Nitratifractor salsuginis str DSM 16511 Cas9, Parvibaculum lavamentivorans Cas9, Roseburia intestinalis Cas9, Neisseria cinera Cas9, Gluconacetobacter diazotrophicus Cas9, Azospirillum B510 Cas9, Spaerochaeta globus str. Buddy cas9, Flavobacterium columnare Cas9, Fluviicola taffensis Cas9, Bacteroides coprophilus Cas9, Mycoplasma mobile Cas9, Lactobacillus farciminis Cas9, Streptococcus pasteurianus Cas9, Lactobacillus johnsonii Cas9, Staphylococcus pseudintermedius Cas9, Filifactor alocis Cas9, Treponema denticola Cas9, Legionella pneumophila str. Paris Cas9, Sutterella wadsworthensis Cas9, and Corynebacter diptheriae Cas9. The term “Cas9” includes a Cas9 polypeptide of any Cas9 family, including any isoform of Cas9. Amino acid sequences of various Cas9 homologs, orthologs, and variants beyond those specifically stated or provided herein are known in the art and are publicly available, within the purview of those skill in the art, and thus within the spirit and scope of this disclosure.

As used herein, the terms “Cas12” or “Cas12 protein” refer to any Cas12 protein including, but not limited to, Cas12 protein such as Cas12a, Cas12b, Cas12c, Cas12d, Cas12e. In some embodiments, a Cas12 protein has an amino acid sequence which is at least 85% (or at least 90%, or at least 95%, or at least 96%, or at least 97%, or at least 98%, or at least 99%) identical to the amino acid sequence of a functional Cas12 protein, particularly the Cas12a/Cpf1 protein from Acidaminococcus sp. strain BV3L6 (Uniprot Entry: U2UMQ6; Uniprot Entry Name: CS12A_ACISB) or the Cas12a/Cpf1 protein from Francisella tularensis (Uniprot Entry: A0Q7Q2; Uniprot Entry Name: CS12A_FRATN). In some embodiments, the Cas12 protein may be a Cas12 polypeptide substantially identical to the protein found in nature, or a Cas12 polypeptide having at least 85% sequence identity (or at least 90% sequence identity, or at least 95% sequence identity, or at least 96% sequence identity, or at least 97% sequence identity, or at least 98% sequence identity, or at least 99% sequence identity) to the Cas12 protein found in nature and having substantially the same biological activity. Examples of Cas12a proteins include, but are not limited to, FnCas12a, AsCas12a, LbCas12a, Lb5Cas12a, HkCas12a, OsCas12a, TsCas12a, BbCas12a, BoCas12a or Lb4Cas12a; the Cas12a is preferably LbCas12a. Examples of Cas12b proteins include, but are not limited to, AacCas12b, Aac2Cas12b, AkCas12b, AmCas12b, AhCas12b, AcCas12b.

As used herein, the phrase “effective amount” refers to the amount of a composition or formulation described herein that will elicit the diagnostic, biological or medical response of a tissue, system, animal, or human that is being sought by the researcher, veterinarian, medical doctor or other clinician.

As used herein, the term “electroporation” refers to a technique in which an electrical field is applied to cells in order to increase the permeability of the cell membrane, allowing chemicals, small molecules, proteins, nucleic acids, etc. to be introduced into the cell.

As used herein, the term “expression cassette” refers to one or more genetic sequences within a vector which can express a RNA, and, in some embodiments, subsequently a protein. The expression cassette comprises at least one promoter and at least one gene of interest. In some embodiments, the expression cassette includes at least one promoter, at least one gene of interest, and at least one additional nucleic acid sequence encoding a molecule for expression (e.g. a RNAi). In some embodiments, expression cassette is positionally and sequentially oriented within the vector such that the nucleic acid in the cassette can be transcribed into RNA, and when necessary, translated into a protein or a polypeptide, undergo appropriate post-translational modifications required for activity in the transformed cell (e.g. transduced stem cell), and be translocated to the appropriate compartment for biological activity by targeting to appropriate intracellular compartments or secretion into extracellular compartments. In some embodiments, the cassette has its 3′ and 5′ ends adapted for ready insertion into a vector, e.g., it has restriction endonuclease sites at each end.

As used herein, the term “functional nucleic acid” refers to molecules having the capacity to reduce expression of a protein by directly interacting with a transcript that encodes the protein. siRNA molecules, ribozymes, and antisense nucleic acids constitute exemplary functional nucleic acids.

As used herein, the term “gene” refers broadly to any segment of DNA associated with a biological function. A gene encompasses sequences including but not limited to a coding sequence, a promoter region, a cis-regulatory sequence, a non-expressed DNA segment is a specific recognition sequence for regulatory proteins, a non-expressed DNA segment that contributes to gene expression, a DNA segment designed to have desired parameters, or combinations thereof.

As used herein, the term “gene silencing” is meant to describe the downregulation, knock-down, degradation, inhibition, suppression, repression, prevention, or decreased expression of a gene, transcript and/or polypeptide product. Gene silencing and interference also describe the prevention of translation of mRNA transcripts into a polypeptide. In some embodiments, translation is prevented, inhibited, or decreased by degrading mRNA transcripts or blocking mRNA translation.

As used herein, the term “gene expression” refers to the cellular processes by which a biologically active polypeptide is produced from a DNA sequence.

As used herein, the term “genome build” refers to successive “versions” of the human genome reference. The latest build of the human reference genome is named GRCh38 (for Genome Research Consortium human build 38) but commonly nicknamed Hg38 (for Human genome build 38).

As used herein, the terms “guide RNA” or “gRNA” refer to a RNA molecule capable of directing a CRISPR effector having nuclease activity to target and cleave a specified target nucleic acid.

As used herein, the terms “hematopoietic cell transplant” or “hematopoietic cell transplantation” refer to bone marrow transplantation, peripheral blood stem cell transplantation, umbilical vein blood transplantation, or any other source of pluripotent hematopoietic stem cells. Likewise, the terms “stem cell transplant,” or “transplant,” refer to a composition comprising stem cells that are in contact with (e.g. suspended in) a pharmaceutically acceptable carrier. Such compositions are capable of being administered to a subject through a catheter.

As used herein, the term “host cell” refers to cells that is to be modified using the methods of the present disclosure. In some embodiments, the host cells are mammalian cells in which the expression vector can be expressed. Suitable mammalian host cells include, but are not limited to, human cells, murine cells, non-human primate cells (e.g. rhesus monkey cells), human progenitor cells or stem cells, 293 cells, HeLa cells, D17 cells, MDCK cells, BHK cells, and Cf2Th cells. In certain embodiments, the host cell comprising an expression vector of the disclosure is a hematopoietic cell, such as hematopoietic progenitor/stem cell (e.g. CD34-positive hematopoietic progenitor/stem cell), a monocyte, a macrophage, a peripheral blood mononuclear cell, a CD4+ T lymphocyte, a CD8+ T lymphocyte, or a dendritic cell. The hematopoietic cells (e.g. CD4+ T lymphocytes, CD8+ T lymphocytes, and/or monocyte/macrophages) to be transduced with an expression vector of the disclosure can be allogeneic, autologous, or from a matched sibling. The hematopoietic progenitor/stem cell are, in some embodiments, CD34-positive and can be isolated from the patient's bone marrow or peripheral blood. The isolated CD34-positive hematopoietic progenitor/stem cell (and/or other hematopoietic cell described herein) is, in some embodiments, transduced with an expression vector as described herein.

As used herein, the terms “hypoxanthine-guanine phosphoribosyltransferase” or “HPRT” refer to an enzyme involved in purine metabolism encoded by the HPRT1 gene (see, for example, SEQ ID NO: 12). HPRT1 is located on the X chromosome, and thus is present in single copy in males. HPRT1 encodes the transferase that catalyzes the conversion of hypoxanthine to inosine monophosphate and guanine to guanosine monophosphate by transferring the 5-phosphorobosyl group from 5-phosphoribosyl 1-pyrophosphate to the purine. The enzyme functions primarily to salvage purines from degraded DNA for use in renewed purine synthesis.

As used herein, the term “indel” refers to a mutation named with the blend of insertion and deletion. It refers to a length difference between two alleles where it is unknowable if the difference was originally caused by a sequence insertion or by a sequence deletion. If the number of nucleotides in the insertion/deletion is not divisible by three, and it occurs in a protein coding region, it is also a frameshift mutation (frameshift mutation will in general cause the reading of the codons after the mutation to code for different amino acid).

As used herein, the term “lentivirus” refers to a genus of retroviruses that are capable of infecting dividing and non-dividing cells. Several examples of lentiviruses include HIV (human immunodeficiency virus: including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which causes immune deficiency and encephalopathy in sub-human primates.

As used herein, the term “lentiviral vector” is used to denote any form of a nucleic acid derived from a lentivirus and used to transfer genetic material into a cell via transduction. The term encompasses lentiviral vector nucleic acids, such as DNA and RNA, encapsulated forms of these nucleic acids, and viral particles in which the viral vector nucleic acids have been packaged.

As used herein, the term “lymphocyte” refers to one or more of the subtypes of a white blood cell in the vertebrate immune system, including T cells, B cells and natural killer (NK) cells. A skilled person would understand that T cells (also referred to as T lymphocytes or CD3+T lymphocytes) may be characterized based on their specific function, that is, helper/effector (CD4 T Cells), cytotoxic (CD8 T Cells), memory, regulatory and gamma delta (γδ) T cells. A skilled person would further understand that types of T-cells may be distinguished by the type and distribution of cell-surface markers. By way of example, subpopulations of T cells may be distinguished by the cell surface markers CD4 and CD8 together with CC chemokine receptor 7 (CCR7) and CD45RA. Such subpopulations may be further distinguished by expression of other cell-surface markers. For example, naive T cell, effector memory (EM), central memory (CM), and effector T cell subsets may be further defined by CCR7 and CD45RA expression, in addition to other markers. In some embodiments, the lymphocyte is a T-cell. In some embodiments, the lymphocyte is a B cell. In some embodiments, the lymphocyte is a natural killer (NK). In some embodiments, the lymphocyte is a primary human T cell. In some embodiments, the lymphocyte is a CD3+ T cell. In some embodiments, the lymphocyte is a CD4+ T cell. In some embodiments, the lymphocyte is a CD8+ T cell. In some embodiments, the lymphocyte is a HLA-DR+ T cell. In some embodiments, the lymphocyte is an αβ T cell. In some embodiments, the lymphocyte is a γδ T cell.

As used herein, the terms “knock down” or “knockdown” when used in reference to an effect of RNAi on gene expression, means that the level of gene expression is inhibited, or is reduced to a level below that generally observed when examined under substantially the same conditions, but in the absence of RNAi.

As used herein, the terms “knock-out” or “knockout” refer to partial or complete suppression of the expression of an endogenous gene. This is generally accomplished by deleting a portion of the gene or by replacing a portion with a second sequence, but may also be caused by other modifications to the gene such as the introduction of stop codons, the mutation of critical amino acids, the removal of an intron junction, etc. Accordingly, a “knock-out” construct is a nucleic acid sequence, such as a DNA construct, which, when introduced into a cell, results in suppression (partial or complete) of expression of a polypeptide or protein encoded by endogenous DNA in the cell. In some embodiments, a “knockout” includes mutations such as, a point mutation, an insertion, a deletion, a frameshift, or a missense mutation.

As used herein, the term “microinjection” refers to a technique for chemicals, small molecules, proteins, nucleic acids, etc. to be introduced into a single cell by insertion of a micropipette into the cell of interest.

As used herein, the terms “multiplicity of infection” or “MOI” means the ratio of agents (e.g. phage or more generally virus, bacteria) to infection targets (e.g. cell). For example, when referring to a group of cells inoculated with virus particles, the multiplicity of infection or MOI is the ratio of the number of virus particles to the number of target cells present in a defined space.

As used herein, the term “minicell” refers to anucleate forms of bacterial cells, engendered by a disturbance in the coordination, during binary fission, of cell division with DNA segregation. Minicells are distinct from other small vesicles that are generated and released spontaneously in certain situations and, in contrast to minicells, are not due to specific genetic rearrangements or episomal gene expression. Minicells of the present disclosure are anucleate forms of E. coli or other bacterial cells, engendered by a disturbance in the coordination, during binary fission, of cell division with DNA segregation. Prokaryotic chromosomal replication is linked to normal binary fission, which involves mid-cell septum formation. In E. coli, for example, mutation of min genes, such as minCD, can remove the inhibition of septum formation at the cell poles during cell division, resulting in production of a normal daughter cell and an anucleate minicell. See de Boer et al., 1992; Raskin & de Boer, 1999; Hu & Lutkenhaus, 1999; Harry, 2001. Minicells are distinct from other small vesicles that are generated and released spontaneously in certain situations and, in contrast to minicells, are not due to specific genetic rearrangements or episomal gene expression. For practicing the present disclosure, it is desirable for minicells to have intact cell walls (“intact minicells”). In addition to min operon mutations, anucleate minicells also are generated following a range of other genetic rearrangements or mutations that affect septum formation, for example in the divIVB1 in B. subtilis. See Reeve and Cornett, 1975; Levin et al., 1992. Minicells also can be formed following a perturbation in the levels of gene expression of proteins involved in cell division/chromosome segregation. For example, overexpression of minE leads to polar division and production of minicells. Similarly, chromosome-less minicells may result from defects in chromosome segregation for example the smc mutation in Bacillus subtilis (Britton et al., 1998), spoOJ deletion in B. subtilis (Ireton et al., 1994), mukB mutation in E. coli (Hiraga et al., 1989), and parC mutation in E. coli (Stewart and D'Ari, 1992). Gene products may be supplied in trans. When over-expressed from a high-copy number plasmid, for example, CafA may enhance the rate of cell division and/or inhibit chromosome partitioning after replication (Okada et al., 1994), resulting in formation of chained cells and anucleate minicells (Wachi et al., 1989; Okada et al., 1993). Minicells can be prepared from any bacterial cell of Gram-positive or Gram-negative origin.

As used herein, the term “mutated” refers to a change in a sequence, such as a nucleotide or amino acid sequence, from a native, wild-type, standard, or reference version of the respective sequence, i.e. the non-mutated sequence. A mutated gene can result in a mutated gene product. A mutated gene product will differ from the non-mutated gene product by one or more amino acid residues. In some embodiments, a mutated gene which results in a mutated gene product can have a sequence identity of about 70%, about 75%, about 80%, about 85%, about 90%, about 95%, or greater to the corresponding non-mutated nucleotide sequence.

As used herein, the term “nanocapsules” refers to nanoparticles having a shell, e.g. a polymeric shell, encapsulating one or more components, e.g. one or more proteins and/or one or more nucleic acids. In some embodiments, the nanocapsules have an average diameter of less than or equal to about 200 nanometers (nm), for example between about 1 to 200 nm, or between about 5 to about 200 nm, or between about 10 to about 150 nm, or 15 to 100 nm, or between about 15 to about 150 nm, or between about 20 to about 125 nm, or between about 50 to about 100 nm, or between about 50 to about 75 nm. In other embodiments, the nanocapsules have an average diameter of between about 10 nm to about 20 nm, about 20 to about 25 nm, about 25 nm to about 30 nm, about 30 nm to about 35 nm, about 35 nm to about 40 nm, about 40 nm to about 45 nm, about 45 nm to about 50 nm, about 50 nm to about 55 nm, about 55 nm to about 60 nm, about 60 nm to about 65 nm, about 70 to about 75 nm, about 75 nm to about 80 nm, about 80 nm to about 85 nm, about 85 nm to about 90 nm, about 90 nm to about 95 nm, about 95 nm to about 100 nm, or about 100 nm to about 110 nm. In some embodiments, the nanocapsules are designed to degrade in about 1 hour, or about 2 hours, or about 3 hours, or about 4 hours, or about 5 hours, or about 6 or about 12 hours, or about 1 day, or about 2 days, or about 1 week, or about 1 month. In some embodiments, the surface of the nanocapsule can have a charge between about 1 to about 15 millivolts (mV) (such as measured in a standard phosphate solution). In other embodiments, the surface of the nanocapsule can have a charge of between about 1 to about 10 mV.

As used herein, the terms “positively charged monomer” or “cationic monomer” refer to monomers having a net positive charge, i.e. +1, +2, +3. In some embodiments, the positively charged monomer is a monomer including positively-charged groups. As used herein, the terms “negatively charged monomer” or “anionic monomer” refer to monomers having a net negative charge, i.e. −1, −2, −3. In some embodiments, the negatively charged monomer is a monomer including negatively-charged groups. As used herein, the term “neutral monomer” refers to monomers having a net neutral charge.

As used herein, the term “polymer” is defined as being inclusive of homopolymers, copolymers, interpenetrating networks, and oligomers. Thus, the term polymer may be used interchangeably herein with the term homopolymers, copolymers, interpenetrating polymer networks, etc. The term “homopolymer” is defined as a polymer derived from a single species of monomer. The term “copolymer” is defined as a polymer derived from more than one species of monomer, including copolymers that are obtained by copolymerization of two monomer species, those obtained from three monomers species (“terpolymers”), those obtained from four monomers species (“quaterpolymers”), etc. The term “copolymer” is further defined as being inclusive of random copolymers, alternating copolymers, graft copolymers, and block copolymers. Copolymers, as that term is used generally, include interpenetrating polymer networks. The term “random copolymer” is defined as a copolymer comprising macromolecules in which the probability of finding a given monomeric unit at any given site in the chain is independent of the nature of the adjacent units. In a random copolymer, the sequence distribution of monomeric units follows Bemoullian statistics. The term “alternating copolymer” is defined as a copolymer comprising macromolecules that include two species of monomeric units in alternating sequence.

As used herein, the term “crosslinker” refers to a bond or moiety which provides a link (e.g. an intramolecular link or intermolecular link) between two or more molecular chains, domains, or other moieties. In some embodiments, a crosslinker is a molecule which forms links between molecular chains to form a connected molecule.

As used herein, the term “operably linked” refers to functional linkage between a nucleic acid expression control sequence (such as a promoter, signal sequence, enhancer or array of transcription factor binding sites) and a second nucleic acid sequence, wherein the expression control sequence affects transcription and/or translation of the nucleic acid corresponding to the second sequence when the appropriate molecules (e.g., transcriptional activator proteins) are bound to the expression control sequence.

As used herein, the term “promoter” refers to a recognition site of a polynucleotide (DNA or RNA) to which an RNA polymerase binds. An RNA polymerase initiates and transcribes polynucleotides operably linked to the promoter. In some embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

As used herein, the terms “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use.

As used herein, the term “retroviruses” refers to viruses having an RNA genome that is reverse transcribed by retroviral reverse transcriptase to a cDNA copy that is integrated into the host cell genome. Retroviral vectors and methods of making retroviral vectors are known in the art. Briefly, to construct a retroviral vector, a nucleic acid encoding a gene of interest is inserted into the viral genome in the place of certain viral sequences to produce a virus that is replication-defective. In order to produce virions, a packaging cell line containing the gag, poi, and env genes but without the LTR and packaging components is constructed (Mann et al., Cell, Vol. 33:153-159, 1983). When a recombinant plasmid containing a cDNA, together with the retroviral LTR and packaging sequences, is introduced into this cell line, the packaging sequence allows the RNA transcript of the recombinant plasmid to be packaged into viral particles, which are then secreted into the culture media. The media containing the recombinant retroviruses is then collected, optionally concentrated, and used for gene transfer.

As used herein, the terms “siRNA” or “small interference RNA” refer to a short double-strand RNA composed of about ten nucleotides to several tens of nucleotides, which induce RNAi (RNA interference), i.e. induce the degradation of the target mRNA or inhibit the expression of the target gene via cleavage of the target snRNA. RNA interference (“RNAi”) is a method of post-transcriptional inhibition of gene expression that is conserved throughout many eukaryotic organisms, and it refers to a phenomenon in which a double-stranded RNA composed of a sense RNA having a sequence homologous to the mRNA of the target gene and an antisense RNA having a sequence complementary thereto is introduced into cells or the like so that it can selectively induce the degradation of the mRNA of the target gene or can inhibit the expression of the target gene. RNAi is induced by a short (i.e., less than about 30 nucleotides) double-stranded RNA molecule present in cells (Fire A. et al., Nature, 391: 806-811, 1998). When siRNA is introduced into cells, the expression of the mRNA of the target gene having a nucleotide sequence complementary to that of the siRNA will be inhibited.

As used herein, the terms “small hairpin RNA” or “shRNA” refer to RNA molecules comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme which is a member of the RNase III family. As used herein, the phrase “post-transcriptional processing” refers to mRNA processing that occurs after transcription and is mediated, for example, by the enzymes and/or Drosha.

As used herein, the term “subject” refers to a mammal such as a human, mouse or primate. Typically, the mammal is a human (Homo sapiens).

As used herein, the term “substantially HPRT deficient” and variations thereof refers to cells, e.g. host cells, where the level of HPRT1 gene expression is reduced by at least about 50%. In some embodiments, the level of HPRT1 gene expression is reduced by at least about 55%. In some embodiments, the level of HPRT1 gene expression is reduced by at least about 60%. In some embodiments, the level of HPRT1 gene expression is reduced by at least about 65%. In some embodiments, the level of HPRT1 gene expression is reduced by at least about 70%. In some embodiments, the level of HPRT1 gene expression is reduced by at least about 75%. In some embodiments, the level of HPRT1 gene expression is reduced by at least about 80%. In some embodiments, the level of HPRT1 gene expression is reduced by at least about 85%. In some embodiments, the level of HPRT1 gene expression is reduced by at least about 90%. In some embodiments, the level of HPRT1 gene expression is reduced by at least about 95%. In other embodiments, residual HPRT1 gene expression is at most about 40%. In other embodiments, residual HPRT1 gene is at most about 35%. In other embodiments, residual HPRT1 gene expression is at most about 30%. In other embodiments, residual HPRT1 gene expression is at most about 25%. In other embodiments, residual HPRT1 gene expression is at most about 20%. In other embodiments, residual HPRT1 gene expression is at most about 15%. In other embodiments, residual HPRT1 gene expression is at most about 10%.

As used herein, the terms “transduce”, or “transduction” refers to the delivery of a gene(s) using a viral or retroviral vector by means of infection rather than by transfection. For example, an anti-HPRT1 gene carried by a retroviral vector (a modified retrovirus used as an expression vector for introduction of nucleic acid into cells) can be transduced into a cell through infection and provirus integration. Thus, a “transduced gene” is a gene that has been introduced into the cell via lentiviral or vector infection and provirus integration. Viral vectors (e.g., “transducing vectors”) transduce genes into “target cells” or host cells.

As used herein, the term “transfection” refers to the process of introducing naked DNA into cells by non-viral methods.

As used herein, the term “transduction” refers to the introduction of foreign DNA into a cell's genome using a viral vector.

As used herein, the terms “treatment,” “treating,” or “treat,” with respect to a specific condition, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment”, as used herein, covers any treatment of a disease or disorder in a subject, particularly in a human, and includes: (a) preventing the disease or disorder from occurring in a subject which may be predisposed to the disease but has not yet been diagnosed as having it; (b) inhibiting the disease or disorder, i.e., arresting its development; and (c) relieving or alleviating the disease or disorder, i.e., causing regression of the disease or disorder and/or relieving one or more disease or disorder symptoms. “Treatment” can also encompass delivery of an agent or administration of a therapy in order to provide for a pharmacologic effect, even in the absence of a disease, disorder or condition. The term “treatment” is used in some embodiments to refer to administration of a compound of the present disclosure to mitigate a disease or a disorder in a host, preferably in a mammalian subject, more preferably in humans. Thus, the term “treatment” can include preventing a disorder from occurring in a host, particularly when the host is predisposed to acquiring the disease but has not yet been diagnosed with the disease; inhibiting the disorder; and/or alleviating or reversing the disorder. As far as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure can occur prior to onset of a disease. The term does not mean that the disease state must be completely avoided.

As used herein, the term “vector” refers to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication or may include sequences sufficient to allow integration into host cell DNA. As will be evident to one of ordinary skill in the art, viral vectors may include various viral components in addition to nucleic acid(s) that mediate entry of the transferred nucleic acid. Numerous vectors are known in the art including, but not limited to, linear polynucleotides, polynucleotides associated with ionic or amphiphilic compounds, plasmids, and viral vectors. Examples of viral vectors include, but are not limited to, adenoviral vectors, adeno-associated virus vectors, retroviral vectors (including lentiviral vectors), and the like.

Expression Vectors

The present disclosure provides, in some embodiments, expression vectors (e.g. lentiviral expression vectors) including at least one nucleic acid sequence for expression. In some embodiments, the expression vectors include a first nucleic acid sequence encoding an agent designed to knockdown the HPRT1 gene or otherwise effectuate a decrease in HPRT1 gene expression. In some embodiments, HPRT1 gene expression is reduced by 80% or more.

In some embodiments, the present disclosure provides an expression vector comprising a first expression control sequence operably linked to a first nucleic acid sequence, the first nucleic acid sequence encoding a shRNA to knockdown hypoxanthine-guanine phosphoribosyltransferase (HPRT), wherein the shRNA has at least 90% identity to the sequence of any one of SEQ ID NOS: 2, 5, 6, and 7. In some embodiments, the shRNA has a nucleic acid sequence having at least 95% identity to the sequence of any one of SEQ ID NOS: 2, 5, 6, and 7. In some embodiments, the shRNA has a nucleic acid sequence having at least 97% identity to the sequence of any one of SEQ ID NOS: 2, 5, 6, and 7. In some embodiments, the shRNA comprises the nucleic acid sequence of any one of SEQ ID NOS: 2, 5, 6, and 7. In some embodiments, the shRNA to knockdown hypoxanthine-guanine phosphoribosyltransferase (HPRT) is the only transgene for expression in the expression vector.

In some embodiments, there is provided an expression vector consisting essentially of a first expression control sequence operably linked to a first nucleic acid sequence as the transgene for expression, the first nucleic acid sequence encoding a shRNA to knockdown hypoxanthine-guanine phosphoribosyl transferase (HPRT), wherein the shRNA has at least 90% identity to the sequence of any of SEQ ID NOS: 2, 5, 6, and 7. Specifically, in some embodiments there is provided an expression vector consisting essentially of a first nucleic acid sequence as the only transgene for expression, the first nucleic acid sequence encoding a shRNA to knockdown hypoxanthine-guanine phosphoribosyl transferase (HPRT), wherein the shRNA has at least 90% identity to the sequence of any of SEQ ID NOS: 2, 5, 6, and 7.

In further aspects, there is provided an expression vector comprising a first expression control sequence operably linked to a first nucleic acid sequence as the transgene, the first nucleic acid sequence encoding a shRNA to knockdown hypoxanthine-guanine phosphoribosyl transferase (HPRT), wherein the shRNA has at least 90% identity to the sequence of any of SEQ ID NOS: 2, 5, 6, and 7, wherein the first nucleic acid sequence is the only element for expression. Specifically, in some embodiments there is provided an expression vector comprising a first nucleic acid sequence as the only transgene for expression, the first nucleic acid sequence encoding a shRNA to knockdown hypoxanthine-guanine phosphoribosyl transferase (HPRT), wherein the shRNA has at least 90% identity to the sequence of any of SEQ ID NOS: 2, 5, 6, and 7.

In some embodiments, the expression vector is a self-inactivating lentiviral vector. In other embodiments, the expression vector is a retroviral vector. A lentiviral genome is generally organized into a 5′ long terminal repeat (LTR), the gag gene, the pol gene, the env gene, the accessory genes (nef, vif, vpr, vpu) and a 3′ LTR. The viral LTR is divided into three regions called U3, R and U5. The U3 region contains the enhancer and promoter elements. The U5 region contains the polyadenylation signals. The R (repeat) region separates the U3 and U5 regions and transcribed sequences of the R region appear at both the 5′ and 3′ ends of the viral RNA. See, for example, “RNA Viruses: A Practical Approach” (Alan J. Cann, Ed., Oxford University Press, (2000)); O Narayan and Clements (1989) J. Gen. Virology, Vol. 70:1617-1639; Fields et al. (1990) Fundamental Virology Raven Press.; Miyoshi H, Blamer U, Takahashi M, Gage F H, Verma I M. (1998) J Virol., Vol. 72(10):8150 7, and U.S. Pat. No. 6,013,516. Examples of lentiviral vectors that have been used to infect HSCs are described in the publications which follows, each of which are hereby incorporated herein by reference in their entireties: Evans et al., Hum Gene Ther., Vol. 10:1479-1489, 1999; Case et al., Proc Natl Acad Sci USA, Vol. 96:2988-2993, 1999; Uchida et al., Proc Natl Acad Sci USA, Vol. 95:11939-11944, 1998; Miyoshi et al., Science, Vol. 283:682-686, 1999; and Sutton et al., J. Virol., Vol. 72:5781-5788, 1998.

In some embodiments, the expression vector is a modified lentivirus, and thus is able to infect both dividing and non-dividing cells. In some embodiments, the modified lentiviral genome lacks genes for lentiviral proteins required for viral replication, thus preventing undesired replication, such as replication in the target cells. In some embodiments, the required proteins for replication of the modified genome are provided in trans in the packaging cell line during production of the recombinant retrovirus or lentivirus.

In some embodiments, the expression vector comprises sequences from the 5′ and 3′ long terminal repeats (LTRs) of a lentivirus. In some embodiments, the vector comprises the R and U5 sequences from the 5′ LTR of a lentivirus and an inactivated or self-inactivating 3′ LTR from a lentivirus. In some embodiments, the LTR sequences are HIV LTR sequences.

Additional components of a lentiviral expression vector (and methods of synthesizing and/or producing such vectors) are disclosed in United States Patent Application Publication No. 2018/0112220, the disclosure of which is hereby incorporated by reference herein in its entirety. In some embodiments, the lentiviral expression vector comprises a TL20c backbone having at least 90% identity to that of SEQ ID NO: 16. In some embodiments, the lentiviral expression vector comprises a TL20c backbone having at least 95% identity to that of SEQ ID NO: 16. In some embodiments, the lentiviral expression vector comprises a nucleic acid sequence having at least 90% identity to that of SEQ ID NO: 17. In some embodiments, the lentiviral expression vector comprises a nucleic acid sequence having at least 90% identity to that of SEQ ID NO: 17. In some embodiments, the lentiviral expression vector comprises at least one of a WPRE element or a Rev Response element (see, for example, SEQ ID NOS: 18 and 19, respectively).

In some embodiments, the lentiviral vectors contemplated herein may be integrative or non-integrating (also referred to as an integration defective lentivirus). As used herein, the term “integration defective lentivirus” or “IDLV” refers to a lentivirus having an integrase that lacks the capacity to integrate the viral genome into the genome of the host cells. In some applications, the use of by an integrating lentivirus vector may avoid potential insertional mutagenesis induced by an integrating lentivirus. Integration defective lentiviral vectors typically are generated by mutating the lentiviral integrase gene or by modifying the attachment sequences of the LTRs (see, e.g., Sarkis et al., Curr. Gene. Ther., 6: 430-437 (2008)). Lentiviral integrase is coded for by the HIV-1 Pol region and the region cannot be deleted as it encodes other critical activities including reverse transcription, nuclear import, and viral particle assembly. Mutations in pol that alter the integrase protein fall into one of two classes: those which selectively affect only integrase activity (Class I); or those that have pleiotropic effects (Class II). Mutations throughout the N and C terminals and the catalytic core region of the integrase protein generate Class II mutations that affect multiple functions including particle formation and reverse transcription. Class I mutations limit their affect to the catalytic activities, DNA binding, linear episome processing and multimerization of integrase. The most common Class I mutation sites are a triad of residues at the catalytic core of integrase, including D64, D116, and E152. Each mutation has been shown to efficiently inhibit integration with a frequency of integration up to four logs below that of normal integrating vectors while maintaining transgene expression of the NILV. Another alternative method for inhibiting integration is to introduce mutations in the integrase DNA attachment site (LTR att sites) within a 12 base-pair region of the U3 region or within an 11 base-pair region of the U5 region at the terminal ends of the 5′ and 3′ LTRs, respectively. These sequences include the conserved terminal CA dinucleotide which is exposed following integrase-mediated end-processing. Single or double mutations at the conserved CA/TG dinucleotide result in up to a three to four log reduction in integration frequency; however, it retains all other necessary functions for efficient viral transduction.

In some embodiments, the vector is an adeno-associated virus (AAV) vector. As used herein, the term “adeno-associated virus (AAV) vector” means an AAV viral particle containing an AAV vector genome (which, in turn, comprises the first and second expression cassettes referred to herein). It is meant to include AAV vectors of all serotypes, preferably AAV-1 through AAV-9, more preferably AAV-1, AAV-2, AAV-4, AAV-5, AAV-6, AAV-7, AAV-8, AAV-9, and combinations thereof. AAV vectors resulting from the combination of different serotypes may be referred to as hybrid AAV vectors. In one embodiment, the AAV vector is selected from the group consisting of AAV-1, AAV-2, AAV-4, AAV-5 and AAV-6, and combinations thereof. In one embodiment, the AAV vector is an AAV-5 vector. In one embodiment, the AAV vector is an AAV-5 vector comprising AAV-2 inverted terminal repeats (ITRs). Also contemplated by the present disclosure are AAV vectors comprising variants of the naturally occurring viral proteins, e.g., one or more capsid proteins.

Components to Effectuate the Knockdown of the HPRT1 Gene

In some embodiments, the nucleic acid sequence encoding the agent designed to knockdown the HPRT1 gene is an RNA interference agent (RNAi). In some embodiments, the RNAi agent is an shRNA, a microRNA, or a hybrid thereof.

RNAi

In some embodiments, the expression vector comprises a first nucleic acid sequence encoding an RNAi. RNA interference is an approach for post-transcriptional silencing of gene expression by triggering degradation of homologous transcripts through a complex multistep enzymatic process, e.g. a process involving sequence-specific double-stranded small interfering RNA (siRNA). A simplified model for the RNAi pathway is based on two steps, each involving a ribonuclease enzyme. In the first step, the trigger RNA (either dsRNA or miRNA primary transcript) is processed into a short, interfering RNA (siRNA) by the RNase II enzymes DICER and Drosha. In the second step, siRNAs are loaded into the effector complex RNA-induced silencing complex (RISC). The siRNA is unwound during RISC assembly and the single-stranded RNA hybridizes with mRNA target. It is believed that gene silencing is a result of nucleolytic degradation of the targeted mRNA by the RNase H enzyme Argonaute (Slicer). If the siRNA/mRNA duplex contains mismatches the mRNA is not cleaved. Rather, gene silencing is a result of translational inhibition.

In some embodiments, the RNAi agent is an inhibitory or silencing nucleic acid. As used herein, a “silencing nucleic acid” refers to any polynucleotide which is capable of interacting with a specific sequence to inhibit gene expression. Examples of silencing nucleic acids include RNA duplexes (e.g. siRNA, shRNA), locked nucleic acids (“LNAs”), antisense RNA, DNA polynucleotides which encode sense and/or antisense sequences of the siRNA or shRNA, DNAzymses, or ribozymes. The skilled artisan will appreciate that the inhibition of gene expression need not necessarily be gene expression from a specific enumerated sequence, and may be, for example, gene expression from a sequence controlled by that specific sequence.

Methods for constructing interfering RNAs are known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA may be assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering RNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. When expressed, such an RNA molecule desirably forms a “hairpin” structure and is referred to herein as an “shRNA.” In some embodiments, the loop region is generally between about 2 and about 10 nucleotides in length (by way of example only, see SEQ ID NO: 20). In other embodiments, the loop region is from about 6 to about 9 nucleotides in length. In some embodiments, the sense region and the antisense region are between about 15 and about 30 nucleotides in length. Following post-transcriptional processing, the small hairpin RNA is converted into a siRNA by a cleavage event mediated by the enzyme DICER, which is a member of the RNase III family. The siRNA is then capable of inhibiting the expression of a gene with which it shares homology. Further details are described by see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); and Yu et al. Proc Natl Acad Sci USA 99:6047-6052, (2002), the disclosures of which are hereby incorporated by reference herein in their entireties.

shRNA

In some embodiments, the first nucleic acid sequence encodes a shRNA targeting an HPRT1 gene. In some embodiments, the first nucleic acid sequence encoding a shRNA targeting an HPRT1 gene has a sequence having at least 90% identity to that of SEQ ID NO: 1 (referred to herein as “sh734”). In yet other embodiments the first nucleic acid sequence encoding a shRNA targeting an HPRT1 gene has a sequence having at least 95% identity to that of SEQ ID NO: 1. In further embodiments, the first nucleic acid sequence encoding a shRNA targeting an HPRT1 gene has a sequence having at least 96% identity to that of SEQ ID NO: 1. In further embodiments, the first nucleic acid sequence encoding a shRNA targeting an HPRT1 gene has a sequence having at least 97% identity to that of SEQ ID NO: 1. In even further embodiments, the first nucleic acid sequence encoding a shRNA targeting an HPRT1 gene has a sequence having at least 98% identity to that of SEQ ID NO: 1. In yet further embodiments, the first nucleic acid sequence encoding a shRNA targeting an HPRT1 gene has a sequence having at least 99% identity to that of SEQ ID NO: 1. In other embodiments, the first nucleic acid sequence encoding a shRNA targeting an HPRT1 gene has the nucleic acid sequence of SEQ ID NO: 1.

In some embodiments, the nucleic acid sequence of SEQ ID NO: 1 may be modified. In some embodiments, modifications include: (i) the incorporation of a hsa-miR-22 loop sequence (e.g. CCUGACCCA) (SEQ ID NO: 21); (ii) the addition of a 5′-3′ nucleotide spacer, such as one having two or three nucleotides (e.g. TA); (iii) a 5′ start modification, such as the addition of one or more nucleotides (e.g. G); and/or (iv) the addition of two nucleotides 5′ and 3′ to the stem and loop (e.g. 5′ A and 3′ T). In general, first generation shRNAs are processed into a heterogenous mix of small RNAs, and the accumulation of precursor transcripts has been shown to induce both sequence-dependent and independent nonspecific off-target effects in vivo. Therefore, based on the current understanding of DICER processing and specificity, design rules were applied design that would optimize the structure of the sh734 and DICER processivity and efficiency (see also Gu, S., Y. Zhang, L. Jin, Y. Huang, F. Zhang, M. C. Bassik, M. Kampmann, and M. A. Kay. 2014. Weak base pairing in both seed and 3′ regions reduce RNAi off-targets and enhances si/shRNA designs. Nucleic Acids Research 42:12169-12176).

In some embodiments, the nucleic acid sequence of SEQ ID NO: 1 is modified by adding two nucleotides 5′ and 3′ (e.g., G and C, respectively) to the hairpin loop (SEQ ID NO: 20), thereby lengthening the guide strand from about 19 nucleotides to about 21 nucleotides in length and replacing the loop with the hsa-miR-22 loop CCUGACCCA (SEQ ID NO: 21), to provide the nucleotide sequence of SEQ ID NO: 2. In some embodiments, the nucleic acid sequence encoding a shRNA targeting an HPRT1 gene has a sequence having at least 90% identity to that of SEQ ID NO: 2. In other embodiments, the first nucleic acid sequence encoding a shRNA targeting an HPRT1 gene has a sequence having at least 95% identity to that of SEQ ID NO: 2. In other embodiments, the first nucleic acid sequence encoding a shRNA targeting an HPRT1 gene has a sequence having at least 96% identity to that of SEQ ID NO: 2. In other embodiments, the first nucleic acid sequence encoding a shRNA targeting an HPRT1 gene has a sequence having at least 97% identity to that of SEQ ID NO: 2. In other embodiments, the first nucleic acid sequence encoding a shRNA targeting an HPRT1 gene has a sequence having at least 98% identity to that of SEQ ID NO: 2. In other embodiments, the first nucleic acid sequence encoding a shRNA targeting an HPRT1 gene has a sequence having at least 99% identity to that of SEQ ID NO: 2. In yet other embodiments, the nucleic acid sequence encoding a shRNA targeting an HPRT1 gene has the sequence of SEQ ID NO: 2. It is believed that the shRNA encoded by SEQ ID NO: 2 achieves similar knockdown of HPRT as compared with SEQ ID NO: 1. Likewise, it is believed that a cell rendered substantially HPRT deficient through the knockdown of HPRT via expression of the shRNA encoded by SEQ ID NO: 2 allows for selection using a thioguanine analog (e.g. 6-TG or 6-MP).

In some embodiments, the RNAi present within the vector encodes for a nucleic acid molecule, such as one having at least 90% sequence identity to one of SEQ ID NO: 3 or SEQ ID NO: 4. In some embodiments, the RNAi present within the vector encodes for a nucleic acid molecule, such as one having at least 95% sequence identity to one of SEQ ID NO: 3 or SEQ ID NO: 4. In some embodiments, the nucleic acid molecules having at least 90% sequence identity to one of SEQ ID NO: 3 or SEQ ID NO: 4 are found in the cytoplasm of a host cell.

In some embodiments, the present disclosure provides for a host cell including at least one nucleic acid molecule having at least 90% sequence identity to one of SEQ ID NO: 3 or SEQ ID NO: 4. In some embodiments, the present disclosure provides for a host cell including at least one nucleic acid molecule having at least 95% sequence identity to one of SEQ ID NO: 3 or SEQ ID NO: 4. In some embodiments, the present disclosure provides for a host cell including at least one nucleic acid molecule having one of SEQ ID NO: 3 or SEQ ID NO: 4.

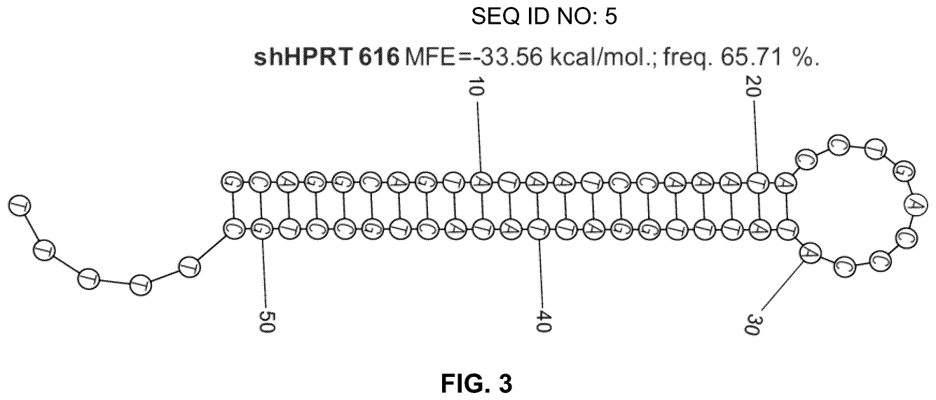

In some embodiments, the first nucleic acid sequence encoding a shRNA targeting an HPRT1 gene has a sequence having at least 80% identity to that of SEQ ID NO: 5 (referred to herein as “shHPRT 616”). In other embodiments, the nucleic acid sequence encoding a shRNA targeting an HPRT1 gene has a sequence having at least 90% identity to that of SEQ ID NO: 5 In yet other embodiments, the nucleic acid sequence encoding a shRNA targeting an HPRT1 gene shRNA has a sequence having at least 95% identity to that of SEQ ID NO: 5. In further embodiments, the nucleic acid sequence encoding a shRNA targeting an HPRT1 gene has a sequence having at least 96% identity to that of SEQ ID NO: 5. In further embodiments, the nucleic acid sequence encoding a shRNA targeting an HPRT1 gene has a sequence having at least 97% identity to that of SEQ ID NO: 5. In even further embodiments, the nucleic acid sequence encoding a shRNA targeting an HPRT1 gene has a sequence having at least 98% identity to that of SEQ ID NO: 5. In yet further embodiments, the nucleic acid sequence encoding a shRNA targeting an HPRT1 gene has a sequence having at least 99% identity to that of SEQ ID NO: 5. In other embodiments, the nucleic acid sequence encoding a shRNA targeting an HPRT1 gene has the sequence of SEQ ID NO: 5 (see also FIG. 3).

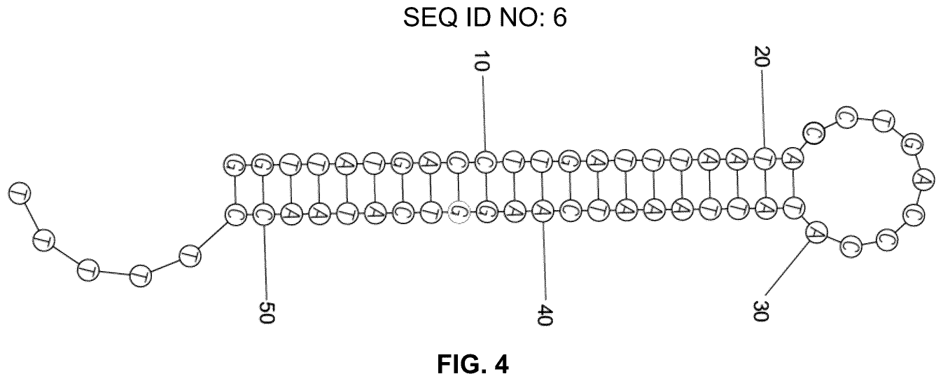

In some embodiments, the first nucleic acid sequence encoding a shRNA targeting an HPRT1 gene has a sequence having at least 80% identity to that of SEQ ID NO: 6 (referred to herein as “shHPRT 211”). In other embodiments, the nucleic acid sequence encoding a shRNA targeting an HPRT1 gene has a sequence having at least 90% identity to that of SEQ ID NO: 6. In yet other embodiments, the nucleic acid sequence encoding a shRNA targeting an HPRT1 gene shRNA has a sequence having at least 95% identity to that of SEQ ID NO: 6. In further embodiments, the nucleic acid sequence encoding a shRNA targeting an HPRT1 gene has a sequence having at least 96% identity to that of SEQ ID NO: 6. In further embodiments, the nucleic acid sequence encoding a shRNA targeting an HPRT1 gene has a sequence having at least 97% identity to that of SEQ ID NO: 6. In even further embodiments, the nucleic acid sequence encoding a shRNA targeting an HPRT1 gene has a sequence having at least 98% identity to that of SEQ ID NO: 6. In yet further embodiments, the nucleic acid sequence encoding a shRNA targeting an HPRT1 gene has a sequence having at least 99% identity to that of SEQ ID NO: 6. In other embodiments, the nucleic acid sequence encoding a shRNA targeting an HPRT1 gene has the sequence of SEQ ID NO: 6 (see also FIG. 4).

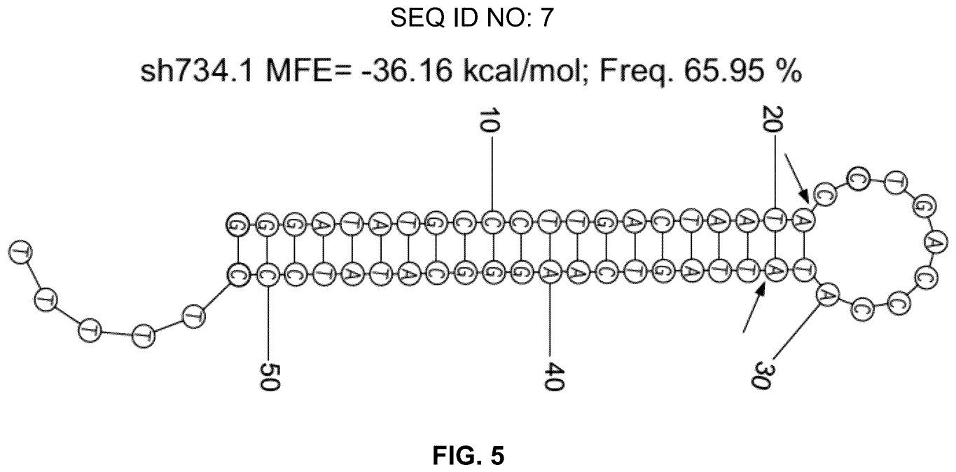

In some embodiments, the nucleic acid sequence encoding a shRNA targeting an HPRT1 gene has a sequence having at least 80% identity to that of SEQ ID NO: 7 (referred to herein as “shHPRT 734.1”) (see also FIG. 5). In other embodiments, the nucleic acid sequence encoding a shRNA targeting an HPRT1 gene has a sequence having at least 90% identity to that of SEQ ID NO: 7. In yet other embodiments, the nucleic acid sequence encoding a shRNA targeting an HPRT1 gene shRNA has a sequence having at least 95% identity to that of SEQ ID NO: 7. In further embodiments, the nucleic acid sequence encoding a shRNA targeting an HPRT1 gene has a sequence having at least 96% identity to that of SEQ ID NO: 7. In further embodiments, the nucleic acid sequence encoding a shRNA targeting an HPRT 1 gene has a sequence having at least 97% identity to that of SEQ ID NO: 7. In even further embodiments, the nucleic acid sequence encoding a shRNA targeting an HPRT1 gene has a sequence having at least 98% identity to that of SEQ ID NO: 7. In yet further embodiments, the nucleic acid sequence encoding a shRNA targeting an HPRT1 gene has a sequence having at least 99% identity to that of SEQ ID NO: 7. In other embodiments, the nucleic acid sequence encoding a shRNA targeting an HPRT1 gene has the sequence of SEQ ID NO: 7 (see also FIG. 5).

MicroRNA

MicroRNAs (miRs) are a group of non-coding RNAs which post-transcriptionally regulate the expression of their target genes. It is believed that these single stranded molecules form a miRNA-mediated silencing complex (miRISC) complex with other proteins which bind to the 3′ untranslated region (UTR) of their target mRNAs so as to prevent their translation in the cytoplasm.

In some embodiments, shRNA sequences are embedded into micro-RNA secondary structures (“micro-RNA based shRNA”). In some embodiments, shRNA nucleic acid sequences targeting HPRT are embedded within micro-RNA secondary structures. In some embodiments, the micro-RNA based shRNAs target coding sequences within HPRT to achieve knockdown of HPRT expression, which is believed to be equivalent to the utilization of shRNA targeting HPRT without attendant pathway saturation and cellular toxicity or off-target effects. In some embodiments, the micro-RNA based shRNA is a de novo artificial microRNA shRNA. The production of such de novo micro-RNA based shRNAs are described by Fang, W. & Bartel, David P. The Menu of Features that Define Primary MicroRNAs and Enable De Novo Design of MicroRNA Genes. Molecular Cell 60, 131-145, the disclosure of which is hereby incorporated by reference herein in its entirety.

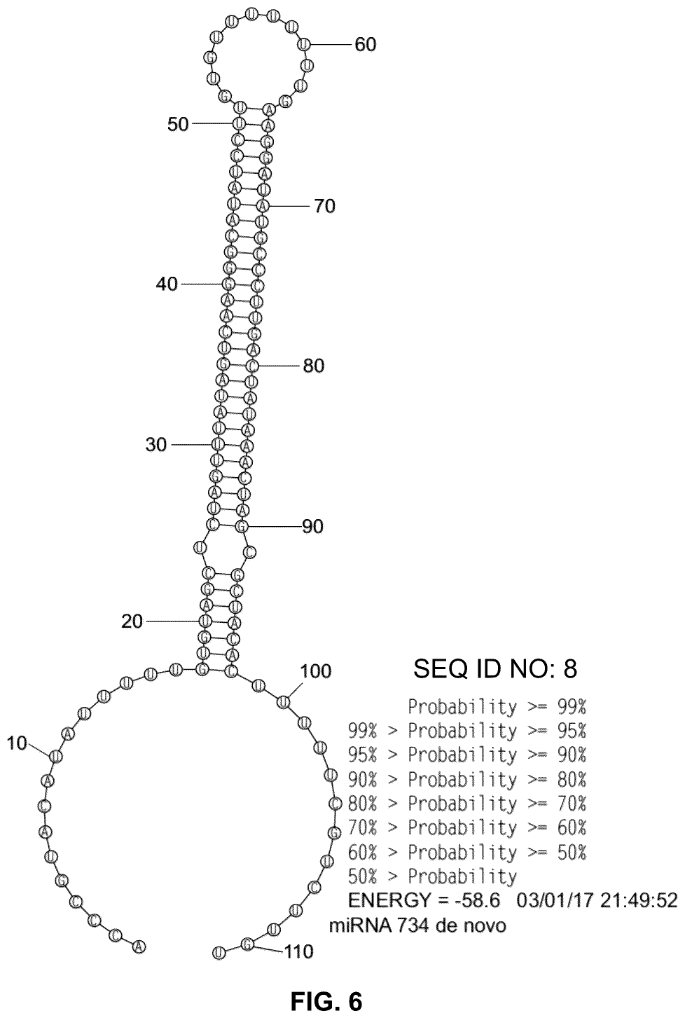

In some embodiments, the micro-RNA based shRNA has a nucleic acid sequence having at least 80% identity to that of SEQ ID NO: 8. In some embodiments, the micro-RNA based shRNA has a nucleic acid sequence having at least 90% identity to that of SEQ ID NO: 8. In some embodiments, the micro-RNA based shRNA has a nucleic acid sequence having at least 95% identity to that of SEQ ID NO: 8. In some embodiments, the micro-RNA based shRNA has a nucleic acid sequence having at least 96% identity to that of SEQ ID NO: 8. In some embodiments, the micro-RNA based shRNA has a nucleic acid sequence having at least 97% identity to that of SEQ ID NO: 8. In some embodiments, the micro-RNA based shRNA has a nucleic acid sequence having at least 98% identity to that of SEQ ID NO: 8. In some embodiments, the micro-RNA based shRNA has a nucleic acid sequence having at least 99% identity to that of SEQ ID NO: 8. In some embodiments, the micro-RNA based shRNA has the sequence of SEQ ID NO: 8 (“miRNA734-Denovo”) (see also FIG. 6). The RNA form of SEQ ID NO: 8 has SEQ ID NO: 22.

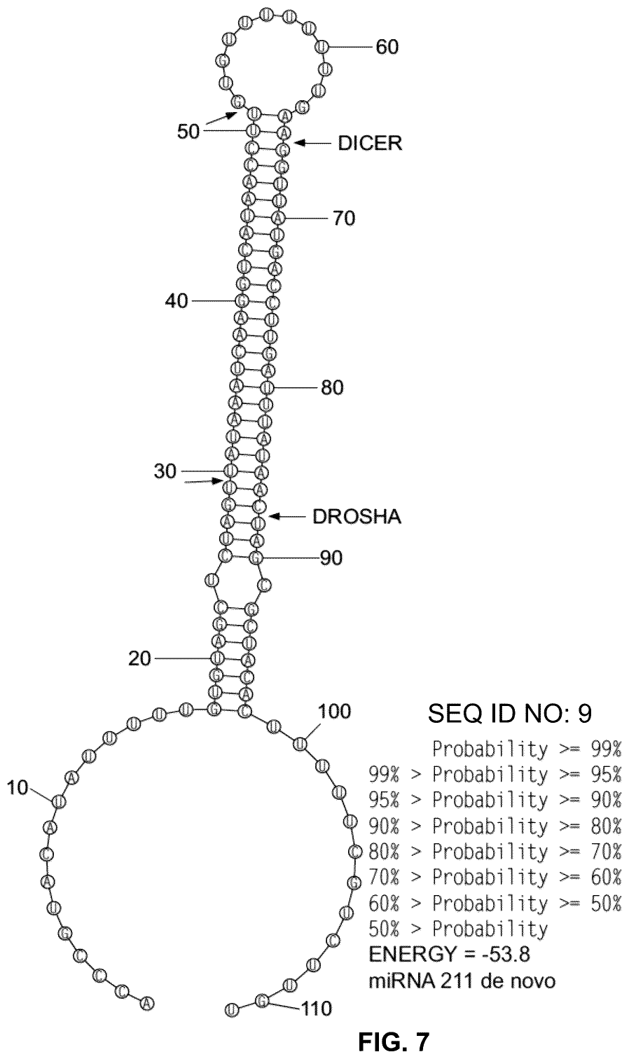

In some embodiments, the micro-RNA based shRNA has a sequence having at least 80% identity to that of SEQ ID NO: 9. In some embodiments, the micro-RNA based shRNA has a nucleic acid sequence having at least 90% identity to that of SEQ ID NO: 9. In some embodiments, the micro-RNA based shRNA has a sequence having at least 95% identity to that of SEQ ID NO: 9. In some embodiments, the micro-RNA based shRNA has a sequence having at least 96% identity to that of SEQ ID NO: 9. In some embodiments, the micro-RNA based shRNA has a sequence having at least 97% identity to that of SEQ ID NO: 9. In some embodiments, the micro-RNA based shRNA has a sequence having at least 98% identity to that of SEQ ID NO: 9. In some embodiments, the micro-RNA based shRNA has a sequence having at least 99% identity to that of SEQ ID NO: 9. In some embodiments, the micro-RNA based shRNA has the nucleic acid sequence of SEQ ID NO: 9 (“miRNA211-Denovo”) (see also FIG. 7). The RNA form of SEQ ID NO: 9 has SEQ ID NO: 23.

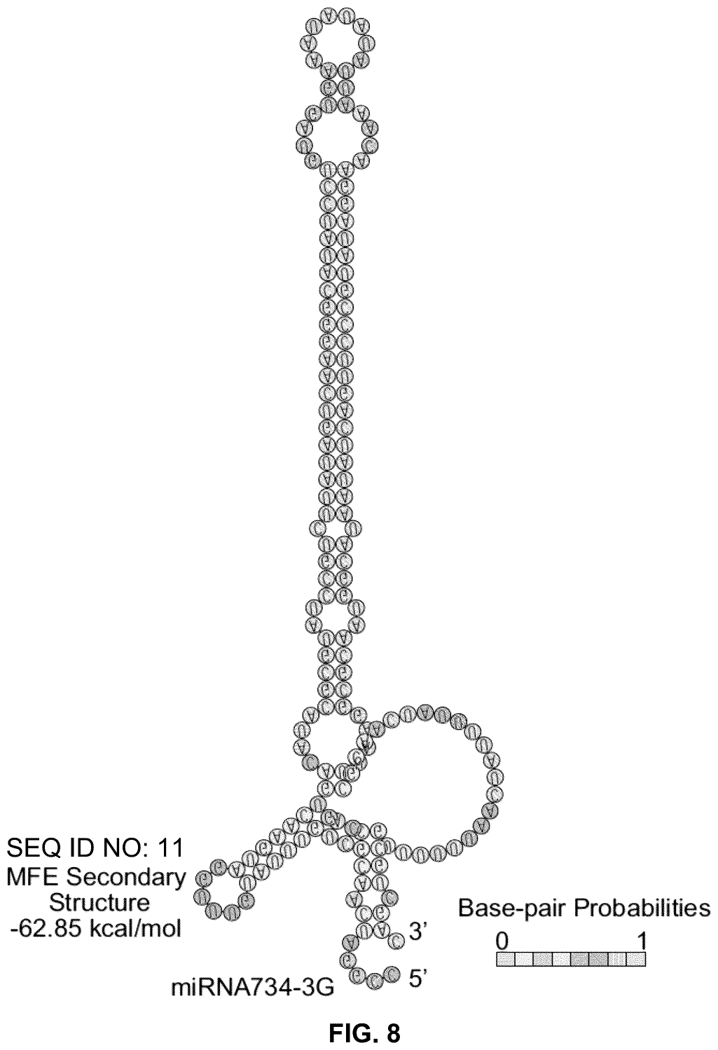

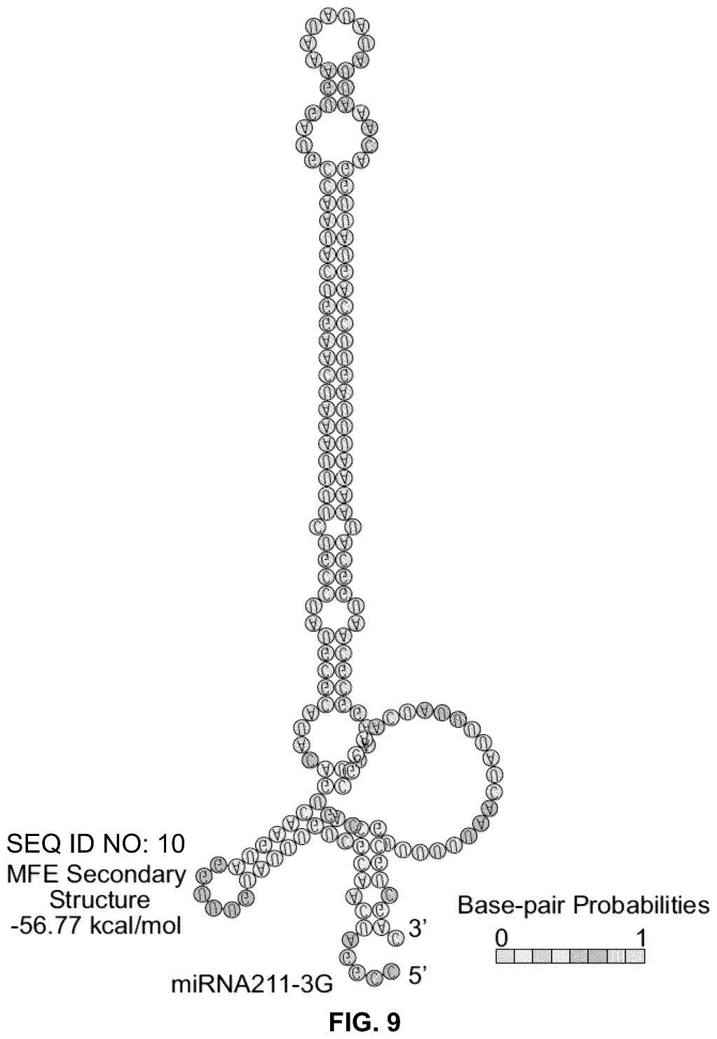

In other embodiments, the micro-RNA based shRNA is a third generation miRNA scaffold modified miRNA 16-2 (hereinafter “miRNA-3G”) (see, e.g., FIGS. 8 and 9). The synthesis of such miRNA-3G molecules is described by Watanabe, C., Cuellar, T. L. & Haley, B. “Quantitative evaluation of first, second, and third generation hairpin systems reveals the limit of mammalian vector-based RNAi,” RNA Biology 13, 25-33 (2016), the disclosure of which is hereby incorporated by reference herein in its entirety.

In some embodiments, the miRNA-3G has a nucleic acid sequence having at least 80% identity to that of SEQ ID NO: 10. In some embodiments, the miRNA-3G has a nucleic acid sequence having at least 90% identity to that of SEQ ID NO: 10. In some embodiments, the miRNA-3G has a sequence having at least 95% identity to that of SEQ ID NO: 10. In some embodiments, the miRNA-3G has a sequence having at least 96% identity to that of SEQ ID NO: 10. In some embodiments, the miRNA-3G has a sequence having at least 97% identity to that of SEQ ID NO: 10. In some embodiments, the miRNA-3G has a sequence having at least 98% identity to that of SEQ ID NO: 10. In some embodiments, the miRNA-3G has a sequence having at least 99% identity to that of SEQ ID NO: 10. In some embodiments, the miRNA-3G has the nucleic acid sequence of SEQ ID NO: 10 (“miRNA211-3G”) (see also FIG. 9).

In some embodiments, the miRNA-3G has a nucleic acid sequence having at least 80% identity to that of SEQ ID NO: 11. In some embodiments, the miRNA-3G has a nucleic acid sequence having at least 90% identity to that of SEQ ID NO: 11. In some embodiments, the miRNA-3G has a nucleic acid sequence having at least 95% identity to that of SEQ ID NO: 11. In some embodiments, the miRNA-3G has a nucleic acid sequence having at least 96% identity to that of SEQ ID NO: 11. In some embodiments, the miRNA-3G has a nucleic acid sequence having at least 97% identity to that of SEQ ID NO: 11. In some embodiments, the miRNA-3G has a nucleic acid sequence having at least 98% identity to that of SEQ ID NO: 11. In some embodiments, the miRNA-3G has a nucleic acid sequence having at least 99% identity to that of SEQ ID NO: 11. In other embodiments, the miRNA-3G has the nucleic acid sequence of SEQ ID NO: 11 (“miRNA734-3G”) (see also FIG. 8).

In some embodiments, the sh734 shRNA is adapted to mimic a miRNA-451 (see SEQ ID NO: 24) structure with a 17 nucleotide base pair stem and a 4-nucleotide loop (miR-451 regulates the drug-transporter protein P-glycoprotein). Notably, this structure does not require processing by DICER. It is believed that the pre-451 mRNA structure is cleaved by Ago2 and subsequently by poly(A)-specific ribonuclease (PARN) to generate the mature miRNA-451 structural mimic. It is believed that Ago-shRNA mimics of the structure of the endogenous miR-451 and may have the advantage of being DICER independent. This is believed to restrict off target effects of passenger loading, with variable 3′-5′ exonucleolytic activity (23-26 nt mature) (see Herrera-Carrillo, E., and B. Berkhout. 2017. DICER-independent processing of small RNA duplexes: mechanistic insights and applications. Nucleic Acids Res. 45:10369-10379). It is also believed that there exist advantages of utilizing alternate DICER independent processing of shRNAs, including efficient reduced off-target effects of single RNAi-active guide, no saturation of cellular RNAi DICER machinery, and shorter RNA duplexes are less likely to trigger innate RIG-I response.

Alternatives to RNAi

As an alternative to the incorporation of a RNAi, in some embodiments, the expression vectors may include a nucleic acid sequence which encodes antisense oligonucleotides that bind sites in messenger RNA (mRNA). Antisense oligonucleotides of the present disclosure specifically hybridize with a nucleic acid encoding a protein and interfere with transcription or translation of the protein. In some embodiments, an antisense oligonucleotide targets DNA and interferes with its replication and/or transcription. In other embodiments, an antisense oligonucleotide specifically hybridizes with RNA, including pre-mRNA (i.e. precursor mRNA which is an immature single strand of mRNA), and mRNA. Such antisense oligonucleotides may affect, for example, translocation of the RNA to the site of protein translation, translation of protein from the RNA, splicing of the RNA to yield one or more mRNA species, and catalytic activity that may be engaged in or facilitated by the RNA. The overall effect of such interference is to modulate, decrease, or inhibit target protein expression.

In some embodiments, the expression vectors incorporate a nucleic acid sequence encoding for an exon skipping agent or exon skipping transgene. As used herein, the phrase “exon skipping transgene” or “exon skipping agent” refers to any nucleic acid that encodes an antisense oligonucleotide that can generate exon skipping. “Exon skipping” refers to an exon that is skipped and removed at the pre-mRNA level during protein production. It is believed that antisense oligonucleotides may interfere with splice sites or regulatory elements within an exon. This can lead to truncated, partially functional, protein despite the presence of a genetic mutation. Generally, the antisense oligonucleotides may be mutation-specific and bind to a mutation site in the pre-messenger RNA to induce exon skipping.

Exon skipping transgenes encode agents that can result in exon skipping, and such agents are antisense oligonucleotides. The antisense oligonucleotides may interfere with splice sites or regulatory elements within an exon to lead to truncated, partially functional, protein despite the presence of a genetic mutation. Additionally, the antisense oligonucleotides may be mutation-specific and bind to a mutation site in the pre-messenger RNA to induce exon skipping. Antisense oligonucleotides for exon skipping are known in the art and are generally referred to as AONs. Such AONs include small nuclear RNAs (“snRNAs”), which are a class of small RNA molecules that are confined to the nucleus and which are involved in splicing or other RNA processing reactions. Examples of antisense oligonucleotides, methods of designing them, and related production methods are disclosed, for example, in U.S. Publication Nos. 20150225718, 20150152415, 20150140639, 20150057330, 20150045415, 20140350076, 20140350067, and 20140329762, the disclosures of which are hereby incorporated by reference herein in their entireties.

In some embodiments, the expression vectors of the present disclosure include a nucleic acid which encodes an exon skipping agent which results in exon skipping during the expression of HPRT or which causes an HPRT duplication mutation (e.g. a duplication mutation in Exon 4) (see Baba S, et al. Novel mutation in HPRT1 causing a splicing error with multiple variations. Nucleosides Nucleotides Nucleic Acids. 2017 Jan. 2; 36(1):1-6, the disclosure of which is hereby incorporated by reference herein in its entirety.

In some embodiments, HPRT may be replaced with a modified mutated sequence by spliceosome trans-splicing, thus facilitating of HPRT. In some embodiments, this (1) requires a mutated coding region to replace the coding sequence in a target RNA, (2) a 5′ or 3′ splice site, and/or (3) a binding domain, i.e., antisense oligonucleotide sequence, which is complementary to the target HPRT RNA. In some embodiments, all three components are required.

Promoters

Various promoters may be used to drive expression of each of the nucleic acid sequences incorporated within the disclosed expression vectors. For example, a first nucleic acid sequence encoding an RNAi (e.g. an anti-HPRT shRNA) may be expressed from a first promoter selected from one of a Pol III promoter or a Pol II promoter. Likewise, and by way of another example, a first nucleic acid sequence encoding a micro-RNA based shRNA to downregulate HPRT may be expressed from a first promoter selected from one of a Pol III promoter or a Pol II promoter. In some embodiments, the promoters may be constitutive promoters or inducible promoters as known to those of ordinary skill in the art. In some embodiments, the promoter includes at least a portion of an HIV LTR (e.g. TAR).

Non-limiting examples of suitable promoters include, but are not limited to, RNA polymerase I (pol I), polymerase II (pol II), or polymerase III (pol III) promoters. By “RNA polymerase III promoter” or “RNA pol III promoter” or “polymerase III promoter” or “pol III promoter” it is meant any invertebrate, vertebrate, or mammalian promoter, e.g., human, murine, porcine, bovine, primate, simian, etc. that, in its native context in a cell, associates or interacts with RNA polymerase III to transcribe its operably linked gene, or any variant thereof, natural or engineered, that will interact in a selected host cell with an RNA polymerase III to transcribe an operably linked nucleic acid sequence. RNA pol III promoters suitable for use in the expression vectors of the disclosure include, but are not limited, to human U6, mouse U6, and human H1 others.

Examples of pol II promoters include, but are not limited to, Efl alpha, CMV, and ubiquitin. Other specific pol II promoters include, but are not limited to, ankyrin promoter (Sabatino D E, et al., Proc Natl Acad Sd USA. (24):13294-9 (2000)), spectrin promoter (Gallagher P G, et al., J Biol Chem. 274(10):6062-73, (2000)), transferrin receptor promoter (Marziali G, et al., Oncogene. 21(52):7933-44, (2002)), band 3/anion transporter promoter (Frazar T F, et al., MoL Cell Biol (14):4753-63, (2003)), band 4.1 promoter (Harrison P R, et al., Exp Cell Res. 155(2):321-44, (1984)), BcI-X1 promoter (Tian C, et al., Blood 15; 101(6):2235-42 (2003)), EKLF promoter (Xue L, et al., Blood. 103(11):4078-83 (2004)). Epub 2004 Feb. 5), ADD2 promoter (Yenerel M N, et al., Exp Hematol. 33(7):758-66 (2005)), DYRK3 promoter (Zhang D, et al., Genomics 85(1): 117-30 (2005)), SOCS promoter (Sarna M K, et al., Oncogene 22(21):3221-30 (2003)), LAF promoter (To M D, et al., bit J Cancer 1; 115(4):568-74, (2005)), PSMA promoter (Zeng H, et al., J Androl (2):215-21, (2005)), PSA promoter (Li H W, et al., Biochem Biophys Res Commun 334(4): 1287-91, (2005)), Probasin promoter (Zhang J, et al., 145(1):134-48, (2004)). Epub 2003 Sep. 18), ELAM-I promoter/E-Selectin (Walton T, et al., Anticancer Res. 18(3A):1357-60, (1998)), Synapsin promoter (Thiel G, et al., Proc Natl Acad Sd USA., 88(8):3431-5(1988)), Willebrand factor promoter (Jahroudi N, Lynch D C. Mol Cell-5zo/.14(2):999-1008, (1994)), FLT1 (Nicklin S A, et al., Hypertension 38(1):65-70, (2001)), Tau promoter (Sadot E, et al., J Mol Biol. 256(5):805-12, (1996)), Tyrosinase promoter (Lillehammer T, et al., Cancer Gene Ther. (2005)), pander promoter (Burkhardt B R, et al., Biochim Biophys Acta. (2005)), neuron-specific enolase promoter (Levy Y S, et al., J Mol Neurosci.21(2):121-32, (2003)), hTERT promoter (Ito H, et al., Hum Gene Ther 16(6):685-98, (2005)), HRE responsive element (Chadderton N, et al., Int J Radiat Oncol Biol Phys.62(1):2U-22, (2005)), lck promoter (Zhang D J, et al., J Immunol. 174(11):6725-31, (2005)), MHCII promoter (De Geest B R, et al., Blood. 101(7):2551-6, (2003), Epub 2002 Nov. 21), and CD1 Ic promoter (Lopez-Rodriguez C, et al., J Biol Chem. 272(46):29120-6 (1997)).

In some embodiments, the promoter driving expression of the agent designed to knockdown HPRT is an RNA pol III promoter. In some embodiments, the promoter driving expression of the agent designed to knockdown HPRT is a 7sk promoter (e.g. a 7SK human 7S K RNA promoter). In some embodiments, the 7sk promoter has the nucleic acid sequence provided by ACCESSION AY578685 (Homo sapiens cell-line HEK-293 7SK RNA promoter region, complete sequence, ACCESSION AY578685).

In some embodiments, the 7sk promoter has a sequence having at least 90% identity to that of SEQ ID NO: 14. In some embodiments, the 7sk promoter has a nucleic acid sequence having at least 95% identity to that of SEQ ID NOS: 14. In some embodiments, the 7sk promoter has a nucleic acid sequence having at least 96% identity to that of SEQ ID NOS: 14. In some embodiments, the 7sk promoter has a nucleic acid sequence having at least 97% identity to that of SEQ ID NOS: 14. In some embodiments, the 7sk promoter has a nucleic acid sequence having at least 98% identity to that of SEQ ID NOS: 14. In some embodiments, the 7sk promoter has a nucleic acid sequence having at least 99% identity to that of SEQ ID NOS: 14. In some embodiments, the 7sk promoter has the nucleic acid sequence set forth in SEQ ID NOS: 14.

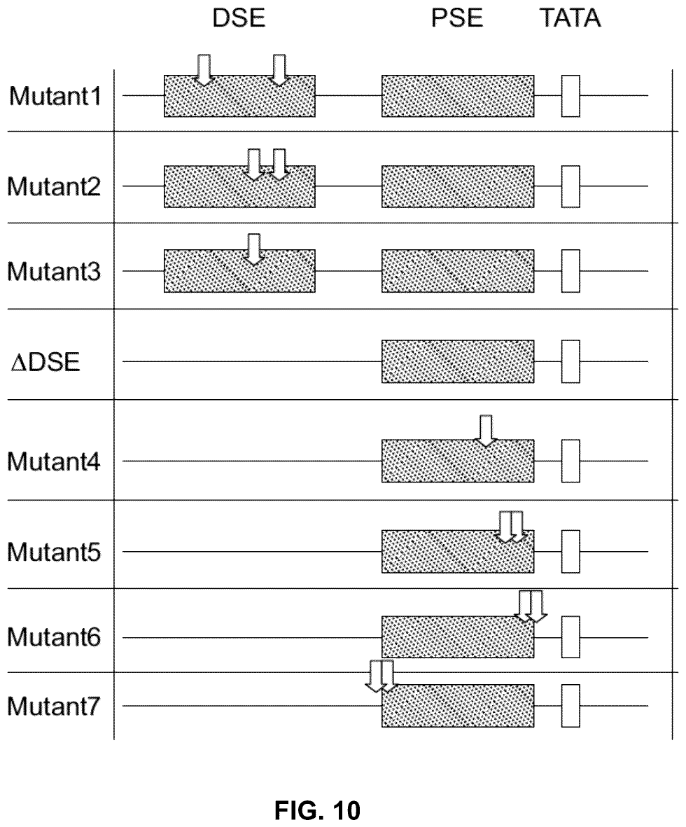

In some embodiments, the 7sk promoter utilized comprises at least one mutation and/or deletion in its nucleic acid sequence in comparison to the 7sk promoter. Suitable 7sk promoter mutations are described in Boyd, D. C., Turner, P. C., Watkins, N.J., Gerster, T. & Murphy, S. Functional Redundancy of Promoter Elements Ensures Efficient Transcription of the Human 7SK Gene in vivo. Journal of Molecular Biology 253, 677-690 (1995), the disclosure of which is hereby incorporated by reference herein in its entirety. In some embodiments, functional mutations or deletions in the 7sk promoter are made in cis-regulatory elements to regulate expression levels of the promoter-driven transgene, including sh734. The mutations described are used to establish the correlation between sh734 expression levels driven by the Pol III promoter and to introduce functionality to undergo stable selection in the presence of 6-TG and/or 6-MP therapy and long-term stability and safety. The location of 7sk promoter mutations are depicted in FIG. 10.

In some embodiments, the 7sk promoter has a nucleic acid sequence having at least 95% identity to that of SEQ ID NOS: 15. In some embodiments, the 7sk promoter has a nucleic acid sequence having at least 96% identity to that of SEQ ID NOS: 15. In some embodiments, the 7sk promoter has a nucleic acid sequence having at least 97% identity to that of SEQ ID NOS: 15. In some embodiments, the 7sk promoter has a nucleic acid sequence having at least 98% identity to that of SEQ ID NOS: 15. In some embodiments, the 7sk promoter has a nucleic acid sequence having at least 99% identity to that of SEQ ID NOS: 15. In some embodiments, the 7sk promoter has the nucleic acid sequence set forth in SEQ ID NOS: 15.

In other embodiments, the promoter is a tissue specific promoter. Several non-limiting examples of tissue specific promoters that may be used include lck (see, for example, Garvin et al., Mol. Cell Biol. 8:3058-3064, (1988)) and Takadera et al., Mol. Cell Biol. 9:2173-2180, (1989)), myogenin (Yee et al., Genes and Development 7:1277-1289 (1993), and thyl (Gundersen et al., Gene 113:207-214, (1992)).

Non-limiting examples of combinations of nucleic acid sequences operably linked to a promoter are set forth in the table which follows:

| | | | shRNA |
|---|---|---|---|
| No. | Promoter Type | Promoter | SEQ ID NO: |
| 1 | Pol III | 7sk | 1 |
| 2 | Pol III | Mutant 7sk with a single mutation | 1 |
| 3 | Pol III | Mutant 7sk with two mutations | 1 |
| 4 | Pol III | Mutant 7sk with three mutations | 1 |
| 5 | Pol III | H1 | 1 |
| 6 | Pol II | EF1a | 5 |
| 7 | Pol II | EF1a | 6 |
| 8 | Pol II | EF1a | 7 |

Production of Vectors

In some embodiments, an expression cassette, such as one including a nucleic acid sequence adapted to knockdown HPRT, is inserted into an expression vector, such as a lentiviral expression vector, to provide a vector having at least one transgene for expression. In some embodiments, the lentiviral expression vector may be selected from the group consisting of pTL20c, pTL20d, FG, pRRL, pCL20, pLKO.1 puro, pLKO.1, pLKO.3G, Tet-pLKO-puro, pSico, pLJM1-EGFP, FUGW, pLVTHM, pLVUT-tTR-KRAB, pLL3.7, pLB, pWPXL, pWPI, EF.CMV.RFP, pLenti CMV Puro DEST, pLenti-puro, pLOVE, pULTRA, pLJM1-EGFP, pLX301, pInducer20, pHIV-EGFP, Tet-pLKO-neo, pLV-mCherry, pCW57.1, pLionII, pSLIK-Hygro, and pInducer10-mir-RUP-PheS. In other embodiments, the lentiviral expression vector may be selected from AnkT9W vector, a T9Ank2W vector, a TNS9 vector, a lentiglobin HPV569 vector, a lentiglobin BB305 vector, a BG-1 vector, a BGM-1 vector, a d432βAγ vector, a mLAβΔγV5 vector, a GLOBE vector, a G-GLOBE vector, a βAS3-FB vector, a V5 vector, a V5m3 vector, a V5m3-400 vector, a G9 vector, and a BCL11 A shmir vector. In some embodiments, the lentiviral expression vector may be selected from the group consisting of pTL20c, pTL20d, FG, pRRL and pCL20. In still other embodiments, the lentiviral expression vector is pTL20c.

In some embodiments, the expression cassette comprises a nucleic acid sequence having at least 95% sequence identity to that of SEQ ID NO: 13. In other embodiments, the expression cassette comprises a nucleic acid sequence having at least 96% sequence identity to that of SEQ ID NO: 13. In other embodiments, the expression cassette comprises a nucleic acid sequence having at least 97% sequence identity to that of SEQ ID NO: 13. In other embodiments, the expression cassette comprises a nucleic acid sequence having at least 98% sequence identity to that of SEQ ID NO: 13. In yet other embodiments, the expression cassette comprises a nucleic acid sequence having at least 99% sequence identity to that of SEQ ID NO: 13. In further embodiments, the expression cassette has the nucleic acid sequence of SEQ ID NO: 13.

In some embodiments, the plasmid has a nucleic acid sequence having at least 90% sequence identity to SEQ ID NO: 17. In some embodiments, the plasmid has a nucleic acid sequence having at least 95% sequence identity to SEQ ID NO: 17. In some embodiments, the plasmid has a nucleic acid sequence having at least 96% sequence identity to SEQ ID NO: 17. In some embodiments, the plasmid has a nucleic acid sequence having at least 97% sequence identity to SEQ ID NO: 17. In some embodiments, the plasmid has a nucleic acid sequence having at least 98% sequence identity to SEQ ID NO: 17. In some embodiments, the plasmid has a nucleic acid sequence having at least 98% sequence identity to SEQ ID NO: 17. In some embodiments, the plasmid has a nucleic acid sequence of SEQ ID NO: 17.

In some embodiments, the plasmid includes a TL20 viral backbone having a nucleic acid sequence having at least 90% sequence identity to that of SEQ ID NO: 16. In some embodiments, the plasmid includes a TL20 viral backbone having a nucleic acid sequence having at least 95% sequence identity to that of SEQ ID NO: 16. In some embodiments, the plasmid includes a TL20 viral backbone having a nucleic acid sequence having at least 96% sequence identity to that of SEQ ID NO: 16. In some embodiments, the plasmid includes a TL20 viral backbone having a nucleic acid sequence having at least 97% sequence identity to that of SEQ ID NO: 16. In some embodiments, the plasmid includes a TL20 viral backbone having a nucleic acid sequence having at least 98% sequence identity to that of SEQ ID NO: 16. In some embodiments, the plasmid includes a TL20 viral backbone having a nucleic acid sequence having at least 99% sequence identity to that of SEQ ID NO: 16. In some embodiments, the plasmid includes a TL20 viral backbone having a nucleic acid sequence of SEQ ID NO: 16.

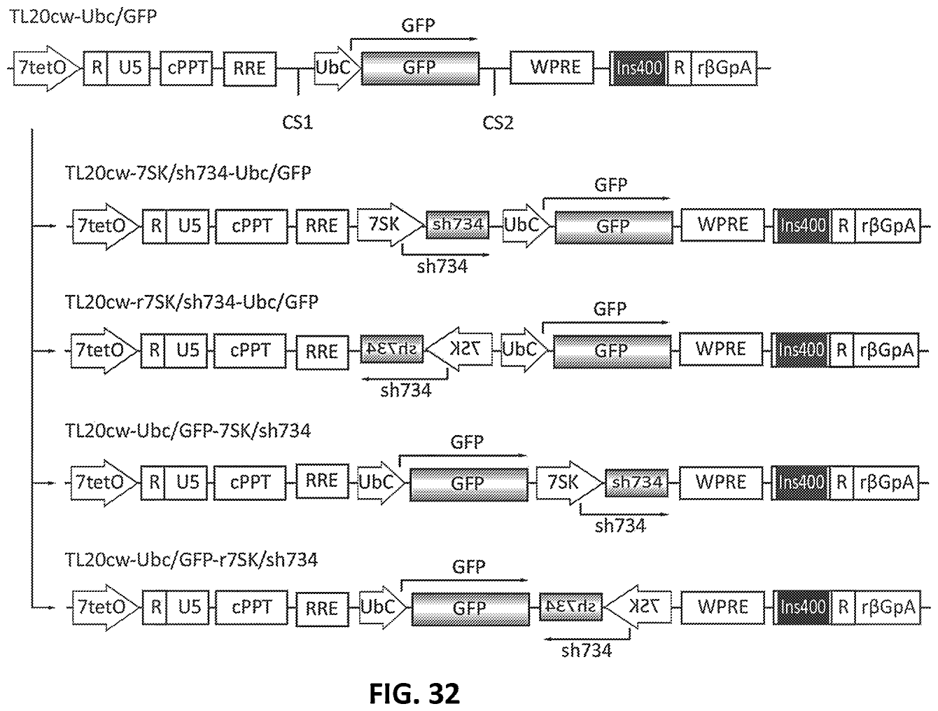

In one or more embodiments, the first nucleic acid sequence encoding a shRNA targeting an HPRT1 gene may be inserted into an expression vector in different orientations relative to other vector elements (compare, for example, the orientations of the 7sk promoter between FIG. 32). For example, the 7sk driven sh734 element may be oriented in the same direction or in opposite directions as compared with a transgene, like the UbC driven GFP described in the Examples. In still other embodiments, the first nucleic acid sequence encoding a shRNA targeting an HPRT1 gene may be inserted into an expression vector in different locations, that is, either upstream or downstream of other vector elements, e.g. upstream or downstream of the UbC driven GFP. It is believed that the different locations and/or orientations of the 7sk expression cassette relative to other vector elements may enhance expression of sh734.

In some embodiments, the 7sk/sh734 expression cassette is located upstream relative to other vector elements, such as the UbC driven GFP.

In some embodiments, the 7sk/sh734 expression cassette is located downstream relative to other vector elements, such as the UbC driven GFP.

In some embodiments, the 7sk/sh734 expression cassette and the other vector elements, such as the UbC driven GFP, are oriented in the same direction.

In some embodiments, the 7sk/sh734 expression cassette and the other vector elements, such as the UbC driven GFP, are oriented in opposing directions.

In some embodiments, the 7sk/sh734 expression cassette is oriented in a forward direction relative the other vector elements, such as the UbC driven GFP.

In some embodiments, the 7sk/sh734 expression cassette is oriented in a reverse direction relative the other vector elements, such as the UbC driven GFP.

In some embodiments, the 7sk/sh734 expression cassette is located upstream and oriented in a forward direction relative the other vector elements, such as the UbC driven GFP.

In some embodiments, the 7sk/sh734 expression cassette is located upstream and oriented in a reverse direction relative the other vector elements, such as the UbC driven GFP.

In some embodiments, the 7sk/sh734 expression cassette is located downstream and oriented in a forward direction relative the other vector elements, such as the UbC driven GFP.

In some embodiments, the 7sk/sh734 expression cassette is located downstream and oriented in a reverse direction relative the other vector elements, such as the UbC driven GFP.

Physical and Non-Viral Delivery of Agents to Downregulate HPRT or to Knockout HPRT

In some embodiments, agents designed to knockdown or knockout the HPRT1 gene (including expression constructs including an RNAi) may be delivered through physical methods. In some embodiments, the physical method is selected from microinjection and electroporation. Electroporation is technique in which an electrical field is applied to cells in order to increase the permeability of the cell membrane, allowing chemicals, small molecules, proteins, nucleic acids, etc. to be introduced into the cell. Microinjection is a technique for chemicals, small molecules, proteins, nucleic acids, etc. to be introduced into a single cell by insertion of a micropipette into the cell of interest. Microinjection provides controlled delivery dosage and targeted delivery to subcellular location(s).

In some embodiments, (i) an endonuclease, and (ii) a guide RNA molecule targeting a sequence within one of Exon 3 or Exon 8 of the HPRT 1 gene are introduced to lymphocytes by electroporation or my microinjection. In some embodiments, the guide RNA molecule has at least 90% sequence identity to any one of SEQ ID NOS: 40-44 and 46-56. In some embodiments, the guide RNA molecule has at least 91% sequence identity to any one of SEQ ID NOS: 40-44 and 46-56. In some embodiments, the guide RNA molecule has at least 92% sequence identity to any one of SEQ ID NOS: 40-44 and 46-56. In some embodiments, the guide RNA molecule has at least 93% sequence identity to any one of SEQ ID NOS: 40-44 and 46-56. In some embodiments, the guide RNA molecule has at least 94% sequence identity to any one of SEQ ID NOS: 40-44 and 46-56. In some embodiments, the guide RNA molecule has at least 95% sequence identity to any one of SEQ ID NOS: 40-44 and 46-56. In some embodiments, the guide RNA molecule has at least 96% sequence identity to any one of SEQ ID NOS: 40-44 and 46-56. In some embodiments, the guide RNA molecule has at least 97% sequence identity to any one of SEQ ID NOS: 40-44 and 46-56. In some embodiments, the guide RNA molecule has at least 98% sequence identity to any one of SEQ ID NOS: 40-44 and 46-56. In some embodiments, the guide RNA molecule has at least 99% sequence identity to any one of SEQ ID NOS: 40-44 and 46-56. In some embodiments, the guide RNA molecule comprises the sequence of any one of SEQ ID NOS: 40-44 and 46-56.

In some embodiments, (i) an endonuclease, and (ii) a guide RNA molecule targeting a sequence within one of Exon 2 of the HPRT 1 gene are introduced to lymphocytes by electroporation or my microinjection. In some embodiments, the guide RNA molecule has at least 90% sequence identity to any one of SEQ ID NOS: 45 and 57-61. In some embodiments, the guide RNA molecule has at least 91% sequence identity to any one of SEQ ID NOS: 45 and 57-61. In some embodiments, the guide RNA molecule has at least 92% sequence identity to any one of SEQ ID NOS: 45 and 57-61. In some embodiments, the guide RNA molecule has at least 93% sequence identity to any one of SEQ ID NOS: 45 and 57-61. In some embodiments, the guide RNA molecule has at least 94% sequence identity to any one of SEQ ID NOS: 45 and 57-61. In some embodiments, the guide RNA molecule has at least 95% sequence identity to any one of SEQ ID NOS: 45 and 57-61. In some embodiments, the guide RNA molecule has at least 96% sequence identity to any one of SEQ ID NOS: 45 and 57-61. In some embodiments, the guide RNA molecule has at least 97% sequence identity to any one of SEQ ID NOS: 45 and 57-61. In some embodiments, the guide RNA molecule has at least 98% sequence identity t to any one of SEQ ID NOS: 45 and 57-61. In some embodiments, the guide RNA molecule has at least 99% sequence identity to any one of SEQ ID NOS: 45 and 57-61. In some embodiments, the guide RNA comprises the sequence of any one of SEQ ID NOS: 45 and 57-61.

In some embodiments, the endonuclease delivered by one or more physical methods comprises a Cas protein. In some embodiments, the Cas protein comprises a Cas9 protein. In some embodiments, the Cas protein comprises a Cas12 protein. In some embodiments, the Cas12 protein is a Cas12a protein. In some embodiments, the Cas12 protein is a Cas12b protein.

In some embodiments, agents designed to knockdown or knockout the HPRT1 gene (including expression constructs including an RNAi) may be delivered through a non-viral delivery vehicle. In some embodiments, the non-viral delivery vehicle is a nanocapsule or other non-viral delivery vehicle. In some embodiments, (i) an endonuclease, and (ii) a guide RNA molecule targeting a sequence within one of Exon 3 or Exon 8 of the HPRT 1 gene are introduced to lymphocytes via a non-viral delivery vehicle, such as a nanocapsule. In some embodiments, the guide RNA molecule has at least 90% sequence identity to any one of SEQ ID NOS: 40-44 and 46-56. In some embodiments, the guide RNA molecule has at least 95% sequence identity to any one of SEQ ID NOS: 40-44 and 46-56. In some embodiments, the guide RNA molecule has at least 97% sequence identity to any one of SEQ ID NOS: 40-44 and 46-56. In some embodiments, the guide RNA molecule has at least 98% sequence identity to any one of SEQ ID NOS: 40-44 and 46-56. In some embodiments, the guide RNA molecule has at least 99% sequence identity to any one of SEQ ID NOS: 40-44 and 46-56.

In some embodiments, (i) an endonuclease, and (ii) a guide RNA molecule targeting a sequence within one of Exon 2 of the HPRT 1 gene are introduced to lymphocytes via a non-viral delivery vehicle, such as a nanocapsule. In some embodiments, the guide RNA molecule has at least 90% sequence identity to any one of SEQ ID NOS: 45 and 57-61. In some embodiments, the guide RNA molecule has at least 95% sequence identity to any one of SEQ ID NOS: 45 and 57-61. In some embodiments, the guide RNA molecule has at least 97% sequence identity to any one of SEQ ID NOS: 45 and 57-61. In some embodiments, the guide RNA molecule has at least 98% sequence identity to any one of SEQ ID NOS: 45 and 57-61. In some embodiments, the guide RNA molecule has at least 99% sequence identity to any one of SEQ ID NOS: 45 and 57-61.

In some embodiments, the endonuclease delivered via a non-viral delivery vehicle comprises a Cas protein. In some embodiments, the Cas protein comprises a Cas9 protein. In some embodiments, the Cas protein comprises a Cas12 protein. In some embodiments, the Cas12 protein is a Cas12a protein. In some embodiments, the Cas12 protein is a Cas12b protein.

Physical delivery or delivery of agents through a non-viral delivery vehicle represents an alternative to effectuating downregulation of HPRT (e.g. HPRT 1) by means of an expressed RNAi or other agent from an expression vector. As described further herein, it is possible to deliver antisense RNA, oligonucleotides designed for exon skipping, or gene editing machinery using nanocapsules or one or more physical methods such electroporation.

In general, a nanocapsule is a vesicular system that exhibits a typical core-shell structure in which active molecules are confined to a reservoir or cavity that is surrounded by a polymer membrane or coating. In some embodiments, the shell of a typical nanocapsule is made of a polymeric membrane or coating. In some embodiments, the nanocapsules are derived from a biodegradable or bioerodable polymeric material, i.e. the nanocapsules are biodegradable and/or erodible polymeric nanocapsules. For example, the components for knockdown and/or knockout be encapsulated within a nanocapsule comprising one or more biodegradable polymers such as polylactide-polyglycolide, poly(orthoesters) and poly(anhydrides). In some embodiments, the polymeric nanocapsules are comprised of two different positively charged monomers, at least one neutral monomer, and a cross-linker. In some embodiments, the nanocapsule is an enzymatically degradable nanocapsule. In some embodiments, the nanocapsule consists of a single-protein core and a thin polymeric shell cross-linked by peptides. In some embodiments, a nanocapsule may be selected such that it is specifically recognizable and able to be cleaved by a protease. In some embodiments, the cleavable cross-linkers include a peptide sequence or structure that is a substrate of a protease or another enzyme.

Examples of nanocapsules includes, without limitation, those described in U.S. Pat. No. 9,782,357; those described in United States Patent Application Publication Nos. 2017/0354613, and 2015/0071999; and those described in PCT Publication Nos. WO2016/085808 and WO2017/205541, the disclosures of which are hereby incorporated by reference herein in their entireties. In some embodiments, the nanocapsules described in the aforementioned publications may be modified to carry and/or encapsulate components for knockdown and/or knockout, e.g. a Cas protein and/or a gRNA. Other suitable nanocapsules, their methods of synthesis, and/or methods of encapsulation, are further disclosed in United States Patent Publication No. 2011/0274682, the disclosure of which is hereby incorporated by reference herein in its entirety. Yet other suitable nanocapsules which may be modified to carry and/or encapsulate components to effectuate knockdown or knockout of HPRT are described in PCT Publication Nos. WO2013/138783, WO2013/033717, and WO2014/093966, the disclosures of which are hereby incorporated by reference herein in their entireties.

In some embodiments, the nanocapsules are adapted to target specific cell types (e.g. T-cells, CD34 hematopoietic stem cells and progenitor cells) in vivo. For example, the nanocapsules may include one or more targeting moieties coupled to a polymer nanocapsule. In some embodiments, the targeting moiety delivers the polymer nanocapsules to a specific cell type, wherein the cell type is selected from the group comprising immune cells, blood cells, cardiac cells, lung cells, optic cells, liver cells, kidney cells, brain cells, cells of the central nervous system, cells of the peripheral nervous system, cancer cells, cells infected with viruses, stem cells, skin cells, intestinal cells, and/or auditory cells. In some embodiments, the targeting moieties are antibodies.

In some embodiments, the nanocapsules further comprise at least one targeting moiety. In some embodiments, the nanocapsules comprise between 2 and between 6 targeting moieties. In some embodiments, the targeting moieties are antibodies. In some embodiments, the targeting moieties target any one of the CD117, CD10, CD34, CD38, CD45, CD123, CD127, CD135, CD44, CD47, CD96, CD2, CD4, CD3, and CD9 markers. In some embodiments, the targeting moiety targets any one of a human mesenchymal stern cell CD marker, including the CD29, CD44, CD90, CD49a-f, CD51, CD73 (SH3), CD105 (SH2), CD106, CD166, and Stro-1 markers. In some embodiments, the targeting moiety targets any one of a human hematopoietic stem cell CD marker including CD34, CD38, CD45RA, CD90, and CD49.

In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127 or FoxP3 and CD44. In some embodiments, the T-cell marker is CD3. In some embodiments, the T-cell marker is CD28.

In some embodiments, co-stimulation with one or more co-stimulating moieties may be used to activate target cells, including T-cells. In some embodiments, co-stimulation may be achieved by activating one or more cell surface markers, including but not limited to CD28, ICOS, CTLA4, PD1, PD1H, and BTLA. In some embodiments, the co-stimulating moieties are antibodies.

A skilled person would appreciate that immune cells may rely on a secondary signal to activate an immune response (i.e. co-stimulation). For example, T cells may require two stimuli to fully activate the immune response. In some embodiments, co-stimulation with one or more co-stimulating moieties may be used to activate target cells, including T-cells. In some embodiments, co-stimulation may be achieved by activating one or more cell surface markers, including but not limited to CD28, ICOS, CTLA4, PD1, PD1H, and BTLA. In some embodiments, the co-stimulating moieties are antibodies.

Suitable payloads for such nanocapsules include synthetic oligonucleotides, shRNAs, miRNAs, and Ago-shRNAs targeting HPRT. In some embodiments, the payloads may be expressed in Pol III or Pol II driven promoter cassettes.

In other embodiments, agents for downregulating HPRT may be formulated within bio-nanocapsules, which are nano-size capsules produced by a genetically engineered microorganism. In some embodiments, a bio-nanocapsule is a virus protein-derived or modified virus protein-derived particle, such as a virus surface antigen particle (e.g., a hepatitis B virus surface antigen (HBsAg) particle). In other embodiments, a bio-nanocapsule is a nano-size capsule comprising a lipid bilayer membrane and a virus protein-derived or modified virus protein-derived particle such as a virus surface antigen particle. Such particles can be purified from eukaryotic cells, such as yeasts, insect cells, and mammalian cells. The size of a capsule may range from between about 10 nm to about 500 nm. In other embodiments, the size of the capsule may range from between about 20 nm to about 250 nm. In yet other embodiments, the size of the capsule may range from between about 80 nm to about 150.

Antisense RNA

Antisense RNA (asRNA) is a single-stranded RNA that is complementary to a messenger RNA (mRNA) strand transcribed within a cell. Without wishing to be bound by any particular theory, it is believed that antisense RNA may be introduced into a cell to inhibit translation of a complementary mRNA by base pairing to it and physically obstructing the translation machinery. Said another way, antisense RNAs are single-stranded RNA molecules that exhibit a complementary relationship to specific mRNAs.

Antisense RNAs may be utilized for gene regulation and specifically target mRNA molecules that are used for protein synthesis. The antisense RNA can physically pair and bind to the complementary mRNA, thus inhibiting the ability of the mRNA to be processed in the translation machinery. In some embodiments, phosphorothioate-modified antisense oligonucleotides may be utilized to target sequences within the coding region of HPRT mRNA. These oligonucleotides can be delivered to specific cell populations and anatomic sites cells using targeted nanoparticles, as described above.

Exon Skipping

As noted herein, exon skipping may be utilized to create a defect within the HPRT1 gene that results in HPRT deficiency. In some embodiments, an oligonucleotide (including a modified oligonucleotide) may be delivered by means of a nanocapsule, the oligonucleotide designed to target un-spliced HPRT mRNA and mediate either premature termination or skipping of an intron required for activity. An HPRT duplication mutation, e.g. e.g. a duplication mutation in Exon 4, (see Baba S, et al., “Novel mutation in HPRT1 causing a splicing error with multiple variations,” Nucleosides Nucleotides Nucleic Acids. 2017 Jan. 2; 36(1):1-6) could be introduced to cause a splicing error and functional inactivation of the HPRT protein. Replacing HPRT with a modified mutated sequence by spliceosome trans-splicing is a potential therapeutic strategy to knockdown HPRT. It is believed that this requires (1) a mutated coding region to replace the coding sequence in target RNA, (2) a 5′ or 3′ splice site, and (3) a binding domain, e.g., an antisense oligonucleotide sequence, which is complementary to target RNA.

The oligonucleotides may be structurally modified such that they are nuclease resistant. In some embodiments, the oligonucleotides have modified backbones or non-natural inter-nucleoside linkages. Such oligonucleotides having modified backbones include those that retain a phosphorus atom in the backbone and those that do not have a phosphorus atom in the backbone. In some embodiments, modified oligonucleotides that do not have a phosphorus atom in their inter-nucleoside backbone can also be considered to be oligonucleotides. In other embodiments, the oligonucleotides are modified such that both the sugar and the inter-nucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Modified oligonucleotides may also contain one or more substituted sugar moieties. Oligonucleotides may also include nucleobase (often referred to in the art simply as “base”) modifications or substitutions. Certain nucleobases are particularly useful for increasing the binding affinity of the oligomeric compounds of the disclosure. These include, without limitation, 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by about 0.6 to about 1.2° C. and are presently preferred base substitutions, even more particularly when combined with 2′-O-methoxyethyl sugar modifications.

Gene Editing to Knockout HPRT

The present disclosure also provides compositions for the knockout of HPRT 1. By way of non-limiting example, isolated cells (e.g. primary T-lymphocytes) may be treated with a HPRT-targeted CRISPR/Cas, e.g. a HPRT-targeted CRISPR/Cas9 RNP, with a HPRT-targeted CRISPR/Cas12a RNP, or with a HPRT-targeted CRISPR/Cas12b RNP.

A “ribonucleoprotein complex” as provided herein refers to a complex or particle including a nucleoprotein and a ribonucleic acid. A “nucleoprotein” as provided herein refers to a protein capable of binding a nucleic acid (e.g., RNA, DNA), Where the nucleoprotein binds a ribonucleic acid, it is referred to as “ribonucleoprotein.” The interaction between the nucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like)

In some embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA, thereby forming a ribonucleoprotein complex. Non-limiting examples of ribonucleoproteins include ribosomes, telomerase, RNAseP, hnRNP, CRISPR associated protein 9 (Cas9) and small nuclear RNPs (snRNPs).

In some embodiments, the ribonucleoprotein may be an enzyme. In some embodiments, the ribonucleoprotein is an endonuclease. Thus, in some embodiments, the ribonucleoprotein complex includes an endonuclease and a ribonucleic acid. In some embodiments, the endonuclease is a CRISPR associated protein 9. In some embodiments, the endonuclease is a CRISPR associated protein 12a. In some embodiments, the endonuclease is a CRISPR associated protein 12b.

In some embodiments, the ribonucleic acid is a guide RNA. Examples of guide RNAs or guide RNA molecules include any of SEQ ID NOS: 25 39 or any one of SEQ ID NOS: 40-61. In some embodiments, the guide RNA includes one or more RNA molecules (e.g. a crRNA which is complementary to a target sequence; and a tracr RNA which services as a binding scaffold for the nuclease).

In some embodiments, the gRNA includes a nucleotide sequence complementary to a target sequence (e.g. a target sequence within Chromosome X, a target sequence with the HPRT 1 gene, a target sequence within Exon 2, a target sequence within Exon 3 of the HPRT 1 gene, or a target sequence within Exon 8 of the HPRT 1 gene) or a portion thereof. A target sequence as provided herein refers to a nucleic acid sequence expressed by a cell. In some embodiments, the target nucleic acid sequence is an exogenous nucleic acid sequence. In some embodiments, the target sequence is an endogenous nucleic acid sequence. In some embodiments, the target sequence forms part of a cellular gene. Thus, in some embodiments, the guide RNA is complementary to a cellular gene or fragment thereof.

In some embodiments, the guide RNA is about 50%, about 55%, about 60%, about 65%, about 70%, about 75%, about 80%, about 85%, about 90%, about 95%, about 96%, about 97%, about 98% or about 99% complementary to the target nucleic acid sequence. In some embodiments, the guide RNA is about 50%, about 55%, about 60%, about 65%, about 70%, about 75%, about 80%, about 85%, about 90%, about 95%, about 96%, about 97%, about 98% or about 99% complementary to the sequence of a cellular gene. In some embodiments, the guide RNA binds a cellular gene sequence.

In some embodiments, the complement of the guide RNA has at least about 50% sequence identity to a target sequence. In some embodiments, the complement of the guide RNA has at least about 55% sequence identity to a target sequence. In some embodiments, the complement of the guide RNA has at least about 60% sequence identity to a target sequence. In some embodiments, the complement of the guide RNA has at least about 65% sequence identity to a target sequence. In some embodiments, the complement of the guide RNA has at least about 70% sequence identity to a target sequence. In some embodiments, the complement of the guide RNA has at least about 75% sequence identity to a target sequence. In some embodiments, the complement of the guide RNA has at least about 80% sequence identity to a target sequence. In some embodiments, the complement of the guide RNA has at least about 85% sequence identity to a target sequence. In some embodiments, the complement of the guide RNA has at least about 90% sequence identity to a target sequence. In some embodiments, the complement of the guide RNA has at least about 95% sequence identity to a target sequence. In some embodiments, the complement of the guide RNA has at least about 96% sequence identity to a target sequence. In some embodiments, the complement of the guide RNA has at least about 97% sequence identity to a target sequence. In some embodiments, the complement of the guide RNA has at least about 98% sequence identity to a target sequence. In some embodiments, the complement of the guide RNA has at least about 99% sequence identity to a target sequence. In some embodiments, the complement of the guide RNA comprises the target sequence.

In some embodiments, the present disclosure provides for a composition which includes a guide RNA which targets a sequence within the human hypoxanthine phosphoribosyltransferase (HPRT) gene (SEQ ID NO: 12). In some embodiments, the composition includes a guide RNA which targets a sequence within Chromosome X of a human at a location ranging from about 134460145 to about 134500668 (based on genome build GRCh38 or the equivalent position in a genome build other than GRCh38 (e.g. a previously known genome build or a future genome build)). In some embodiments, the composition includes a guide RNA which targets a sequence having a location within Chromosome X ranging from about 134460145 to about 134500668 (based on genome build GRCh38 or the equivalent position in a genome build other than GRCh38), and wherein the sequence targeted has a length ranging from about 14 to about 28 consecutive base pairs. In some embodiments, the composition includes a guide RNA which targets a sequence having a location within Chromosome X ranging from about 134460145 to about 134500668 (based on genome build GRCh38 or the equivalent position in a genome build other than GRCh38), and wherein the sequence targeted has a length ranging from about 15 to about 26 consecutive base pairs. In some embodiments, the composition includes a guide RNA which targets a sequence having a location within Chromosome X ranging from about 134460145 to about 134500668 (based on genome build GRCh38 or the equivalent position in a genome build other than GRCh38), and wherein the sequence targeted has a length ranging from about 16 to about 24 consecutive base pairs. In some embodiments, the composition includes a guide RNA which targets a sequence having a location within Chromosome X ranging from about 134460145 to about 134500668 (based on genome build GRCh38 or the equivalent position in a genome build other than GRCh38), and wherein the sequence targeted has a length ranging from about 17 to about 22 consecutive base pairs. In some embodiments, the composition includes a guide RNA which targets a sequence having a location within Chromosome X ranging from about 134460145 to about 134500668 (based on genome build GRCh38 or the equivalent position in a genome build other than GRCh38), and wherein the sequence targeted has a length ranging from about 18 to about 22 consecutive base pairs. While locations within Chromosome X are referenced herein to Genome Reference Consortium Human Build 38 (GRCh38), a person skilled in the art would understand that these referenced locations may be transposed to equivalent locations in alternative human genome builds or assemblies.

In some embodiments, the composition includes a gRNA having a nucleotide sequence which has at least 90% sequence identity to a target sequence located within Chromosome X at a position ranging from between about 134460145 to about 134500668 (based on genome build GRCh38 or the equivalent position in a genome build other than GRCh38). In some embodiments, the composition includes a gRNA having a nucleotide sequence which has at least 95% sequence identity to a target sequence located within Chromosome X at a position ranging from between about 134460145 to about 134500668 (based on genome build GRCh38 or the equivalent position in a genome build other than GRCh38). In some embodiments, the composition includes a gRNA having a nucleotide sequence which has at least 96% sequence identity to a target sequence located within Chromosome X at a position ranging from between about 134460145 to about 134500668 (based on genome build GRCh38 or the equivalent position in a genome build other than GRCh38). In some embodiments, the composition includes a gRNA having a nucleotide sequence which has at least 97% sequence identity to a target sequence located within Chromosome X at a position ranging from between about 134460145 to about 134500668 (based on genome build GRCh38 or the equivalent position in a genome build other than GRCh38). In some embodiments, the composition includes a gRNA having a nucleotide sequence which has at least 98% sequence identity to a target sequence located within Chromosome X at a position ranging from between about 134460145 to about 134500668 (based on genome build GRCh38 or the equivalent position in a genome build other than GRCh38). In some embodiments, the composition includes a gRNA having a nucleotide sequence which has at least 99% sequence identity to a target sequence located within Chromosome X at a position ranging from between about 134460145 to about 134500668 (based on genome build GRCh38 or the equivalent position in a genome build other than GRCh38).

In some embodiments, a complement of a target sequence within Chromosome X at a position ranging from between about 134460145 to about 134500668 (based on genome build GRCh38 or the equivalent position in a genome build other than GRCh38) has least 90% identity to any one of SEQ ID NOS: 25-39 or to any one of SEQ ID NOS: 40-61. In some embodiments, a complement of a target sequence within Chromosome X at a position ranging from between about 134460145 to about 134500668 (based on genome build GRCh38 or the equivalent position in a genome build other than GRCh38) has least 91% identity to any one of SEQ ID NOS: 25-39 or to any one of SEQ ID NOS: 40-61. In some embodiments, a complement of a target sequence within Chromosome X at a position ranging from between about 134460145 to about 134500668 (based on genome build GRCh38 or the equivalent position in a genome build other than GRCh38) has least 92% identity to any one of SEQ ID NOS: 25-39 or to any one of SEQ ID NOS: 40-61. In some embodiments, a complement of a target sequence within Chromosome X at a position ranging from between about 134460145 to about 134500668 (based on genome build GRCh38 or the equivalent position in a genome build other than GRCh38) has least 93% identity to any one of SEQ ID NOS: 25-39 or to any one of SEQ ID NOS: 40-61. In some embodiments, a complement of a target sequence within Chromosome X at a position ranging from between about 134460145 to about 134500668 (based on genome build GRCh38 or the equivalent position in a genome build other than GRCh38) has least 94% identity to any one of SEQ ID NOS: 25-39 or to any one of SEQ ID NOS: 40-61. In some embodiments, a complement of a target sequence within Chromosome X at a position ranging from between about 134460145 to about 134500668 (based on genome build GRCh38 or the equivalent position in a genome build other than GRCh38) has least 95% identity to any one of SEQ ID NOS: 25-39 or to any one of SEQ ID NOS: 40-61.

In some embodiments, a complement of a target sequence within Chromosome X at a position ranging from between about 134460145 to about 134500668 (based on genome build GRCh38 or the equivalent position in a genome build other than GRCh38) has least 96% identity to any one of SEQ ID NOS: 25-39 or to any one of SEQ ID NOS: 40-61. In some embodiments, a complement of a target sequence within Chromosome X at a position ranging from between about 134460145 to about 134500668 (based on genome build GRCh38 or the equivalent position in a genome build other than GRCh38) has least 97% identity to any one of SEQ ID NOS: 25-39 or to any one of SEQ ID NOS: 40-61. In some embodiments, a complement of a target sequence within Chromosome X at a position ranging from between about 134460145 to about 134500668 (based on genome build GRCh38 or the equivalent position in a genome build other than GRCh38) has least 98% identity to any one of SEQ ID NOS: 25-39 or to any one of SEQ ID NOS: 40-61. In some embodiments, a complement of a target sequence within Chromosome X at a position ranging from between about 134460145 to about 134500668 (based on genome build GRCh38 or the equivalent position in a genome build other than GRCh38) has least 99% identity to any one of SEQ ID NOS: 25-39 or to any one of SEQ ID NOS: 40-61.

In some embodiments, the composition includes a guide RNA which targets a sequence within Chromosome X of a human at a location ranging from about 134475181 to about 134475364 (based on genome build GRCh38 or the equivalent position in a genome build other than GRCh38). In some embodiments, the composition includes a guide RNA which targets a sequence having a location within Chromosome X ranging from about 134475181 to about 134475364 (based on genome build GRCh38 or the equivalent position in a genome build other than GRCh38), and wherein the sequence targeted has a length ranging from about 14 to about 28 consecutive base pairs. In some embodiments, the composition includes a guide RNA which targets a sequence having a location within Chromosome X ranging from about 134475181 to about 134475364 (based on genome build GRCh38 or the equivalent position in a genome build other than GRCh38), and wherein the sequence targeted has a length ranging from about 15 to about 26 consecutive base pairs. In some embodiments, the composition includes a guide RNA which targets a sequence having a location within Chromosome X ranging from about 134475181 to to about 134475364 (based on genome build GRCh38 or the equivalent position in a genome build other than GRCh38), and wherein the sequence targeted has a length ranging from about 16 to about 24 consecutive base pairs. In some embodiments, the composition includes a guide RNA which targets a sequence having a location within Chromosome X ranging from about 134475181 to to about 134475364 (based on genome build GRCh38 or the equivalent position in a genome build other than GRCh38), and wherein the sequence targeted has a length ranging from about 17 to about 24 consecutive base pairs. In some embodiments, the composition includes a guide RNA which targets a sequence having a location within Chromosome X ranging from about 134475181 to to about 134475364, and wherein the sequence targeted has a length ranging from about 18 to about 24 consecutive base pairs.

In some embodiments, the composition includes a gRNA having a nucleotide sequence which has at least 90% sequence identity to a target sequence located within Chromosome X at a position ranging from between about 134475181 to about 134475364 (based on genome build GRCh38 or the equivalent position in a genome build other than GRCh38). In some embodiments, the composition includes a gRNA having a nucleotide sequence which has at least 95% sequence identity to a target sequence located within Chromosome X at a position ranging from between about 134475181 to about 134475364 (based on genome build GRCh38 or the equivalent position in a genome build other than GRCh38). In some embodiments, the composition includes a gRNA having a nucleotide sequence which has at least 96% sequence identity to a target sequence located within Chromosome X at a position ranging from between about 134475181 to about 134475364 (based on genome build GRCh38 or the equivalent position in a genome build other than GRCh38). In some embodiments, the composition includes a gRNA having a nucleotide sequence which has at least 97% sequence identity to a target sequence located within Chromosome X at a position ranging from between about 134475181 to about 134475364 (based on genome build GRCh38 or the equivalent position in a genome build other than GRCh38). In some embodiments, the composition includes a gRNA having a nucleotide sequence which has at least 98% sequence identity to a target sequence located within Chromosome X at a position ranging from between about 134475181 to about 134475364 (based on genome build GRCh38 or the equivalent position in a genome build other than GRCh38). In some embodiments, the composition includes a gRNA having a nucleotide sequence which has at least 99% sequence identity to a target sequence located within Chromosome X at a position ranging from between about 134475181 to about 134475364 (based on genome build GRCh38 or the equivalent position in a genome build other than GRCh38).

In some embodiments, the composition includes a guide RNA which targets a sequence within Chromosome X of a human at a location ranging from about 134498608 to about 134498684 (based on genome build GRCh38 or the equivalent position in a genome build other than GRCh38). In some embodiments, the composition includes a guide RNA which targets a sequence having a location within Chromosome X ranging from about 134498608 to about 134498684 (based on genome build GRCh38 or the equivalent position in a genome build other than GRCh38), and wherein the sequence targeted has a length ranging from about 14 to about 28 consecutive base pairs. In some embodiments, the composition includes a guide RNA which targets a sequence having a location within Chromosome X ranging from about 134498608 to about 134498684 (based on genome build GRCh38 or the equivalent position in a genome build other than GRCh38), and wherein the sequence targeted has a length ranging from about 15 to about 26 consecutive base pairs. In some embodiments, the composition includes a guide RNA which targets a sequence having a location within Chromosome X ranging from about 134498608 to about 134498684 (based on genome build GRCh38 or the equivalent position in a genome build other than GRCh38), and wherein the sequence targeted has a length ranging from about 16 to about 24 consecutive base pairs. In some embodiments, the composition includes a guide RNA which targets a sequence having a location within Chromosome X ranging from about 134498608 to about 134498684 (based on genome build GRCh38 or the equivalent position in a genome build other than GRCh38), and wherein the sequence targeted has a length ranging from about 17 to about 24 consecutive base pairs. In some embodiments, the composition includes a guide RNA which targets a sequence having a location within Chromosome X ranging from about 134498608 to about 134498684 (based on genome build GRCh38 or the equivalent position in a genome build other than GRCh38), and wherein the sequence targeted has a length ranging from about 18 to about 24 consecutive base pairs.

In some embodiments, the composition includes a gRNA having a nucleotide sequence which has at least 90% sequence identity to a target sequence located within Chromosome X at a position ranging from between about 134498608 to about 134498684 (based on genome build GRCh38 or the equivalent position in a genome build other than GRCh38). In some embodiments, the composition includes a gRNA having a nucleotide sequence which has at least 95% sequence identity to a target sequence located within Chromosome X at a position ranging from between about 134498608 to about 134498684 (based on genome build GRCh38 or the equivalent position in a genome build other than GRCh38). In some embodiments, the composition includes a gRNA having a nucleotide sequence which has at least 96% sequence identity to a target sequence located within Chromosome X at a position ranging from between about 134498608 to about 134498684 (based on genome build GRCh38 or the equivalent position in a genome build other than GRCh38). In some embodiments, the composition includes a gRNA having a nucleotide sequence which has at least 97% sequence identity to a target sequence located within Chromosome X at a position ranging from between about 134498608 to about 134498684 (based on genome build GRCh38 or the equivalent position in a genome build other than GRCh38). In some embodiments, the composition includes a gRNA having a nucleotide sequence which has at least 98% sequence identity to a target sequence located within Chromosome X at a position ranging from between about 134498608 to about 134498684 (based on genome build GRCh38 or the equivalent position in a genome build other than GRCh38). In some embodiments, the composition includes a gRNA having a nucleotide sequence which has at least 99% sequence identity to a target sequence located within Chromosome X at a position ranging from between about 134498608 to about 134498684 (based on genome build GRCh38 or the equivalent position in a genome build other than GRCh38).

In some embodiments, the guide RNA has a nucleotide sequence having at least 85% sequence identity to any one of SEQ IDS NO: 25-39. In some embodiments, the guide RNA has a nucleotide sequence having at least 85% sequence identity to any one of SEQ IDS NO: 25-39. In some embodiments, the guide RNA has a nucleotide sequence having at least 90% sequence identity to any one of SEQ IDS NO: 25-39. In some embodiments, the guide RNA has a nucleotide sequence having at least 95% sequence identity to any one of SEQ IDS NO: 25-39. In some embodiments, the guide RNA has a nucleotide sequence having at least 97% sequence identity to any one of SEQ IDS NO: 25-39. In some embodiments, the guide RNA has a nucleotide sequence having at least 99% sequence identity to any one of SEQ IDS NO: 25-39. In some embodiments, the guide RNA has a nucleotide sequence comprising any one of SEQ IDS NO: 25-39.

In some embodiments, the composition includes a guide RNA which targets a sequence within Chromosome X of a human at a location ranging from about 134473409 to about 134473460 (based on genome build GRCh38 or the equivalent position in a genome build other than GRCh38). In some embodiments, the composition includes a guide RNA which targets a sequence having a location within Chromosome X ranging from about 134473409 to about 134473460 (based on genome build GRCh38 or the equivalent position in a genome build other than GRCh38), and wherein the sequence targeted has a length ranging from about 14 to about 28 consecutive base pairs. In some embodiments, the composition includes a guide RNA which targets a sequence having a location within Chromosome X ranging from about 134473409 to about 134473460 (based on genome build GRCh38 or the equivalent position in a genome build other than GRCh38), and wherein the sequence targeted has a length ranging from about 15 to about 26 consecutive base pairs. In some embodiments, the composition includes a guide RNA which targets a sequence having a location within Chromosome X ranging from about 134473409 to about 134473460 (based on genome build GRCh38 or the equivalent position in a genome build other than GRCh38), and wherein the sequence targeted has a length ranging from about 16 to about 24 consecutive base pairs. In some embodiments, the composition includes a guide RNA which targets a sequence having a location within Chromosome X ranging from about 134473409 to about 134473460 (based on genome build GRCh38 or the equivalent position in a genome build other than GRCh38), and wherein the sequence targeted has a length ranging from about 17 to about 24 consecutive base pairs. In some embodiments, the composition includes a guide RNA which targets a sequence having a location within Chromosome X ranging from about 134473409 to about 134473460 (based on genome build GRCh38 or the equivalent position in a genome build other than GRCh38), and wherein the sequence targeted has a length ranging from about 18 to about 24 consecutive base pairs.

In some embodiments, the composition includes a gRNA having a nucleotide sequence which has at least 90% sequence identity to a target sequence located within Chromosome X at a position ranging from between about 134473409 to about 134473460 (based on genome build GRCh38 or the equivalent position in a genome build other than GRCh38). In some embodiments, the composition includes a gRNA having a nucleotide sequence which has at least 95% sequence identity to a target sequence located within Chromosome X at a position ranging from between about 134473409 to about 134473460 (based on genome build GRCh38 or the equivalent position in a genome build other than GRCh38). In some embodiments, the composition includes a gRNA having a nucleotide sequence which has at least 96% sequence identity to a target sequence located within Chromosome X at a position ranging from between about 134473409 to about 134473460 (based on genome build GRCh38 or the equivalent position in a genome build other than GRCh38). In some embodiments, the composition includes a gRNA having a nucleotide sequence which has at least 97% sequence identity to a target sequence located within Chromosome X at a position ranging from between about 134473409 to about 134473460 (based on genome build GRCh38 or the equivalent position in a genome build other than GRCh38). In some embodiments, the composition includes a gRNA having a nucleotide sequence which has at least 98% sequence identity to a target sequence located within Chromosome X at a position ranging from between about 134473409 to about 134473460 (based on genome build GRCh38 or the equivalent position in a genome build other than GRCh38). In some embodiments, the composition includes a gRNA having a nucleotide sequence which has at least 99% sequence identity to a target sequence located within Chromosome X at a position ranging from between about 134473409 to about 134473460 (based on genome build GRCh38 or the equivalent position in a genome build other than GRCh38).

In some embodiments, the guide RNA has a nucleotide sequence having at least 85% sequence identity to any one of SEQ IDS NO: 40-56. In some embodiments, the guide RNA has a nucleotide sequence having at least 85% sequence identity to any one of SEQ IDS NO: 40-56. In some embodiments, the guide RNA has a nucleotide sequence having at least 90% sequence identity to any one of SEQ IDS NO: 40-56. In some embodiments, the guide RNA has a nucleotide sequence having at least 91% sequence identity to any one of SEQ IDS NO: 40-56. In some embodiments, the guide RNA has a nucleotide sequence having at least 92% sequence identity to any one of SEQ IDS NO: 40-56. In some embodiments, the guide RNA has a nucleotide sequence having at least 93% sequence identity to any one of SEQ IDS NO: 40-56. In some embodiments, the guide RNA has a nucleotide sequence having at least 94% sequence identity to any one of SEQ IDS NO: 40-56. In some embodiments, the guide RNA has a nucleotide sequence having at least 95% sequence identity to any one of SEQ IDS NO: 40-56. In some embodiments, the guide RNA has a nucleotide sequence having at least 96% sequence identity to any one of SEQ IDS NO: 40-56. In some embodiments, the guide RNA has a nucleotide sequence having at least 97% sequence identity to any one of SEQ IDS NO: 40-56. In some embodiments, the guide RNA has a nucleotide sequence having at least 98% sequence identity to any one of SEQ IDS NO: 40-56. In some embodiments, the guide RNA has a nucleotide sequence having at least 99% sequence identity to any one of SEQ IDS NO: 40-56. In some embodiments, the guide RNA has a nucleotide sequence comprising any one of SEQ IDS NO: 40-56.

In some embodiments, the guide RNA has a nucleotide sequence having at least 85% sequence identity to any one of SEQ IDS NO: 57-61. In some embodiments, the guide RNA has a nucleotide sequence having at least 85% sequence identity to any one of SEQ IDS NO: 57-61. In some embodiments, the guide RNA has a nucleotide sequence having at least 90% sequence identity to any one of SEQ IDS NO: 57-61. In some embodiments, the guide RNA has a nucleotide sequence having at least 95% sequence identity to any one of SEQ IDS NO: 57-61. In some embodiments, the guide RNA has a nucleotide sequence having at least 96% sequence identity to any one of SEQ IDS NO: 57-61. In some embodiments, the guide RNA has a nucleotide sequence having at least 97% sequence identity to any one of SEQ IDS NO: 57-61. In some embodiments, the guide RNA has a nucleotide sequence having at least 98% sequence identity to any one of SEQ MS NO: 57-61. In some embodiments, the guide RNA has a nucleotide sequence having at least 99% sequence identity to any one of SEQ IDS NO: 57-61. In some embodiments, the guide RNA has a nucleotide sequence comprising any one of SEQ IDS NO: 57-61.

In some embodiments, the endonuclease is Cas9 and the ribonucleic acid is a guide RNA. In some embodiments, the endonuclease is Cas9 and the ribonucleic acid is a guide RNA having a nucleotide sequence having at least 85% sequence identity to any one of SEQ ID NOS: 25-39. In some embodiments, the endonuclease is Cas9 and the ribonucleic acid is a guide RNA having a nucleotide sequence having at least 90% sequence identity to any one of SEQ ID NOS: 25-39. In some embodiments, the endonuclease is Cas9 and the ribonucleic acid is a guide RNA having a nucleotide sequence having at least 91% sequence identity to any one of SEQ ID NOS: 25-39. In some embodiments, the endonuclease is Cas9 and the ribonucleic acid is a guide RNA having a nucleotide sequence having at least 92% sequence identity to any one of SEQ ID NOS: 25-39. In some embodiments, the endonuclease is Cas9 and the ribonucleic acid is a guide RNA having a nucleotide sequence having at least 93% sequence identity to any one of SEQ ID NOS: 25-39. In some embodiments, the endonuclease is Cas9 and the ribonucleic acid is a guide RNA having a nucleotide sequence having at least 94% sequence identity to any one of SEQ ID NOS: 25-39. In some embodiments, the endonuclease is Cas9 and the ribonucleic acid is a guide RNA having a nucleotide sequence having at least 95% sequence identity to any one of SEQ ID NOS: 25-39. In some embodiments, the endonuclease is Cas9 and the ribonucleic acid is a guide RNA having a nucleotide sequence having at least 96% sequence identity to any one of SEQ ID NOS: 25-39. In some embodiments, the endonuclease is Cas9 and the ribonucleic acid is a guide RNA having a nucleotide sequence having at least 97% sequence identity to any one of SEQ ID NOS: 25-39. In some embodiments, the endonuclease is Cas9 and the ribonucleic acid is a guide RNA having a nucleotide sequence having at least 98% sequence identity to any one of SEQ ID NOS: 25-39. In some embodiments, the endonuclease is Cas9 and the ribonucleic acid is a guide RNA having a nucleotide sequence having at least 99% sequence identity to any one of SEQ ID NOS: 25-39. In some embodiments, the endonuclease is Cas9 and the ribonucleic acid is a guide RNA having a nucleotide comprising any one of SEQ ID NOS: 25-39. In some embodiments, the endonuclease is Cas9 and the ribonucleic acid is a guide RNA having a nucleotide comprising SEQ ID NO: 25. In some embodiments, the endonuclease is Cas9 and the ribonucleic acid is a guide RNA having a nucleotide comprising SEQ ID NO: 26. In some embodiments, the endonuclease is Cas9 and the ribonucleic acid is a guide RNA having a nucleotide comprising SEQ ID NO: 27. In some embodiments, the endonuclease is Cas9 and the ribonucleic acid is a guide RNA having a nucleotide comprising SEQ ID NO: 28. In some embodiments, the endonuclease is Cas9 and the ribonucleic acid is a guide RNA having a nucleotide comprising SEQ ID NO: 29. In some embodiments, the endonuclease is Cas9 and the ribonucleic acid is a guide RNA having a nucleotide comprising SEQ ID NO: 30. In some embodiments, the endonuclease is Cas9 and the ribonucleic acid is a guide RNA having a nucleotide comprising SEQ ID NO: 31. In some embodiments, the endonuclease is Cas9 and the ribonucleic acid is a guide RNA having a nucleotide comprising SEQ ID NO: 32. In some embodiments, the endonuclease is Cas9 and the ribonucleic acid is a guide RNA having a nucleotide comprising SEQ ID NO: 33. In some embodiments, the endonuclease is Cas9 and the ribonucleic acid is a guide RNA having a nucleotide comprising SEQ ID NO: 34. In some embodiments, the endonuclease is Cas9 and the ribonucleic acid is a guide RNA having a nucleotide comprising SEQ ID NO: 35. In some embodiments, the endonuclease is Cas9 and the ribonucleic acid is a guide RNA having a nucleotide comprising SEQ ID NO: 36. In some embodiments, the endonuclease is Cas9 and the ribonucleic acid is a guide RNA having a nucleotide comprising SEQ ID NO: 37.

In some embodiments, the endonuclease is Cas9 and the ribonucleic acid is a guide RNA having a nucleotide sequence having at least 85% sequence identity to any one of SEQ ID NOS: 40-49. In some embodiments, the endonuclease is Cas9 and the ribonucleic acid is a guide RNA having a nucleotide sequence having at least 90% sequence identity to any one of SEQ ID NOS: 40-49. In some embodiments, the endonuclease is Cas9 and the ribonucleic acid is a guide RNA having a nucleotide sequence having at least 91% sequence identity to any one of SEQ ID NOS: 40-49. In some embodiments, the endonuclease is Cas9 and the ribonucleic acid is a guide RNA having a nucleotide sequence having at least 92% sequence identity to any one of SEQ ID NOS: 40-49. In some embodiments, the endonuclease is Cas9 and the ribonucleic acid is a guide RNA having a nucleotide sequence having at least 93% sequence identity to any one of SEQ ID NOS: 40-49. In some embodiments, the endonuclease is Cas9 and the ribonucleic acid is a guide RNA having a nucleotide sequence having at least 94% sequence identity to any one of SEQ NOS: 40-49. In some embodiments, the endonuclease is Cas9 and the ribonucleic acid is a guide RNA having a nucleotide sequence having at least 95% sequence identity to any one of SEQ ID NOS: 40-49. In some embodiments, the endonuclease is Cas9 and the ribonucleic acid is a guide RNA having a nucleotide sequence having at least 96% sequence identity to any one of SEQ ID NOS: 40-49. In some embodiments, the endonuclease is Cas9 and the ribonucleic acid is a guide RNA having a nucleotide sequence having at least 97% sequence identity to any one of SEQ ID NOS: 40-49. In some embodiments, the endonuclease is Cas9 and the ribonucleic acid is a guide RNA having a nucleotide sequence having at least 98% sequence identity to any one of SEQ ID NOS: 40-49. In some embodiments, the endonuclease is Cas9 and the ribonucleic acid is a guide RNA having a nucleotide sequence having at least 99% sequence identity to any one of SEQ ID NOS: 40-49. In some embodiments, the endonuclease is Cas9 and the ribonucleic acid is a guide RNA having a nucleotide comprising any one of SEQ ID NOS: 40-49.

In some embodiments, the endonuclease is Cas12a and the ribonucleic acid is a guide RNA. In some embodiments, the endonuclease is Cas12a and the ribonucleic acid is a guide RNA having a nucleotide sequence having at least 85% sequence identity to any one of SEQ ID NOS: 25-39. In some embodiments, the endonuclease is Cas12a and the ribonucleic acid is a guide RNA having a nucleotide sequence having at least 90% sequence identity to any one of SEQ ID NOS: 25-39. In some embodiments, the endonuclease is Cas12a and the ribonucleic acid is a guide RNA having a nucleotide sequence having at least 91% sequence identity to any one of SEQ ID NOS: 25-39. In some embodiments, the endonuclease is Cas12a and the ribonucleic acid is a guide RNA having a nucleotide sequence having at least 92% sequence identity to any one of SEQ ID NOS: 25-39. In some embodiments, the endonuclease is Cas12a and the ribonucleic acid is a guide RNA having a nucleotide sequence having at least 93% sequence identity to any one of SEQ ID NOS: 25-39. In some embodiments, the endonuclease is Cas12a and the ribonucleic acid is a guide RNA having a nucleotide sequence having at least 94% sequence identity to any one of SEQ ID NOS: 25-39. In some embodiments, the endonuclease is Cas12a and the ribonucleic acid is a guide RNA having a nucleotide sequence having at least 95% sequence identity to any one of SEQ ID NOS: 25-39. In some embodiments, the endonuclease is Cas12a and the ribonucleic acid is a guide RNA having a nucleotide sequence having at least 96% sequence identity to any one of SEQ ID NOS: 25-39. In some embodiments, the endonuclease is Cas12a and the ribonucleic acid is a guide RNA having a nucleotide sequence having at least 97% sequence identity to any one of SEQ ID NOS: 25-39. In some embodiments, the endonuclease is Cas12a and the ribonucleic acid is a guide RNA having a nucleotide sequence having at least 98% sequence identity to any one of SEQ ID NOS: 25-39. In some embodiments, the endonuclease is Cas12a and the ribonucleic acid is a guide RNA having a nucleotide sequence having at least 99% sequence identity to any one of SEQ ID NOS: 25-39. In some embodiments, the endonuclease is Cas12a and the ribonucleic acid is a guide RNA having a nucleotide comprising any one of SEQ ID NOS: 25-39.

In some embodiments, the endonuclease is Cas12a and the ribonucleic acid is a guide RNA having a nucleotide sequence having at least 85% sequence identity to any one of SEQ ID NOS: 40-49. In some embodiments, the endonuclease is Cas12a and the ribonucleic acid is a guide RNA having a nucleotide sequence having at least 90% sequence identity to any′ one of SEQ ID NOS: 40-49. In some embodiments, the endonuclease is Cas12a and the ribonucleic acid is a guide RNA having a nucleotide sequence having at least 91% sequence identity to any one of SEQ ID NOS: 40-49. In some embodiments, the endonuclease is Cas12a and the ribonucleic acid is a guide RNA having a nucleotide sequence having at least 92% sequence identity to any one of SEQ ID NOS: 40-49. In some embodiments, the endonuclease is Cas12a and the ribonucleic acid is a guide RNA having a nucleotide sequence having at least 93% sequence identity to any one of SEQ ID NOS: 40-49. In some embodiments, the endonuclease is Cas12a and the ribonucleic acid is a guide RNA having a nucleotide sequence having at least 94% sequence identity to any one of SEQ ID NOS: 40-49. In some embodiments, the endonuclease is Cas12a and the ribonucleic acid is a guide RNA having a nucleotide sequence having at least 95% sequence identity to any one of SEQ ID NOS: 40-49. In some embodiments, the endonuclease is Cas12a and the ribonucleic acid is a guide RNA having a nucleotide sequence having at least 96% sequence identity to any one of SEQ ID NOS: 40-49. In some embodiments, the endonuclease is Cas12a and the ribonucleic acid is a guide RNA having a nucleotide sequence having at least 97% sequence identity to any one of SEQ ID NOS: 40-49. In some embodiments, the endonuclease is Cas12a and the ribonucleic acid is a guide RNA having a nucleotide sequence having at least 98% sequence identity to any one of SEQ ID NOS: 40-49. In some embodiments, the endonuclease is Cas12a and the ribonucleic acid is a guide RNA having a nucleotide sequence having at least 99% sequence identity to any one of SEQ ID NOS: 40-49. In some embodiments, the endonuclease is Cas12a and the ribonucleic acid is a guide RNA having a nucleotide comprising any one of SEQ ID NOS: 40-49.

In some embodiments, the endonuclease is Cas12a and the ribonucleic acid is a guide RNA having a nucleotide sequence having at least 85% sequence identity to any one of SEQ ID NOS: 50-61. In some embodiments, the endonuclease is Cas12a and the ribonucleic acid is a guide RNA having a nucleotide sequence having at least 90% sequence identity to any one of SEQ ID NOS: 50-61. In some embodiments, the endonuclease is Cas12a and the ribonucleic acid is a guide RNA having a nucleotide sequence having at least 91% sequence identity to any one of SEQ ID NOS: 50-61. In some embodiments, the endonuclease is Cas12a and the ribonucleic acid is a guide RNA having a nucleotide sequence having at least 92% sequence identity to any one of SEQ ID NOS: 50-61. In some embodiments, the endonuclease is Cas12a and the ribonucleic acid is a guide RNA having a nucleotide sequence having at least 93% sequence identity to any one of SEQ ID NOS: 50-61. In some embodiments, the endonuclease is Cas12a and the ribonucleic acid is a guide RNA having a nucleotide sequence having at least 94% sequence identity to any one of SEQ ID NOS: 50-61. In some embodiments, the endonuclease is Cas12a and the ribonucleic acid is a guide RNA having a nucleotide sequence having at least 95% sequence identity to any one of SEQ ID NOS: 50-61. In some embodiments, the endonuclease is Cas12a and the ribonucleic acid is a guide RNA having a nucleotide sequence having at least 96% sequence identity to any one of SEQ ID NOS: 50-61. In some embodiments, the endonuclease is Cas12a and the ribonucleic acid is a guide RNA having a nucleotide sequence having at least 97% sequence identity to any one of SEQ ID NOS: 50-61. In some embodiments, the endonuclease is Cas12a and the ribonucleic acid is a guide RNA having a nucleotide sequence having at least 98% sequence identity to any one of SEQ ID NOS: 50-61. In some embodiments, the endonuclease is Cas12a and the ribonucleic acid is a guide RNA having a nucleotide sequence having at least 99% sequence identity to any one of SEQ ID NOS: 50-61. In some embodiments, the endonuclease is Cas12a and the ribonucleic acid is a guide RNA having a nucleotide comprising any one of SEQ ID NOS: 40-49.

In some embodiments, the endonuclease is Cas12b and the ribonucleic acid is a guide RNA. In some embodiments, the endonuclease is Cas1.2b and the ribonucleic acid is a guide RNA having a nucleotide sequence having at least 85% sequence identity to any one of SEQ ID NOS: 25-39. In some embodiments, the endonuclease is Cas12b and the ribonucleic acid is a guide RNA having a nucleotide sequence having at least 90% sequence identity to any one of SEQ ID NOS: 25-39. In some embodiments, the endonuclease is Cas1.2b and the ribonucleic acid is a guide RNA having a nucleotide sequence having at least 91% sequence identity to any one of SEQ ID NOS: 25-39. In some embodiments, the endonuclease is Cas12b and the ribonucleic acid is a guide RNA having a nucleotide sequence having at least 92% sequence identity to any one of SEQ ID NOS: 25-39. In some embodiments, the endonuclease is Cas12b and the ribonucleic acid is a guide RNA having a nucleotide sequence having at least 93% sequence identity to any one of SEQ ID NOS: 25-39. In some embodiments, the endonuclease is Cas12b and the ribonucleic acid is a guide RNA having a nucleotide sequence having at least 94% sequence identity to any one of SEQ ID NOS: 25-39. In some embodiments, the endonuclease is Cas1.2b and the ribonucleic acid is a guide RNA having a nucleotide sequence having at least 95% sequence identity to any one of SEQ ID NOS: 25-39. In some embodiments, the endonuclease is Cas12b and the ribonucleic acid is a guide RNA having a nucleotide sequence having at least 96% sequence identity to any one of SEQ ID NOS: 25-39. In some embodiments, the endonuclease is Cas12b and the ribonucleic acid is a guide RNA having a nucleotide sequence having at least 97% sequence identity to any one of SEQ ID NOS: 25-39. In some embodiments, the endonuclease is Cas12b and the ribonucleic acid is a guide RNA having a nucleotide sequence having at least 98% sequence identity to any one of SEQ ID NOS: 25-39. In some embodiments, the endonuclease is Cas12b and the ribonucleic acid is a guide RNA having a nucleotide sequence having at least 99% sequence identity to any one of SEQ ID NOS: 25-39. In some embodiments, the endonuclease is Cas12b and the ribonucleic acid is a guide RNA having a nucleotide comprising any one of SEQ ID NOS: 25-39.

In some embodiments, the endonuclease is Cas12b and the ribonucleic acid is a guide RNA having a nucleotide sequence having at least 85% sequence identity to any one of SEQ ID NOS: 40-49. In some embodiments, the endonuclease is Cas12b and the ribonucleic acid is a guide RNA having a nucleotide sequence having at least 90% sequence identity to any one of SEQ ID NOS: 40-49. In some embodiments, the endonuclease is Cas12b and the ribonucleic acid is a guide RNA having a nucleotide sequence having at least 91% sequence identity to any one of SEQ ID NOS: 40-49. In some embodiments, the endonuclease is Cas12b and the ribonucleic acid is a guide RNA having a nucleotide sequence having at least 92% sequence identity to any one of SEQ ID NOS: 40-49. In some embodiments, the endonuclease is Cas12b and the ribonucleic acid is a guide RNA having a nucleotide sequence having at least 93% sequence identity to any one of SEQ ID NOS: 40-49. In some embodiments, the endonuclease is Cas12b and the ribonucleic acid is a guide RNA having a nucleotide sequence having at least 94% sequence identity to any one of SEQ ID NOS: 40-49. In some embodiments, the endonuclease is Cas12b and the ribonucleic acid is a guide RNA having a nucleotide sequence having at least 95% sequence identity to any one of SEQ ID NOS: 40-49. In some embodiments, the endonuclease is Cas12b and the ribonucleic acid is a guide RNA having a nucleotide sequence having at least 96% sequence identity to any one of SEQ ID NOS: 40-49. In some embodiments, the endonuclease is Cas12b and the ribonucleic acid is a guide RNA having a nucleotide sequence having at least 97% sequence identity to any one of SEQ ID NOS: 40-49. In some embodiments, the endonuclease is Cas12b and the ribonucleic acid is a guide RNA having a nucleotide sequence having at least 98% sequence identity to any one of SEQ ID NOS: 40-49. In some embodiments, the endonuclease is Cas12b and the ribonucleic acid is a guide RNA having a nucleotide sequence having at least 99% sequence identity to any one of SEQ ID NOS: 40-49. In some embodiments, the endonuclease is Cas12b and the ribonucleic acid is a guide RNA having a nucleotide comprising any one of SEQ ID NOS: 40-49.

Host Cells

The present disclosure also provides a host cell comprising the novel expression vectors of the present disclosure. A “host cell” or “target cell” means a cell that is to be transformed (i.e. transduced or transfected) using the compositions, e.g. expression vectors or nanocapsules, of the present disclosure. In some embodiments, the host cell is rendered substantially HPRT deficient after transduction with an expression vector encoding a nucleic adapted to knockdown HPRT. In other embodiments, the host cell is rendered substantially HPRT deficient after transfection with a nanocapsule including components designed to effectuate knockout of HPRT. Methods of transducing host cells with an expression vector to knockdown HPRT or transfecting host cells with a nanocapsule to knockout HPRT are described in co-pending U.S. patent application Ser. No. 16/038,643, the disclosure of which is hereby incorporated by reference herein in its entirety. In some embodiments, the host cells are isolated and/or purified.

In some embodiments, the host cells are mammalian cells in which the expression vector can be expressed. Suitable mammalian host cells include, but are not limited to, human cells, murine cells, non-human primate cells (e.g. rhesus monkey cells), human progenitor cells or stem cells, 293 cells, HeLa cells, D17 cells, MDCK cells, BHK cells, and Cf2Th cells. In certain embodiments, the host cell comprising an expression vector of the disclosure is a hematopoietic cell, such as hematopoietic progenitor/stem cell (e.g. CD34-positive hematopoietic progenitor/stem cell), a monocyte, a macrophage, a peripheral blood mononuclear cell, a CD4+ T lymphocyte, a CD8+ T lymphocyte, or a dendritic cell.

The hematopoietic cells (e.g. CD4+ T lymphocytes, CD8+ T lymphocytes, and/or monocyte/macrophages) to be transduced with an expression vector or transfected with a nanocapsule of the present disclosure can be allogeneic, autologous, or from a matched sibling. The hematopoietic progenitor/stem cell are, in some embodiments, CD34-positive and can be isolated from the patient's bone marrow or peripheral blood. The isolated CD34-positive hematopoietic progenitor/stem cell (and/or other hematopoietic cell described herein) is, in some embodiments, transduced with an expression vector as described herein.

In some embodiments, the modified host cells are combined with a pharmaceutically acceptable carrier. In some embodiments, the host cells or transduced host cells are formulated with PLASMA-LYTE A (e.g. a sterile, nonpyrogenic isotonic solution for intravenous administration; where one liter of PLASMA-LYTE A has an ionic concentration of 140 mEq sodium, 5 mEq potassium, 3 mEq magnesium, 98 mEq chloride, 27 mEq acetate, and 23 me gluconate). In other embodiments, the host cells or transduced host cells are formulated in a solution of PLASMA-LYTE A, the solution comprising between about 8% and about 10% dimethyl sulfoxide (DMSO). In some embodiments, the less than about 2×107 host cells/transduced host cells are present per mL of a formulation including PLASMA-LYTE A and DMSO.

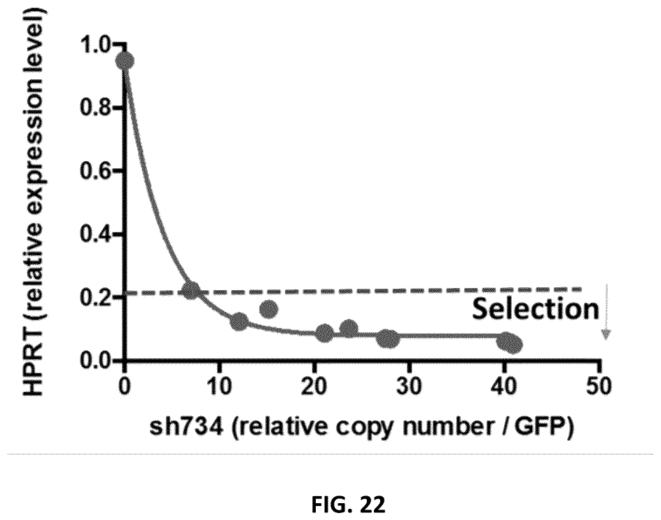

In some embodiments, the host cells are rendered substantially HPRT deficient after transduction with an expression vector according to the present disclosure. In some embodiments, the level of HPRT1 gene expression is reduced by at least about 80%. It is believed that cells having 20% or less residual HPRT1 gene expression are sensitive to a purine analog, such as 6-TG or 6-MP, allowing for their selection with the purine analog (see, for example, FIG. 22). In some embodiments, the host cells include a nucleic acid molecule having at least 90% identity to at least one of SEQ ID NO: 3 or SEQ ID NO: 4. In some embodiments, the host cells include a nucleic acid molecule having at least 95% identity to at least one of SEQ ID NO: 3 or SEQ ID NO: 4. In some embodiments, the host cells include a nucleic acid molecule comprising at least one of SEQ ID NO: 3 or SEQ ID NO: 4.

In some embodiments, transduction of host cells may be increased by contacting the host cell, in vitro, ex vivo, or in vivo, with an expression vector of the present disclosure and one or more compounds that increase transduction efficiency. For example, in some embodiments, the one or more compounds that increase transduction efficiency are compounds that stimulate the prostaglandin EP receptor signaling pathway, i.e. one or more compounds that increase the cell signaling activity downstream of a prostaglandin EP receptor in the cell contacted with the one or more compounds compared to the cell signaling activity downstream of the prostaglandin EP receptor in the absence of the one or more compounds. In some embodiments, the one or more compounds that increase transduction efficiency are a prostaglandin EP receptor ligand including, but not limited to, prostaglandin E2 (PGE2), or an analog or derivative thereof. In other embodiments, the one or more compounds that increase transduction efficiency include, but are not limited to, RetroNectin (a 63 kD fragment of recombinant human fibronectin fragment, available from Takara); Lentiboost (a membrane-sealing poloxamer, available from Sirion Biotech), Protamine Sulphate, Cyclosporin H, and Rapamycin. In yet other embodiments, the one or more compounds that increase transduction efficiency include poloxamers (e.g. poloxamer F127).

In some embodiments, the host cells are prepared by contacting the host cells with a composition comprising components to knockout the HPRT1 gene from the host cells. In some embodiments, the components to knockout the HPRT1 gene comprise an endonuclease (e.g. Cas9, Cas12a, or Cas12b) and a guide RNA molecule having at least 85% sequence identity to any one of SEQ ID NOS: 25-39 or SEQ ID NOS: 40-61. In some embodiments, the components to knockout the HPRT1 gene comprise an endonuclease (e.g. Cas9, Cas12a, or Cas12b) and a guide RNA molecule having at least 90% sequence identity to any one of SEQ ID NOS: 25-39 or SEQ ID NOS: 40-61. In some embodiments, the components to knockout the HPRT1 gene comprise an endonuclease (e.g. Cas9, Cas12a, or Cas12b) and a guide RNA molecule having at least 91% sequence identity to any one of SEQ ID NOS: 25-39 or SEQ ID NOS: 40-61. In some embodiments, the components to knockout the HPRT1 gene comprise an endonuclease (e.g. Cas9, Cas12a, or Cas12b) and a guide RNA molecule having at least 92% sequence identity to any one of SEQ ID NOS: 25-39 or SEQ ID NOS: 40-61. In some embodiments, the components to knockout the HPRT1 gene comprise an endonuclease (e.g. Cas9, Cas12a, or Cas12b) and a guide RNA molecule having at least 93% sequence identity to any one of SEQ ID NOS: 25-39 or SEQ ID NOS: 40-61. In some embodiments, the components to knockout the HPRT1 gene comprise an endonuclease (e.g. Cas9, Cas12a, or Cas12b) and a guide RNA molecule having at least 94% sequence identity to any one of SEQ ID NOS: 25-39 or SEQ ID NOS: 40-61. In some embodiments, the components to knockout the HPRT1 gene comprise an endonuclease (e.g. Cas9, Cas12a, or Cas12b) and a guide RNA molecule having at least 95% sequence identity to any one of SEQ ID NOS: 25-39 or SEQ ID NOS: 40-61. In some embodiments, the components to knockout the HPRT1 gene comprise an endonuclease (e.g. Cas9, Cas12a, or Cas12b) and a guide RNA molecule having at least 96% sequence identity to any one of SEQ ID NOS: 25-39 or SEQ ID NOS: 40-61. In some embodiments, the components to knockout the HPRT1 gene comprise an endonuclease (e.g. Cas9, Cas12a, or Cas12b) and a guide RNA molecule having at least 97% sequence identity to any one of SEQ ID NOS: 25-39 or SEQ ID NOS: 40-61. In some embodiments, the components to knockout the HPRT1 gene comprise an endonuclease (e.g. Cas9, Cas12a, or Cas12b) and a guide RNA molecule having at least 98% sequence identity to any one of SEQ ID NOS: 25-39 or SEQ ID NOS: 40-61. In some embodiments, the components to knockout the HPRT 1 gene comprise an endonuclease (e.g. Cas9, Cas12a, or Cas12b) and a guide RNA molecule having at least 99% sequence identity to any one of SEQ ID NOS: 25-39 or SEQ ID NOS: 40-61. In some embodiments, the components to knockout the HPRT1 gene comprise an endonuclease (e.g. Cas9, Cas12a, or Cas12b) and a guide RNA molecule comprising any one of SEQ ID NOS: 25-39 or SEQ ID NOS: 40-61.

Pharmaceutical Compositions

The present disclosure also provides for compositions, including pharmaceutical compositions, comprising one or more expression vectors and/or non-viral delivery vehicles (e.g. nanocapsules) as disclosed herein. In some embodiments, pharmaceutical compositions comprise an effective amount of at least one of the expression vectors and/or non-viral delivery vehicles as described herein and a pharmaceutically acceptable carrier. For instance, in certain embodiments, the pharmaceutical composition comprises an effective amount of an expression vector and a pharmaceutically acceptable carrier. An affective amount can be readily determined by those skilled in the art based on factors such as body size, body weight, age, health, sex of the subject, ethnicity, and viral titers.

In another aspect of the present disclosure is a pharmaceutical composition comprising (a) an expression vector, including a nucleic acid sequence encoding a shRNA targeting an HPRT1 gene; and (b) a pharmaceutically acceptable carrier. In some embodiments, the pharmaceutical composition is formulated as an emulsion. In some embodiments, the pharmaceutical composition is formulated within micelles. In some embodiments, the pharmaceutical composition is encapsulated within a polymer. In some embodiments, the pharmaceutical composition is encapsulated within a liposome. In some embodiments, the pharmaceutical composition is encapsulated within minicells or nanocapsules.

In some embodiments, a pharmaceutical composition comprises (a) a population of nanocapsules, each nanocapsule including a payload to adapted knockout HPRT (e.g. a Cas9 protein, a Cas12a protein, a Cas12b protein and/or a gRNA, such as a gRNA of any one of SEQ ID NOS: 25-39); and (b) a pharmaceutically acceptable carrier. In some embodiments, the nanocapsule is a polymer nanocapsule. In some embodiments, the polymer nanocapsule further comprises at least one targeting moiety to facilitate delivery of the ribonucleoprotein or ribonucleoprotein complex to a particular type of cell. In some embodiments, the polymer nanocapsule is erodible or biodegradable. In some embodiments, the polymer nanocapsule includes a pH sensitive cross-linker. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127 or FoxP3 and CD44. In some embodiments, the T-cell marker is CD3. In some embodiments, the T-cell marker is CD28.

In some embodiments, a pharmaceutical composition comprises (a) a population of nanocapsules, each nanocapsule including a payload to adapted knockout HPRT (e.g. a Cas9 protein, a Cas12a protein, a Cas12b protein and/or a gRNA, such as a gRNA having at least 90% sequence identity to, at least 91% sequence identity to, at least 92% sequence identity to, at least 93% sequence identity to, at least 94% sequence identity to, at least 95% sequence identity to, at least 96% sequence identity to, at least 97% sequence identity to, at least 98% sequence identity to, at least 99% sequence identity to any one of SEQ ID NOS: 40-61); and (b) a pharmaceutically acceptable carrier. In some embodiments, the nanocapsule is a polymer nanocapsule. In some embodiments, the polymer nanocapsule further comprises at least one targeting moiety to facilitate delivery of the ribonucleoprotein or ribonucleoprotein complex to a particular type of cell. In some embodiments, the polymer nanocapsule is erodible or biodegradable. In some embodiments, the polymer nanocapsule includes a pH sensitive cross-linker. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127 or FoxP3 and CD44. In some embodiments, the T-cell marker is CD3. In some embodiments, the T-cell marker is CD28.

In some embodiments, the polymer nanocapsule has a size ranging from between about 50 nm to about 250 nm. In some embodiments, the polymer nanocapsule has an average diameter of less than or equal to about 200 nanometers (nm). In some embodiments, the polymer nanocapsule has an average diameter of between about 1 to 200 nm. In some embodiments, the polymer nanocapsule has an average diameter of between about 5 to about 200 nm. In some embodiments, the polymer nanocapsule has an average diameter of between about 10 to about 150 nm, or 15 to 100 nm. In some embodiments, the polymer nanocapsule has an average diameter of between about 15 to about 150 mu. In some embodiments, the polymer nanocapsule has an average diameter of between about 20 to about 125 nm. In some embodiments, the polymer nanocapsule has an average diameter of between about 50 to about 100 nm. In some embodiments, the polymer nanocapsule has an average diameter of between about 50 to about 75 nm. In some embodiments, the surface of the nanocapsule can have a charge between about 1 to about 15 millivolts (mV) (such as measured in a standard phosphate solution). In other embodiments, the surface of the nanocapsule can have a charge between about 1 to about 10 mV.

The phrases “pharmaceutically acceptable” or “pharmacologically acceptable” refer to molecular entities and compositions that do not produce adverse, allergic, or other untoward reactions when administered to an animal or a human. For example, an expression vector may be formulated with a pharmaceutically acceptable carrier. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. Methods for the formulation of compounds with pharmaceutical carriers are known in the art and are described in, for example, in Remington's Pharmaceutical Science, (17th ed. Mack Publishing Company, Easton, Pa. 1985); and Goodman & Gillman's: The Pharmacological Basis of Therapeutics (11th Edition, McGraw-Hill Professional, 2005); the disclosures of each of which are hereby incorporated herein by reference in their entirety.

In some embodiments, the pharmaceutical compositions may comprise any of the expression vectors, nanocapsules, or compositions disclosed herein in any concentration that allows the silencing nucleic acid administered to achieve a concentration in the range of from about 0.1 mg/kg to about 1 mg/kg. In some embodiments, the pharmaceutical compositions may comprise the expression vector in an amount of from about 0.1% to about 99.9% by total weight of the pharmaceutical composition. In some embodiments, the pharmaceutical compositions may comprise the expression vector in an amount of from about 0.1% to about 90% by total weight of the pharmaceutical composition. Pharmaceutically acceptable carriers suitable for inclusion within any pharmaceutical composition include water, buffered water, saline solutions such as, for example, normal saline or balanced saline solutions such as Hank's or Earle's balanced solutions), glycine, hyaluronic acid etc. The pharmaceutical composition may be formulated for parenteral administration, such as intravenous, intramuscular or subcutaneous administration. Pharmaceutical compositions for parenteral administration may comprise pharmaceutically acceptable sterile aqueous or non-aqueous solutions, dispersions, suspensions or emulsions as well as sterile powders for reconstitution into sterile injectable solutions or dispersions. Examples of suitable aqueous and non-aqueous carriers, solvents, diluents or vehicles include water, ethanol, polyols (such as glycerol, propylene glycol, polyethylene glycol, etc.), carboxymethylcellulose and mixtures thereof, vegetable oils (such as olive oil), injectable organic esters (e.g. ethyl oleate).

The pharmaceutical composition may be formulated for oral administration. Solid dosage forms for oral administration may include, for example, tablets, dragees, capsules, pills, and granules. In such solid dosage forms, the composition may comprise at least one pharmaceutically acceptable carrier such as sodium citrate and/or dicalcium phosphate and/or fillers or extenders such as starches, lactose, sucrose, glucose, mannitol, and silicic acid; binders such as carboxylmethylcellulose, alginates, gelatin, polyvinylpyrrolidone, sucrose and acacia; humectants such as glycerol; disintegrating agents such as agar-agar, calcium carbonate, potato or tapioca starch, alginic acid, silicates, and sodium carbonate; wetting agents such as acetyl alcohol, glycerol monostearate; absorbants such as kaolin and bentonite clay; and/or lubricants such as talc, calcium stearate, magnesium stearate, solid polyethylene glycol, sodium lauryl sulfate, and mixtures thereof. Liquid dosage forms for oral administration may include, for example, pharmaceutically acceptable emulsions, solutions, suspensions, syrups and elixirs. Liquid dosages may include inert diluents such as water or other solvents, solubilizing agents and/or emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (such as, for example, cottonseed oil, corn oil, germ oil, castor oil, olive oil, sesame oil), glycerol, tetrahydrofurfuryl alcohol, polyethylene glycols and fatty acid esters of sorbitan, and mixtures thereof.

The pharmaceutical compositions may comprise penetration enhancers to enhance their delivery. Penetration enhancers may include fatty acids such as oleic acid, lauric acid, capric acid, myristic acid, palmitic acid, stearic acid, linoleic acid, linolenic acid, dicaprate, reclineate, monoolein, dilaurin, caprylic acid, arachidonic acid, glyceryl 1-monocaprate, mono and di-glycerides and physiologically acceptable salts thereof. The compositions may further include chelating agents such as, for example, ethylenediaminetetraacetic acid (EDTA), citric acid, salicylates (e.g. sodium salicylate, 5-methoxysalicylate, homovanilate).

The pharmaceutical compositions may comprise any of the expression vectors disclosed herein in an encapsulated form. For example, the expression vectors may be encapsulated by biodegradable polymers such as polylactide-polyglycolide, poly(orthoesters) and poly(anhydrides), or may be encapsulated in liposomes or dispersed within a microemulsion. Liposomes may be, for example, lipofectin or lipofectamine. In another example, a composition may comprise the expression vectors disclosed herein in or on anucleated bacterial minicells (Giacalone et al, Cell Microbiology 2006, 8(10): 1624-33). The expression vectors disclosed herein may be combined with nanoparticles.

Stable Producer Cell Lines

In another aspect of the present disclosure is a stable producer cell line for generating viral titer, wherein the stable producer cell line is derived from one of a GPR, GPRG, GPRT, GPRGT, or GPRT-G packing cell line. In some embodiments, the stable producer cell line is derived from the GPRT-G cell line. In some embodiments, the stable producer cell line is generated by (a) synthesizing an expression vector by cloning at least a nucleic acid sequence encoding an anti-HPRT shRNA into a recombinant plasmid (i.e. the synthesized vector may be any one of the vectors described herein); (b) generating DNA fragments from the synthesized vector; (c) forming a concatemeric array from (i) the generated DNA fragments from the synthesized vector, and (ii) from DNA fragments derived from an antibiotic resistance cassette plasmid; (d) transfecting one of the packaging cell lines with the formed concatemeric array; and (e) isolating the stable producer cell line. Additional methods of forming a stable producer cell line are disclosed in International Application No. PCT/US2016/031959, filed May 12, 2016, the disclosure of which is hereby incorporated by reference herein in its entirety.

Kits

In some embodiments is a kit comprising an expression vector or a composition comprising an expression vector as described herein. The kit may include a container, where the container may be a bottle comprising the expression vector or composition in an oral or parenteral dosage form, each dosage form comprising a unit dose of the expression vector. The kit may comprise a label or the like, indicating treatment of a subject according to the methods described herein. Likewise, in other embodiments is a kit comprising a composition comprising a population of nanocapsules including a payload adapted to knockout HPRT as described herein.

In some embodiments, the kit may include additional active agents. The additional active agents may be housed in a container separate from the container housing the vector or composition comprising the vector. For example, in some embodiments, the kit may comprise one or more doses of a purine analog (e.g. 6-TG or 6-MP) and optionally instructions for dosing the purine analog for conditioning and/or chemoselection (as those steps are described further herein). In other embodiments, the kit may comprise one or more doses of MTX or MPA and optionally instructions for dosing the MTX or MPA for negative selection as described herein.

Preparation of Substantially Hprt-Deficient Lymphocytes (“Modified Lymphocytes”)

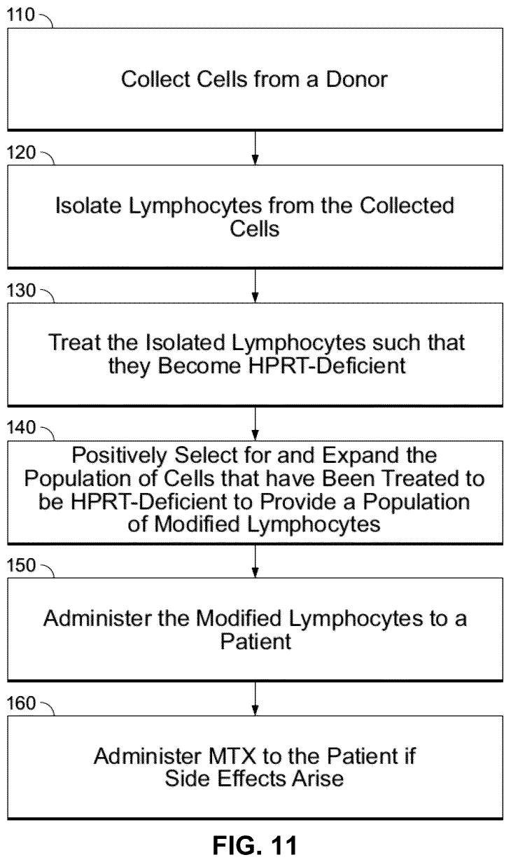

In one aspect of the present disclosure is a method of producing HPRT-deficient lymphocytes, e.g. T-cells (also referred to herein as “modified lymphocytes” or “modified T-cells”). With reference to FIG. 11, host cells, namely lymphocytes (e.g. T-cells), are first collected from a donor (step 110). In embodiments where hematopoietic stem cells (HSC) are also collected from a donor, the lymphocytes, e.g. T-cells, may be collected from the same donor from which the HSC graft is collected or from a different donor. In these embodiments, the cells may be collected at the same time or at a different time as the cells for the HSC graft. In some embodiments, the cells are collected from the same mobilized peripheral blood HSC harvest. In some embodiments, this could be a CD34-negative fraction (CD34-positive cells collected as per standard of care for donor graft), or a portion of the CD34-positive HSC graft if a progenitor T-cell graft is envisaged.

The skilled artisan will appreciate that the cells may be collected by any means. For example, the cells may be collected by apheresis, leukapheresis, or merely through a simple venous blood draw. In embodiments where the HSC graft is collected contemporaneously with the cells for modification, the HSC graft is cryopreserved so as to allow time for manipulation and testing of the lymphocytes, e.g. T-cells, collected. Non-limiting examples of T-cells include T helper T-cells (e.g. Th1, Th2, Th9, Th17, Th22, Tfh), regulatory T-cells, natural killer T-cells, gamma delta T-cells, and cytotoxic lymphocytes (CTLs).

Following collection of the cells, the lymphocytes, e.g. T-cells, are isolated (step 120). The lymphocytes, e.g. T-cells, may be isolated from the aggregate of cells collected by any means known to those of ordinary skill in the art. For example, CD3+ cells may be isolated from the collected cells via CD3 microbeads and the MACS separation system (Miltenyi Biotec). It is believed that the CD3 marker is expressed on all T-cells and is associated with the T-cell receptor. It is believed that about 70 to about 80% of human peripheral blood lymphocytes and about 65-85% of thymocytes are CD3+. In some embodiments, the CD3+ cells are magnetically labeled with CD3 MicroBeads. Then the cell suspension is loaded onto a MACS Column which is placed in the magnetic field of a MACS Separator. The magnetically labeled CD3+ cells are retained on the column. The unlabeled cells run through and this cell fraction is depleted of CD3+ cells. After removal of the column from the magnetic field, the magnetically retained CD3+ cells can be eluted as the positively selected cell fraction.

Alternatively, CD62L+ T-cells may be isolated from the collected cells is via an IBA life sciences CD62L Fab Streptamer Isolation Kit. Isolation of human CD62L+ T-cells is performed by positive selection. PBMCs are labeled with magnetic CD62L Fab Streptamers. Labeled cells are isolated in a strong magnet where they migrate toward the tube wall on the side of the magnet. This CD62L positive cell fraction is collected and cells are liberated from all labeling reagents by addition of biotin in a strong magnet. The magnetic Streptamers migrate toward the tube wall and the label-free cells remain in the supernatant. Biotin is removed by washing. The resulting cell preparation is highly enriched with CD62L+ T-cells with a purity of more than 90%. No depletion steps and no columns are needed.

In alternative embodiments, the lymphocytes, e.g. T-cells, are not isolated at step 120, but rather the aggregate of cells collected at step 110 are used for subsequent modification. While in some embodiments the aggregate of cells may be used for subsequent modification, in some instances the method of modification may be specific for a particular cell population within the total aggregate of cells. This could be done in a number of ways; for example, targeting genetic modification to a particular cell type by targeting gene vector delivery, or by targeting expression of, for example a shRNA to HPRT to a particular cell type, i.e., T-cells.

Following isolation of the T-cells, the T-cells are treated to decrease HPRT activity (step 130), i.e. to decrease expression of the HPRT1 gene. For example, the T-cells may be treated such that they have about 50% or less residual HPRT1 gene expression, about 45% or less residual HPRT1 gene expression, about 40% or less residual HPRT1 gene expression, about 35% or less residual HPRT1 gene expression, about 30% or less residual HPRT1 gene expression, about 25% or less residual HPRT1 gene expression, about 20% or less residual HPRT1 gene expression, about 15% or less residual HPRT1 gene expression, about 10% or less residual HPRT1 gene expression, or about 5% or less residual HPRT1 gene expression.

The lymphocytes, e.g. T-cells, may be modified according to several methods. In some embodiments, T-cells may be modified by transduction with an expression vector, e.g. a lentiviral vector, encoding a shRNA targeted to the HPRT1 gene, such as described herein. For example, an expression vector may comprise a first expression control sequence operably linked to a first nucleic acid sequence, the first nucleic acid sequence encoding a shRNA to knockdown HPRT, wherein the shRNA has at least 90% identity to the sequence of any of SEQ ID NOS: 2, 5, 6, and 7. By way of another example, an expression vector may comprise a first expression control sequence operably linked to a first nucleic acid sequence, the first nucleic acid sequence encoding a shRNA to knockdown HPRT, wherein the shRNA has at least 90% identity to the sequence of any of SEQ ID NOS: 8, 9, 10, and 11. In some embodiments, the expression vector is encapsulated within a nanocapsule.

In some embodiments, the expression vector may include a first nucleic acid sequence encoding an endonuclease and a second nucleic acid sequence encoding a guide RNA. In some embodiments, the first nucleic acid sequence encodes Cas9. In some embodiments, the first nucleic acid sequence encodes Cas12a. In some embodiments, the first nucleic acid sequence encodes Cas12b. In some embodiments, the second nucleic acid sequence encode a guide RNA having at least 90% sequence identity to any one of SEQ ID NOS: 25-39. In some embodiments, the second nucleic acid sequence encode a guide RNA having at least 91% sequence identity to any one of SEQ ID NOS: 25-39. In some embodiments, the second nucleic acid sequence encode a guide RNA having at least 92% sequence identity to any one of SEQ ID NOS: 25-39. In some embodiments, the second nucleic acid sequence encode a guide RNA having at least 93% sequence identity to any one of SEQ ID NOS: 25-39. In some embodiments, the second nucleic acid sequence encode a guide RNA having at least 94% sequence identity to any one of SEQ ID NOS: 25-39. In some embodiments, the second nucleic acid sequence encode a guide RNA having at least 95% sequence identity to any one of SEQ ID NOS: 25-39. In some embodiments, the second nucleic acid sequence encode a guide RNA having at least 96% sequence identity to any one of SEQ ID NOS: 25-39. In some embodiments, the second nucleic acid sequence encode a guide RNA having at least 97% sequence identity to any one of SEQ ID NOS: 25-39. In some embodiments, the second nucleic acid sequence encode a guide RNA having at least 98% sequence identity to any one of SEQ ID NOS: 25-39. In some embodiments, the second nucleic acid sequence encode a guide RNA having at least 99% sequence identity to any one of SEQ ID NOS: 25-39. In some embodiments, the second nucleic acid sequence encode a guide RNA having any one of SEQ ID NOS: 25-39.

In some embodiments, the second nucleic acid sequence encode a guide RNA having at least 90% sequence identity to any one of SEQ ID NOS: 40-61. In some embodiments, the second nucleic acid sequence encode a guide RNA having at least 91% sequence identity to any one of SEQ ID NOS: 40-61. In some embodiments, the second nucleic acid sequence encode a guide RNA having at least 92% sequence identity to any one of SEQ ID NOS: 40-61. In some embodiments, the second nucleic acid sequence encode a guide RNA having at least 93% sequence identity to any one of SEQ ID NOS: 40-61. In some embodiments, the second nucleic acid sequence encode a guide RNA having at least 94% sequence identity to any one of SEQ ID NOS: 40-61. In some embodiments, the second nucleic acid sequence encode a guide RNA having at least 95% sequence identity to any one of SEQ ID NOS: 40-61. In some embodiments, the second nucleic acid sequence encode a guide RNA having at least 96% sequence identity to any one of SEQ ID NOS: 40-61. In some embodiments, the second nucleic acid sequence encode a guide RNA having at least 97% sequence identity to any one of SEQ ID NOS: 40-61. In some embodiments, the second nucleic acid sequence encode a guide RNA having at least 98% sequence identity to any one of SEQ ID NOS: 40-61. In some embodiments, the second nucleic acid sequence encode a guide RNA having at least 99% sequence identity to any one of SEQ ID NOS: 40-61. In some embodiments, the second nucleic acid sequence encode a guide RNA having any one of SEQ ID NOS: 40-61.

In some embodiments, the lymphocytes, e.g. T-cells, may be modified by transfection with an endonuclease and a guide RNA. In some embodiments, the lymphocytes, e.g. T-cells, may be modified by transfection with a particle including endonuclease and a guide RNA. In some embodiments, the lymphocytes, e.g. T-cells, may be modified by transfection with a nanocapsule including a payload adapted to knockout HPRT, i.e. a gene editing approach may be used to knockout HPRT. In some embodiments, the lymphocytes, e.g. T-cells, may be modified by transfection with a targeted nanocapsule including a payload adapted to knockout HPRT, i.e. a gene editing approach may be used to knockout HPRT.

For example, T-cells may be treated with a HPRT-targeted CRISPR/Cas9 RNP, a CRISPR/Cas12a RNP, or a CRISPR/Cas12b RNP, as described herein. In some embodiments, the nanocapsule may include a guide RNA having at least 90% sequence identity to any one of SEQ ID NOS: 25-39. In other embodiments, the nanocapsule may include a guide RNA having at least 95% sequence identity to any one of SEQ ID NOS: 25-39. In some embodiments, the nanocapsule may include a guide RNA having at least 90% sequence identity to any one of SEQ ID NOS: 40-61. In other embodiments, the nanocapsule may include a guide RNA having at least 95% sequence identity to any one of SEQ ID NOS: 40-61.

After the T-cells are modified at step 130, the population of HPRT-deficient T-cells is selected for and/or expanded (step 140). In some embodiments, the culture may concurrently select for and expand cells with enhanced capacity for engraftment (e.g. central memory or T stem cell phenotype). In some embodiments, the culture period is less than 14 days. In some embodiments, the culture period is less than 7 days.

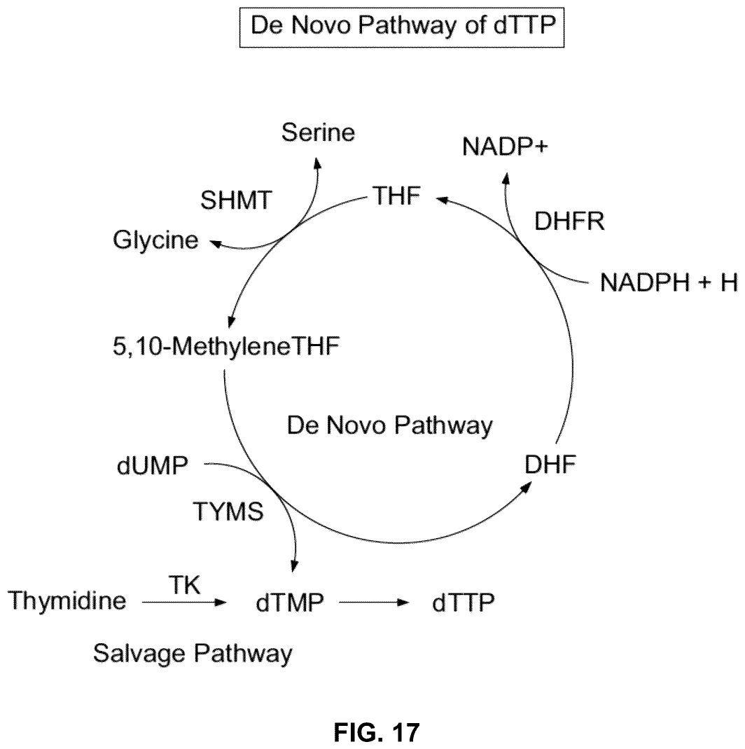

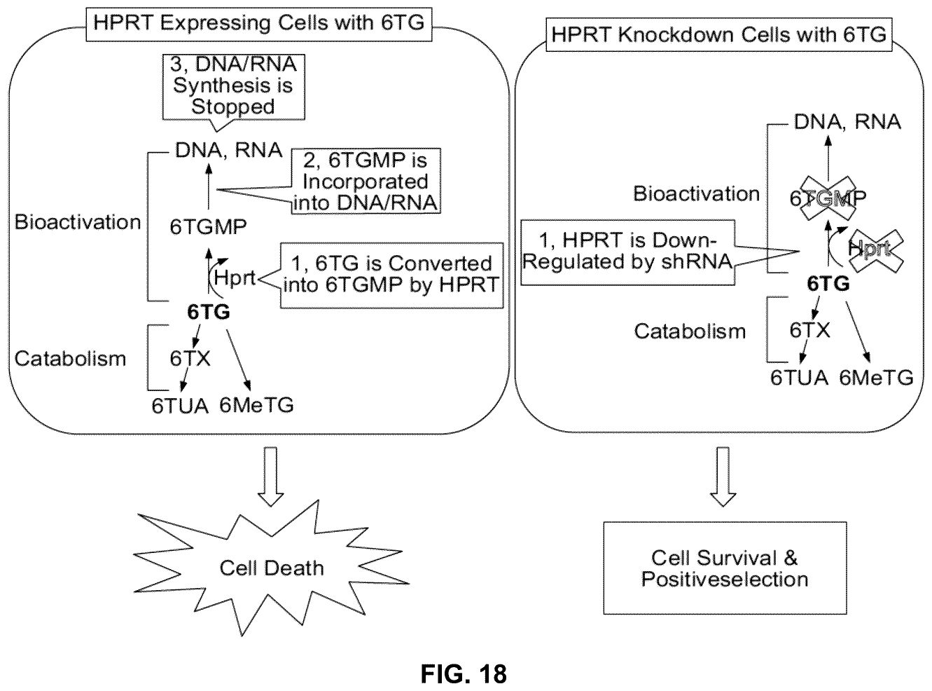

In some embodiments, the step of selecting for and expanding cells comprises treating the population of HPRT-deficient (or substantially HPRT-deficient) lymphocytes, e.g. T-cells, ex vivo with a guanosine analog antimetabolite (such as 6-thioguanine (6-TG), 6-mercaptopurine (6-MP), or azathiopurine (AZA). In some embodiments, the lymphocytes, e.g. T-cells, are cultured in the presence of 6-thioguanine (“6-TG”), thus killing cells which have not been modified at step 130. 6-TG is a guanine analog that can interfere with dGTP biosynthesis in the cell. Thio-dG can be incorporated into DNA during replication in place of guanine, and when incorporated, often becomes methylated. This methylation can interfere with proper mis-match DNA repair and can result in cell cycle arrest, and/or initiate apoptosis. 6-TG has been used clinically to treat patients with certain types of malignancies due to its toxicity to rapidly dividing cells. In the presence of 6-TG, HPRT is the enzyme responsible for the integration of 6-TG into DNA and RNA in the cell, resulting in blockage of proper polynucleotide synthesis and metabolism (see FIG. 18). On the other hand, the salvage pathway is blocked in HPRT-deficient cells (see FIG. 18). Cells thus use the de novo pathway for purine synthesis (see FIG. 17). However, in HPRT wild type cells, cells use the salvage pathway and 6-TG is converted to 6-TGMP in the presence of HPRT. 6-TGMP is converted by phosphorylation to thioguanine diphosphate (TGDP) and thioguanine triphosphate (TGTP). Simultaneously deoxyribosyl analogs are formed, via the enzyme ribonucleotide reductase. Given that 6-TG is highly cytotoxic, it can be used as a selection agent to kill cells with a functional HPRT enzyme.

The generated HPRT-deficient cells are then contacted with a purine analog ex vivo. For the knockdown approach, it is believed that there still may be residual HPRT in the cells and that HPRT-knockdown cells can tolerate a range of purine analog but will be killed at high dosages/amounts. In this situation, the concentration of purine analogs used for ex vivo selection ranges from about 15 μM to about 200 nM. In some embodiments, the concentration of purine analogs used for ex vivo selection ranges from about 10 μM to about 50 nM. In some embodiments, the concentration of purine analogs used for ex vivo selection ranges from about 5 μM to about 50 nM. In some embodiments, the concentration ranges from about 2.5 μM to about 10 nM. In other embodiments, the concentration ranges from about 2 μM to about 5 nM. In yet other embodiments, the concentration ranges from about 1 μM to about 1 nM.

For the knockout approach, it is believed that HPRT may be totally eliminated or near totally eliminated from HPRT-knockout cells and the generated HPRT-deficient cells will be highly tolerant to purine analogs. In some embodiments, the concentration of purine analogs used for ex vivo selection in this case ranges from about 200 μM to about 5 nM. In some embodiments, the concentration of purine analogs used for ex vivo selection in this case ranges from about 100 μM to about 20 nM. In some embodiments, the concentration ranges from 80 μM about to about 10 nM. In other embodiments, the concentration ranges from about 60 μM to about 10 nM. In yet other embodiments, the concentration ranges from about 40 μM to about 20 nM.

In other embodiments, modification of the cells (e.g. through knockdown or knockout of HPRT) may be efficient enough such that ex vivo selection for the HPRT-deficient cells is not necessary, i.e. selection with 6-TG or other like compound is not required.

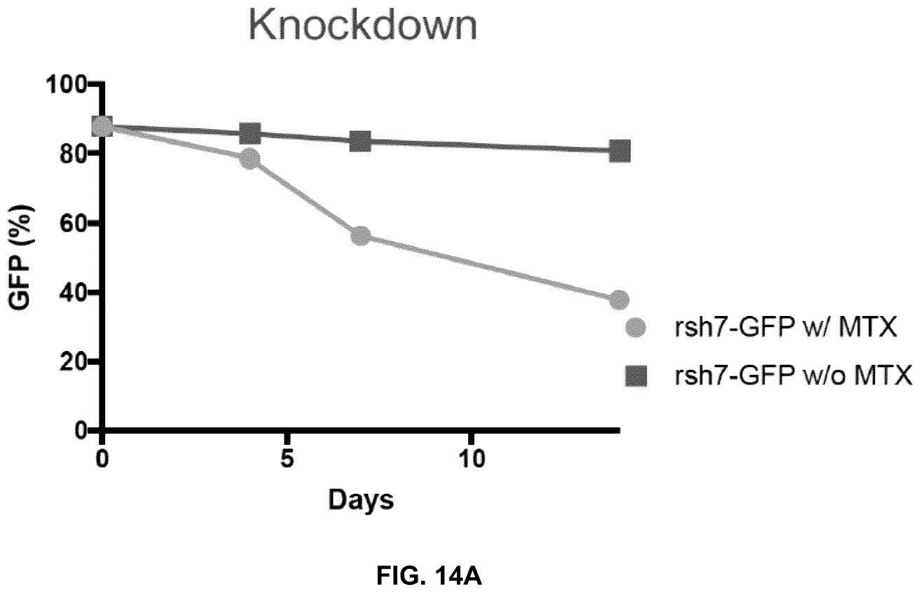

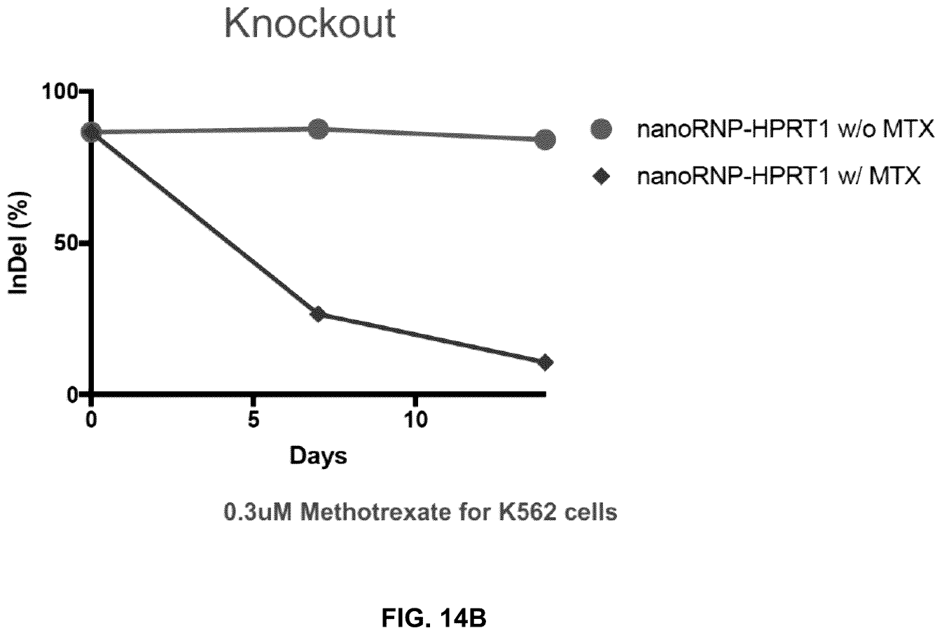

In some embodiments, the generated HPRT-deficient cells are contacted with both a purine analog and with allopurinol which is an inhibitor of xanthine oxidase (XO). By inhibiting XO, more available 6-TG to be metabolized by HPRT. When 6-TG is metabolized by HPRT it forms 6-TGNs which are the toxic metabolites to the cells (6-TGN encompasses monophosphate (6-TGMP), diphosphate (6-TGDP) and triphosphate (6-TGTP)) (see FIG. 14). (see, for example, Curkovic et. al., Low allopurinol doses are sufficient to optimize azathioprine therapy in inflammatory bowel disease patients with inadequate thiopurine metabolite concentrations. Eur J Clin Pharmacol. 2013 August; 69(8):1521-31; Gardiner et. al. Allopurinol might improve response to azathioprine and 6-mercaptopurine by correcting an unfavorable metabolite ratio. J Gastroenterol Hepatol. 2011 January; 26(1):49-54; Seinen et. al. The effect of allopurinol and low-dose thiopurine combination therapy on the activity of three pivotal thiopurine metabolizing enzymes: results from a prospective pharmacological study. J Crohns Colitis. 2013 November; 7(10):812-9; and Wall et. al. Addition of Allopurinol for Altering Thiopurine Metabolism to Optimize Therapy in Patients with Inflammatory Bowel Disease. Pharmacotherapy. 2018 February; 38(2):259-270, the disclosures of each are hereby incorporated by reference herein in their entireties).

In some embodiments, allopurinol is introduced to the generated HPRT-deficient cells prior to introduction of the purine along. In other embodiments, allopurinol is introduced to the generated HPRT-deficient cells simultaneously with the introduction of the purine along. In yet other embodiments, allopurinol is introduced to the generated HPRT-deficient cells following the introduction of the purine along.

Following selection and expansion, the modified lymphocytes, e.g. T-cells, product is tested. In some embodiments, the modified lymphocytes, e.g. T-cells, product is tested according to standard release testing (e.g. activity, mycoplasma, viability, stability, phenotype, etc.; see Molecular Therapy: Methods & Clinical Development Vol. 4 Mar. 2017 92-101, the disclosure of which is hereby incorporated by reference herein in its entirety).

In other embodiments, the modified lymphocytes, e.g. T-cells, product is tested for sensitivity to a dihydrofolate reductase inhibitor (e.g. MTX or MPA). Dihydrofolate reductase inhibitors, including both MTX and MPA, are believed to inhibit de novo synthesis of purines but have different mechanisms of action. For example, it is believed that MTX competitively inhibits dihydrofolate reductase (DHFR), an enzyme that participates in tetrahydrofolate (THF) synthesis. DHFR catalyzes the conversion of dihydrofolate to active tetrahydrofolate. Folic acid is needed for the de novo synthesis of the nucleoside thymidine, required for DNA synthesis. Also, folate is essential for purine and pyrimidine base biosynthesis, so synthesis will be inhibited. Mycophenolic acid (MPA) is potent, reversible, non-competitive inhibitor of inosine-5′-monophosphate dehydrogenase (IMPDH), an enzyme essential to the de novo synthesis of guanosine-5′-monophosphate (GMP) from inosine-5′-monophosphate (IMP).

Dihydrofolate reductase inhibitors, including both MTX or MPA, therefore inhibit the synthesis of DNA, RNA, thymidylates, and proteins. MTX or MPA blocks the de novo pathway by inhibiting DHFR. In HPRT−/− cell, there is no salvage or de novo pathway functional, leading to no purine synthesis, and therefore the cells die. However, the HPRT wild type cells have a functional salvage pathway, their purine synthesis takes place and the cells survive. In some embodiments, the modified lymphocytes, e.g. T-cells, are substantially HPRT-deficient. In some embodiments, at least about 70% of the modified lymphocyte, e.g. T-cells, population is sensitive to MTX or MPA. In some embodiments, at least about 75% of the modified lymphocyte, e.g. T-cells, population is sensitive to MTX or MPA. In some embodiments, at least about 80% of the modified lymphocyte, e.g. T-cells, population is sensitive to MTX or MPA. In some embodiments, at least about 85% of the modified lymphocyte, e.g. T-cells, population is sensitive to MTX or MPA. In other embodiments, at least about 90% of the modified lymphocyte, e.g. T-cells, population is sensitive to MTX or MPA. In yet other embodiments, at least about 95% of the modified lymphocyte, e.g. T-cells, population is sensitive to MTX or MPA. In yet other embodiments, at least about 97% of the modified lymphocyte, e.g. T-cells, population is sensitive to MTX or MPA.

In some embodiments, an alternative agent may be used in place of either MTX or MPA, including, but not limited to ribavarin (IMPDH inhibitor); VX-497 (IMPDH inhibitor) (see Jain J, VX-497: a novel, selective IMPDH inhibitor and immunosuppressive agent, J Pharm Sci. 2001 May; 90(5):625-37); lometrexol (DDATHF, LY249543) (GAR and/or AICAR inhibitor); thiophene analog (LY254155) (GAR and/or AICAR inhibitor), furan analog (LY222306) (GAR and/or AICAR inhibitor) (see Habeck et al., A Novel Class of Monoglutamated Antifolates Exhibits Tight-binding Inhibition of Human Glycinamide Ribonucleotide Formyltransferase and Potent Activity against Solid Tumors, Cancer Research 54, 1021-2026, February 1994); DACTHF (GAR and/or AICAR inhibitor) (see Cheng et. al. Design, synthesis, and biological evaluation of 10-methanesulfonyl-DDACTHF, 10-methanesulfonyl-5-DACTHF, and 10-methylthio-DDACTHF as potent inhibitors of GAR Tfase and the de novo purine biosynthetic pathway; Bioorg Med Chem. 2005 May 16; 13(10):3577-85); AG2034 (GAR and/or AICAR inhibitor) (see Boritzki et. al. AG2034: a novel inhibitor of glycinamide ribonucleotide formyltransferase, Invest New Drugs. 1996; 14(3):295-303); LY309887 (GAR and/or AICAR inhibitor) ((2S)-2-[[5-[2-[(6R)-2-amino-4-oxo-5,6,7,8-tetrahydro-1H-pyrido[2,3-d]pyrimidin-6-yl]ethyl]thiophene-2-carbonyl]amino]pentanedioic acid); alimta (LY231514) (GAR and/or AICAR inhibitor) (see Shih et. al. LY231514, a pyrrolo[2,3-d]pyrimidine-based antifolate that inhibits multiple folate-requiring enzymes, Cancer Res. 1997 Mar. 15; 57(6):1116-23); dmAMT (GAR and/or AICAR inhibitor), AG2009 (GAR and/or AICAR inhibitor); forodesine (Immucillin H, BCX-1777; trade names Mundesine and Fodosine) (inhibitor of purine nucleoside phosphorylase [PNP]) (see Kicska et. al., Immucillin H, a powerful transition-state analog inhibitor of purine nucleoside phosphorylase, selectively inhibits human T lymphocytes (T-cells), PNAS Apr. 10, 2001. 98 (8) 4593-4598); and immucillin-G (inhibitor of purine nucleoside phosphorylase [PNP]).

Given the sensitivity to MTX or MPA of the modified T-cells produced according to steps 110 through 140, MTX or MPA (or another dihydrofolate reductase inhibitor) may be used to selectively eliminate HPRT-deficient cells, as described herein. In some embodiments, an analog or derivative of MTX or MPA may be substituted for MTX or MPA. Derivatives of MTX are described in U.S. Pat. No. 5,958,928 and in PCT Publication No. WO/2007/098089, the disclosures of which are hereby incorporated by reference herein in their entireties.

Methods of Treatment

In some embodiments, the modified lymphocytes, e.g. T-cells, prepared according to steps 110 to 140 are administered to a patient (step 150). In some embodiments, the modified lymphocytes, e.g. T-cells, (or CAR T-cells or TCR T-cells as described herein) are provided to the patient in a single administration (e.g. a single bolus, or administration over a set time period, for example and infusion over about 1 to 4 hours or more). In other embodiments, multiple administrations of the modified lymphocytes, e.g. T-cells, are made. If multiple doses of the modified lymphocytes, e.g. T-cells, are administered, each dose may be the same or different (e.g. escalating doses, decreasing doses).

In some embodiments, an amount of the dose of modified T-cells is determined based on the CD3-positive T-cell content/kg of the subject's body weight. In some embodiments, the total dose of modified T-cells ranges from about 0.1×10⁶/kg body weight to about 730×10⁶/kg body weight. In other embodiments, the total dose of modified T-cells ranges from about 1×10⁶/kg body weight to about 500×10⁶/kg body weight. In yet other embodiments, the total dose of modified T-cells ranges from about 1×10⁶/kg body weight to about 400×10⁶/kg body weight. In further embodiments, the total dose of modified T-cells ranges from about 1×10⁶/kg body weight to about 300×10⁶/kg body weight. In yet further embodiments, the total dose of modified T-cells ranges from about 1×10⁶/kg body weight to about 200×10⁶/kg body weight.

Where multiple doses are provided, the frequency of dosing may range from about 1 week to about 36 weeks. Likewise, where multiple doses are provided, each dose of modified T-cells ranges from about 0.1×10⁶/kg body weight to about 240×10⁶/kg body weight. In other embodiments, each dose of modified T-cells ranges from about 0.1×10⁶/kg body weight to about 180×10⁶/kg body weight. In other embodiments, each dose of modified T-cells ranges from about 0.1×10⁶/kg body weight to about 140×10⁶/kg body weight. In other embodiments, each dose of modified T-cells ranges from about 0.1×10⁶/kg body weight to about 100×10⁶/kg body weight. In other embodiments, each dose of modified T-cells ranges from about 0.1×10⁶/kg body weight to about 60×10⁶/kg body weight. Other dosing strategies are described by Gozdzik J et al., Adoptive therapy with donor lymphocyte infusion after allogenic hematopoietic SCT in pediatric patients, Bone Marrow Transplant, 2015 January; 50(1):51-5), the disclosure of which is hereby incorporated by reference in its entirety.

The modified lymphocytes, e.g. T-cells, may be administered alone or as part of an overall treatment strategy. In some embodiments, the modified lymphocytes, e.g. T-cells, are administered following an HSC transplant, such as about 2 to about 4 weeks after the HSC transplant. For example, in some embodiments, the modified lymphocytes, e.g. T-cells, are administered after administration of an HSC transplant to help prevent or mitigate post-transplant immune deficiency. It is believed that the modified lymphocytes, e.g. T-cells, may provide a short term (e.g. about 3 to about 9 month) immune reconstitution and/or protection. As another example, and in other embodiments, the modified lymphocytes, e.g. T-cells, are administrated as part of cancer therapy to help induce a graft-versus-malignancy (GVM) effect or a graft-versus-tumor (GVT) effect. As a further example, the modified T-cells are CAR-T cells or TCR-modified T-cells which are HPRT-deficient, and which are administered as part of a cancer treatment strategy. Administration of the modified lymphocytes, e.g. T-cells, according to each of these treatment avenues are described in more detail herein. Of course, the skilled artisan will appreciate that other treatments for any underlying condition may occur prior to, subsequent to, or concurrently with administration of the modified lymphocytes, e.g. T-cells.

Administration of lymphocytes, e.g. T-cells, to a patient may result in unwanted side effects, including those recited herein. For example, graft-versus-host disease may occur after a patient is treated with lymphocytes, including modified T-cells (e.g. via knockdown or knockout of HPRT). In some aspects of the present disclosure, following administration of the modified lymphocytes, e.g. T-cells, at step 150, the patient is monitored for the onset of any side effects, including, but not limited to, GvHD. Should any side effects arise, such as GvHD (or symptoms of GvHD), MTX or MPA is administered to the patient (in vivo) at step 160 to remove at least a portion of the modified lymphocytes, e.g. T-cells, in an effort to suppress, reduce, control, or otherwise mitigate side effects, e.g. GvHD. In some embodiments, MTX or MPA is administered in a single dose. In other embodiments, multiple does of MTX and/or MPA are administered.

It is believed that the modified lymphocytes, e.g. T-cells, of the present disclosure (once selected for ex vivo and administered to the patient or mammalian subject), may serve as a modulatable “on”/“off” switch given their sensitivity to dihydrofolate reductase inhibitors (including both MTX or MPA). The modulatable switch allows for regulation of immune system reconstitution by selectively killing at least a portion of the modified lymphocytes, e.g. T-cells, in vivo through the administration of MTX to the patient should any side effects occur. This modulatable switch may be further regulated by administering further modified lymphocytes, e.g. T-cells, to the patient following MTX administration to allow further immune system reconstitution after side effects have been reduced or otherwise mitigated. Likewise, the modulatable switch allows for regulation of a graft-versus-malignancy effect by selectively killing at least a portion of the modified lymphocytes, e.g. T-cells, in vivo through the administration of MTX should any side effects occur. Again, the GVM effect may be fine-tuned by subsequently dosing further aliquots of modified lymphocytes, e.g. T-cells, to the patient once side effects are reduced or otherwise mitigated. This same principle applies to CAR-T cell therapy or therapy with TCR-modified T-cells, where again the CAR-T cells or TCR-modified T-cells may be selectively turned on/off through MTX administration. In view of this, the person of ordinary skill in the art will appreciate that any medical professional overseeing treatment of a patient can balance immune system reconstitution and/or the GVM effect while keeping side effects at bay or within tolerable or acceptable ranges. By virtue of the above, patient treatment may be enhanced while mitigating adverse effects.

In some embodiments, an amount of MTX administered ranges from about 2 mg/m²/infusion to about 100 mg/m²/infusion. In some embodiments, an amount of MTX administered ranges from about 2 mg/m²/infusion to about 90 mg/m²/infusion. In some embodiments, an amount of MTX administered ranges from about 2 mg/m²/infusion to about 80 mg/m²/infusion. In some embodiments, an amount of MTX administered ranges from about 2 mg/m²/infusion to about 70 mg/m²/infusion. In some embodiments, an amount of MTX administered ranges from about 2 mg/m²/infusion to about 60 mg/m²/infusion. In some embodiments, an amount of MTX administered ranges from about 2 mg/m²/infusion to about 50 mg/m²/infusion. In some embodiments, an amount of MTX administered ranges from about 2 mg/m²/infusion to about 40 mg/m²/infusion. In some embodiments, an amount of MTX administered ranges from about 2 mg/m²/infusion to about 30 mg/m²/infusion. In some embodiments, an amount of MTX administered ranges from about 20 mg/m²/infusion to about 20 mg/m²/infusion. In some embodiments, an amount of MTX administered ranges from about 2 mg/m²/infusion to about 10 mg/m²/infusion. In some embodiments, an amount of MTX administered ranges from about 2 mg/m²/infusion to about 8 mg/m²/infusion. In other embodiments, an amount of MTX administered ranges from about 2.5 mg/m²/infusion to about 7.5 mg/m²/infusion. In yet other embodiments, an amount of MTX administered is about 5 mg/m²/infusion. In yet further embodiments, an amount of MTX administered is about 7.5 mg/m²/infusion.

In some embodiments, between 2 and 6 infusions are made, and the infusions may each comprise the same dosage or different dosages (e.g. escalating dosages, decreasing dosages, etc.). In some embodiments, the administrations may be made on a weekly basis, or a bi-monthly basis.

In yet other embodiments, the amount of MTX administered is titrated such that uncontrolled side effects, e.g. GvHD, is resolved, while preserving at least some modified lymphocytes, e.g. T-cells, and their concomitant effects on reconstituting the immune system, targeting cancer, or inducing the GVM effect. In this regard, it is believed that at least some of the benefit of the modified lymphocytes, e.g. T-cells, may still be recognized while ameliorating side effects, e.g. GvHD. In some embodiments, additional modified lymphocytes, e.g. T-cells, are administered following treatment with MTX, i.e. following resolution, suppression, or control of the side effects, e.g. GvHD.

In some embodiments, the subject receives doses of MTX prior to administration of the modified lymphocytes, e.g. T-cells, such as to control or prevent side effects after HSC transplantation. In some embodiments, existing treatment with MTX is halted prior to administration of the modified lymphocytes, e.g. T-cells, and then resumed, at the same or different dosage (and using a same or different dosing schedule), upon onset of side effects following treatment with the modified lymphocytes, e.g. T-cells. In this regard, the skilled artisan can administer MTX on an as-need basis and consistent with the standards of care known in the medical industry.

Additional Treatment Strategies

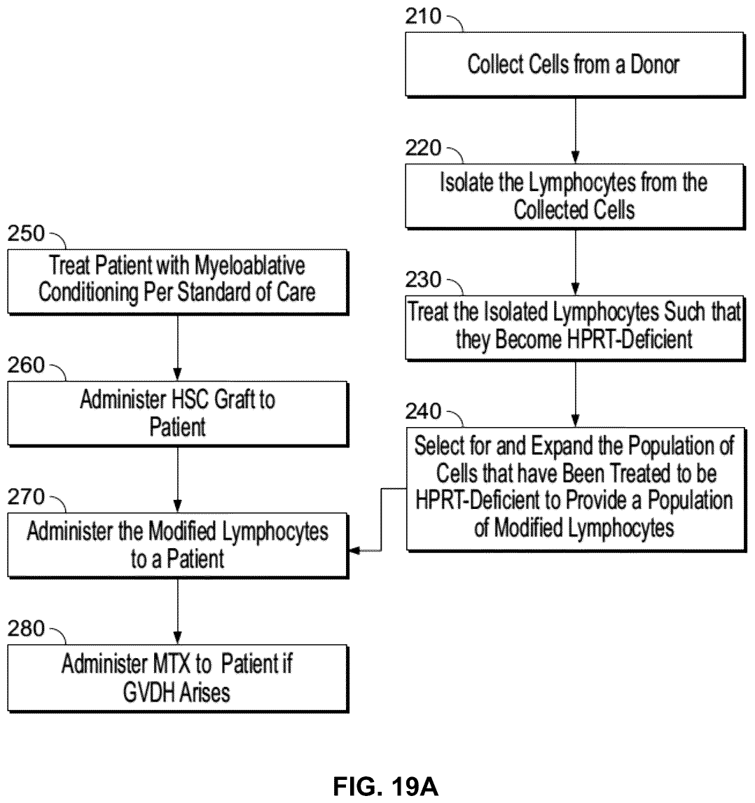

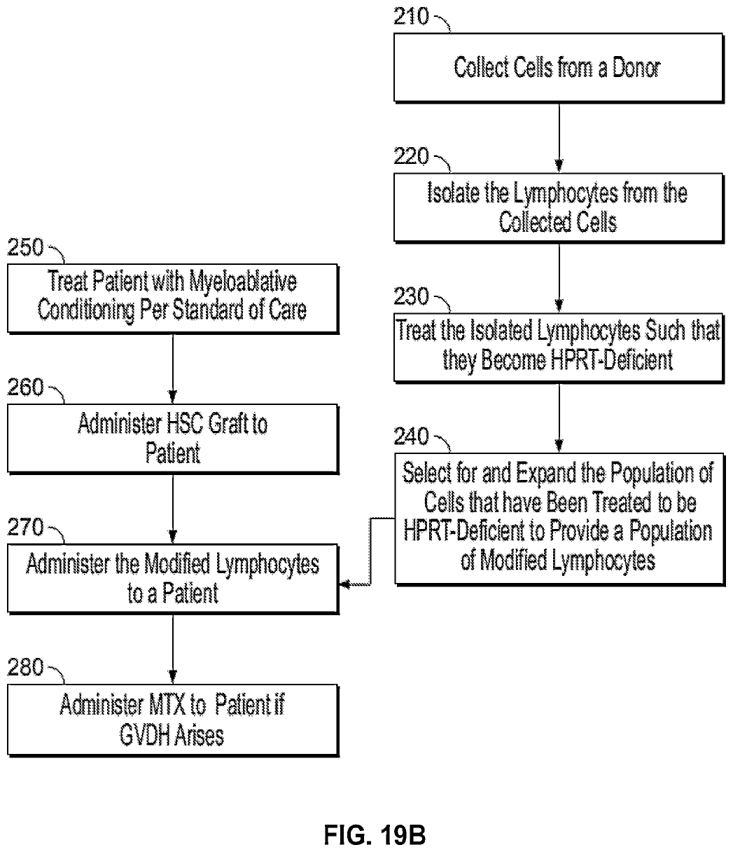

FIGS. 19A and B illustrate one method of reducing, suppressing, or controlling GvHD upon onset of symptoms. Initially, cells are collected from a donor at step 210. The cells may be collected from the same donor that provided the HSC for grafting (see step 260) or from a different donor. Lymphocytes are then isolated from the collected cells (step 220) and treated such that they become HPRT-deficient (step 230) (i.e. via knockdown or knockout of HPRT). Methods of treating the isolated cells are set forth herein. To arrive at a population of modified lymphocytes, e.g. T-cells, that are substantially HPRT deficient, the treated cells are positively selected for and expanded (step 240), such as described herein. The modified lymphocytes, e.g. T-cells, are then stored for later use. Prior to receiving the HSC graft (step 260), patients are treated with myeloablative conditioning as per the standard of care (step 250) (e.g. high-dose conditioning radiation, chemotherapy, and/or treatment with a purine analog; or low-dose conditioning radiation, chemotherapy, and/or treatment with a purine analog). In some embodiments, the patient is treated with the HSC graft (step 260) between about 24 and about 96 hours following treatment with the conditioning regimen.

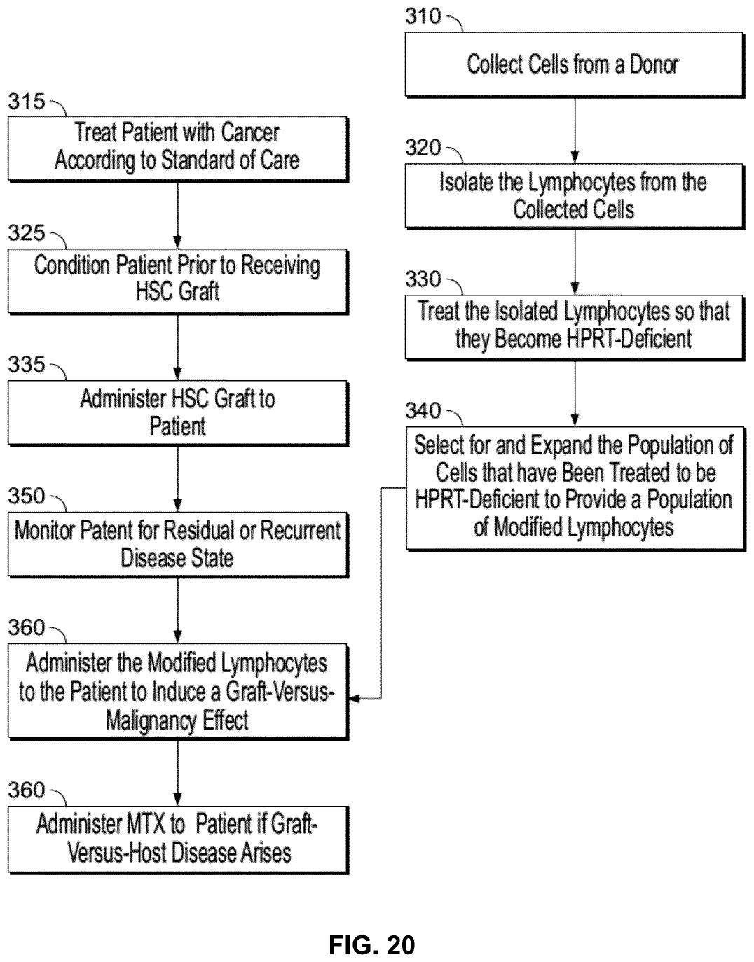

FIG. 20 illustrates one method of reducing, suppressing, or controlling GvHD upon onset of symptoms. Initially, cells are collected from a donor at step 310. The cells may be collected from the same donor that provided the HSC for grafting (see step 335) or from a different donor. Lymphocytes are then isolated from the collected cells (step 320) and treated such that they become HPRT-deficient (step 330). Methods of treating the isolated cells are set forth herein. To arrive at a population of modified lymphocytes, e.g. T-cells, that are substantially HPRT deficient, the treated cells are selected for and expanded (step 340), such as described herein. The modified lymphocytes, e.g. T-cells, are then stored for later use. A patient having cancer, for example a hematological cancer, may be treated according to the standard of care available to the patient at the time of presentation and staging of the cancer (e.g. radiation and/or chemotherapy, including biologics) (step 315). The patient may also be a candidate for HSC transplantation and, if so, a conditioning regimen (step 325) is implemented (e.g. by high-dose conditioning radiation or chemotherapy). It is believed that for malignancy, in some embodiments, one wishes to “wipe out” the blood system completely, or as close to completely as possible, thus, to killing off as many malignant cells as possible. The goals of such a conditioning regimen being to treat the cancer cells intensively, thereby making a cancer recurrence less likely, inactivate the immune system to reduce the chance of a stem cell graft rejection, and enable donor cells to travel to the marrow. In some embodiments, conditioning includes administration of one or more of cyclophosphamide, cytarabine (AraC), etoposide, melphalan, busulfan, or high-dose total body irradiation. The patient is then treated with an allogenic HSC graft (step 335). In some embodiments, the allogenic HSC graft induces at least a partial GVM, GVT, or GVL effect. Following grafting, the patient is monitored (step 350) for residual or recurrent disease. Should such residual or recurrent disease present itself, the modified lymphocytes, e.g. T-cells, (produce at step 340) are administered to the patient (step 360) such that a GVM, GVT, or GVT effect may be induced. The modified lymphocytes, e.g. T-cells, may be infused in a single administration of over a course of several administrations. In some embodiments, the modified lymphocytes, e.g. T-cells, are administered between about 1 day and about 21 days after the HSC graft. In some embodiments, the modified T-cells are administered between about 1 day and about 14 days after the HSC graft. In some embodiments, the modified lymphocytes, e.g. T-cells, are administered between about 1 day and about 7 days after the HSC graft. In some embodiments, the modified lymphocytes, e.g. T-cells, are administered between about 2 days and about 4 days after the HSC graft. In some embodiments, the modified lymphocytes, e.g. T-cells, are administered contemporaneously with the HSC graft or within a few hours of the HSC graft (e.g. 1, 2, 3, or 4 hours after the HSC graft).

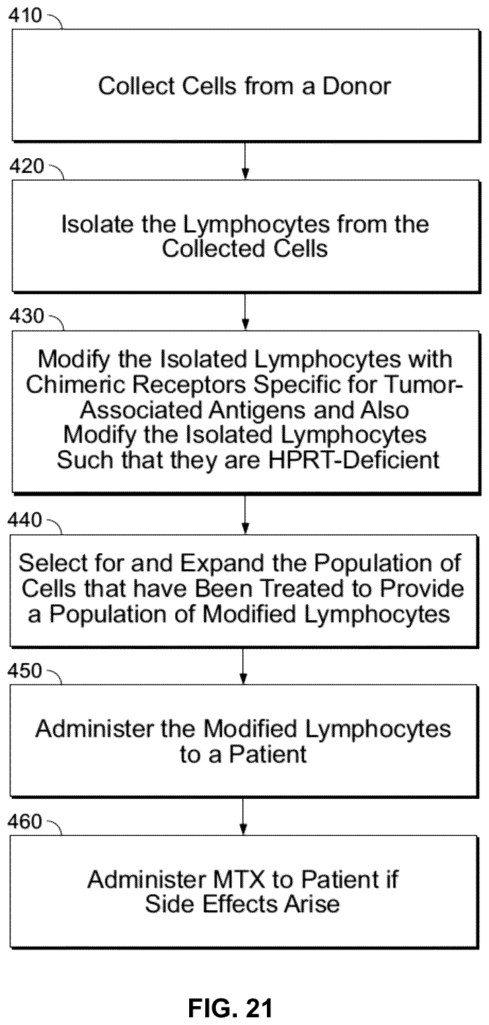

In another aspect of the present disclosure is a method of treating a patient having cancer by administering modified CAR T cells to a patient in need thereof, the modified CAR T cells being HPRT-deficient. FIG. 21 illustrates one method of treating a patient having cancer and subsequently reducing, suppressing, or controlling any deleterious side effects. Initially, cells are collected from a donor at step 410. Lymphocytes are then isolated from the collected cells (step 420) and modified to provide CAR T-cells that are HPRT-deficient.

### EXAMPLES

### Example 1—HPRT Knockdown Versus Knockout with 6-TG Selection

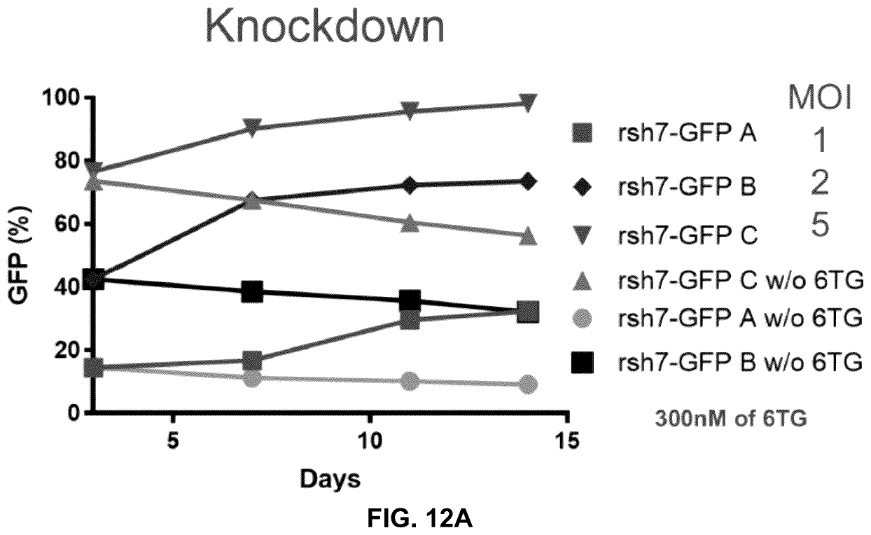

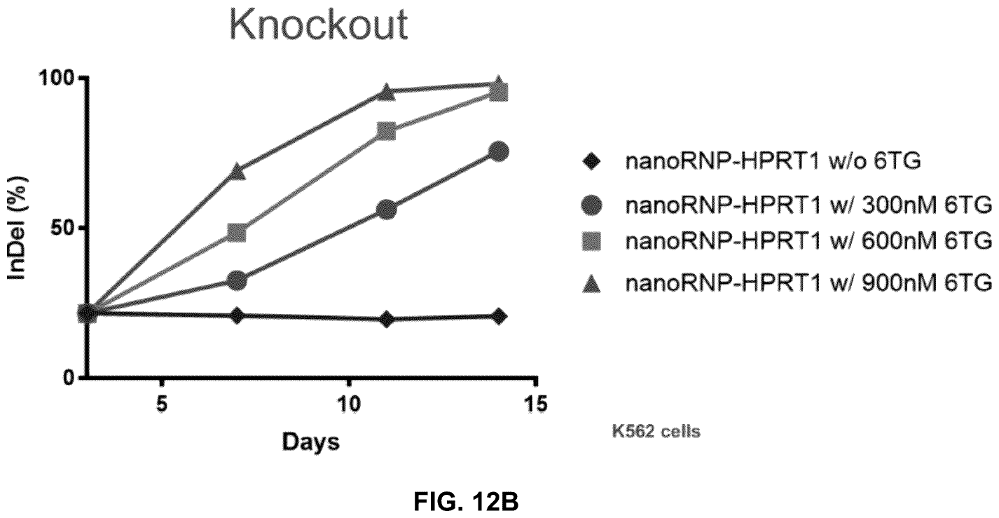

K562 cells were transduced with an expression vector including a nucleic acid sequence designed to knockdown HPRT and a nucleic acid sequence encoding the green fluorescent protein (GFP) (MOI=1/2/5); or were transfected with a nanocapsule including CRISPR/Cas9 and a sgRNA to knockout HPRT (100 ng/5×10⁴ cells) at day zero (0). 6-TG was added into the medium from day 3 through day 14. The medium was refreshed every 3 to 4 days. GFP was analyzed on a flow machine and the InDel % as analyzed with a T7E1 assay. FIG. 12A illustrates that the GFP+ population of transduced K562 cells increased from day 3 to day 14 under treatment of 6-TG; while the GFP+ population was almost steady without treatment. FIG. 12B illustrates that the HPRT knockout population of K562 cells increased from day 3 to 14 under treatment with 6-TG and higher dosages (900 nM) of 6-TG led to faster selection as compared with a dosage of 300/600 nM of 6-TG. It should be noted that the 6-TG selection process occurred faster on HPRT knockout cells as compared with the HPRT knockdown cells (MOI=1) at the same concentration of 300 nM of 6-TG from day 3 to day 14. The difference between knockdown and knockout could be explained by some level of residual HPRT by the RNAi knockdown approach as compared with the full elimination of HPRT by the knockout approach (see also FIG. 22). Therefore, HPRT-knockout cells were believed to have a much higher tolerance against 6-TG and were believed to grow much faster at higher dosages of 6-TG (900 nM) compared with HPRT-knockdown cells.

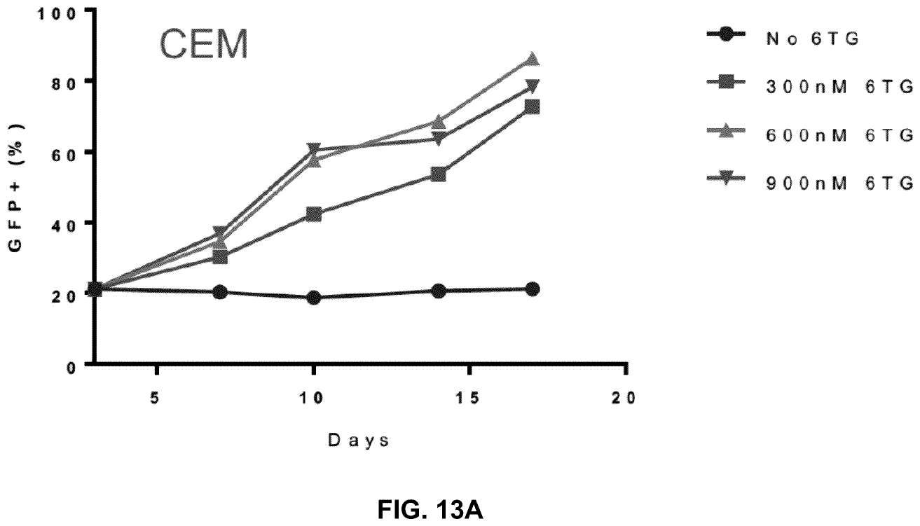

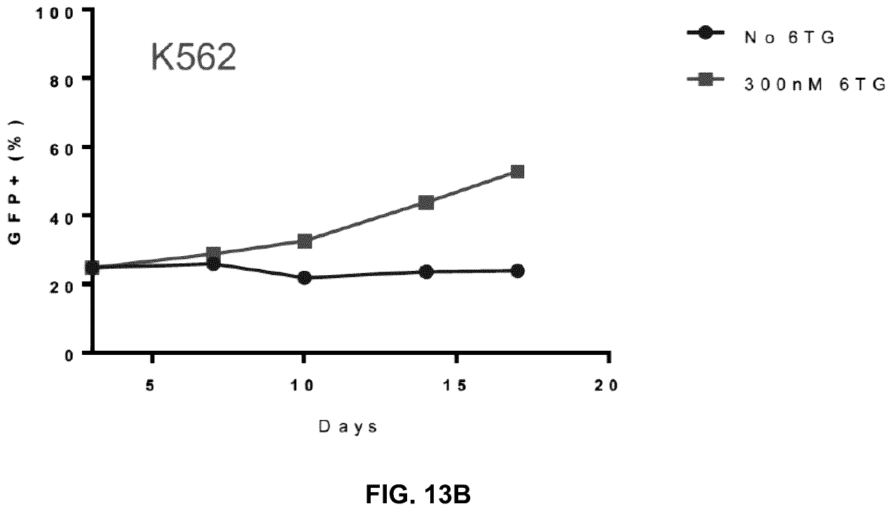

CEM cells were transduced with an expression vector including a nucleic acid sequence designed to knockdown HPRT and a nucleic acid sequence encoding the green fluorescent protein or transfected with a nanocapsule including CRISPR/Cas9 and a sgRNA to HPRT at day 0. 6-TG was added into the medium from day 3 to day 17. The medium was refreshed every 3 to 4 days. GFP as analyzed on a flow machine and the InDel % is analyzed by a T7E1 assay. FIG. 13A illustrates that the GFP+ population of transduced K562 cells increased from day 3 to day 17 under treatment of 6-TG while GFP+ population was almost steady without. FIG. 13B shows that HPRT knockout population of CEM cells increased from day 3 to 17 under treatment of 6-TG and that a higher dosage (900 nM) of 6-TG leads to a faster selection as compared with a dosage of 300/600 nM of 6-TG. It should be noted that 6-TG selection process occurred faster on HPRT knockout cells rather than HPRT knockdown cells (MOI=1) at the same concentration of 6-TG from day 3 to day 17.

### Example 2—Negative Selection with MTX or MPA

Transduced or transfected K562 cells (such as those from Example 1) were cultured with or without MTX from day 0 to day 14. The medium was refreshed every 3 to 4 days. GFP was analyzed on a flow machine and the InDel % was analyzed by T7E1 assay. FIG. 14A shows that the GFP-population of transduced K562 cells decreased under the treatment of 0.3 μM of MTX. On the other hand, the population of cells was steady without MTX. FIG. 14B illustrates that the transfected K562 cells were eliminated under treatment with 0.3 μM of MTX at a faster pace as compared with the HPRT-knockdown population.

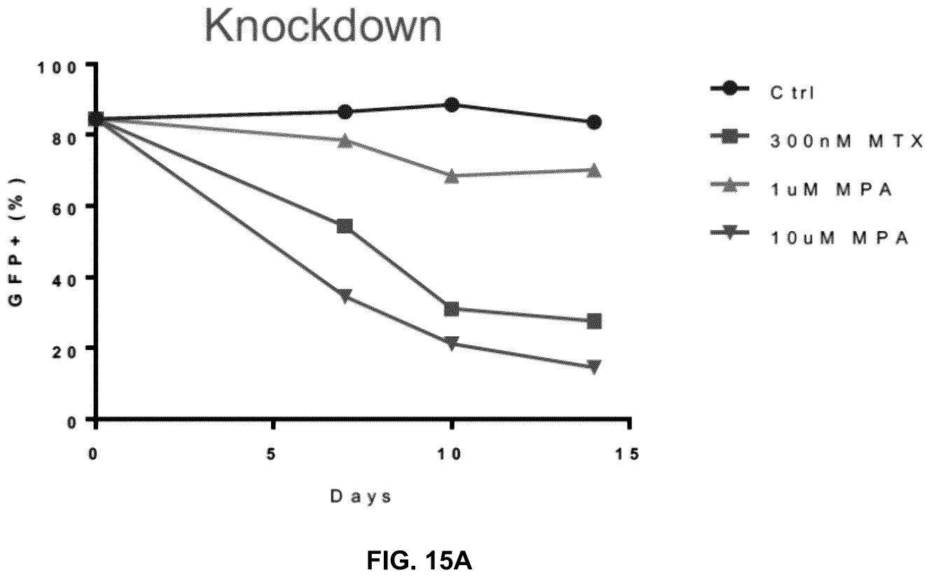

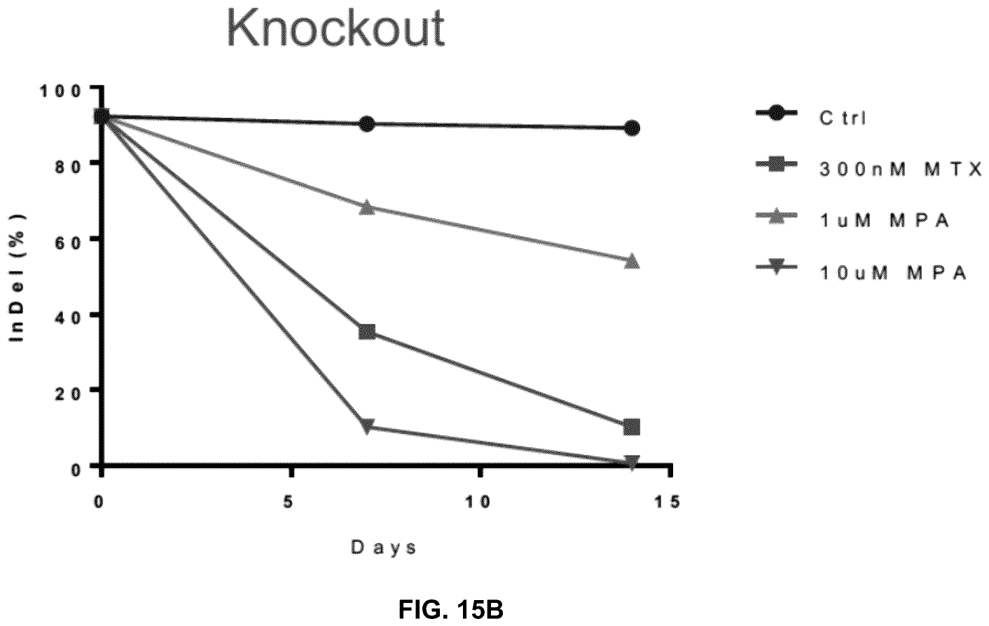

Transduced or transfected CEM cells (such as those from Example 1) were cultured with or without MTX from day 0 to day 14. The medium was refreshed every 3 to 4 days. GFP was analyzed on flow machine and InDel % was analyzed by T7E1 assay. FIG. 15A shows the GFP-population of transduced K562 decreased under the treatment of 1 μM of MPA or 0.3 μM of MTX or 10 μM of MPA while the population of cells was steady for the untreated group. FIG. 15B illustrates that the HPRT knockout population of CEM cells were eliminated at a faster pace under the treatment of 1 μM of MPA or 0.3 μM of MTX or 10 μM of MPA.

### Example 3—Negative Selection with MTX for K562 Cells

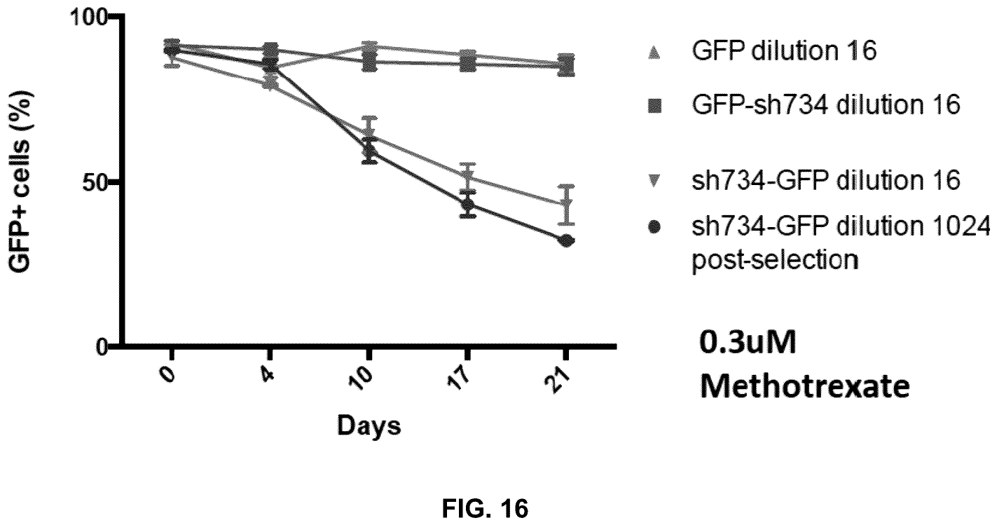

K562 cells were transduced with either (i) a TL20cw-GFP virus soup at dilution factor of 16, (ii) a TL20cw-Ubc/GFP-7SK/sh734 virus soup at a dilution factor of 16 (one sequentially encoding GFP and a shRNA designed to knockdown HPRT); or (iii) a TL20cw-7SK/sh734-UBC/GFP virus soup at a dilution factor of 16 (one sequentially encoding a shRNA designed to knockdown HPRT and GFP) (see FIG. 16). K562 cells were also transduced by a TL20cw-7SK/sh734-UBC/GFP virus soup at a dilution factor of 1024 (one encoding a nucleic acid encoding a shRNA designed to knockdown HPRT) (also shown in FIG. 16). Three days following transduction, all cells were cultured with medium containing 0.3 μM of MTX. As illustrated in FIG. 16, starting from greater than 90% of GFP+ population, GFP or GFP-sh734 transduced cells did not show a reduction in the GFP+ population while the sh734-GFP-transduced cells showed deselection of the GFP+ population (at both high dilution (1024) and low dilution (16) levels). The relative sh734 expression per vector copy number (VCN) for sh734-GFP-transduced cells and GFP-sh734-transduced cells were measured. The results suggested that methotrexate could only deselect cells transduced with a sh734-high-expression lentiviral vector (TL20cw-7SK/sh734-UBC/GFP) and not with a sh734-low-expression lentiviral vector (TL20cw-UBC/GFP-7SK/sh734). This example demonstrated that different vector designs (even those having the same shRNA) had an impact on the expression of the shRNA hairpin and could be used to determine whether transduced cells could be selected by treatment with MTX.

### Example 4—Transfection/Transduction, Selection and Expansion of Modified T cells

Primary human T-cell purification will be performed using peripheral blood mononuclear cells (PBMC) derived from bulk buffy packs (Australian Red Cross Blood Service), allowing enrichment of sufficient numbers of T-cells for down-stream applications. The remaining cells will be cryopreserved for future T cell function analyses, such as assessment of T cell proliferation in response to allo-antigens. Purified T-cells will be stimulated in vitro with immobilized anti-CD3 and recombinant human (rh)IL-2 (as per current published protocols Prommersberger et. al., Antibody-Based CAR T Cells Produced by Lentiviral Transduction, Current Protocols, March 2020, e93, https://doi.org/10.1002/cpim.93) for 48 hours followed by transduction with lentivirus, or transfection with DNA-containing nanoparticles, for modification of the HPRT1 gene. These modified T-cells will be cultured (2-3 days) followed by further expansion for up to 14 days in the presence of rhlL-2. Throughout the culture conditions, samples will be collected for assessment of the proportion of cells having successfully undergone gene-modification as determined by detection of a fluorescent tracer (e.g. GFP), as well as quantitative RT-PCR (qPCR) for detection of HPRT1 gene expression levels.

At 14-days post gene-modification, selection of gene-modified T-cells will be performed using 6-thioguanine (6-TG) to assess the dose required for negative selection of all non-modified T-cells. Titration of the 6-TG dose will also allow assessment of the potential donor-dependent sensitivity to this selection method, and how this may relate to the known TPMT genotype-dependent sensitivity to purine analogues. Investigation of 6-TG dose titration will also serve to assess the potential for dose-window variability based on the levels of shRNA expression. Selection will be followed by expansion of the modified T-cells with a selection of various cytokine combinations (IL-2/IL-7/IL-15/IL-21). The expanded T cell population will finally be tested for sensitivity to “kill switch” activation via the use of methotrexate.

### Example 5—Functional Assessment of Modified Human Primary T-Cells

The functional capacity of the modified T-cells will be assessed using in vitro methods to gain understanding of the potential consequences of gene-modification and culture conditions. T cell subtype proportions within the culture will be phenotyped, including assessment of naïve T-cells, effector T cell subtypes, memory T cell subtypes, regulatory T-cells etc. and including cell surface T-cells markers such as CD3/CD4/CD7/CD8/CD25/CD27/CD28/CD45RA/RO/CD56/CD62L/CD127 or FoxP3 and CD44). The potential development of T cell exhaustion as a consequence of extended culture conditions will also be assessed using flow cytometry. The functional capacity of the gene-modified T-cells to react to viral peptides will be assessed using T cell proliferation and cytokine release assays. This functional response to viral peptides from viruses such as Epstein Barr Virus (EBV) and cytomegalovirus (CMV) is believed to be particularly relevant, as these are the main viruses re-activated in the context of immune suppression and are relevant for patients in the clinic.

Finally, each of the donor modified T cell cultures will be assessed for alloreactivity against haplo-identical donor PBMCs cryopreserved (and genotyped) using in vitro proliferation assays. This is designed to mimic and measure the potential alloreactivity in a transplant context. The functional capacity of the regulatory T cell compartment within the gene-modified T cell pool could potentially also be assessed in this context.

### Example 6—Phenotypic and Functional Characterization of “Residual” Gene-Modified T Cell Populations Post Methotrexate Dosing

An understanding of the capacity for remaining gene-modified cells present within recipient post kill-switch induction and resolution of conditions such as GvHD or CRS, is the ability of the gene-modified cells to expand and re-constitute in appropriate numbers and with relevant function. The minimum threshold level for donor cell depletion followed by appropriate expandability and functional activity will therefore be important to understand. Clinical trials performed by third parties have shown that kill switch activation results in >99% depletion of donor T-cells in vivo within 2 hours, and that resolution of symptoms of GvHD and CRS occurs within 24-48 hours. In addition, the <1% of modified cells remaining in recipients are capable of re-expanding without resulting in re-activation of GvHD or CRS. The hypothesis that activation of the kill switch results in preferential death of actively expanding donor allo-reactive T-cells, therefore resulting in depletion of the T cell repertoire that has the potential to lead to relapse of GvHD/CRS. Ex vivo analysis of the re-expanded cells shows that the remaining repertoire is capable of recognizing and responding to viral antigens, indicating that the recipients will not be immunocompromised. In addition, the recipients in these trials remained disease-free to 100 days, with data not yet available beyond this limited follow-up period. Finally, it will be determined whether or not the residual/re-expanded populations remain susceptible to kill switch induction in a second pass if depletion of these cells is required at a later time due to donor cell related complications.

### Example 7—In Vivo Proof-of-Concept Studies in Mouse Models

Animal studies will be conducted to explore the in vivo behavior and properties of modified T-cells in both GvHD-resistant and GvHD-sensitive humanized NSG mice. Initial studies will aim to evaluate T cell engraftment and the MTX-induced “kill-switch” function in the modified T-cells. These studies will be conducted in GvHD-resistant mice. Studies to follow will aim to establish a mouse model of GvHD, providing a clinically-relevant in vivo setting in which to test the “kill-switch.” With a clear understanding of T-cell dose, distribution and function together with an understanding of MTX responsiveness, an in vivo POC study will be conducted in GvHD-sensitive mice receiving a leukemia-challenge. It will be shown that modified T-cells can be manipulated by triggering the MTX “kill-switch” to minimize GvHD while maintaining the ability to mount a GVT response.

### Example 8—Understanding T Cell Dose, Engraftment, Distribution, Survival and Methotrexate-Sensitivity

MHC KO NSG mice (GvHD-resistant) will be transplanted with different doses of modified T-cells, in order to establish an optimal T cell dose for sustained engraftment. At different time points the distribution of T-cells in lymphoid and non-lymphoid organs will be analyzed.

Using the optimal T-cell dose determined as noted herein (i), mice will be treated with different doses of methotrexate twenty-four to forty-eight hours following engraftment. The number of remaining T-cells in lymphoid and non-lymphoid organs will be determined in an analysis time-course designed to understand how rapidly the modified T-cells are eliminated.

A parallel study will be initiated to explore the longevity of the modified T-cell graft and MTX sensitivity of these T-cells over time. Mice receiving an optimal dose of modified T-cells will be aged for six months and then the MTX-induced “kill-switch” will be triggered. The number of remaining T-cells in lymphoid and non-lymphoid organs will be determined at the previously determined optimal analysis time. It will be shown how well modified T-cells could respond to the MTX “kill-switch” when triggered at a later time point, as may be the case in late-onset Acute or Chronic GvHD.

### Example 9—Establishment and Characterization of a GvHD Mouse Model and Analysis of Modified T-Cell Graft

To initiate GvHD, irradiation conditioned NSG mice will be transplanted with the optimal dose of modified T-cells within 2-24h post conditioning. From published literature, GvHD develops in these mice by about day 25 (body posture, activity, fur and skin condition and weight loss monitored) with disease end point reached by ˜day 55 (>20% weight loss with clinical symptoms of GvHD). Should disease progression be significantly slower or more aggressive, T-cell doses higher and lower respectively, than the optimal dose could be tested (approx. 10⁶-10⁷ T-cells based on literature).

With T-cell dose and disease kinetics optimized, T cell engraftment will be explored in a time-course analysis. T cell seeding of different organs is a feature of the GvHD and this will be explored in our model. The time course analysis time points will be determined by the onset and severity of GvHD observed.

When the modified T-cells are clearly detectable in lymphoid organs, T-cell (CD4+ and CD8+) functionality will be analyzed. T-cells will be stimulated in vitro with various stimuli (e.g. PMA, CD3/CD28) and analyzed for phenotype, proliferation, cytokine production and ex vivo anti-tumor cytotoxicity. T-cells will be specifically analyzed for their ability to respond to viral peptides e.g. CMV, EBV & FLU (Proimmune ProMix CEF peptide pool) as a measure of their ability to respond to latent virus reactivation.

### Example 10—Activation of Methotrexate-Induce “Kill Switch” in GvHD Model

NSG mice will be irradiated and transplanted with modified T-cells as per previously determined optimal conditions. At the acute or chronic phase of GvHD, mice will be administered with different doses of MTX including an optimal dose. The percentage of modified T-cells in peripheral blood will be determined weekly until the end of the experiments. GvHD development will be monitored to confirm if mice can be rescued from developing progressive disease. Infiltration of modified T-cells into various organs will be quantified to understand the severity of GvHD at a systemic level.

### Example 11—Modified T-Cells POC in a GVT/GvHD Mouse Model

NSG mice will be irradiated and transplanted with modified T-cells as per previously determined optimal conditions. Within twenty-four post-irradiation, mice will receive a dose of P815 H2-Kd cell line to establish leukemia. The P815 cells will be previously transduced to express GFP for in vivo biodistribution and assessment of tumor growth. At the onset of GvHD mice will be treated with the optimal dose of MTX and disease progression as well as leukemia burden will be monitored until the end of the experiment.

### Example 12—HPRT Knockout Guide RNAs

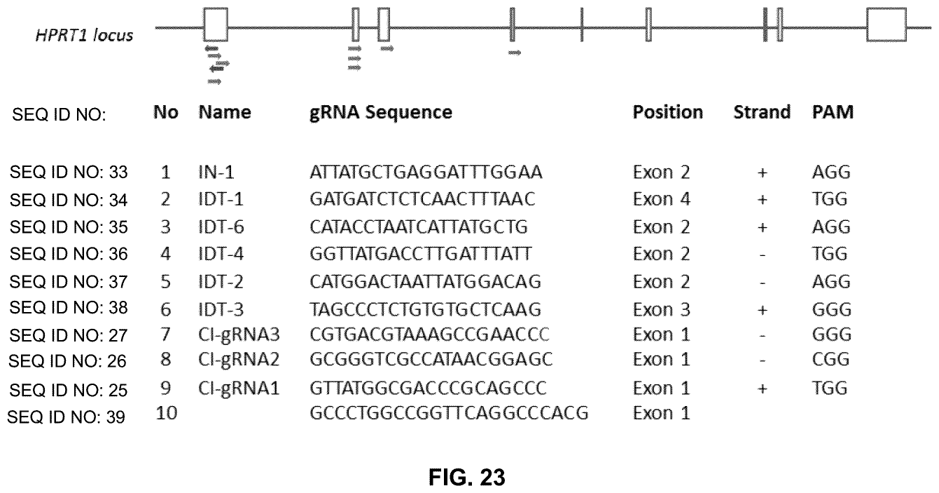

FIG. 23 and the Table which follows set forth the various guide RNAs which were examined for on target and off target effects. “IDT-4” (SEQ ID NO:36) was elected as a lead gRNA for remaining Knockout experiments, such as those described herein. SEQ ID NO: 39 (“Nat Paper”) was derived from Yoshioka, S. et al. (2016). Development of a mono-promoter-driven CRISPR/Cas9 system in mammalian cells. Scientific Reports, 5, 18341, the disclosure of which is hereby incorporated by reference herein in its entirety.

| SEQ | | |
|---|---|---|
| ID NO: | Name | Nucleotide Sequence |
| 33 | IN-1 | ATTATGCTGAGGATTTGGAA |
| 34 | IDT-1 | GATGATCTCTCTCAACTTTAAC |
| 35 | IDT-6 | CATACCTAATCATTATGCTG |
| 36 | IDT-4 | GGTTATGACCTTGATTTATT |
| 37 | IDT-2 | CATGGACTAATTATGGACAG |
| 38 | IDT-3 | TAGCCCTCTGTGTGCTCAAG |
| 27 | Cl-gRNA3 | CGTGACGTAAAGCCGAACCC |
| 26 | Cl-gRNA2 | GCGGGTCGCCATAACGGAGC |
| 25 | Cl-gRNA1 | GTTATGGCGACCCGCAGCCC |
| 39 | Nat Paper | GCCCTGGCCGGTTCAGGCCCACG |

### Example 13—HPRT Targeted Knockout Resistance to 6-TG

Method

Jurkat cells were electroporated with a ribonucleoprotein (RNP) complex containing guide RNA (gRNA; GS-4; designated IDT-4) together with the Cas9 and tracrRNA. Cells which were confirmed to be transfected with the gRNA via tracr RNA were purified using fluorescence activated cells sorting (FACS) and subsequently cultured for 72 hours. Increasing concentrations of 6-thioguanine (6-TG) were then administered to the transduced cultured cells to assess resistance. Wild type (unmodified) Jurkat cells were used as a control.

Results

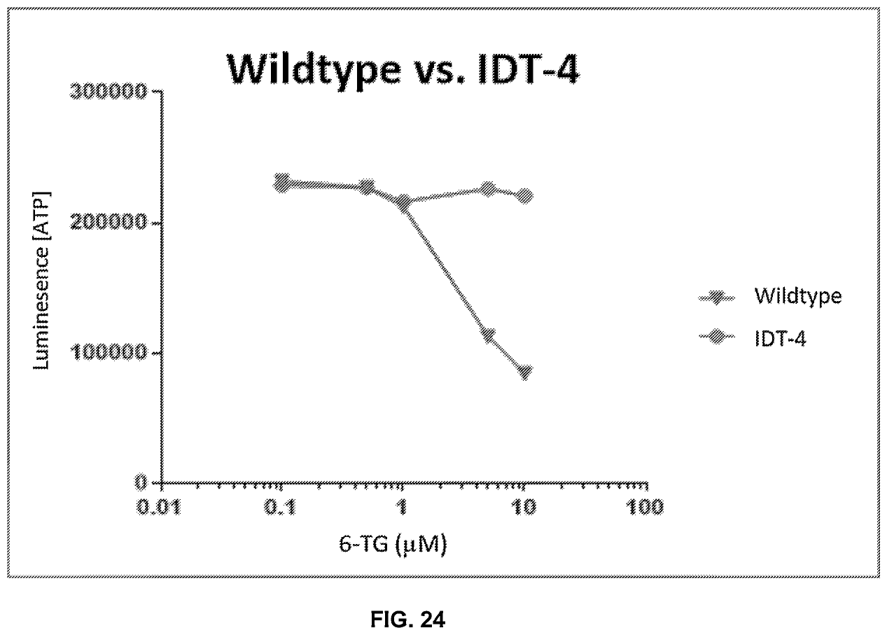

Luminescence (ATP detection) was used to assess cell viability. IDT-4 modified cells demonstrated resistance to increasing doses of 6-TG (tested up to 10 uM). Unmodified Jurkat cells (wild type) showed a decrease in cell viability with increasing concentrations of 6-TG (see FIG. 24).

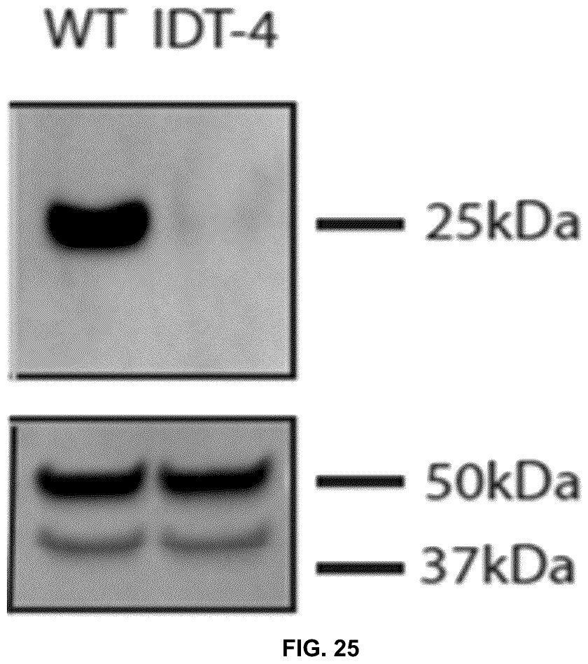

Unmodified (WT) and IDT-4 modified Jurkat cells were also analyzed for HPRT protein using Western blot (see FIG. 25). Unmodified Jurkat cells (WT) showed detectable levels of HPRT at the expected size (25 kDA) while IDT-4 modified cells had undetectable levels of HPRT. Actin protein detection (bottom panel) was used as a protein loading control.

### Example 14—Long-Term Cell Viability/Survival

Method

HPRT Knockout Jurkat cells (modified with guide RNA IDT-4; as described Example 1; designated HPRT−/−) were mixed together with Jurkat cells modified to express GFP alone (WT GFP) in approximately equal proportions.

Cells were cultured under standard culture conditions for 18 days before re-assessing the proportion of GFP+ cells in the culture.

Results

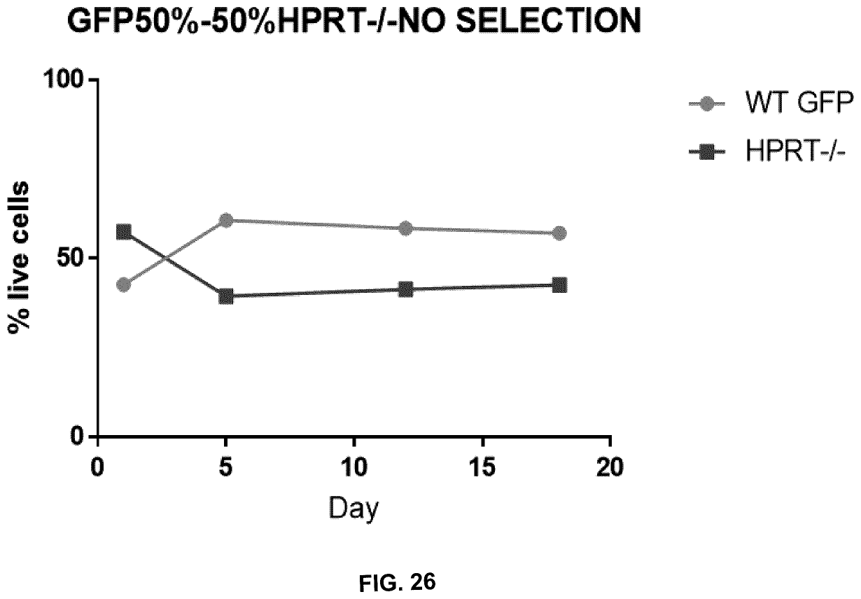

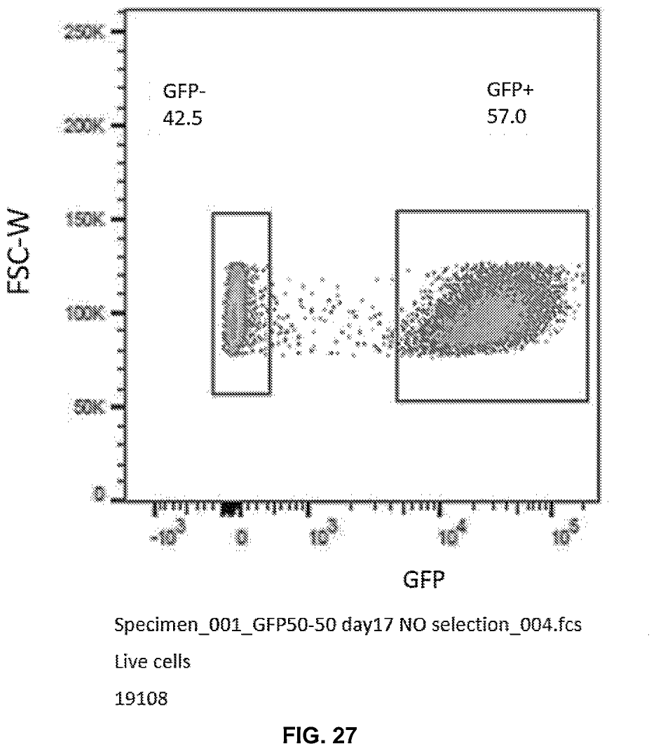

GFP proportion of cells at Day 18 was substantially similar to Day 1 (see FIG. 26), with no significant changes in the starting proportion of GFP+ wild type cells over time (see FIG. 27), indicating there is no survival advantage or disadvantage to cells being deficient for the HPRT protein. This further confirms that survival advantage in modified cells in presence of 6-TG is due to the absence of active HPRT enzyme.

### Example 15—HPRT Knockout Jurkat Cells—Methotrexate Sensitivity

Method & Results

(A) MTX Dose Response

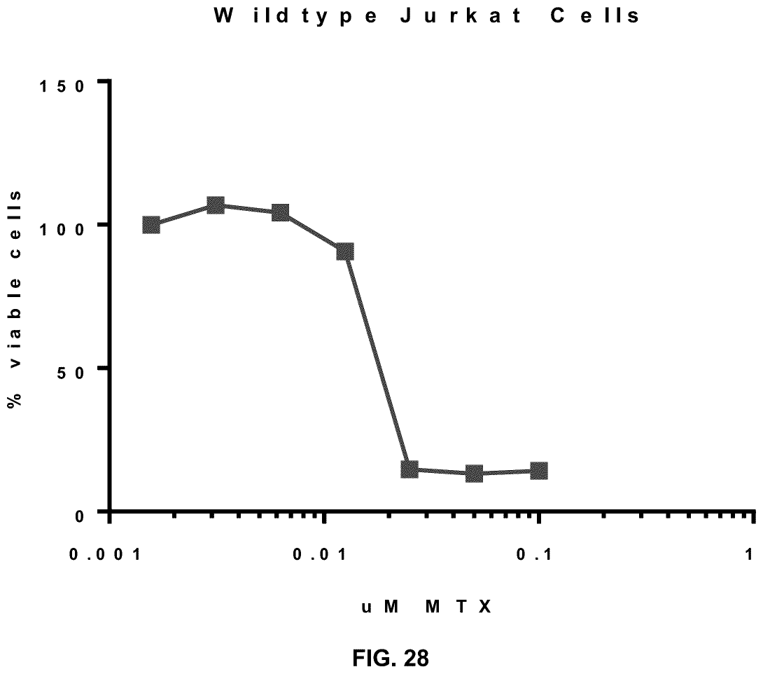

Jurkat cells (unmodified; WT) were cultured with increasing concentrations of methotrexate (MTX) to determine the MTX dosage window required to kill WT cells (see FIG. 28). A dose range of between 0.00625 and 0.025 μM MTX was selected for subsequent assessment of HPRT Knockout (IDT-4 modified) Jurkat cells.

(B) HPRT Knockout MTX Dose Response

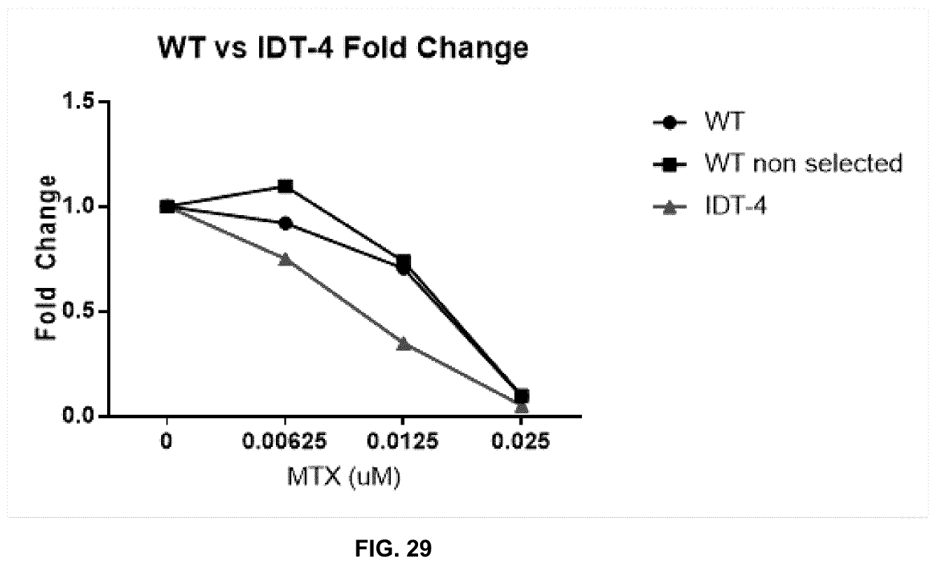

Dose response of HPRT Knockout (IDT-4 modified) Jurkat cells to MTX was compared to unmodified Jurkat cells (WT) to determine sensitivity to MTX (see FIG. 29). HPRT Knockout (IDT-4 modified) Jurkat cells demonstrated increased sensitivity to MTX at concentrations of 0.00625 and 0.0125 μM compared to wild type cells when cultured for 5 days.

### Example 16—HPRT Knockdown Jurkat Cells

Method

Jurkat T cells were modified with lentiviral vectors (A) TL20cw-7SK/sh734-UbC/GFP or (B) TL20cw-UbC/GFP-7SK/sh734. Jurkat cells were transduced with respective lentiviral vectors using 1 ml of un-diluted virus containing medium (VCM) together with 8 ng/ml of polybrene by centrifuging at 2,500 rpm for 90 minutes at room temperature followed by incubating for 60 minutes at 37° C. The cells were then cultured for 4 days post-transduction and removal of the VCM before using flow cytometry to determine the transduction efficiency (GFP positive cells).

Results

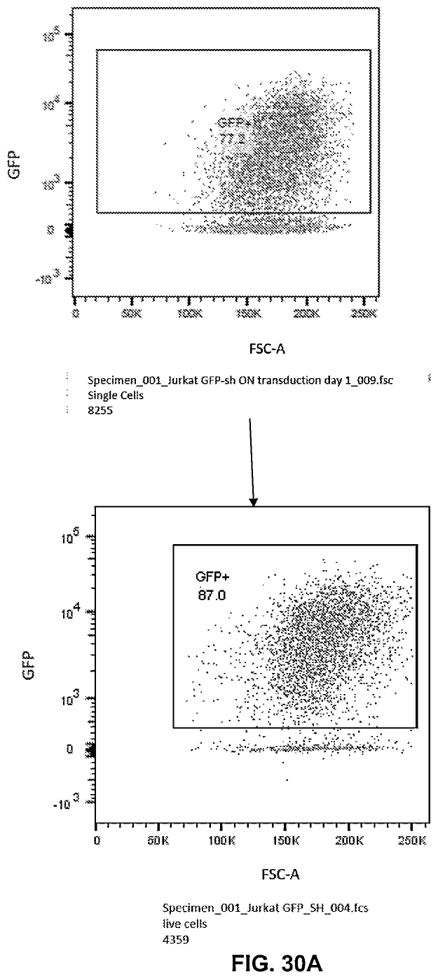

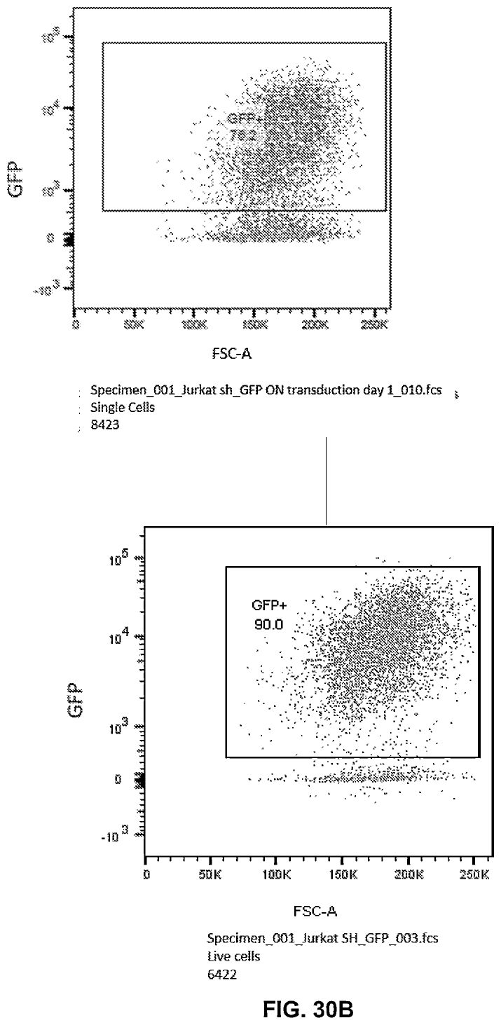

The Jurkat cells demonstrated a high transduction efficiency at day 4 post spin-inoculation, with the sh734-GFP (see FIG. 30A) resulting in 76.2% GFP+ cells at day 4, and the GFP-sh734 virus (see FIG. 30B) resulting in 77.2% GFP+ cells. Modified Jurkat cells were placed under 6-TG selection (10 uM, based on previous data generated assessing the sensitivity of wild-type unmodified Jurkat cells to 6-TG) for 3 days. Selection protocol resulted in an increase for each of the modified cells lines to 87% (se FIG. 30A) and 90% (see FIG. 30B) GFP+ cells, indicating death of the unmodified cells and enhanced survival of the sh734 containing cells.

### Example 17—HPRT Knockdown CEM T Cells

Method

CEM T cells were modified with the lentiviral vectors TL20cw-7SK/sh734-UbC/GFP (sh734-GFP) and TL20cw-UbC/GFP-7SK/sh734 (GFP-sh734).

CEM cells were spin-infected with 1 ml of undiluted virus containing medium (VCM) together with 10 ng/ml polybrene by centrifuging at 2,500 rpm for 90 minutes at room temperature followed by incubation for 60 minutes at 37° C. The proportion of GFP+ cells was determined after 4 days by flow cytometry. Transduction efficiencies were relatively low.

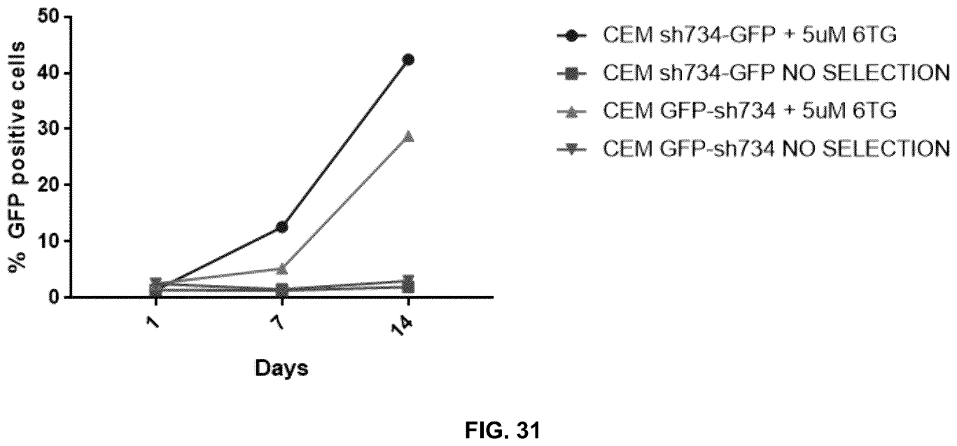

Modified CEM cells were subjected to 6-TG selection with 5 uM 6-TG for a total of 17 days. Cells containing the sh734 were successfully selected by 6-TG, increasing to 28.8% GFP+ in the case of sh734-GFP and 42.4% GFP+ in the case of GFP-sh734, indicating that these cells had a survival advantage over non-transduced cells (see FIG. 31).

### Example 18—Vector Production—HPRT Knockdown

Candidate vectors were prepared by insertion of an expression cassette comprising 7SK/sh734 into a pTL20cw vector (see, e.g., FIG. 32). Specifically, vectors listed in the Table which follows comprising the short hair pin, were generated.

| | Relative location/orientation |
|---|---|
| Vector | of 7SK/sh734 |
| TL20cw-7SK/sh734-UbC/GFP | upstream/forward |
| TL20cw-r7SK/sh734-UbC/GFP | upstream/reverse |
| TL20cw-UbC/GFP-7SK/sh734 | downstream/forward |
| TL20cw-UbC/GFP-r7SK/sh734 | downstream/reverse |

### Example 19—Transduction/Transfection

K562 or Jurkat cells were transduced with a vector including a nucleic acid sequence designed to knockdown HPRT and a nucleic acid sequence encoding the green fluorescent protein (GFP) (MOI from 0.1-5); or were transfected with a nanocapsule comprising CRISPR/Cas9 and a sgRNA to HPRT (100 ng/5×10⁴ cells).

### Example 20—Knockdown of HPRT and 6-TG Resistance

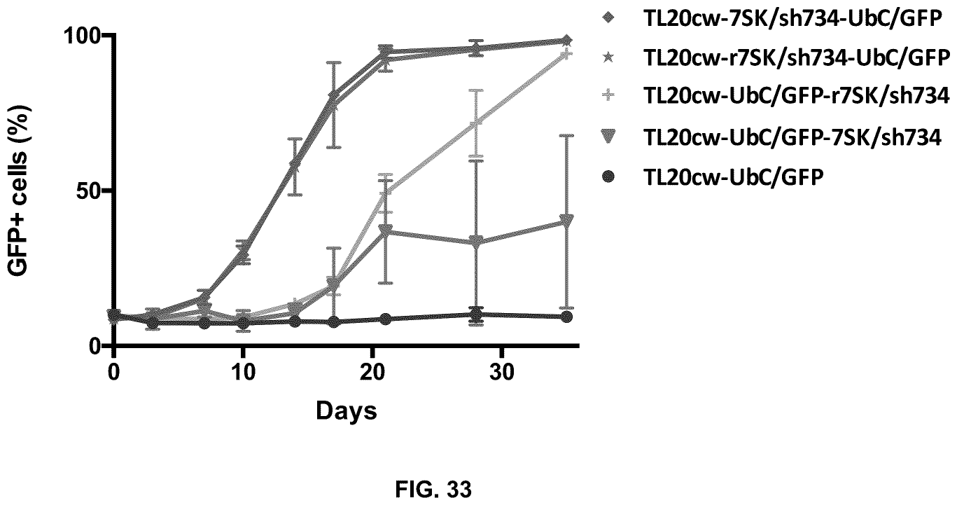

6-TG stock solution was added into the medium containing transduced/transfected K562 or Jurkat cells at day 3 or 4 post-transduction/transfection. 6-TG was maintained until day 14 or longer to a final concentration e.g. 300 nM for K562 cell and 2.5 uM for Jurkat cell. The medium was refreshed every 3 to 4 days. GFP was analyzed on flow machine, VCN was analyzed by VCN ddPCR assay and InDel % as analyzed with T7E1 assay. Results are provided in the Table which follows in FIG. 33.

| | Day 0 | | | | Day 35 | | |
|---|---|---|---|---|---|---|---|
| | | | sh734/ | | | sh734/ | |
| | | % | % | Rel. | % | % | Rel. |
| Vectors | Dilution | GFP+ | GFP+ | HPRT | GFP+ | GFP+ | HPRT |
| Mock | Mock | 0.4 | | 1 | 0.7 | | 1 |
| TL20cw-UbC/GFP | 1024 | 10.4 | ND | 0.95 | 9.1 | N/A | N/A |
| TL20cw-7SK/sh734- | 1024 | 8.3 | 3.03 | 1.19 | 99.6 | 21.06 | 0.087 |
| UbC/GFP | | | | | | | |
| TL20cw-r7SK/sh734- | 1024 | 8.2 | 4.49 | 0.71 | 95.4 | 27.48 | 0.070 |
| UbC/GFP | | | | | | | |
| TL20cw-UbC/GFP- | 1024 | 12.4 | 0.87 | 0.94 | 59.6 | 6.98 | 0.223 |
| 7SK/sh734 | | | | | | | |
| TL20cw-UbC/GFP- | 1024 | 10.1 | 0.27 | 0.72 | 98 | 15.17 | 0.163 |
| r7SK/sh734 | | | | | | | |

### Example 21—HPRT Knockout Guide RNAs

The Table which follows sets forth various guide RNA molecules which were studied, such as described in Examples 22-29 herein.

| | | SEQ ID | Corresponding |
|---|---|---|---|
| gRNA # | sgRNA guide and PAM | NO: | HPRT 1 target |
| gRNA 12 | GCCCCCCTTGAGCACACAGAGGG | 40 | Exon 4 |
| gRNA 13 | AGCCCCCCTTGAGCACACAGAGG | 41 | Exon 3 |
| gRNA 15 | GATGTGATGAAGGAGATGGGAGG | 42 | Exon 3 |
| gRNA 16 | CTGATAAAATCTACAGTCATAGG | 43 | Exon 3 |
| gRNA 17 | GTAGCCCTCTGTGTGCTCAAGGG | 44 | Exon 3 |
| gRNA 18 | TTATGCTGAGGATTTGGAAAGGG | 45 | Exon 2 |
| gRNA 19 | GTGCTTTGATGTAATCCAGCAGG | 46 | Exon 3 |
| gRNA 20 | TGAAGTATTCATTATAGTCAAGG | 47 | Exon 8 |
| gRNA 21 | TATCCTACAACAAACTTGTCTGG | 48 | Exon 8 |
| gRNA 22 | GAAGTATTCATTATAGTCAAGGG | 49 | Exon 8 |

### Example 22—in Silico Methods for Guide RNA Selection

Guide RNAs were developed based on in silico testing. In particular, the in silico design strategy used the following methods to arrive at the second generation gRNA:
(i) Identifying targeted exons in gene by using data and annotations collected by (University of California Santa Cruz) UCSC Genomics Institute
(a) Gene domains annotated in Pfam
(b) Evolutional conservation score PhyloP; and
(c) Clinical variants in the ClinVar database that lead to loss-of-function of HPRT1
(ii) Predict on-target efficiency with published methods
(a) Azimuth
(b) CHOPCHOP
(c) CRISPOR
(d) CCTop
(e) CRISTA
(iii) Predict frameshift knockout probability with published methods
(a) Out-of-Frame score
(b) Lindel
(c) inDelphi
(iv) Predict off-target sites with published methods
(a) Elevation (the same paper as Azimuth)
(b) CHOPCHOP (as CHOPCHOP above)
(c) Cas-OFFinder
(v) Ranking of off-target severity using annotations in
(a) ClinVar database (as in 1c)
(b) OncoKB (web page)

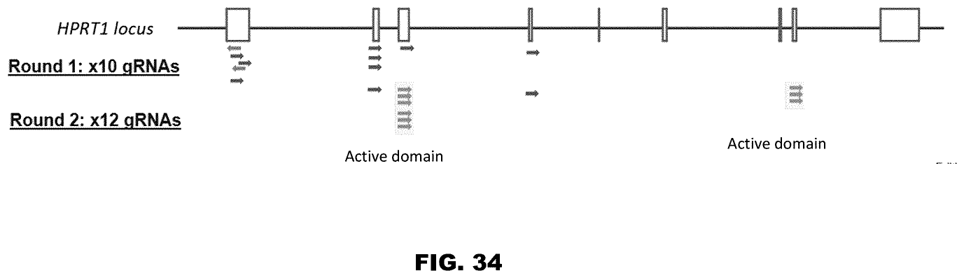

Based on the in silico testing conducted, Applicant identified ten guide RNAs targeting Exons 3 or 8 of HPRT 1 for further analysis (see, e.g., SEQ ID NOS: 40-49). FIG. 34 illustrates the exons within HPRT 1 that are targeted with the guide RNAs of the present disclosure, where the “Round 2” guide RNAs include those having SEQ ID NOS: 40-49.

The coordinates of HPRT1 exon 3 and 8 on human genome version GRCh38 are set forth below (chromosome, start, end):

| | | | |
|---|---|---|---|
| chrX | 134475181 | 134475364 | HPRT1_exon_3 |
| chrX | 134498608 | 134498684 | HPRT1_exon_8 |

While locations within Chromosome X are referenced herein to Genome Reference Consortium Human Build 38 (GRCh38), a person skilled in the art would understand that these referenced locations may be transposed to equivalent locations in alternative human genome builds or assemblies.

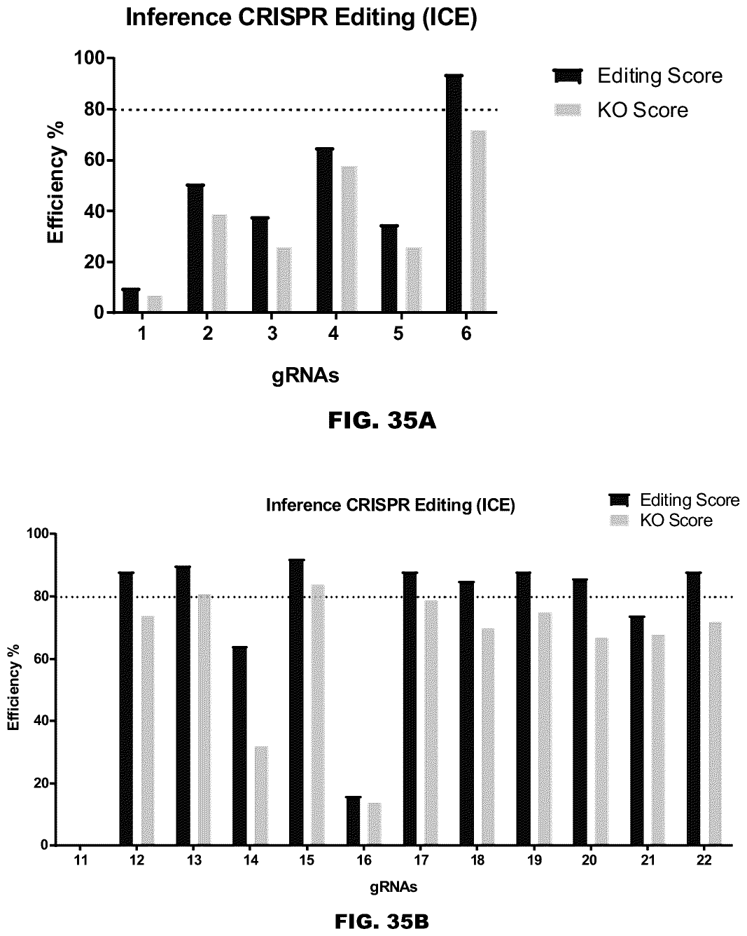

Synthego's Interference CRISPR Editing (ICE) tool using Sanger sequencing data was used as part of in silico design strategy, and the results of such analysis are illustrated in FIGS. 35A and 35B (where gRNA12 through gRNA22 correspond to SEQ ID NOS: 40-49 (see Example 21); and where gRNA23 through gRNA34 correspond to SEQ ID NOS: 50-61).

### Example 23—Screening and Validation in CEM Cells

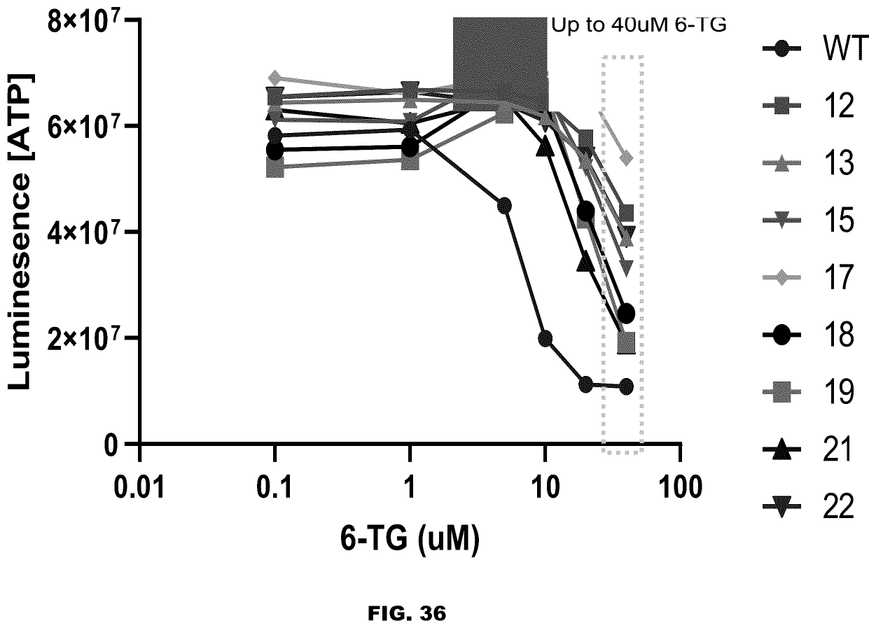

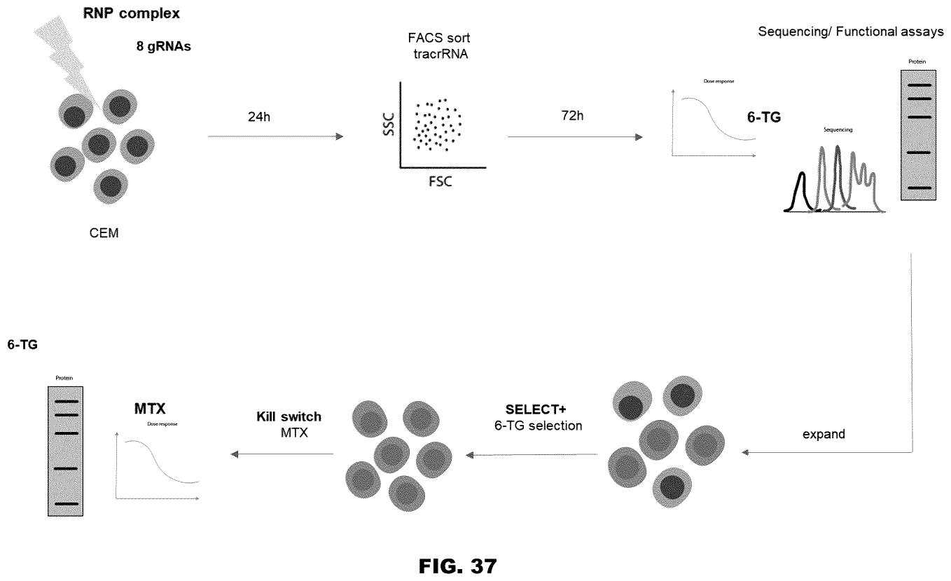

The top candidate guide RNAs from Example 21 were screen in Jurkat cell lines (n=1) and validated in CEM cell lines (n=2). For instance, Jurkat cells were electroporated with a ribonucleoprotein (RNP) complex containing guide RNA (e.g. an RNP including a guide RNA having any one of SEQ ID NOS: 40-49) together with the Cas9 and tracrRNA. The methodology and analysis was the same for both Jurkat cell lines and CEM cell lines (see also, FIG. 37). Following electroporation, cell viability was measured at different concentrations of 6-TG (see FIG. 36). The indel and knockout (KO) scores are provided in the tables below:

| | |
|---|---|
| Run #1 | Run #2 |

| | | Indel | KO | Indel | KO |
|---|---|---|---|---|---|
| gRNA | Cell type | (%) | score | (%) | score |
| 12 | CEM | 28 | 19 | 58 | 51 |
| 15 | CEM | 33 | 32 | 21 | 20 |
| 17 | CEM | | | 86 | 79 |
| 18 | CEM | 32 | 29 | 27 | 24 |
| 19 | CEM | 0 | 0 | 0 | 0 |
| 21 | CEM | 9 | 9 | 5 | 5 |
| 22 | CEM | 75 | 68 | 85 | 80 |

| | |
|---|---|
| gRNA | Round 1 |

| | | | |
|---|---|---|---|
| 13 | Primary T cells (#9) | 43 | 43 |
| 13 | Primary T cells (#10) | 19 | 19 |
| 21 | Primary T cells (#9) | 1 | 1 |
| 21 | Primary T cells (#10) | 0 | 0 |

Indel Percentage—The editing efficiency (percentage of the pool with non-wild type sequence) as determined by comparing the edited trace to the control trace. In the ICE algorithm, potential editing outcomes are proposed and fitted to the observed data using linear regression.

Knockout Score—Represents the proportion of cells that have either a frameshift or 21+bp indel. This score is a useful measure for those who are interested in understanding how many of the contributing indels are likely to result in a functional Knockout (KO) of the targeted gene.

### Example 24—CEM T Cells Modified Using Guide RNA Directed Against HPRT1 Show Resistance to 6-Thioguanine

CEM T cell leukemia cells were electroporated (1600V, 10 ms pulse width, 3 pulses by using the Neon Transfection system) with ribonucleoprotein (RNP) complexes containing guide RNA (see Example 21) designed in-house and obtained from Integrated DNA Technologies (IDT), together with Cas9 and tracrRNA. Cells confirmed to be transfected with the gRNA via the use of the tracr RNA (24 hours post electroporation) were purified using fluorescence activated cells sorting (FACS) prior to culture for a further 72 hours, followed by assessment of cell survival when challenged with increasing concentrations of 6-thioguanine (6-TG) compared to wild type (unmodified) CEM cells.

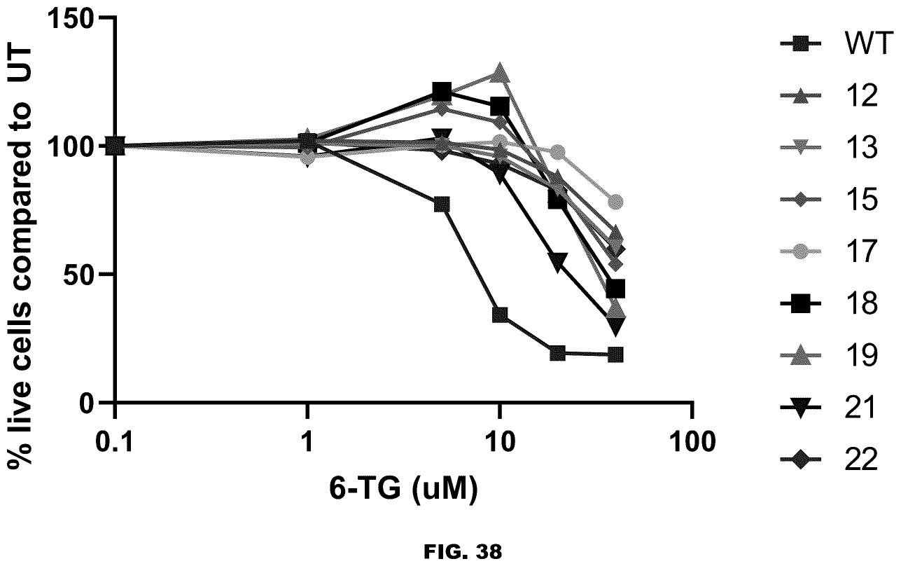

Guide 21 modified cells demonstrated intermediate resistance to 6-TG challenge compared to the other modified cells (see FIG. 38). The modified cells were shown to survive increasing doses of 6-TG (tested up to 40 μM); while unmodified CEM cells showed a decrease in cell viability. These data indicate that the modified cells have an increased resistance to the toxic effects of 6-TG.

ICE scoring is illustrated in the Table which follows. Guide 15, 18, and 21 showed efficient editing; while guide 19 showed zero editing.

| | Cell | INDEL | KO |
|---|---|---|---|
| gRNA | Type | (%) | score |
| 22 | CEM | 85 | 80 |
| 17 | CEM | 86 | 79 |
| 12 | CEM | 58 | 51 |
| 13 | CEM | 40-80? | 40-80? |
| 18 | CEM | 27 | 24 |
| 15 | CEM | 21 | 20 |
| 21 | CEM | 5 | 5 |
| 19 | CEM | 0 | 0 |

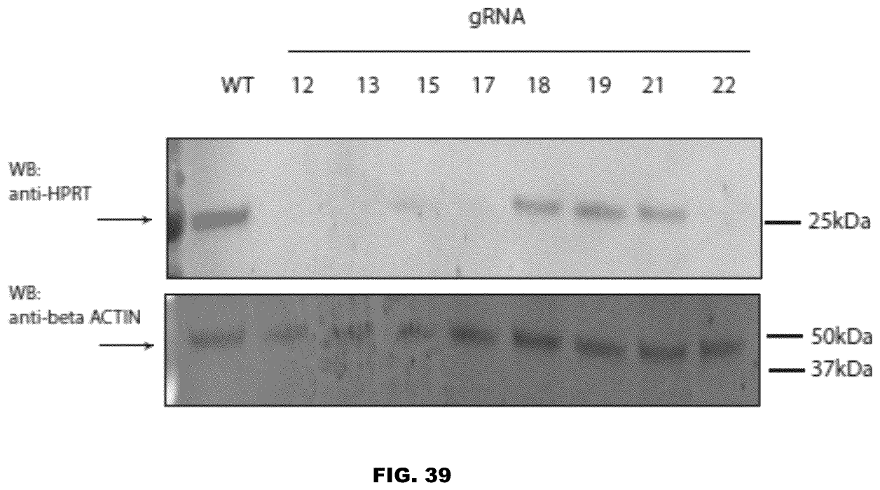

Unmodified and modified CEM cells were also used for isolation of protein and detection of the HPRT protein using Western blot (FIG. 39, top panel). Unmodified CEM cells (WT) showed detectable levels of HPRT at the expected size (25 kDA) while modified cells had undetectable levels of HPRT (guides, 12,13, 15 17 and 22) or evidence of HPRT protein levels that were reduced compared to wildtype (guides, 15, 18, 19 and 21). Actin protein detection (FIG. 39, bottom panel) was used as a protein loading control.

### Example 25—CEM T Cells Modified Using Guide RNA Directed Against HPRT1 can be Successfully Selected with 6-Thioguanine

CEM T cell leukemia cells were electroporated (1600V, 10 ms pulse width, 3 pulses by using the Neon Transfection system) with ribonucleoprotein (RNP) complexes containing guide RNA (see Example 21) designed in-house and obtained from Integrated DNA Technologies (IDT), together with Cas9 and tracrRNA. Cells confirmed to be transfected with the gRNA via the use of the tracr RNA (24 hours post electroporation) were purified using fluorescence activated cells sorting (FACS) prior to culture in 10 μM 6-TG for 1 week replenished with fresh 6-TG every 48-72 hours.

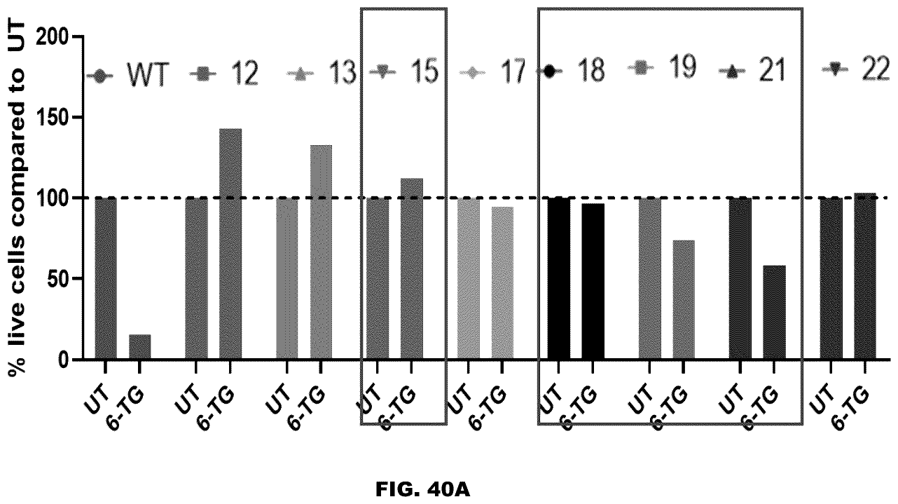

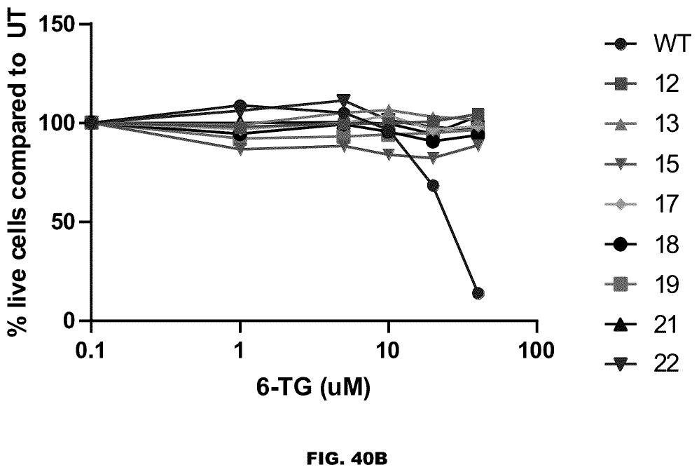

Cell viability analysis at this time-point showed that, compared to wild-type (WT) cells, modified CEM were resistant to 6-TG, with guides previously demonstrated have detectable HPRT protein expression (see FIG. 40A guides 15, 18, 19, 21; boxed) showed more variable viability. After 10 days in the presence of 10 uM 6-TG, replenished with fresh 6-TG every 48-72 hours, each cell line was challenged with increasing concentrations of 6-thioguanine (6-TG) and cell viability was compared to wild type (unmodified) CEM cells (see FIG. 40B). The 6-TG selected modified cells were all shown to survive increasing doses of 6-TG up to 40 μM while unmodified CEM cells showed a decrease in cell viability, with approximately 10% viability at 40 uM.

### Example 26—CEM T Cells Modified Using Guide RNA Directed Against HPRT1 Show Loss of HPRT Protein by Western Blot

CEM T cell leukemia cells were electroporated (1600V, 10 ms pulse width, 3 pulses by using the Neon Transfection system) with ribonucleoprotein (RNP) complexes containing guide RNA (gRNA; legend) designed in-house and obtained from Integrated DNA Technologies (IDT), together with Cas9 and tracrRNA. Cells confirmed to be transfected with the gRNA via the use of the tracr RNA (24 hours post electroporation) were purified using fluorescence activated cells sorting (FACS) prior to culture for 10 days in the presence of 10 uM 6-TG, replenished with fresh 6-TG every 48-72 hours.

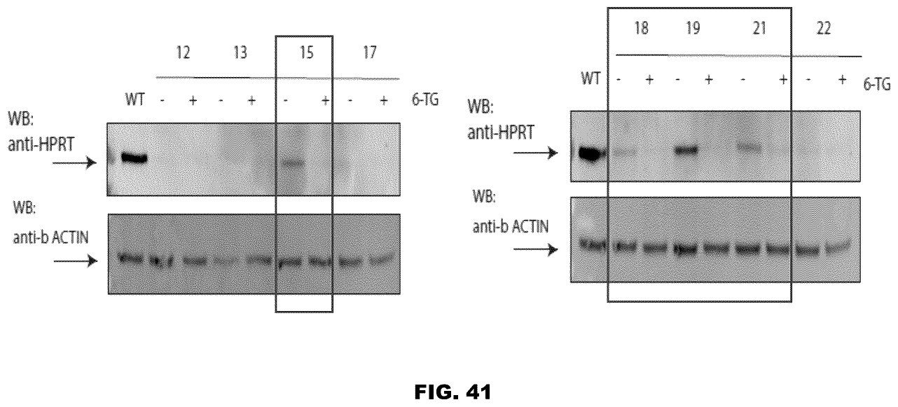

Following the selection period, protein was extracted from each cell and used for western blot-based detection of the HPRT protein. Detection of beta-actin was used as a protein loading control (Actin Antibody (ACTBD11B7): sc-81178. Santa Cruz). HPRT detection using an anti-HPRT antibody (Anti-HPRT antibody (ab245397) Abcam) showed the presence of a band at approximately 25 kDA, which corresponds to the expected size of the HPRT protein. CEM cells modified with guides 12, 13, 17 and 22 showed that HPRT protein was non-detectable within 72 hours of electroporation (−) and remained undetectable after 10 days of 6-TG selection (10 uM; +). CEM cells modified with guides 15, 18, 19 and 21 had detectable levels of HPRT protein 72 hours post-electroporation, though reduced compared to wildtype cells. After 10 days of selection, each of these CEM lines showed limited to no detectable levels of HPRT, indicating that modified cells had been successfully selected by 6-TG (see FIG. 41).

### Example 27—CEM T Cells Modified Using Guide RNA Directed Against HPRT1 Demonstrate Altered Sensitivity to Methotrexate

CEM T cell leukemia cells modified by 8 guide RNAs targeting the HPRT1 gene were cultured for 1 week in the presence of 10 μM 6-TG, replenished with fresh 6-TG every 48-72 hours. Following the selection period and confirmation of HPRT protein reduction, the CEM cells were tested for their sensitivity to a dose range of the de novo purine synthesis pathway, methotrexate (MTX) for 3 days in the presence of thymidine 16 μM (T1895 Sigma).

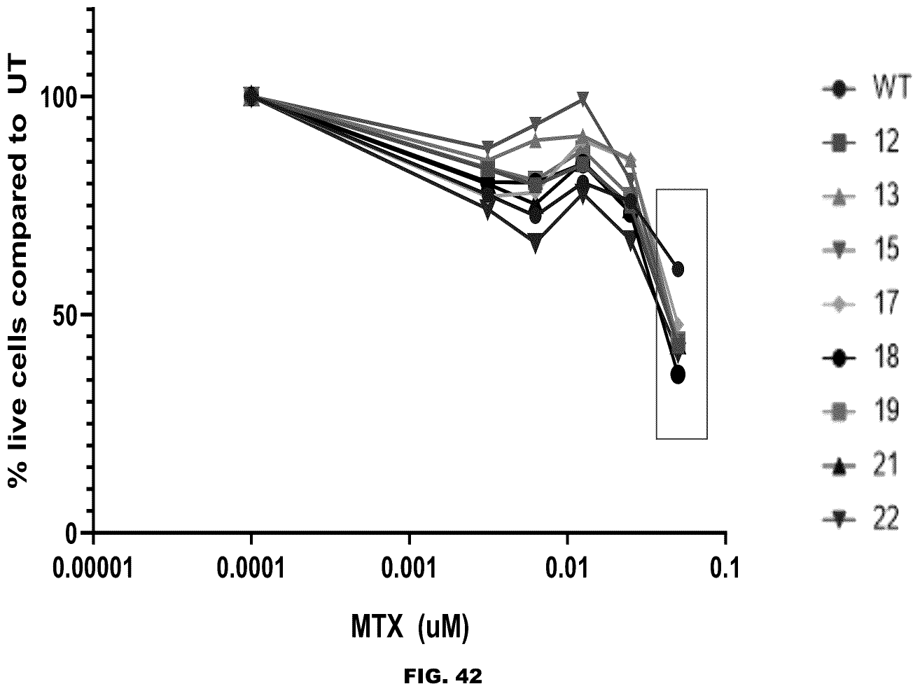

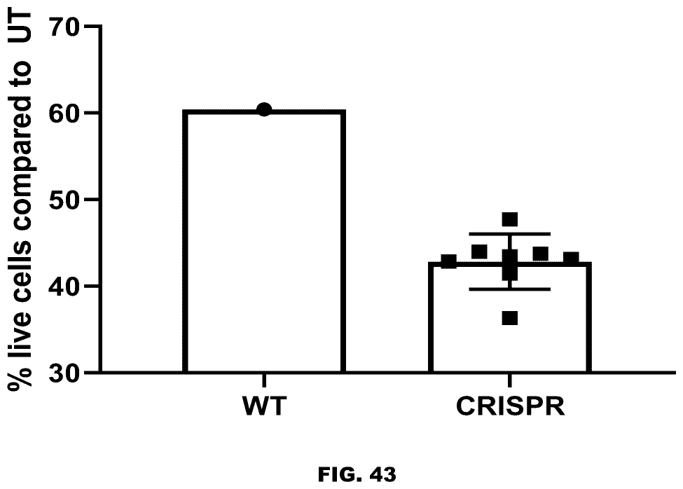

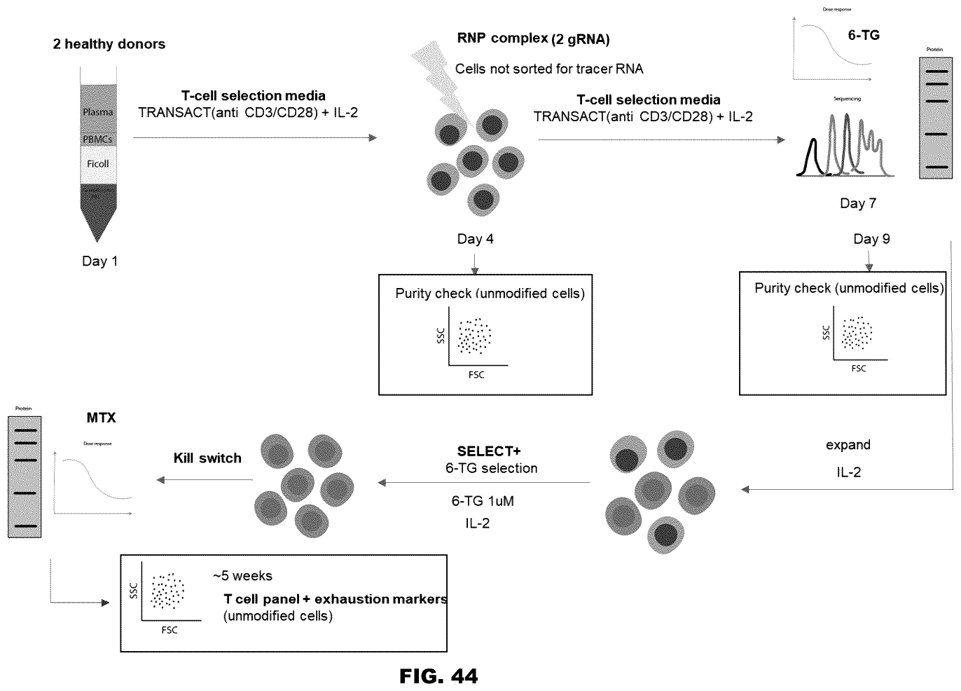

Compared to wild type cells, all of the lines showed increased sensitivity to 0.05 um MTX (see FIG. 42). The viability of the cells at 0.5 μM MTX (FIG. 43) demonstrated that while 60% of wild type (WT) cells were viable, the CRISPR/Cas9 modified CEM cells showed a reduction in the proportion of viable cells ranging from about 20% to about 50% depending on the modified line, demonstrating increased sensitivity of the modified cells, and successful induction of the kill-switch in these cells.

### Example 28—Primary Human T Cells Modified Using Guide RNA Directed Against HPRT1 Show Resistance to 6-TG Compared to Unmodified Cells

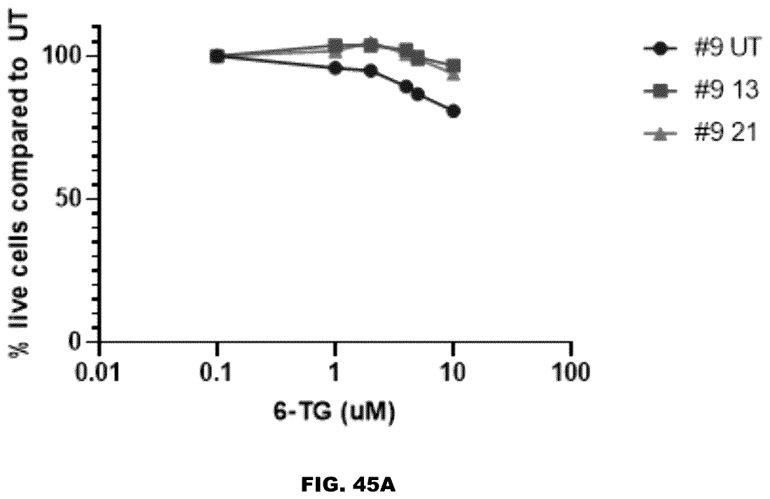

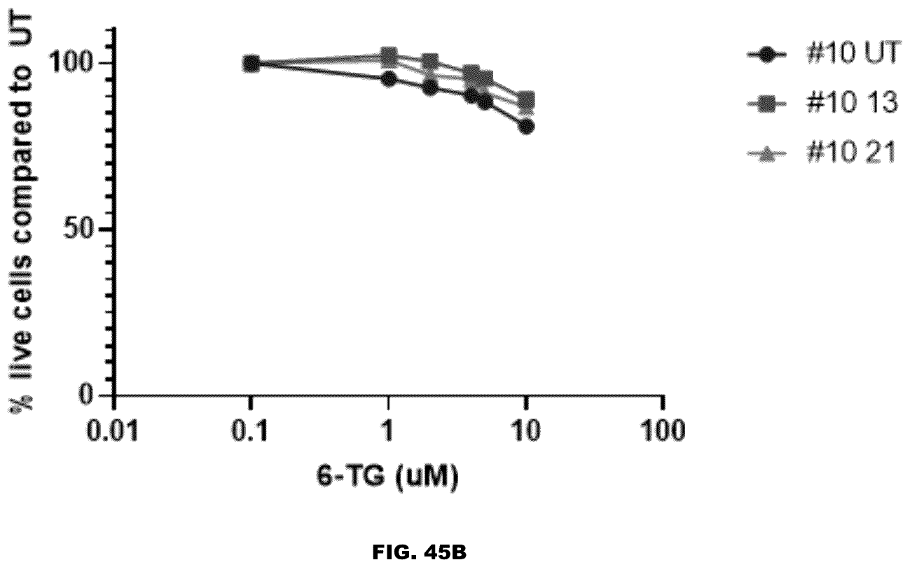

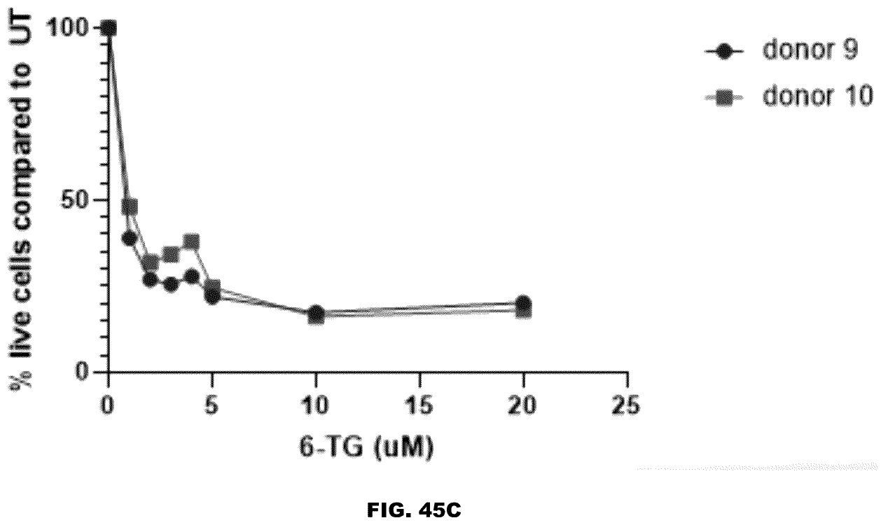

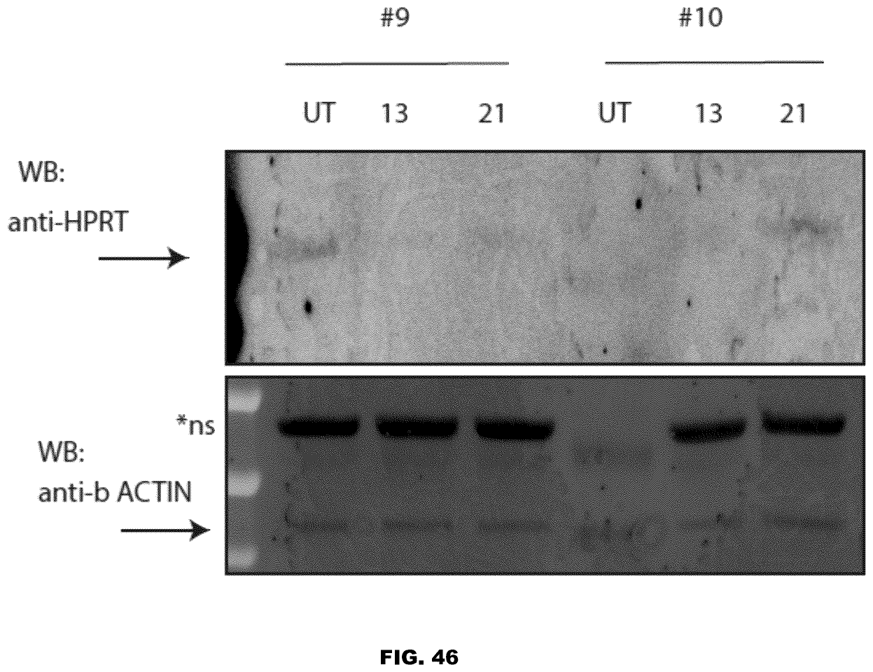

FIG. 44 illustrates a method of modifying primary T-cells in accordance with one embodiment of the present disclosure. FIGS. 45A-45C illustrate 6-TG dose responses seven days following the modification of the primary T-cells in accordance with the modification methods described herein. In particular, FIGS. 45A and 45B illustrate the targeting of Exon 3 (guide RNA 13) and Exon 8 (guide RNA 21). The references “#9” and “#10” denote T-cell number and UT control (electroporated without RNP). FIG. 45C illustrates unmodified cells from donor #9 and donor #10 tested with increasing concentrations of 6-TG (day 4). It was observed that the modified primary T-cells were very sensitive to low doses of 6-TG. ICE Scores are shown below. A Western Blot 72 hours after electroporation is illustrated in FIG. 46 (lower protein levels were loaded onto the gel; residual HPRT protein was observed for guide RNA 21; “#9” and “#10” denote T-cell donor number and UT control, i.e. electroporated without RNP).

| | Cell | INDEL | KO |
|---|---|---|---|
| gRNA | Type | (%) | score |
| 13 | Primary T cells #9 | 43 | 43 |
| 13 | Primary T cells #10 | 19 | 19 |
| 21 | Primary T cells #9 | 1 | 1 |
| 21 | Primary T cells #10 | 0 | 0 |

Peripheral blood mononuclear cells (PBMC) were isolated from Australian Red Cross Blood donor samples (n=2) via density centrifugation. The resulting cells were stimulated using TransAct reagent (Miltenyi Biotech; as per manufacturers' protocol) which results in stimulation of the cells via the T cell receptor (CD3) and the co-stimulatory molecule CD28 in the presence of the cytokine interleukin (IL)-2. On day 4 post cell stimulation, cells were electroporated (1600V, 10 ms pulse width, 3 pulses by using the Neon Transfection system) with ribonucleoprotein (RNP) complexes containing guide RNA (gRNA) 13 or 21 together with Cas9 prior to resuspending cells in media containing IL-2 with repeated stimulation using TransAct. By day 7, the original mixed PBMC population was determined to be primarily CD3+ T cells (flow cytometry; data not shown).

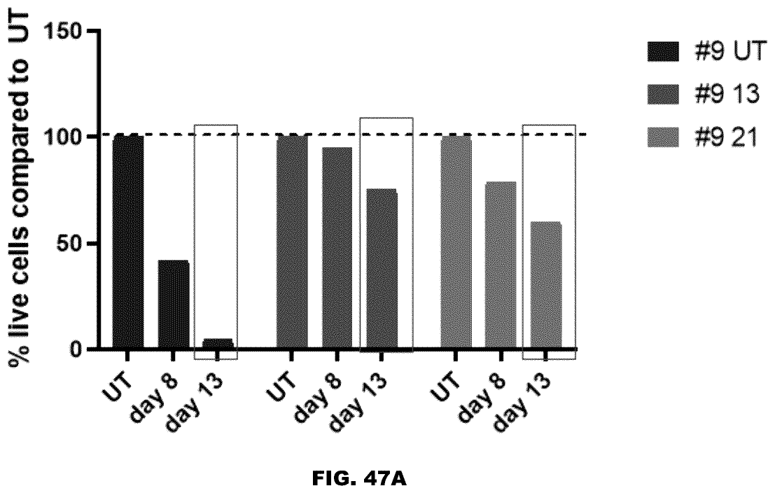

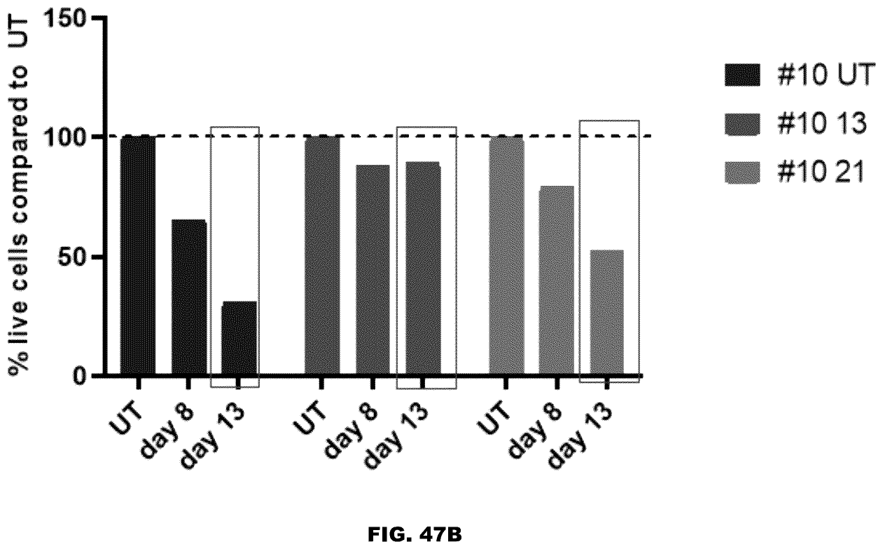

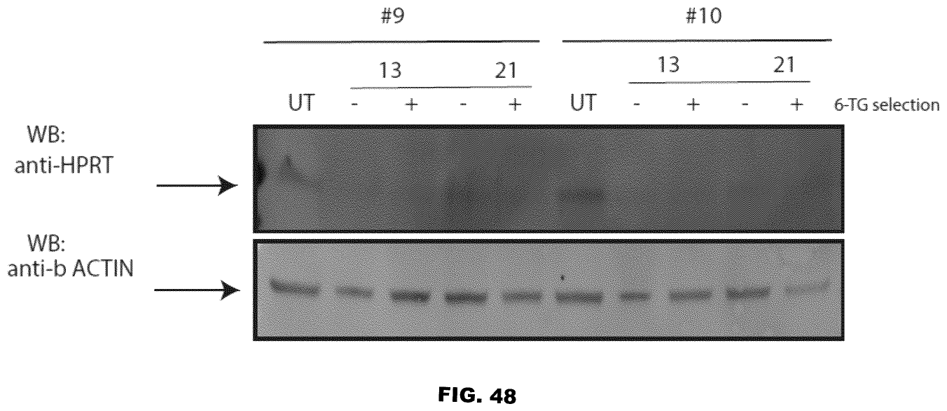

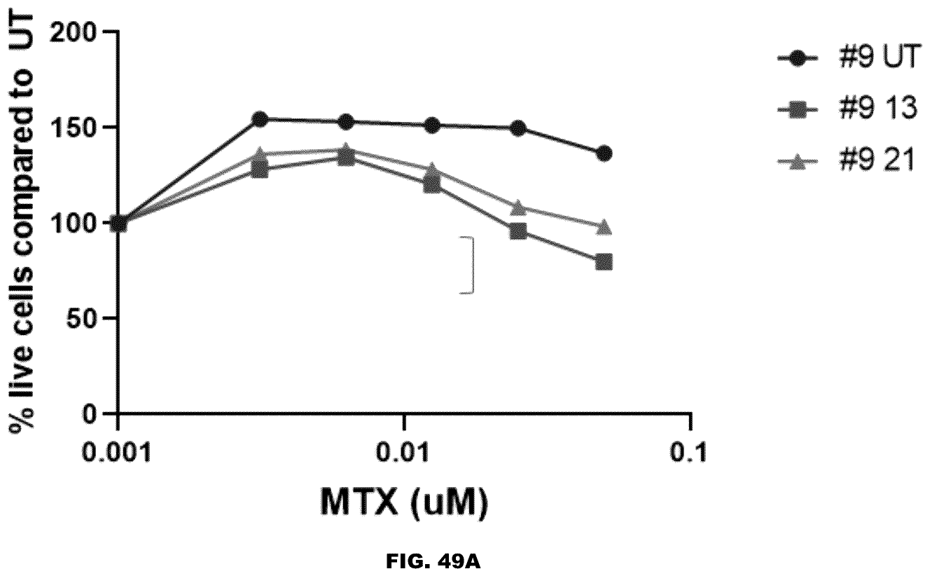

The cells were then cultured for about 7 and about 12 days in the presence of 1 uM 6-TG, with 6-TG replenished every about 2 to about 3 days. At each of these time points, the cells were assessed for the proportion of viable cells compared to untreated (UT) controls. This data showed that, while wild-type (WT) unmodified cells from both donors (Donor #9, FIG. 47A; Donor #10, FIG. 47B, where #9 and #10 denote T-cell donor number and UT control (electroporated without RNP)) showed reduced viability at day 8 and day 13, primary human T cells modified with both guides showed increased viability suggesting increased resistance to the effects of 6-TG. In both samples, guide 13 out-performed guide 21 in terms of the proportion of viable cells, suggesting less efficient targeting of the HPRT1 gene, and this correlated with a knockout efficiency of about 43% and about 1%, respectively (ICE scoring). The Western Blot depicted in FIG. 48 shows that 6-TG treatment was able to select for the HPRT knockout population.

### Example 29—Primary Human T Cells Modified Using Guide RNA Directed Against HPRT1 Demonstrate Increased Sensitivity to Methotrexate (MTX)

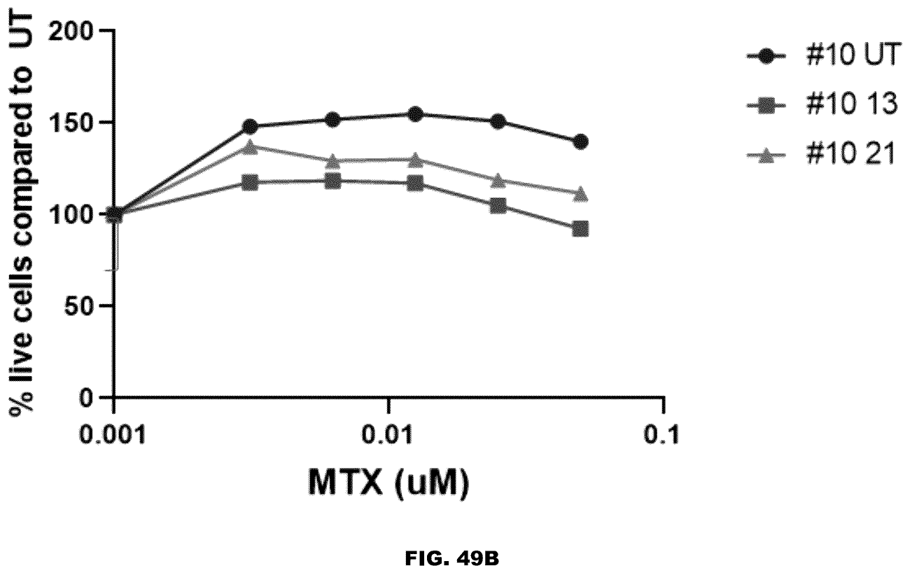

Primary human T cells modified using guide RNAs targeting the HPRT1 gene and subsequently placed under selection pressure for 12 days with 1 μM 6-TG, with 6-TG replenished every 2 to 3 days, were subjected to a dose range of MTX in the presence of thymidine 16 μM for 2 days and assessed for cell viability. Compared to wild-type non-modified cells (UT), the primary human T cells modified with guides 13 and 21 demonstrated increased sensitivity to MTX suggesting kill-switch induction (see FIGS. 49A and 49B, which illustrate the difference in sensitivity to MTX between WT and modified cells, and where “#9” and “#10” denote T-cell donor number and UT control (electroporated without RNP)).

### Example 30—HPRT Knockout Guide RNAs

As described in Example 22 above, the Applicant identified further guide RNAs targeting Exons 2, 3 or 8 of HPRT 1 (see, e.g., SEQ ID NOS: 50-61).

Synthego's Interference CRISPR Editing (ICE) tool using Sanger sequencing data was used as part of in silico design strategy. The Table which follows sets forth various guide RNA molecules targeting HPRT 1.

| SEQ | | Corresponding | |
|---|---|---|---|
| ID NO. | sgRNA guide and PAM | HPRT 1 target | Position# |
| 50 | TAGCCCCCCTTGAGCACACA | Exon 3 | 134475243 |
| 51 | ATGTAATCCAGCAGGTCAGC | Exon 3 | 134475273 |
| 52 | TATTCAGTGCTTTGATGTAA | Exon 3 | 134475287 |
| 53 | CTGACCTGCTGGATTACATC | Exon 3 | 134475292 |
| 54 | TCAGACTGAAGAGCTATTGT | Exon 3 | 134475364 |
| 55 | AAATTCCAGACAAGTTTGTT | Exon 8 | 134498639 |
| 56 | TTGTAGGATATGCCCTTGAC | Exon 8 | 134498657 |
| 57 | CAAATCCTCAGCATAATGAT | Exon 2 | 134473409 |
| 58 | TTTTGCATACCTAATCATTA | Exon 2 | 134473414 |
| 59 | CATACCTAATCATTATGCTG | Exon 2 | 134473419 |
| 60 | GAAAGGGTGTTTATTCCTCA | Exon 2 | 134473447 |
| 61 | TTCCTCATGGACTAATTATG | Exon 2 | 134473460 |

# Position within Chromosome X are referenced herein to Genome Reference Consortium Human Build 38 (GRCh38).

In some embodiments, the components to knockout the HPRT1 gene comprise a Cas12a and a guide RNA comprising SEQ ID NOS. 50-61.

### Additional Embodiments

In a first additional embodiment is a method of providing benefits of a lymphocyte infusion to a patient in need of treatment thereof while mitigating side effects comprising: generating HPRT deficient lymphocytes from a donor sample; positively selecting for the HPRT deficient lymphocytes ex vivo to provide a population of modified lymphocytes; administering an HSC graft to the patient; administering the population of modified lymphocytes to the patient following the administration of the HSC graft; and optionally administering methotrexate (MTX) if the side effects arise. In some embodiments, the patient treated receives the benefit of receiving T-cells to fight infection, support engraftment, and prevent disease relapse. In addition, should GvHD occur, T-cells may be removed through administration of one or more doses of MTX.

In some embodiments, the HPRT deficient lymphocytes are generated through knockout of the HPRT1 gene, such as by transfection of lymphocytes with a population of nanocapsules including a payload adapted to knockout HPRT (e.g. a payload including a guide RNA having the sequence of any one of SEQ ID NOS: 25-39). In other embodiments, the HPRT deficient lymphocytes are generated through knockdown of the HPRT1 gene, such as by transduction of lymphocytes with an expression vector including a nucleic acid sequence encoding an RNA interference agent (e.g. a nucleic acid encoding a shRNA having the sequence of any one of SEQ ID NOS: 1, 2, and 5-11). In some embodiments, the positive selection comprises contacting the generated HPRT deficient lymphocytes with a purine analog (e.g. 6-thioguanine (6-TG), 6-mercaptopurine (6-MP), or azathioprine (AZA)). In some embodiments, the positive selection comprises contacting the generated HPRT deficient lymphocytes with a purine analog and a second agent (e.g. allopurinol). In some embodiments, the purine analog is 6-TG. In some embodiments, the modified lymphocytes are administered as a single bolus. In some embodiments, the modified lymphocytes are administered as multiple doses. In some embodiments, each dose comprises between about 0.1×10⁶ cells/kg to about 240×10⁶ cells/kg. In some embodiments, the MTX is optionally administered upon diagnosis of GvHD. In some embodiments, an amount of MTX administered ranges from about 2 mg/m²/infusion to about 8 mg/m²/infusion. In some embodiments, the MTX is administered in titrated doses.

It is believed that the methods of the present disclosure exploits the purine salvage pathway via modification of the gene encoding the enzyme hypoxanthine-guanine phosphoribosyl transferase (HPRT), which facilitates the recycling of purines. Inhibition of HPRT expression via either gene knockout or gene knockdown renders the modified cells solely dependent on the de novo purine biosynthesis pathway for survival. In non-modified cells, delivery of the purine analogue 6-thioguanine (6-TG), which is converted through HPRT, ultimately leads to accumulation of 6-thioguanine nucleotides (6-TGN), which are toxic to the cell via several mechanisms including incorporation into DNA during S-phase. Inhibition of the HPRT enzyme in the gene modified cells and subsequent treatment with 6-TG, a drug already used in the treatment of various leukemias as well as severe inflammatory diseases, provides these cells with a survival advantage over non-modified cells, and therefore a mechanism by which to select modified cells in vitro and potentially in vivo. In addition, inhibition of the de novo purine biosynthesis pathway in these HPRT enzyme deficient cells, such as with methotrexate (MTX), results in cell apoptosis (due to an also non-functional purine salvage pathway), thereby providing a mechanism by which another approved drug can be used as a “kill switch” inducer in modified cells.

In a second additional embodiment is a composition including a component which reduces or eliminates HPRT expression in hematopoietic stem cells (“HSCs”). In some embodiments, the HSCs are lymphoid cells. In some embodiments, the lymphoid cells are T-cells. In some embodiments, the composition includes a first component which effectuates a knockdown of the HPRT1 gene. In other embodiments, the composition includes a first component which effectuates a knockout of the HPRT1 gene. In some embodiments, the composition includes a lentiviral expression vector including a first nucleic acid encoding an agent adapted to knockdown the HPRT1 gene (e.g. an RNA interference agent (RNAi)). In some embodiments, the lentiviral expression vector may be incorporated within a nanocapsule, such as one adapted to target HSCs.

In a third additional embodiment is an expression vector including a nucleic acid sequence encoding an RNAi to effectuate knockdown of HPRT. In some embodiments, the lentiviral expression vectors are suitable for producing selectable genetically modified cells, such as HSCs. In some embodiments, the HSCs transduced ex vivo may be administered to a patient in need of treatment. In some embodiments, the nucleic acid encoding the RNAi encodes a small hairpin ribonucleic acid molecule (“shRNA”) targeting HPRT1. In some embodiments, the first nucleic acid sequence encoding the shRNA targeting the HPRT 1 gene has a sequence having at least 90% identity to that of SEQ ID NO: 1, and wherein the first nucleic acid sequence is operably linked to a 7sk promoter or a mutated variant thereof. In some embodiments, the first nucleic acid sequence encoding the shRNA targeting the HPRT1 gene has a sequence having at least 95% identity to that of SEQ ID NO: 1, and wherein the first nucleic acid sequence is operably linked to a 7sk promoter or a mutated variant thereof. In some embodiments, the first nucleic acid sequence encoding the shRNA targeting the HPRT1 gene has a sequence having at least 97% identity to that of SEQ ID NO: 1, and wherein the first nucleic acid sequence is operably linked to a 7sk promoter or a mutated variant thereof. In some embodiments, the first nucleic acid sequence encoding the shRNA targeting the HPRT1 gene has a sequence of SEQ ID NO: 1, and wherein the first nucleic acid sequence is operably linked to a 7sk promoter or a mutated variant thereof.

In some embodiments, the first nucleic acid sequence encoding the shRNA targeting the HPRT1 gene has a sequence having at least 90% identity to that of SEQ ID NO: 2. In some embodiments, the first nucleic acid sequence encoding the shRNA targeting the HPRT1 gene has a sequence having at least 95% identity to that of SEQ ID NO: 2. In some embodiments, the first nucleic acid sequence encoding the shRNA targeting the HPRT 1 gene has a sequence having at least 97% identity to that of SEQ ID NO: 2. In some embodiments, the first nucleic acid sequence encoding the shRNA targeting the HPRT1 gene has a sequence of SEQ ID NO: 2.

In some embodiments, the first nucleic acid sequence encoding the shRNA targeting the HPRT1 gene has a sequence having at least 80% identity to any one of SEQ ID NOS: 5, 6, and 7. In some embodiments, the first nucleic acid sequence encoding the shRNA targeting the HPRT1 gene has a sequence having at least 90% identity to any one of SEQ ID NOS: 5, 6, and 7. In some embodiments, the first nucleic acid sequence encoding the shRNA targeting the HPRT1 gene has a sequence having at least 95% identity to any one of SEQ ID NOS: 5, 6, and 7. In some embodiments, the first nucleic acid sequence encoding the shRNA targeting the HPRT1 gene has a sequence having at least 97% identity to any one of SEQ ID NOS: 5, 6, and 7. In some embodiments, the first nucleic acid sequence encoding the shRNA targeting the HPRT1 gene has a sequence of any one of SEQ ID NOS: 5, 6, and 7.

In some embodiments, the first nucleic acid sequence is operably linked to a Pol III promoter. In some embodiments, the Pol III promoter is a Homo sapiens cell-line HEK-293 7sk RNA promoter (see, for example, SEQ ID NO: 14). In some embodiments, the Pol III promoter is a 7sk promoter which includes a single mutation in its nucleic acid sequence as compared with SEQ ID NO: 14. In some embodiments, the Pol III promoter is a 7sk promoter which includes multiple mutations in its nucleic acid sequence as compared with SEQ ID NO: 14. In some embodiments, the Pol III promoter is a 7sk promoter which includes a deletion in its nucleic acid sequence as compared with SEQ ID NO: 14. In some embodiments, the Pol III promoter is a 7sk promoter which includes both a mutation and a deletion in its nucleic acid sequence as compared with SEQ ID NO: 14. In some embodiments, the first nucleic acid sequence is operably linked to promoter having at least 95% identity to that of SEQ ID NO: 14. In some embodiments, the first nucleic acid sequence is operably linked to promoter having at least 97% identity to that of SEQ ID NO: 14. In some embodiments, the first nucleic acid sequence is operably linked to promoter having at least 98% identity to that of SEQ ID NO: 14. In some embodiments, the first nucleic acid sequence is operably linked to promoter having at least 99% identity to that of SEQ ID NO: 14. In some embodiments, the first nucleic acid sequence is operably linked to a promoter having SEQ ID NO: 14.

In a fourth additional embodiment is a lentiviral expression vector comprising a nucleic acid sequence encoding a micro-RNA based shRNA targeting a HPRT1 gene. In some embodiments, the nucleic acid sequence encoding the micro-RNA based shRNA targeting the HPRT1 gene has a sequence having at least 80% identity to any one of SEQ ID NOS: 8, 9, 10, and 11. In some embodiments, the nucleic acid sequence encoding the micro-RNA based shRNA targeting the HPRT1 gene has a sequence having at least 90% identity to any one of SEQ ID NOS: 8, 9, 10, and 11. In some embodiments, the nucleic acid sequence encoding the micro-RNA based shRNA targeting the HPRT1 gene has a sequence having at least 95% identity to any one of SEQ ID NOS: 8, 9, 10, and 11. In some embodiments, the nucleic acid sequence encoding the micro-RNA based shRNA targeting the HPRT1 gene has a sequence having at least 97% identity to any one of SEQ ID NOS: 8, 9, 10, and 11. In some embodiments, the nucleic acid sequence encoding the micro-RNA based shRNA targeting the HPRT1 gene has a sequence of any one of SEQ ID NOS: 8, 9, 10, and 11. In some embodiments, the nucleic acid sequence encoding the micro-RNA based shRNA targeting the HPRT1 gene is operably linked to a Pol III or Pol II promoter, including any of those described herein.

In a fifth additional embodiment is a polynucleotide sequence including (a) a first portion encoding an shRNA targeting HPRT; and (b) a second portion encoding a first promoter driving expression of the sequence encoding the shRNA targeting HPRT. In some embodiments, the polynucleotide further comprises (c) a third portion encoding a central polypurine tract element; and (d) a fourth portion encoding a Rev response element (SEQ ID NO: 19). In some embodiments, the polynucleotide sequence further comprises a WPRE element (e.g. the WPRE element comprising SEQ ID NO: 18). In some embodiments, the polynucleotide sequence further comprises an insulator.

In a sixth additional embodiment are HSCs (e.g. CD34⁺ HSCs) which have been transduced with an expression vector or transfected with a nanocapsule, each including an agent designed to reduce HPRT expression (e.g. an RNAi for knockdown of HPRT). In some embodiments, the HSCs are T-cells. In some embodiments, the transduced HSCs constitute a cell therapy product which may be administered to a subject in need of treatment thereof, e.g. a patient treated with the transduced HSCs received the benefit of receiving cells (such as T-cells that can be expanded ex vivo) to fight infection, support engraftment, and prevent disease relapse.

In a seventh additional embodiment is a host cell transduced with any one of an expression vector, and wherein the host cell is HPRT deficient. In some embodiments, the host cell is a T-cell. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of SEQ ID NO: 1.

In an eight additional embodiment is a pharmaceutical composition comprising the host cell, wherein the host cell is formulated with a pharmaceutically acceptable carrier or excipient. In some embodiments, the host cell is an HPRT deficient host cell derived by transducing a hostel cell with an expression vector. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of SEQ ID NO: 1.

In a ninth additional embodiment is a method of generating HPRT-deficient cells comprising: transducing a population of host cells with an expression vector, and positively selecting for the HPRT-deficient cells by contacting the population of the transduced host cells with at least a purine analog. In some embodiments, the purine analog is selected from the group consisting of 6-TG and 6-mercaptopurin. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of SEQ ID NO: 1.

In a tenth additional embodiment is a method of providing benefits of a lymphocyte infusion to a patient in need of treatment thereof while mitigating side effects comprising: generating HPRT deficient lymphocytes from a donor sample, wherein the HPRT deficient lymphocytes are generating by transducing lymphocytes within the donor sample with an expression vector, positively selecting for the HPRT deficient lymphocytes ex vivo to provide a population of modified lymphocytes; administering an HSC graft to the patient; administering a therapeutically effective amount of the population of modified lymphocytes to the patient following the administration of the HSC graft; and optionally administering a dihydrofolate reductase inhibitor if the side effects arise. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of SEQ ID NO: 1.

In an eleventh additional embodiment is a method of providing benefits of a lymphocyte infusion to a patient in need of treatment thereof while mitigating side effects comprising: generating HPRT deficient lymphocytes from a donor sample, wherein the HPRT deficient lymphocytes are generating by transducing lymphocytes within the donor sample with an expression vector; positively selecting for the HPRT deficient lymphocytes ex vivo to provide a population of modified lymphocytes; and administering the population of modified lymphocytes to the patient contemporaneously with or after an administration of an HSC graft. In some embodiments, the method further comprises administering to the patient one or more doses of a dihydrofolate reductase inhibitor. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of SEQ ID NO: 1.

In a twelfth additional embodiment is a method of treating a hematological cancer in a patient in need of treatment thereof comprising: generating HPRT deficient lymphocytes from a donor sample, wherein the HPRT deficient lymphocytes are generating by transducing lymphocytes within the donor sample with an expression vector; positively selecting for the HPRT deficient lymphocytes ex vivo to provide a population of modified lymphocytes; inducing at least a partial graft versus malignancy effect by administering an HSC graft to the patient; and administering the population of modified lymphocytes to the patient following the detection of residual disease or disease recurrence. In some embodiments, the method further comprises administering to the patient at least one dose of a dihydrofolate reductase inhibitor to suppress at least one symptom of GvHD or CRS. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of SEQ ID NO: 1.

In a thirteenth additional embodiment is a method of treating a patient with hypoxanthine-guanine phosphoribosyl transferase (HPRT) deficient lymphocytes including the steps of: (a) isolating lymphocytes from a donor subject; (b) transducing the isolated lymphocytes with an expression vector; (c) exposing the transduced isolated lymphocytes to an agent which positively selects for HPRT deficient lymphocytes to provide a preparation of modified lymphocytes; (d) administering a therapeutically effective amount of the preparation of the modified lymphocytes to the patient following hematopoietic stem-cell transplantation; and (e) optionally administering methotrexate or mycophenolic acid following the development of graft-versus-host disease (GvHD) in the patient. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of SEQ ID NO: 1.

In a fourteenth additional embodiment is a method of providing benefits of a lymphocyte infusion to a patient in need of treatment thereof while mitigating side effects comprising: generating substantially HPRT deficient lymphocytes from a donor sample, wherein the substantially HPRT deficient lymphocytes are generating by transfecting lymphocytes within the donor sample with a delivery vehicle including an endonuclease and a gRNA targeting HPRT; positively selecting for the substantially HPRT deficient lymphocytes ex vivo to provide a population of modified lymphocytes; administering an HSC graft to the patient; administering a therapeutically effective amount of the population of modified lymphocytes to the patient following the administration of the HSC graft; and optionally administering MTX if the side effects arise.

In a fifteenth additional embodiment is a method of providing benefits of a lymphocyte infusion to a patient in need of treatment thereof while mitigating side effects comprising: generating substantially HPRT deficient lymphocytes from a donor sample, wherein the substantially HPRT deficient lymphocytes are generating by transfecting lymphocytes within the donor sample with a delivery vehicle including a Cas protein (e.g. Cas9, Cas12a, Cas12b) and a gRNA targeting the HPRT1 gene; positively selecting for the substantially HPRT deficient lymphocytes ex vivo to provide a population of modified lymphocytes; administering an HSC graft to the patient; administering a therapeutically effective amount of the population of modified lymphocytes to the patient following the administration of the HSC graft; and optionally administering MTX if the side effects arise.

In a sixteenth additional embodiment is a lymphocyte transduced with an expression vector comprising a first expression control sequence operably linked to a first nucleic acid sequence, the first nucleic acid sequence encoding a shRNA to knockdown HPRT, wherein the shRNA has at least 90% identity to the sequence of any one of SEQ ID NOS: 2, 5, 6, 7, 8, 9, 10, and 11. In some embodiments, the shRNA has at least 95% identity to the sequence of any one of SEQ ID NOS: 2, 5, 6, 7, 8, 9, 10, and 11. In some embodiments, the shRNA has at least 97% identity to the sequence of any one of SEQ ID NOS: 2, 5, 6, 7, 8, 9, 10, and 11. In some embodiments, the shRNA comprises the sequence of any one of SEQ ID NOS: 2, 5, 6, 7, 8, 9, 10, and 11. In some embodiments, the lymphocyte is rendered substantially HPRT deficient following transduction with the expression vector. In some embodiments, the lymphocyte is a T-cell.

In a seventeenth additional embodiment of the present disclosure is an expression vector comprising a first expression control sequence operably linked to a first nucleic acid sequence, the first nucleic acid sequence encoding a shRNA to knockdown hypoxanthine-guanine phosphoribosyl transferase (HPRT), wherein the shRNA has at least 90% identity to the sequence of any one of SEQ ID NOS: 2, 5, 6, and 7. In some embodiments, the shRNA has a nucleic acid sequence having at least 95% identity to the sequence of any one of SEQ ID NOS: 2, 5, 6, and 7. In some embodiments, the shRNA has a nucleic acid sequence having at least 97% identity to the sequence of any one of SEQ ID NOS: 2, 5, 6, and 7. In some embodiments, the shRNA comprises the nucleic acid sequence of any one of SEQ ID NOS: 2, 5, 6, and 7.

In some embodiments, the first expression control sequence comprises a Pol III promoter or a Pol II promoter. In some embodiments, the Pol III promoter is a 7sk promoter, a mutated 7sk promoter, an H1 promoter, or an EF1a promoter. In some embodiments, the 7sk promoter has a nucleic acid sequence having at least 95% sequence identity to that of SEQ ID NO: 14. In some embodiments, the 7sk promoter has a nucleic acid sequence having at least 97% sequence identity to that of SEQ ID NO: 14. In some embodiments, the 7sk promoter comprises the nucleic acid sequence of SEQ ID NO: 14. In some embodiments, the mutated 7sk promoter has a nucleic acid sequence having at least 95% sequence identity to that of SEQ ID NO: 15. In some embodiments, the mutated 7sk promoter has a nucleic acid sequence having at least 97% sequence identity to that of SEQ ID NO: 15. In some embodiments, the mutated 7sk promoter comprises the nucleic acid sequence of SEQ ID NO: 15.

In a eighteenth additional embodiment of the present disclosure is an expression vector comprising a first expression control sequence operably linked to a first nucleic acid sequence, the first nucleic acid sequence encoding a shRNA to knockdown HPRT, wherein the shRNA has at least 90% sequence identity to the sequence of any one of SEQ ID NOS: 8, 9, 10, and 11. In some embodiments, the shRNA has a nucleic acid sequence having at least 95% identity to the sequence of any one of SEQ ID NOS: 8, 9, 10, and 11. In some embodiments, the shRNA has a nucleic acid sequence having at least 97% identity to the sequence of any one of SEQ ID NOS: 8, 9, 10, and 11. In some embodiments, the shRNA has a nucleic acid sequence of any one of SEQ ID NOS: 8, 9, 10, and 11.

In some embodiments, the first expression control sequence comprises a Pol III promoter or a Pol II promoter. In some embodiments, the Pol III promoter is a 7sk promoter, a mutated 7sk promoter, an H1 promoter, or an EF1a promoter. In some embodiments, the 7sk promoter has a nucleic acid sequence having at least 95% sequence identity to that of SEQ ID NO: 14. In some embodiments, the 7sk promoter has a nucleic acid sequence having at least 97% sequence identity to that of SEQ ID NO: 14. In some embodiments, the 7sk promoter comprises the nucleic acid sequence of SEQ ID NO: 14. In some embodiments, the mutated 7sk promoter has a nucleic acid sequence having at least 95% sequence identity to that of SEQ ID NO: 15. In some embodiments, the mutated 7sk promoter has a nucleic acid sequence having at least 97% sequence identity to that of SEQ ID NO: 15. In some embodiments, the mutated 7sk promoter comprises the nucleic acid sequence of SEQ ID NO: 15.

In a nineteenth additional embodiment of the present disclosure is a host cell transduced with an expression vector comprising a first expression control sequence operably linked to a first nucleic acid sequence, the first nucleic acid sequence encoding a shRNA to knockdown HPRT, wherein the shRNA has at least 90% identity to the sequence of any one of SEQ ID NOS: 2, 5, 6, 7, 8, 9, 10, and 11. In some embodiments, the shRNA has at least 95% identity to the sequence of any one of SEQ ID NOS: 2, 5, 6, 7, 8, 9, 10, and 11. In some embodiments, the shRNA has at least 97% identity to the sequence of any one of SEQ ID NOS: 2, 5, 6, 7, 8, 9, 10, and 11. In some embodiments, the shRNA comprises the sequence of any one of SEQ ID NOS: 2, 5, 6, 7, 8, 9, 10, and 11. In some embodiments, the host cell is rendered substantially HPRT deficient following transduction with the expression vector. In some embodiments, the host cell is a lymphocyte, e.g. a T-cell.

In a twentieth additional embodiment of the present disclosure is a pharmaceutical composition comprising a host cell, wherein the host cell is formulated with a pharmaceutically acceptable carrier or excipient, the host cell having been transduced with an expression vector comprising a first expression control sequence operably linked to a first nucleic acid sequence, the first nucleic acid sequence encoding a shRNA to knockdown HPRT, wherein the shRNA has at least 90% identity to the sequence of any of SEQ ID NOS: 2, 5, 6, 7, 8, 9, 10, and 11. In some embodiments, the shRNA has at least 95% identity to the sequence of any of SEQ ID NOS: 2, 5, 6, 7, 8, 9, 10, and 11. In some embodiments, the shRNA has at least 97% identity to the sequence of any of SEQ ID NOS: 2, 5, 6, 7, 8, 9, 10, and 11. In some embodiments, the shRNA comprises the sequence of any one of SEQ ID NOS: 2, 5, 6, 7, 8, 9, 10, and 11. In some embodiments, the host cell is rendered substantially HPRT deficient following transduction with the expression vector. In some embodiments, the host cell is a lymphocyte, e.g. a T-cell.

In a twenty-first additional embodiment of the present disclosure is a method of generating substantially HPRT-deficient cells comprising: transducing a population of host cells with an expression vector, and positively selecting for the HPRT-deficient cells by contacting the population of the transduced host cells with at least a purine analog. In some embodiments, the purine analog is selected from the group consisting of 6-thioguanine (6-TG) and 6-mercaptopurin. In some embodiments, the expression vector comprises a first expression control sequence operably linked to a first nucleic acid sequence, the first nucleic acid sequence encoding a shRNA to knockdown HPRT, wherein the shRNA has at least 90% identity to the sequence of any of SEQ ID NOS: 2 and 5-11. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of any of SEQ ID NOS: 2 and 5-11. In some embodiments, the expression vector comprises a first expression control sequence operably linked to a first nucleic acid sequence, the first nucleic acid sequence encoding a shRNA to knockdown HPRT, wherein the shRNA has at least 97% identity to the sequence of any of SEQ ID NOS: 2 and 5-11. In some embodiments, the shRNA comprises the sequence of any one of SEQ ID NOS: 2, 5, 6, 7, 8, 9, 10, and 11.

In a twenty-second additional embodiment aspect of the present disclosure is a method of providing benefits of a lymphocyte infusion to a patient in need of treatment thereof while mitigating side effects comprising: generating substantially HPRT deficient lymphocytes from a donor sample, wherein the substantially HPRT deficient lymphocytes are generating by transducing lymphocytes within the donor sample with an expression vector; positively selecting for the substantially HPRT deficient lymphocytes ex vivo to provide a population of modified lymphocytes; administering an HSC graft to the patient; administering a therapeutically effective amount of the population of modified lymphocytes to the patient following the administration of the HSC graft; and optionally administering a dihydrofolate reductase inhibitor if the side effects arise. In some embodiments, the expression vector comprises a first expression control sequence operably linked to a first nucleic acid sequence, the first nucleic acid sequence encoding a shRNA to knockdown HPRT, wherein the shRNA has at least 90% identity to the sequence of any of SEQ ID NOS: 2 and 5-11. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of any of SEQ ID NOS: 2 and 5-11. In some embodiments, the expression vector comprises a first expression control sequence operably linked to a first nucleic acid sequence, the first nucleic acid sequence encoding a shRNA to knockdown HPRT, wherein the shRNA has at least 97% identity to the sequence of any of SEQ ID NOS: 2 and 5-11. In some embodiments, the shRNA comprises the sequence of any one of SEQ ID NOS: 2, 5, 6, 7, 8, 9, 10, and 11.

In some embodiments, the dihydrofolate reductase inhibitor is selected from the group consisting of methotrexate (MTX) or mycophenolic acid (MPA). In some embodiments, the positive selection comprises contacting the generated substantially HPRT deficient lymphocytes with a purine analog. In some embodiments, the purine analog is 6-TG. In some embodiments, an amount of 6-TG ranges from between about 1 to about 15 μg/mL.

In some embodiments, the positive selection comprises contacting the generated substantially HPRT deficient lymphocytes with both a purine analog and allopurinol. In some embodiments, the modified lymphocytes are administered as a single bolus. In some embodiments, multiple doses of the modified lymphocytes are administered to the patient. In some embodiments, each dose of the modified lymphocytes comprises between about 0.1×10⁶ cells/kg to about 240×10⁶ cells/kg. In some embodiments, a total dosage of modified lymphocytes comprises between about 0.1×10⁶ cells/kg to about 730×10⁶ cells/kg.

In an twenty-third additional embodiment of the present disclosure is a method of providing benefits of a lymphocyte infusion to a patient in need of treatment thereof while mitigating side effects comprising: generating substantially HPRT deficient lymphocytes from a donor sample, wherein the substantially HPRT deficient lymphocytes are generating by transducing lymphocytes within the donor sample with an expression vector; positively selecting for the substantially HPRT deficient lymphocytes ex vivo to provide a population of modified lymphocytes; and administering a therapeutically effective amount of population of modified lymphocytes to the patient contemporaneously with or after an administration of an HSC graft. In some embodiments, the method further comprises administering to the patient one or more doses of a dihydrofolate reductase inhibitor. In some embodiments, the expression vector comprises a first expression control sequence operably linked to a first nucleic acid sequence, the first nucleic acid sequence encoding a shRNA to knockdown HPRT, wherein the shRNA has at least 90% identity to the sequence of any of SEQ ID NOS: 2 and 5-11. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of any of SEQ ID NOS: 2 and 5-11. In some embodiments, the expression vector comprises a first expression control sequence operably linked to a first nucleic acid sequence, the first nucleic acid sequence encoding a shRNA to knockdown HPRT, wherein the shRNA has at least 97% identity to the sequence of any of SEQ ID NOS: 2 and 5-11. In some embodiments, the shRNA comprises the sequence of any one of SEQ ID NOS: 2, 5, 6, 7, 8, 9, 10, and 11.

In some embodiments, the dihydrofolate reductase inhibitor is selected from the group consisting of MTX or MPA. In some embodiments, the positive selection comprises contacting the generated substantially HPRT deficient lymphocytes with a purine analog. In some embodiments, the purine analog is 6-TG. In some embodiments, an amount of 6-TG ranges from between about 1 to about 15 μg/mL. 6-TG. In some embodiments, the positive selection comprises contacting the generated substantially HPRT deficient lymphocytes with both a purine analog and allopurinol. In some embodiments, the modified lymphocytes are administered as a single bolus. In some embodiments, multiple doses of the modified lymphocytes are administered to the patient. In some embodiments, each dose of the modified lymphocytes comprises between about 0.1×10⁶ cells/kg to about 240×10⁶ cells/kg. In some embodiments, a total dosage of modified lymphocytes comprises between about 0.1×10⁶ cells/kg to about 730×10⁶ cells/kg.

In an twenty-fourth additional embodiment of the present disclosure is a method of treating a hematological cancer in a patient in need of treatment thereof comprising: generating substantially HPRT deficient lymphocytes from a donor sample, wherein the substantially HPRT deficient lymphocytes are generating by transducing lymphocytes within the donor sample with an expression vector; positively selecting for the substantially HPRT deficient lymphocytes ex vivo to provide a population of modified lymphocytes; inducing at least a partial graft versus malignancy effect by administering an HSC graft to the patient; and administering a therapeutically effective amount of population of modified lymphocytes to the patient following the detection of residual disease or disease recurrence. In some embodiments, the method further comprises administering to the patient at least one dose of a dihydrofolate reductase inhibitor to suppress at least one symptom of GvHD or CRS. In some embodiments, the expression vector comprises a first expression control sequence operably linked to a first nucleic acid sequence, the first nucleic acid sequence encoding a shRNA to knockdown HPRT, wherein the shRNA has at least 90% identity to the sequence of any of SEQ ID NOS: 2 and 5-11. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of any of SEQ ID NOS: 2 and 5-11. In some embodiments, the expression vector comprises a first expression control sequence operably linked to a first nucleic acid sequence, the first nucleic acid sequence encoding a shRNA to knockdown HPRT, wherein the shRNA has at least 97% identity to the sequence of any of SEQ ID NOS: 2 and 5-11. In some embodiments, the shRNA comprises the sequence of any one of SEQ ID NOS: 2, 5, 6, 7, 8, 9, 10, and 11.

In some embodiments, the dihydrofolate reductase inhibitor is selected from the group consisting of MTX or MPA. In some embodiments, the positive selection comprises contacting the generated substantially HPRT deficient lymphocytes with a purine analog. In some embodiments, the purine analog is 6-TG. In some embodiments, an amount of 6-TG ranges from between about 1 to about 15 μg/mL. In some embodiments, the positive selection comprises contacting the generated substantially HPRT deficient lymphocytes with both a purine analog and allopurinol. In some embodiments, the modified lymphocytes are administered as a single bolus. In some embodiments, multiple doses of the modified lymphocytes are administered to the patient. In some embodiments, each dose of the modified lymphocytes comprises between about 0.1×106 cells/kg to about 240×106 cells/kg. In some embodiments, a total dosage of modified lymphocytes comprises between about 0.1×106 cells/kg to about 730×106 cells/kg.

In a twenty-fifth additional embodiment of the present disclosure is a method of providing benefits of a lymphocyte infusion to a patient in need of treatment thereof while mitigating side effects comprising: generating substantially HPRT deficient lymphocytes from a donor sample, wherein the substantially HPRT deficient lymphocytes are generating by transfecting lymphocytes within the donor sample with a delivery vehicle including components adapted to knockout HPRT; positively selecting for the substantially HPRT deficient lymphocytes ex vivo to provide a population of modified lymphocytes; administering an HSC graft to the patient; administering a therapeutically effective amount of the population of modified lymphocytes to the patient following the administration of the HSC graft; and optionally administering MTX if the side effects arise.

In some embodiments, the components adapted to knockout HPRT comprise a guide RNA having at least 90% sequence identity to any one of SEQ ID NOS: 25-39. In some embodiments, the components adapted to knockout HPRT comprise a guide RNA having at least 95% sequence identity to any one of SEQ ID NOS: 25-39. In some embodiments, the components adapted to knockout HPRT comprise a guide RNA targeting a nucleic acid sequence selected from the group consisting of SEQ ID NOS: 25-39. In some embodiments, the components adapted to knockout HPRT comprises a Cas protein. In some embodiments, the Cas protein comprises a Cas9 protein. In some embodiments, the Cas protein comprises a Cas12 protein. In some embodiments, the Cas12 protein is a Cas12a protein. In some embodiments, the Cas12 protein is a Cas12b protein. In some embodiments, the components adapted to knockout HPRT comprise a guide RNA having at least 90% identity to any one of SEQ ID NOS: 25-39, and a Cas protein (e.g. a Cas9 protein, a Cas12a protein, or a Cas12b protein). In some embodiments, the components adapted to knockout HPRT comprise a guide RNA having at least 95% identity to any one of SEQ ID NOS: 25-39, and a Cas protein (e.g. a Cas9 protein, a Cas12a protein, or a Cas12b protein). In some embodiments, the delivery vehicle is a nanocapsule. In some embodiments, the delivery vehicle is a nanocapsule comprising one or more targeting moieties.

In some embodiments, the method further comprises administering to the patient one or more doses of a dihydrofolate reductase inhibitor. In some embodiments, the dihydrofolate reductase inhibitor is selected from the group consisting of MTX or MPA. In some embodiments, the positive selection comprises contacting the generated substantially HPRT deficient lymphocytes with a purine analog. In some embodiments, the purine analog is 6-TG. In some embodiments, an amount of 6-TG ranges from between about 1 to about 15 μg/mL. In some embodiments, the positive selection comprises contacting the generated substantially HPRT deficient lymphocytes with both a purine analog and allopurinol.

In some embodiments, the modified lymphocytes are administered as a single bolus. In some embodiments, multiple doses of the modified lymphocytes are administered to the patient. In some embodiments, each dose of the modified lymphocytes comprises between about 0.1×106 cells/kg to about 240×106 cells/kg. In some embodiments, total dosage of modified lymphocytes comprises between about 0.1×106 cells/kg to about 730×106 cells/kg.

In an twenty-sixth additional embodiment of the present disclosure is a method of treating a hematological cancer in a patient in need of treatment thereof comprising: generating substantially HPRT deficient lymphocytes from a donor sample, wherein the substantially HPRT deficient lymphocytes are generating by transfecting lymphocytes within the donor sample with a delivery vehicle including components adapted to knockout HPRT; positively selecting for the substantially HPRT deficient lymphocytes ex vivo to provide a population of modified lymphocytes; inducing at least a partial graft versus malignancy effect by administering an HSC graft to the patient; and administering a therapeutically effective amount of the population of modified lymphocytes to the patient following the detection of residual disease or disease recurrence.

In some embodiments, the components adapted to knockout HPRT comprise a guide RNA having at least 90% sequence identity to any one of SEQ ID NOS: 25-39. In some embodiments, the components adapted to knockout HPRT comprise a guide RNA having at least 95% sequence identity to any one of SEQ ID NOS: 25-39. In some embodiments, the components adapted to knockout HPRT comprise a guide RNA targeting a nucleic acid sequence selected from the group consisting of SEQ ID NOS: 25-39. In some embodiments, the components adapted to knockout HPRT comprise a Cas protein. In some embodiments, the Cas protein comprises a Cas9 protein. In some embodiments, the Cas protein comprises a Cas12 protein. In some embodiments, the Cas12 protein is a Cas12a protein. In some embodiments, the Cas12 protein is a Cas12b protein. In some embodiments, the components adapted to knockout HPRT comprise a guide RNA having at least 90% identity to any one of SEQ ID NOS: 25-39, and a Cas protein (e.g. a Cas9 protein, a Cas12a protein, or a Cas12b protein). In some embodiments, the Cas12 protein is a Cas12b protein. In some embodiments, the components adapted to knockout HPRT comprise a guide RNA having at least 95% identity to any one of SEQ ID NOS: 25-39, and a Cas protein (e.g. a Cas9 protein, a Cas12a protein, or a Cas12b protein). In some embodiments, the delivery vehicle is a nanocapsule. In some embodiments, the delivery vehicle is a nanocapsule comprising one or more targeting moieties.

In some embodiments, the method further comprises administering to the patient at least one dose of a dihydrofolate reductase inhibitor to suppress at least one symptom of GvHD or CRS. In some embodiments, the dihydrofolate reductase inhibitor is selected from the group consisting of MTX or MPA. In some embodiments, the positive selection comprises contacting the generated substantially HPRT deficient lymphocytes with a purine analog. In some embodiments, the purine analog is 6-TG. In some embodiments, an amount of 6-TG ranges from between about 1 to about 15 μg/mL. In some embodiments, the positive selection comprises contacting the generated substantially HPRT deficient lymphocytes with both a purine analog and allopurinol. In some embodiments, the modified lymphocytes are administered as a single bolus. In some embodiments, multiple doses of the modified lymphocytes are administered to the patient. In some embodiments, each dose of the modified lymphocytes comprises between about 0.1×10⁶ cells/kg to about 240×10⁶ cells/kg. In some embodiments, a total dosage of modified lymphocytes comprises between about 0.1×10⁶ cells/kg to about 730×10⁶ cells/kg.

In an twenty-seventh additional embodiment of the present disclosure is a method of treating a patient with hypoxanthine-guanine phosphoribosyl transferase (HPRT) deficient lymphocytes including the steps of: (a) isolating lymphocytes from a donor subject; (b) transducing the isolated lymphocytes with an expression vector; (c) exposing the transduced isolated lymphocytes to an agent which positively selects for HPRT deficient lymphocytes to provide a preparation of modified lymphocytes; (d) administering a therapeutically effective amount of the preparation of the modified lymphocytes to the patient following hematopoietic stem-cell transplantation; and (e) optionally administering methotrexate or mycophenolic acid following the development of graft-versus-host disease (GvHD) in the patient. In some embodiments, the expression vector comprises a first expression control sequence operably linked to a first nucleic acid sequence, the first nucleic acid sequence encoding a shRNA to knockdown HPRT, wherein the shRNA has at least 90% identity to the sequence of any one of SEQ ID NOS: 2 and 5-11. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of any one of SEQ ID NOS: 2 and 5-11. In some embodiments, the expression vector comprises a first expression control sequence operably linked to a first nucleic acid sequence, the first nucleic acid sequence encoding a shRNA to knockdown HPRT, wherein the shRNA has at least 97% identity to the sequence of any one of SEQ ID NOS: 2 and 5-11. In some embodiments, the shRNA comprises the sequence of any one of SEQ ID NOS: 2, 5, 6, 7, 8, 9, 10, and 11.

In some embodiments, the dihydrofolate reductase inhibitor is selected from the group consisting of MTX or MPA. In some embodiments, the agent which positively selects for the HPRT deficient lymphocytes comprises a purine analog. In some embodiments, the purine analog is 6-TG. In some embodiments, an amount of 6-TG ranges from between about 1 to about 15 μg/mL.

In an additional embodiment of the present disclosure is a method of treating a patient with HPRT deficient lymphocytes including the steps of: (a) isolating lymphocytes from a donor subject; (b) contacting the isolated lymphocytes with a delivery vehicle including components adapted to knockout HPRT to provide a population of HPRT deficient lymphocytes; (c) exposing the population of HPRT deficient lymphocytes to an agent which positively selects for HPRT deficient lymphocytes to provide a preparation of modified lymphocytes; (d) administering a therapeutically effective amount of the preparation of the modified lymphocytes to the patient following hematopoietic stem-cell transplantation; and (e) optionally administering a dihydrofolate reductase inhibitor following the development of graft-versus-host disease (GvHD) in the patient.

In some embodiments, the dihydrofolate reductase inhibitor is selected from the group consisting of MTX or MPA. In some embodiments, the agent which positively selects for the HPRT deficient lymphocytes comprises a purine analog. In some embodiments, the purine analog is 6-TG. In some embodiments, an amount of 6-TG ranges from between about 1 to about 15 μg/mL.

In some embodiments, the components adapted to knockout HPRT comprise a guide RNA having at least 90% sequence identity to any one of SEQ ID NOS: 25-39. In some embodiments, the components adapted to knockout HPRT comprise a guide RNA having at least 95% sequence identity to any one of SEQ ID NOS: 25-39. In some embodiments, the components adapted to knockout HPRT comprise a guide RNA targeting a nucleic acid sequence selected from the group consisting of SEQ ID NOS: 25-39. In some embodiments, the components adapted to knockout HPRT further comprises a Cas protein. In some embodiments, the Cas protein comprises a Cas9 protein. In some embodiments, the Cas protein comprises a Cas12 protein. In some embodiments, the Cas12 protein is a Cas12a protein. In some embodiments, the Cas12 protein is a Cas12b protein. In some embodiments, the delivery vehicle is a nanocapsule. In some embodiments, the delivery vehicle is a nanocapsule comprising one or more targeting moieties.

In a twenty-ninth additional embodiment of the present disclosure is a use of a preparation of modified lymphocytes for providing the benefits of a lymphocyte infusion to a subject in need of treatment thereof following hematopoietic stem-cell transplantation, wherein the preparation of the modified lymphocytes are generated by: (a) isolating lymphocytes from a donor subject; (b) transducing the isolated lymphocytes with an expression vector; and (c) exposing the transduced isolated lymphocytes to an agent which positively selects for HPRT deficient lymphocytes to provide the preparation of modified lymphocytes. In some embodiments, the expression vector comprises a first expression control sequence operably linked to a first nucleic acid sequence, the first nucleic acid sequence encoding a shRNA to knockdown HPRT, wherein the shRNA has at least 90% identity to the sequence of any of SEQ ID NOS: 2 and 5-11. In some embodiments, the expression vector comprises a first expression control sequence operably linked to a first nucleic acid sequence, the first nucleic acid sequence encoding a shRNA to knockdown HPRT, wherein the shRNA has at least 95% identity to the sequence of any of SEQ ID NOS: 2 and 5-11. In some embodiments, the expression vector comprises a first expression control sequence operably linked to a first nucleic acid sequence, the first nucleic acid sequence encoding a shRNA to knockdown HPRT, wherein the shRNA has at least 97% identity to the sequence of any of SEQ ID NOS: 2 and 5-11. In some embodiments, the shRNA comprises the sequence of any one of SEQ ID NOS: 2, 5, 6, 7, 8, 9, 10, and 11.

In a thirtieth additional embodiment of the present disclosure is a use of a preparation of modified lymphocytes for providing the benefits of a lymphocyte infusion to a subject in need of treatment thereof following hematopoietic stem-cell transplantation, wherein the preparation of the modified lymphocytes are generated by: (a) isolating lymphocytes from a donor subject; (b) contacting the isolated lymphocytes with a delivery vehicle including components adapted to knockout HPRT to provide a population of HPRT deficient lymphocytes; and (c) exposing the population of HPRT deficient lymphocytes to an agent which positively selects for HPRT deficient lymphocytes to provide a preparation of modified lymphocytes. In some embodiments, the delivery vehicle is a nanocapsule. In some embodiments, the nanocapsule comprises a gRNA having at least 90% sequence identity to any one of SEQ ID NOS: 25-39 and a Cas protein (e.g. a Cas9 protein, a Cas12a protein, or a Cas12b protein). In some embodiments, the nanocapsule comprises a gRNA having at least 95% sequence identity to any one of SEQ ID NOS: 25-39 and a Cas protein (e.g. a Cas9 protein, a Cas12a protein, or a Cas12b protein).

In a thirty-first additional embodiment of the present disclosure is a pharmaceutical composition comprising (i) a lentiviral expression vector, wherein the lentiviral expression vector includes a first expression control sequence operably linked to a first nucleic acid sequence, the first nucleic acid sequence encoding a shRNA to knockdown hypoxanthine-guanine phosphoribosyl transferase (HPRT), wherein the shRNA has at least 90% identity to the sequence of any of SEQ ID NOS: 2, 5, 6, 7, 8, 9, 10, and 11; and (ii) a pharmaceutically acceptable carrier or excipient. In some embodiments, the shRNA has at least 95% identity to the sequence of any of SEQ ID NOS: 2, 5, 6, 7, 8, 9, 10, and 11. In some embodiments, the shRNA has at least 97% identity to the sequence of any of SEQ ID NOS: 2, 5, 6, 7, 8, 9, 10, and 11. In some embodiments, the shRNA comprises the sequence of any one of SEQ ID NOS: 2, 5, 6, 7, 8, 9, 10, and 11.

In a thirty-second additional embodiment of the present disclosure is a kit comprising (i) a guide-RNA having at least 90% sequence identity to any one of SEQ ID NOS: 25-39; and (ii) a Cas protein. In some embodiments, the Cas protein is selected from the group consisting of a Cas9 protein and a Cas12 protein. In some embodiments, the guide-RNA has at least 95% sequence identity to any one of SEQ ID NOS: 25-39. In some embodiments, the guide-RNA has at least 97% sequence identity to any one of SEQ ID NOS: 25-39. In some embodiments, the guide-RNA comprises the sequence of any one of SEQ ID NOS: 25-39. In some embodiments, the Cas protein is Cas9. In some embodiments, the Cas protein is Cas12a. In some embodiments, the Cas protein is Cas12b.

In a thirty-third additional embodiment of the present disclosure is a nanocapsule comprising (i) a gRNA having at least 90% sequence identity to any one of SEQ ID NOS: 25-39; and (ii) a Cas protein. In some embodiments, the Cas protein is selected from the group consisting of a Cas9 protein and a Cas12 protein. In some embodiments, the guide-RNA has at least 95% sequence identity to any one of SEQ ID NOS: 25-39. In some embodiments, the guide-RNA has at least 97% sequence identity to any one of SEQ ID NOS: 25-39. In some embodiments, the guide-RNA comprises the sequence of any one of SEQ ID NOS: 25-39. In some embodiments, the nanocapsules comprise at least one targeting moiety. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127 or FoxP3 and CD44. In some embodiments, the T-cell marker is CD3. In some embodiments, the T-cell marker is CD28. In some embodiments, the nanocapsule comprises a polymeric shell. In some embodiments, the polymeric nanocapsules are comprised of two different positively charged monomers, at least one neutral monomer, and a cross-linker. In some embodiments, the polymeric nanocapsule is free of monomers having an imidazole group.

In a thirty-fourth additional embodiment of the present disclosure is a host cell transfected with a nanocapsule, wherein the nanocapsule comprises (i) a gRNA having at least 90% sequence identity to any one of SEQ ID NOS: 25-39; and (ii) a Cas protein. In some embodiments, the Cas protein is selected from the group consisting of a Cas9 protein and a Cas12 protein. In some embodiments, the guide-RNA has at least 95% sequence identity to any one of SEQ ID NOS: 25-39. In some embodiments, the guide-RNA has at least 97% sequence identity to any one of SEQ ID NOS: 25-39. In some embodiments, the guide-RNA comprises the sequence of any one of SEQ ID NOS: 25-39. In some embodiments, the nanocapsule comprises at least one targeting moiety. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127 or FoxP3 and CD44. In some embodiments, the T-cell marker is CD3. In some embodiments, the T-cell marker is CD28. In some embodiments, the nanocapsule comprises a polymeric shell. In some embodiments, the nanocapsules are comprised of two different positively charged monomers, at least one neutral monomer, and a cross-linker. In some embodiments, the polymeric nanocapsule is free of monomers having an imidazole group.

In a thirty-fifth additional embodiment of the present disclosure is a use of a preparation of modified lymphocytes for providing the benefits of a lymphocyte infusion to a subject in need of treatment thereof following hematopoietic stem-cell transplantation, wherein the preparation of the modified lymphocytes are generated by: (a) isolating lymphocytes from a donor subject; (b) contacting the isolated lymphocytes with nanocapsules, the nanocapsules comprising (i) a gRNA having at least 90% sequence identity to any one of SEQ ID NOS: 25-39; and (ii) a Cas protein; and (c) exposing the population of HPRT deficient lymphocytes to an agent which positively selects for HPRT deficient lymphocytes to provide a preparation of modified lymphocytes. In some embodiments, the gRNA comprises the sequence of any one of SEQ ID NOS: 25-39.

In a thirty-sixth additional embodiment of the present disclosure is a nanocapsule comprising an expression vector comprising a first expression control sequence operably linked to a first nucleic acid sequence, the first nucleic acid sequence encoding a shRNA to knockdown HPRT, wherein the shRNA has at least 90% identity to the sequence of any one of SEQ ID NOS: 2, 5, 6, 7, 8, 9, 10, and 11. In some embodiments, the shRNA has at least 95% identity to the sequence of anyone of SEQ ID NOS: 2, 5, 6, 7, 8, 9, 10, and 11. In some embodiments, the shRNA has at least 97% identity to the sequence of any one of SEQ ID NOS: 2, 5, 6, 7, 8, 9, 10, and 11. In some embodiments, the shRNA comprises the sequence of any one of SEQ ID NOS: 2, 5, 6, 7, 8, 9, 10, and 11. In some embodiments, the nanocapsules comprise at least one targeting moiety. In some embodiments, the nanocapsule comprises a polymeric shell. In some embodiments, the polymeric nanocapsules are comprised of two different positively charged monomers, at least one neutral monomer, and a cross-linker. In some embodiments, the at least one targeting moiety targets a T-cell marker. In some embodiments, the T-cell marker is selected from CD3, CD4, CD7, CD8, CD25, CD27, CD28, CD45RA, RO, CD56, CD62L, CD127 or FoxP3 and CD44. In some embodiments, the T-cell marker is CD3. In some embodiments, the T-cell marker is CD28.

### Further Embodiments

Further Embodiment 1 A method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA molecule targeting a sequence within one of Exon 3 or Exon 8 of the HPRT 1 gene; (b) positively selecting for the population of substantially HPRT deficient lymphocytes ex vivo to provide a population of modified lymphocytes; and (c) administering a therapeutically effective amount of the population of modified lymphocytes to the patient.

Further Embodiment 2 The method of further embodiment 1, further comprising administering an HSC graft to the patient.

Further Embodiment 3 The method of further embodiment 2, wherein the HSC graft is administered prior to, contemporaneously with, or following the administration of the population of modified lymphocytes.

Further Embodiment 4 The method of further embodiment 1, wherein the guide RNA molecule targets a sequence within Exon 3 of the HPRT 1 gene.

Further Embodiment 5 The method of further embodiment 1, wherein the guide RNA molecule targets a sequence within Exon 8 of the HPRT 1 gene.

Further Embodiment 6 The method of further embodiment 1, wherein the guide RNA molecule targeting the sequence within the one of Exon 3 or Exon 8 of the HPRT 1 gene has at least 90% sequence identity to any one of SEQ ID NOS: 40-44 and 46-56.

Further Embodiment 7 The method of further embodiment 1, wherein the guide RNA molecule targeting the sequence within the one of Exon 3 or Exon 8 of the HPRT 1 gene has at least 95% sequence identity to any one of SEQ ID NOS: 40-44 and 46-56.

Further Embodiment 8 The method of further embodiment 1, wherein the guide RNA molecule targeting the sequence within the one of Exon 3 or Exon 8 of the HPRT 1 gene has at least 97% sequence identity to any one of SEQ ID NOS: 40-44 and 46-56.

Further Embodiment 9 The method of further embodiment 1, wherein the guide RNA molecule targeting the sequence within the one of Exon 3 or Exon 8 of the HPRT 1 gene comprises any one of SEQ IDS: 40-44 and 46-56.

Further Embodiment 10 The method of any one of the preceding further embodiments, wherein the endonuclease comprises a Cas protein.

Further Embodiment 11 The method of further embodiment 10, wherein the Cas protein comprises a Cas9 protein.

Further Embodiment 12 The method of further embodiment 10, wherein the Cas protein comprises a Cas12 protein.

Further Embodiment 13 The method of further embodiment 12, wherein the Cas12 protein is a Cas12a protein.

Further Embodiment 14 The method of further embodiment 12, wherein the Cas12 protein is a Cas12b protein.

Further Embodiment 15 The method of any one of the preceding further embodiments, wherein the lymphocytes obtained from the donor sample are transfected or transduced with a viral delivery vehicle, a non-viral delivery vehicle, or through a physical method.

Further Embodiment 16 The method of further embodiment 15, wherein the physical method is selected from microinjection and electroporation.

Further Embodiment 17 The method of further embodiment 15, wherein the non-viral delivery vehicle is a nanocapsule.

Further Embodiment 18 The method of further embodiment 17, wherein the nanocapsule comprises at least one targeting moiety.

Further Embodiment 19 The method of further embodiment 18, wherein the at least one targeting moiety targets a cluster of differentiation marker selected from the group consisting of CD3, CD4, CD7, CD8, CD25, CD27, CD28, CD45R A, RO, CD56, CD62L, CD127, FoxP3, and CD44.

Further Embodiment 20 The method of any one of the preceding further embodiments, further comprising activating one or more cell surface markers selected from the group consisting of CD28, ICOS, CTLA4, PD1, PD1H, and BTLA.

Further Embodiment 21 The method of further embodiment 15, wherein the viral delivery vehicle is an expression vector, and wherein the expression vector includes a first nucleic acid sequence encoding for the endonuclease and a second nucleic acid encoding for the guide RNA molecule.

Further Embodiment 22 The method of further embodiment 21, wherein the expression vector is a lentiviral expression vector.

Further Embodiment 23 The method of any one of the preceding further embodiments, wherein a level of HPRT1 gene expression within the population of substantially HPRT deficient lymphocytes is reduced by at least about 70% as compared with the donor lymphocytes which have not been transfected.

Further Embodiment 24 The method of any one of the preceding further embodiments, wherein a level of HPRT1 gene expression within the population of substantially HPRT deficient lymphocytes is reduced by at least about 80% as compared with the donor lymphocytes which have not been transfected.

Further Embodiment 25 The method of any one of the preceding further embodiments, wherein a level of HPRT1 gene expression within the population of substantially HPRT deficient lymphocytes is reduced by at least about 90% as compared with the donor lymphocytes which have not been transfected.

Further Embodiment 26 The method of any one of the preceding further embodiments, wherein the positive selection comprises contacting the generated population of substantially HPRT deficient lymphocytes with a purine analog.

Further Embodiment 27 The method of further embodiment 26, wherein the purine analog is selected from the group consisting of 6-TG and 6-MP.

Further Embodiment 28 The method of further embodiment any one of further embodiments 26 to 27, wherein an amount of the purine analog ranges from between about 1 to about 15 μg/mL.

Further Embodiment 29 The method of any one of the preceding further embodiments, wherein the positive selection comprises contacting the generated population of substantially HPRT deficient lymphocytes with both a purine analog and allopurinol.

Further Embodiment 30 The method of any one of further embodiments, wherein at least about 70% of the population of modified lymphocytes are sensitive to a dihydrofolate reductase inhibitor.

Further Embodiment 31 The method of any one of the preceding further embodiments, further comprising administering to the patient one or more doses of a dihydrofolate reductase inhibitor.

Further Embodiment 32 The method of further embodiment 31, wherein the dihydrofolate reductase inhibitor is selected from the group consisting of MTX or MPA.

Further Embodiment 33 The method of any one of the preceding further embodiments, wherein the population modified lymphocytes are administered as a single bolus.

Further Embodiment 34 The method of any one of the preceding further embodiments, wherein multiple doses of the population of modified lymphocytes are administered to the patient.

Further Embodiment 35 The method of further embodiment 34, wherein each dose of the multiple doses comprises between about 0.1×10⁶ cells/kg to about 240×10⁶ cells/kg. Further Embodiment 36 The method of further embodiment 35, wherein a total dosage comprises between about 0.1×10⁶ cells/kg to about 730×10⁶ cells/kg.

Further Embodiment 37 A method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA molecule targeting a sequence within Chromosome X located between about 134475181 to about 134475364 or between about 134498608 to about 134498684 based on genome build GRCh38 or the equivalent positions in a genome build other than GRCh38; (b) positively selecting for the population of substantially HPRT deficient lymphocytes ex vivo to provide a population of modified lymphocytes; (c) administering a therapeutically effective amount of the population of modified lymphocytes to the patient.

Further Embodiment 38 The method of further embodiment 37, further comprising administering an HSC graft to the patient.

Further Embodiment 39 The method of further embodiment 38, wherein the HSC graft is administered prior to, contemporaneously with, or following the administration of the population of modified lymphocytes.

Further Embodiment 40 The method of any one of further embodiments 37 to 39, wherein the guide RNA molecule is at least about 85% complementary to the sequence within Chromosome X located between about 134475181 to about 134475364 based on genome build GRCh38 or the equivalent position in a genome build other than GRCh38.

Further Embodiment 41 The method of any one of further embodiments 37 to 39, wherein the guide RNA molecules targets the sequence within Chromosome X located between about 134475181 to about 134475364 based on genome build GRCh38 or the equivalent position in a genome build other than GRCh38.

Further Embodiment 42 The method of further embodiment 41, wherein the sequence targeted has a length ranging from between about 14 nucleotides to about 30 nucleotides.

Further Embodiment 43 The method of further embodiment 41, wherein the sequence targeted has a length ranging from between about 18 nucleotides to about 26 nucleotides.

Further Embodiment 44 The method of further embodiment 41, wherein the sequence targeted has a length ranging from between about 21 nucleotides to about 25 nucleotides.

Further Embodiment 45 The method of any one of further embodiments 37 to 39, wherein the guide RNA molecule is at least about 85% complementary to the sequence within Chromosome X located between about 134498608 to about 134498684 or the equivalent position in a genome build other than GRCh38.

Further Embodiment 46 The method of any one of further embodiments 37 to 39, wherein the guide RNA molecules targets the sequence within Chromosome X located between about 134498608 to about 134498684 or the equivalent position in a genome build other than GRCh38.

Further Embodiment 47 The method of further embodiment 46, wherein the sequence targeted has a length ranging from between about 14 nucleotides to about 30 nucleotides.

Further Embodiment 48 The method of further embodiment 46, wherein the sequence targeted has a length ranging from between about 18 nucleotides to about 26 nucleotides.

Further Embodiment 49 The method of further embodiment 46, wherein the sequence targeted has a length ranging from between about 21 nucleotides to about 25 nucleotides.

Further Embodiment 50 The method of any one of further embodiments 37 to 39, wherein the guide RNA molecule has at least 90% sequence identity to any one of SEQ ID NOS: 40-44 and 46-56.

Further Embodiment 51 The method of any one of further embodiments 37 to 39, wherein the guide RNA molecule gene has at least 95% sequence identity to any one of SEQ ID NOS: 40-44 and 46-56.

Further Embodiment 52 The method of any one of further embodiments 37 to 39, wherein the guide RNA molecule has at least 97% sequence identity to any one of SEQ ID NOS: 40-44 and 46-56.

Further Embodiment 53 The method of any one of further embodiments 37 to 39, wherein the guide RNA molecule comprises any one of SEQ ID NOS: 40-44 and 46-56.

Further Embodiment 54 The method of any one of further embodiments 37 to 53, wherein the endonuclease comprises a Cas protein.

Further Embodiment 55 The method of further embodiment 54, wherein the Cas protein comprises a Cas9 protein.

Further Embodiment 56 The method of further embodiment 54, wherein the Cas protein comprises a Cas12 protein.

Further Embodiment 57 The method of further embodiment 56, wherein the Cas12 protein is a Cas12a protein.

Further Embodiment 58 The method of further embodiment 56, wherein the Cas12 protein is a Cas12b protein.

Further Embodiment 59 The method of any one of further embodiments 37 to 58, wherein the lymphocytes obtained from a donor sample are transfected or transduced with a viral delivery vehicle, a non-viral delivery vehicle, or through a physical method.

Further Embodiment 60 The method of further embodiment 59, wherein the physical method is selected from microinjection and electroporation.

Further Embodiment 61 The method of further embodiment 59, wherein the non-viral delivery vehicle is a nanocapsule.

Further Embodiment 62 The method of further embodiment 61, wherein the nanocapsule comprises at least one targeting moiety.

Further Embodiment 63 The method of further embodiment 62, wherein the at least one targeting moiety targets a cluster of differentiation marker selected from the group consisting of CD3, CD4, CD7, CD8, CD25, CD27, CD28, CD45RA, RO, CD56, CD62L, CD127, FoxP3, and CD44.

Further Embodiment 64 The method of any one of further embodiments 37 to 63, further comprising activating one or more cell surface markers selected from the group consisting of CD28, ICOS, CTLA4, PD1, PD1H, and BTLA.

Further Embodiment 65 The method of any one of further embodiments 37 to 58, wherein the viral delivery vehicle is an expression vector, and wherein the expression vector includes a first nucleic acid sequence encoding for the endonuclease and a second nucleic acid encoding for the guide RNA molecule.

Further Embodiment 66 The method of further embodiment 65, wherein the expression vector is a lentiviral expression vector.

Further Embodiment 67 The method of any one of further embodiments 37 to 66, wherein a level of HPRT1 gene expression within the population of substantially HPRT deficient lymphocytes is reduced by at least about 70% as compared with the donor lymphocytes which have not been transfected.

Further Embodiment 68 The method of any one of further embodiments 37 to 66, wherein a level of HPRT1 gene expression within the population of substantially HPRT deficient lymphocytes is reduced by at least about 80% as compared with the donor lymphocytes which have not been transfected.

Further Embodiment 69 The method of any one of further embodiments 37 to 66, wherein a level of HPRT1 gene expression within the population of substantially HPRT deficient lymphocytes is reduced by at least about 90% as compared with the donor lymphocytes which have not been transfected.

Further Embodiment 70 The method of any one of further embodiments 37 to 66, wherein the positive selection comprises contacting the generated population of substantially HPRT deficient lymphocytes with a purine analog.

Further Embodiment 71 The method of further embodiment 70, wherein the purine analog is selected from 6-TG and 6-MP.

Further Embodiment 72 The method of any one of further embodiments 70 to 71, wherein an amount of the purine analog ranges from between about 1 to about 15 μg/mL.

Further Embodiment 73 The method of any one of further embodiments 37 to 69, wherein the positive selection comprises contacting the generated population of substantially HPRT deficient lymphocytes with both a purine analog and allopurinol.

Further Embodiment 74 The method of any one of further embodiments 37 to 73, wherein at least about 70% of the modified lymphocytes are sensitive to a dihydrofolate reductase inhibitor.

Further Embodiment 75 The method of any one of further embodiments 37 to 74, further comprising administering to the patient one or more doses of a dihydrofolate reductase inhibitor.

Further Embodiment 76 The method of further embodiment 75, wherein the dihydrofolate reductase inhibitor is selected from the group consisting of MTX or MPA.

Further Embodiment 77 The method of any one of further embodiments 37 to 76, wherein the modified lymphocytes are administered as a single bolus.

Further Embodiment 78 The method of any one of further embodiments 37 to 76, wherein multiple doses of the modified lymphocytes are administered to the patient.

Further Embodiment 79 The method of any one of further embodiments 37 to 78, wherein each dose of the multiple doses comprises between about 0.1×10⁶ cells/kg to about 240×10⁶ cells/kg.

Further Embodiment 80 The method of further embodiment 79, wherein a total dosage comprises between about 0.1×10⁶ cells/kg to about 730×10⁶ cells/kg.

Further Embodiment 81 A method of treating a hematological cancer in a patient in need of treatment thereof comprising: (a) generating a population of substantially HPRT deficient lymphocytes by transfecting or transducing lymphocytes obtained from a donor sample with (i) an endonuclease, and (ii) a guide RNA molecule targeting a sequence within one of Exon 3 or Exon 8 of the HPRT 1 gene; (b) positively selecting for the population of substantially HPRT deficient lymphocytes ex vivo to provide a population of modified lymphocytes; (c) inducing at least a partial graft versus malignancy effect by administering an HSC graft to the patient; and (d) administering a therapeutically effective amount of the population of modified lymphocytes to the patient following the detection of residual disease or disease recurrence.

Further Embodiment 82 The method of further embodiment 81, wherein the guide RNA molecule targets a sequence within Chromosome X located between about 134475181 to about 134475364 based on genome build GRCh38 or an equivalent position in a genome build other than GRCh38.

Further Embodiment 83 The method of further embodiment 82, wherein the guide RNA molecule is at least about 85% complementary to the sequence within Chromosome X located between about 134475181 to about 134475364 based on genome build GRCh38 or an equivalent position in a genome build other than GRCh38.

Further Embodiment 84 The method of further embodiment 82, wherein the sequence targeted has a length ranging from between about 14 nucleotides to about 30 nucleotides.

Further Embodiment 85 The method of further embodiment 82, wherein the sequence targeted has a length ranging from between about 18 nucleotides to about 26 nucleotides.

Further Embodiment 86 The method of further embodiment 82, wherein the sequence targeted has a length ranging from between about 21 nucleotides to about 25 nucleotides.

Further Embodiment 87 The method of further embodiment 81, wherein the guide RNA molecules targets the sequence within Chromosome X located between about 134498608 to about 134498684 based on genome build GRCh38 or an equivalent position in a genome build other than GRCh38.

Further Embodiment 88 The method of further embodiment 87, wherein the guide RNA molecule is at least about 85% complementary to the sequence within Chromosome X located between about 134498608 to about 134498684 based on genome build GRCh38 or an equivalent position in a genome build other than GRCh38.

Further Embodiment 89 The method of further embodiment 87, wherein the sequence targeted has a length ranging from between about 14 nucleotides to about 30 nucleotides.

Further Embodiment 90 The method of further embodiment 87, wherein the sequence targeted has a length ranging from between about 18 nucleotides to about 26 nucleotides.

Further Embodiment 91 The method of further embodiment 87, wherein the sequence targeted has a length ranging from between about 21 nucleotides to about 25 nucleotides.

Further Embodiment 92 The method of further embodiment 81, wherein the guide RNA molecule has at least 90% sequence identity to any one of SEQ ID NOS: 40-44 and 46-56.

Further Embodiment 93 The method of further embodiment 81, wherein the guide RNA molecule gene has at least 95% sequence identity to any one of SEQ ID NOS: 40-44 and 46-56.

Further Embodiment 94 The method of further embodiment 81, wherein the guide RNA molecule has at least 97% sequence identity to any one of SEQ ID NOS: 40-44 and 46-56.

Further Embodiment 95 The method of further embodiment 81, wherein the guide RNA molecule comprises any one of SEQ ID NOS: 40-44 and 46-56.

Further Embodiment 96 The method of further embodiment 81, wherein the Cas protein comprises a Cas9 protein.

Further Embodiment 97 The method of further embodiment 81, wherein the Cas protein comprises a Cas12 protein.

Further Embodiment 98 The method of further embodiment 81, wherein the Cas12 protein is a Cas12a protein.

Further Embodiment 99 The method of further embodiment 81, wherein the Cas12 protein is a Cas12b protein.

Further Embodiment 100 The method of any one of further embodiments 81 to 99, wherein the lymphocytes obtained from the donor sample are transfected or transduced with a viral delivery vehicle, a non-viral delivery vehicle, or through a physical method.

Further Embodiment 101 The method of further embodiment 100, wherein the physical method is selected from microinjection and electroporation.

Further Embodiment 102 The method of further embodiment 100, wherein the non-viral delivery vehicle is a nanocapsule.

Further Embodiment 103 The method of further embodiment 102, wherein the nanocapsule comprises at least one targeting moiety.

Further Embodiment 104 The method of further embodiment 103, wherein the at least one targeting moiety targets a cluster of differentiation marker selected from the group consisting of CD3, CD4, CD7, CD8, CD25, CD27, CD28, CD45R A, RO, CD56, CD62L, CD127, FoxP3, and CD44.

Further Embodiment 105 The method of any one of further embodiment 100, wherein the viral delivery vehicle is an expression vector, and wherein the expression vector includes a first nucleic acid sequence encoding for the endonuclease and a second nucleic acid encoding for the guide RNA molecule.

Further Embodiment 106 The method of further embodiment 105, wherein the expression vector is a lentiviral expression vector.

Further Embodiment 107 The method of any one of further embodiments 81 to 106, wherein a level of HPRT1 gene expression within the population of substantially HPRT deficient lymphocytes is reduced by at least about 70% as compared with the donor lymphocytes which have not been transfected.

Further Embodiment 108 The method of any one of further embodiments 81 to 106, wherein a level of HPRT1 gene expression within the population of substantially HPRT deficient lymphocytes is reduced by at least about 80% as compared with the donor lymphocytes which have not been transfected.

Further Embodiment 109 The method of any one of further embodiments 81 to 106, wherein a level of HPRT1 gene expression within the population of substantially HPRT deficient lymphocytes is reduced by at least about 90% as compared with the donor lymphocytes which have not been transfected.

Further Embodiment 110 The method of any one of further embodiments 81 to 109, wherein the positive selection comprises contacting the generated population of substantially HPRT deficient lymphocytes with a purine analog.

Further Embodiment 111 The method of further embodiment 110, wherein the purine analog is selected from the group consisting of 6-TG and 6-MP.

Further Embodiment 112 The method of any one of further embodiments 110, wherein an amount of the purine analog ranges from between about 1 to about 15 μg/mL.

Further Embodiment 113 The method of any one of further embodiments 81 to 109, wherein the positive selection comprises contacting the generated population of substantially HPRT deficient lymphocytes with both a purine analog and allopurinol.

Further Embodiment 114 The method of any one of further embodiments 81 to 113, wherein at least about 70% of the modified lymphocytes are sensitive to a dihydrofolate reductase inhibitor.

Further Embodiment 115 The method of any one of further embodiments 81 to 113, further comprising administering to the patient one or more doses of a dihydrofolate reductase inhibitor.

Further Embodiment 116 The method of further embodiment 115, wherein the dihydrofolate reductase inhibitor is selected from the group consisting of MTX or MPA.

Further Embodiment 117 The method of any one of further embodiments 81 to 116, wherein the modified lymphocytes are administered as a single bolus.

Further Embodiment 118 The method of any one of further embodiments 81 to 116, wherein multiple doses of the modified lymphocytes are administered to the patient.

Further Embodiment 119 The method of further embodiment 118, wherein each dose of the multiple doses comprises between about 0.1×10⁶ cells/kg to about 240×10⁶ cells/kg.

Further Embodiment 120 The method of further embodiment 118, wherein a total dosage comprises between about 0.1×10⁶ cells/kg to about 730×10⁶ cells/kg.

Further Embodiment 121 A method of treating a patient with HPRT deficient lymphocytes including the steps of: (a) isolating lymphocytes from a donor subject; (b) contacting the isolated lymphocytes with (i) an endonuclease, and (ii) a guide RNA molecule targeting a sequence within one of Exon 3 or Exon 8 of the HPRT 1 gene; (c) exposing the population of HPRT deficient lymphocytes to an agent which positively selects for HPRT deficient lymphocytes to provide a preparation of modified lymphocytes; (d) administering a therapeutically effective amount of the preparation of the modified lymphocytes to the patient following hematopoietic stem-cell transplantation; and (e) optionally administering a dihydrofolate reductase inhibitor following the development of graft-versus-host disease (GvHD) in the patient.

Further Embodiment 122 The method of further embodiment 121, wherein the dihydrofolate reductase inhibitor is selected from the group consisting of MTX or MPA.

Further Embodiment 123 The method of any one of further embodiments 121 to 122, wherein the agent which positively selects for the HPRT deficient lymphocytes comprises a purine analog.

Further Embodiment 124 The method of further embodiment 123, wherein the purine analog is selected from the group consisting of 6-TG and 6-MP.

Further Embodiment 125 The method of further embodiment 123, wherein the amount of purine analog ranges from between about 1 to about 15 μg/mL.

Further Embodiment 126 The method of further embodiment 121, wherein the guide RNA molecule targets a sequence within Chromosome X located between about 134475181 to about 134475364 based on genome build GRCh38. or an equivalent position in a genome build other than GRCh38

Further Embodiment 127 The method of further embodiment 126, wherein the guide RNA molecule is at least about 85% complementary to the sequence within Chromosome X located between about 134475181 to about 134475364 based on genome build GRCh38 or an equivalent position in a genome build other than GRCh38.

Further Embodiment 128 The method of further embodiment 126, wherein the sequence targeted has a length ranging from between about 14 nucleotides to about 30 nucleotides.

Further Embodiment 129 The method of further embodiment 126, wherein the sequence targeted has a length ranging from between about 18 nucleotides to about 26 nucleotides.

Further Embodiment 130 The method of further embodiment 126, wherein the sequence targeted has a length ranging from between about 21 nucleotides to about 25 nucleotides.

Further Embodiment 131 The method of further embodiment 121, wherein the guide RNA molecules targets the sequence within Chromosome X located between about 134498608 to about 134498684 based on genome build GRCh38 or an equivalent position in a genome build other than GRCh38.

Further Embodiment 132 The method of further embodiment 131, wherein the guide RNA molecule is at least about 85% complementary to the sequence within Chromosome X located between about 134498608 to about 134498684 based on genome build GRCh38 or an equivalent position in a genome build other than GRCh38.

Further Embodiment 133 The method of further embodiment 131, wherein the sequence targeted has a length ranging from between about 14 nucleotides to about 30 nucleotides.

Further Embodiment 134 The method of further embodiment 131, wherein the sequence targeted has a length ranging from between about 18 nucleotides to about 26 nucleotides.

Further Embodiment 135 The method of further embodiment 131, wherein the sequence targeted has a length ranging from between about 21 nucleotides to about 25 nucleotides.

Further Embodiment 136 The method of further embodiment 121, wherein the guide RNA molecule has at least 90% sequence identity to any one of SEQ ID NOS: 40-44 and 46-56.

Further Embodiment 137 The method of further embodiment 121, wherein the guide RNA molecule gene has at least 95% sequence identity to any one of SEQ ID NOS: 40-44 and 46-56.

Further Embodiment 138 The method of further embodiment 121, wherein the guide RNA molecule has at least 97% sequence identity to any one of SEQ ID NOS: 40-44 and 46-56.

Further Embodiment 139 The method of further embodiment 121, wherein the guide RNA molecule comprises any one of SEQ ID NOS: 40-44 and 46-56.

Further Embodiment 140 The method of further embodiment 121, wherein the Cas protein comprises a Cas9 protein.

Further Embodiment 141 The method of further embodiment 121, wherein the Cas protein comprises a Cas12 protein.

Further Embodiment 142 The method of further embodiment 141, wherein the Cas12 protein is a Cas12a protein.

Further Embodiment 143 The method of further embodiment 141, wherein the Cas12 protein is a Cas12b protein.

Further Embodiment 144 The method of any one of further embodiments 121 to 143, wherein the lymphocytes obtained from the donor sample are contacted with a viral delivery vehicle comprising, a non-viral delivery vehicle, or through a physical method.

Further Embodiment 145 The method of further embodiment 144, wherein the physical method is selected from microinjection and electroporation

Further Embodiment 146 The method of further embodiment 144, wherein the non-viral delivery vehicle is a nanocapsule.

Further Embodiment 147 The method of further embodiment 146, wherein the nanocapsule comprises at least one targeting moiety.

Further Embodiment 148 The method of further embodiment 147, wherein the at least one targeting moiety targets a cluster of differentiation marker selected from the group consisting of CD3, CD4, CD7, CD8, CD25, CD27, CD28, CD45RA, RO, CD56, CD62L, CD127, FoxP3, and CD44.

Further Embodiment 149 The method of any one of further embodiments 121 to 148, wherein the delivery vehicle is an expression vector, and wherein the expression vector includes a first nucleic acid sequence encoding for the endonuclease and a second nucleic acid encoding for the guide RNA molecule.

Further Embodiment 150 The method of further embodiment 121 to 149, wherein the expression vector is a lentiviral expression vector.

Further Embodiment 151 The method of any one of further embodiments 121 to 150, wherein the preparation is administered as a single bolus.

Further Embodiment 152 The method of any one of further embodiments 121 to 150, wherein multiple doses of the preparation are administered to the patient.

Further Embodiment 153 The method of further embodiment 152, wherein each dose of the preparation comprises between about 0.1×10⁶ cells/kg to about 240×10⁶ cells/kg.

Further Embodiment 154 The method of further embodiment 152, wherein a total dosage of preparation comprises between about 0.1×10⁶ cells/kg to about 730×10⁶ cells/kg.

Further Embodiment 155 Use of a preparation of modified lymphocytes for providing the benefits of a lymphocyte infusion to a subject in need of treatment thereof, wherein the preparation of the modified lymphocytes are generated by: (a) isolating lymphocytes from a donor subject; (b) contacting the isolated lymphocytes with comprising (i) an endonuclease, and (ii) a guide RNA molecule targeting a sequence within one of Exon 3 or Exon 8 of the HPRT 1 gene to provide a population of substantially HPRT deficient lymphocytes; and (c) exposing the population of HPRT deficient lymphocytes to an agent which positively selects for HPRT deficient lymphocytes to provide a preparation of modified lymphocytes.

Further Embodiment 156 The use of further embodiment 155, wherein the subject is in need of treatment following hematopoietic stem cell transplantation.

Further Embodiment 157 The use of the preparation of further embodiment 155 or 156, wherein the guide RNA molecule targets a sequence within Chromosome X located between about 134475181 to about 134475364 based on genome build GRCh38 or an equivalent position in a genome build other than GRCh38.

Further Embodiment 158 The use of the preparation of further embodiment 157, wherein the guide RNA molecule is at least about 85% complementary to the sequence within Chromosome X located between about 134475181 to about 134475364 based on genome build GRCh38 or an equivalent position in a genome build other than GRCh38.

Further Embodiment 159 The use of the preparation of further embodiment 157, wherein the sequence targeted has a length ranging from between about 14 nucleotides to about 30 nucleotides.

Further Embodiment 160 The use of the preparation of further embodiment 157, wherein the sequence targeted has a length ranging from between about 18 nucleotides to about 26 nucleotides.

Further Embodiment 161 The use of the preparation of further embodiment 157, wherein the sequence targeted has a length ranging from between about 21 nucleotides to about 25 nucleotides.

Further Embodiment 162 The use of the preparation of any one of further embodiments 155 and 156, wherein the guide RNA molecules targets the sequence within Chromosome X located between about 134498608 to about 134498684 based on genome build GRCh38 or an equivalent position in a genome build other than GRCh38.

Further Embodiment 163 The use of the preparation of further embodiment 162, wherein the guide RNA molecule is at least about 85% complementary to the sequence within Chromosome X located between about 134498608 to about 134498684 based on genome build GRCh38 or an equivalent position in a genome build other than GRCh38.

Further Embodiment 164 The use of the preparation of further embodiment 162, wherein the sequence targeted has a length ranging from between about 14 nucleotides to about 30 nucleotides.

Further Embodiment 165 The use of the preparation of further embodiment 162, wherein the sequence targeted has a length ranging from between about 18 nucleotides to about 26 nucleotides.

Further Embodiment 166 The use of the preparation of further embodiment 162, wherein the sequence targeted has a length ranging from between about 21 nucleotides to about 25 nucleotides.

Further Embodiment 167 The use of the preparation of further embodiment 155, wherein the guide RNA molecule has at least 90% sequence identity to any one of SEQ ID NOS: 40-44 and 46-56.

Further Embodiment 168 The use of the preparation of further embodiment 155, wherein the guide RNA molecule gene has at least 95% sequence identity to any one of SEQ ID NOS: 40-44 and 46-56.

Further Embodiment 169 The use of the preparation of further embodiment 155, wherein the guide RNA molecule has at least 97% sequence identity to any one of SEQ ID NOS: 40-44 and 46-56.

Further Embodiment 170 The use of the preparation of further embodiment 155, wherein the guide RNA molecule comprises any one of SEQ ID NOS: 40-44 and 46-56.

Further Embodiment 171 A kit comprising: (i) a guide RNA molecule having at least 90% sequence identity to any one of SEQ ID NOS: 40-49; and (ii) a Cas protein.

Further Embodiment 172 The kit of further embodiment 171, wherein the Cas protein is selected from the group consisting of a Cas9 protein and a Cas12 protein.

Further Embodiment 173 The kit of any one of further embodiments 171 to 172, wherein the guide RNA molecule has at least 95% sequence identity to any one of SEQ ID NOS: 40-56.

Further Embodiment 174 The kit of any one of further embodiments 171 to 172, wherein the guide RNA molecule has at least 97% sequence identity to any one of SEQ ID NOS: 40-56.

Further Embodiment 175 The kit of any one of further embodiments 171 to 172, wherein the guide RNA molecule has at least 99% sequence identity to any one of SEQ ID NOS: 40-56.

Further Embodiment 176 The kit of any one of further embodiments 171 to 172, wherein the guide RNA molecule comprises any one of SEQ ID NOS: 40-56.

Further Embodiment 177 A kit comprising: (i) a guide RNA molecule which targets a sequence within Chromosome X located between about 134475181 to about 134475364 based on genome build GRCh38 or an equivalent position in a genome build other than GRCh38, and (ii) a Cas protein.

Further Embodiment 178 The kit of further embodiment 177, wherein the Cas protein is selected from the group consisting of a Cas9 protein and a Cas12 protein.

Further Embodiment 179 The kit of further embodiment 177, wherein the guide RNA molecule is at least about 85% complementary to the sequence within Chromosome X located between about 134475181 to about 134475364 based on genome build GRCh38 or the equivalent position in a genome build other than GRCh38.

Further Embodiment 180 The kit of further embodiment 177, wherein the sequence targeted has a length ranging from between about 18 nucleotides to about 26 nucleotides.

Further Embodiment 181 The kit of further embodiment 177, wherein the sequence targeted has a length ranging from between about 21 nucleotides to about 25 nucleotides.

Further Embodiment 182 A kit comprising: (i) a guide RNA molecule which targets a sequence within Chromosome X located between about 134498608 to about 134498684 based on genome build GRCh38 or an equivalent position in a genome build other than GRCh38, and (ii) a Cas protein.

Further Embodiment 183 The kit of further embodiment 182, wherein the Cas protein is selected from the group consisting of a Cas9 protein and a Cas12 protein.

Further Embodiment 184 The kit of further embodiment 182, wherein the guide RNA molecule is at least about 85% complementary to the sequence within Chromosome X located between about 134498608 to about 134498684 based on genome build GRCh38 or the equivalent position in a genome build other than GRCh38.

Further Embodiment 185 The kit of further embodiment 182 wherein the sequence targeted has a length ranging from between about 18 nucleotides to about 26 nucleotides.

Further Embodiment 186 The kit of further embodiment 182, wherein the sequence targeted has a length ranging from between about 21 nucleotides to about 25 nucleotides.

Further Embodiment 187 A nanocapsule comprising (i) a guide RNA molecule having at least 90% sequence identity to any one of SEQ ID NOS: 40-61; and (ii) a Cas protein.

Further Embodiment 188 The nanocapsule of further embodiment 187, wherein the Cas protein is selected from the group consisting of a Cas9 protein and a Cas12 protein.

Further Embodiment 189 The nanocapsule of any one of further embodiments 187 to 188, wherein the guide-RNA has at least 95% sequence identity to any one of SEQ ID NOS: 40-61.

Further Embodiment 190 The nanocapsule of any one of further embodiments 187 to 188, wherein the guide-RNA has at least 98% sequence identity to any one of SEQ ID NOS: 40-61.

Further Embodiment 191 The nanocapsule of any one of further embodiments 187 to 188, wherein the nanocapsules comprise at least one targeting moiety.

Further Embodiment 192 The nanocapsule of further embodiment 191, wherein the at least one targeting moiety targets a cluster of differentiation marker selected from the group consisting of CD3, CD4, CD7, CD8, CD25, CD27, CD28, CD45RA, RO, CD56, CD62L, CD127 or FoxP3 and CD44.

Further Embodiment 193 The nanocapsule of any one of further embodiments 187 to 192, wherein the nanocapsule comprises a polymeric shell.

Further Embodiment 194 The nanocapsule of any one of further embodiments 187 to 193, wherein polymeric nanocapsules are comprised of two different positively charged monomers, at least one neutral monomer, and a cross-linker.

Further Embodiment 195 A host cell transfected with the nanocapsule of any one of further embodiments 187-194.

Further Embodiment 196 The host cell of further embodiment 195, wherein the host cell is a primary T-lymphocyte.

Further Embodiment 197 The host cell of further embodiment 195, wherein the host cell is a CEM cell.

Further Embodiment 198 Use of a preparation of modified lymphocytes for providing the benefits of a lymphocyte infusion to a subject in need of treatment thereof following hematopoietic stem-cell transplantation, wherein the preparation of the modified lymphocytes are generated by: (a) isolating lymphocytes from a donor subject; (b) contacting the isolated lymphocytes with the nanocapsules of any one of further embodiments 187 to 194, and (c) exposing the population of HPRT deficient lymphocytes to an agent which positively selects for HPRT deficient lymphocytes to provide a preparation of modified lymphocytes.

Further Embodiment 199 A kit comprising the nanocapsules of any one of further embodiments 187 to 194, and a dihydrofolate reductase inhibitor.

Further Embodiment 200 The kit of further embodiment 199, wherein the dihydrofolate reductase inhibitor is MTX or MPA.

Further Embodiment 201 A method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA molecule targeting a sequence within one of Exon 2, Exon 3 or Exon 8 of the HPRT 1 gene; (b) positively selecting for the population of substantially HPRT deficient lymphocytes ex vivo to provide a population of modified lymphocytes; and (c) administering a therapeutically effective amount of the population of modified lymphocytes to the patient following the administration of the HSC graft.

Further Embodiment 202 The method of further embodiment 201, further comprising administering an HSC graft to the patient.

Further Embodiment 203 The method of further embodiment 202, wherein the HSC graft is administered to prior to, contemporaneously with or after administration of the population of modified lymphocytes.

Further Embodiment 204 The method of further embodiment 201, wherein the guide RNA molecule targets a sequence within Exon 2 of the HPRT 1 gene.

Further Embodiment 205 The method of further embodiment 201, wherein the guide RNA molecule targets a sequence within Exon 3 of the HPRT 1 gene.

Further Embodiment 206 The method of further embodiment 201, wherein the guide RNA molecule targets a sequence within Exon 8 of the HPRT 1 gene.

Further Embodiment 207 The method of further embodiment 201, wherein the guide RNA molecule targeting the sequence within the one of Exon 2, Exon 3 or Exon 8 of the HPRT 1 gene has at least 90% sequence identity to any one of SEQ ID NOS: 40-61.

Further Embodiment 208 The method of further embodiment 201, wherein the guide RNA molecule targeting the sequence within the one of Exon 2, Exon 3 or Exon 8 of the HPRT 1 gene has at least 95% sequence identity to any one of SEQ ID NOS: 40-61.

Further Embodiment 209 The method of further embodiment 201, wherein the guide RNA molecule targeting the sequence within the one of Exon 2, Exon 3 or Exon 8 of the HPRT 1 gene has at least 97% sequence identity to any one of SEQ ID NOS: 40-61.

Further Embodiment 210 The method of further embodiment 201, wherein the guide RNA molecule targeting the sequence within the one of Exon 2, Exon 3 or Exon 8 of the HPRT 1 gene comprises any one of SEQ ID NOS: 40-61.

Further Embodiment 211 The method of any one of further embodiments −201 to 210, wherein the endonuclease comprises a Cas protein.

Further Embodiment 212 The method of further embodiment 211, wherein the Cas protein comprises a Cas9 protein.

Further Embodiment 213 The method of further embodiment 211, wherein the Cas protein comprises a Cas12 protein.

Further Embodiment 214 The method of further embodiment 213, wherein the Cas12 protein is a Cas12a protein.

Further Embodiment 215 The method of further embodiment 213, wherein the Cas12 protein is a Cas12b protein.

Further Embodiment 216 The method of any one of further embodiments 201 to 215, wherein the lymphocytes obtained from the donor sample are transfected or transduced with a viral delivery vehicle, a non-viral delivery vehicle, or through a physical method.

Further Embodiment 217 The method of further embodiment 216, wherein the physical method is selected from microinjection and electroporation.

Further Embodiment 218 The method of further embodiment 216, wherein the non-viral delivery vehicle is a nanocapsule.

Further Embodiment 219 The method of further embodiment 218, wherein the nanocapsule comprises at least one targeting moiety.

Further Embodiment 220 The method of further embodiment 219, wherein the at least one targeting moiety targets a cluster of differentiation marker selected from the group consisting of CD3, CD4, CD7, CD8, CD25, CD27, CD28, CD45RA, RO, CD56, CD62L, CD127, FoxP3 and CD44.

Further Embodiment 221 A method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA having at least 90% sequence identity to any one of SEQ ID NOS: 40-61; (b) positively selecting for the population of substantially HPRT deficient lymphocytes ex vivo to provide a population of modified lymphocytes; and (c) administering a therapeutically effective amount of the population of modified lymphocytes to the patient.

Further Embodiment 222 The method of further embodiment 221, further comprising administering an HSC graft to the patient.

Further Embodiment 223 The method of further embodiment 222, wherein the HSC graft is administered to prior to, contemporaneously with, or after the administration of the population of modified lymphocytes.

Further Embodiment 224 The method of further embodiment 221, wherein the guide RNA has at least 95% sequence identity to any one of SEQ ID NOS: 40-61.

Further Embodiment 225 The method of further embodiment 221, wherein the guide RNA has at least 97% sequence identity to any one of SEQ ID NOS: 40-61.

Further Embodiment 226 The method of further embodiment 221, wherein the guide RNA has at least 99% sequence identity to any one of SEQ ID NOS: 40-4961 Further Embodiment 227 The method of further embodiment 221, wherein the guide RNA comprises any one of SEQ ID NOS: 40-61.

Further Embodiment 228 A method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA molecule targeting a sequence within Exon 2 of the HPRT 1 gene; (b) positively selecting for the population of substantially HPRT deficient lymphocytes ex vivo to provide a population of modified lymphocytes; (c) administering an HSC graft to the patient; and (d) administering a therapeutically effective amount of the population of modified lymphocytes to the patient following the administration of the HSC graft.

Further Embodiment 229 The method of further embodiment 228, wherein the guide RNA molecule targeting Exon 2 of the HPRT 1 gene has at least 90% sequence identity to any one of SEQ ID NOS: 45 and 57-61.

Further Embodiment 230 The method of further embodiment 228, wherein the guide RNA molecule targeting Exon 2 of the HPRT 1 gene has at least 95% sequence identity to any one of SEQ ID NOS: 45 and 57-61.

Further Embodiment 231 The method of further embodiment 228, wherein the guide RNA molecule targeting Exon 2 of the HPRT 1 gene has at least 97% sequence identity to any one of SEQ ID NOS: 45 and 57-61.

Further Embodiment 232 The method of further embodiment 228, wherein the guide RNA molecule targeting Exon 2 of the HPRT 1 gene has at least 99% sequence identity to any one of SEQ ID NOS: 45 and 57-61.

Further Embodiment 233 The method of further embodiment 228, wherein the guide RNA molecule targeting Exon 2 of the HPRT 1 gene comprises any one of SEQ ID NOS: 45 and 57-61.

Further Embodiment 234 A method of treating a hematological cancer in a patient in need of treatment thereof comprising: (a) generating a population of substantially HPRT deficient lymphocytes by transfecting or transducing lymphocytes obtained from a donor sample with (i) an endonuclease, and (ii) a guide RNA molecule targeting a sequence within Exon 2 of the HPRT 1 gene; (b) positively selecting for the population of substantially HPRT deficient lymphocytes ex vivo to provide a population of modified lymphocytes; (c) inducing at least a partial graft versus malignancy effect by administering an HSC graft to the patient; and (d) administering a therapeutically effective amount of the population of modified lymphocytes to the patient following the detection of residual disease or disease recurrence.

Further Embodiment 235 The method of further embodiment 234, wherein the guide RNA molecule targeting Exon 2 of the HPRT 1 gene has at least 90% sequence identity to any one of SEQ ID NOS: 45 and 57-61.

Further Embodiment 236 The method of further embodiment 234, wherein the guide RNA molecule targeting Exon 2 of the HPRT 1 gene has at least 95% sequence identity to any one of SEQ ID NOS: 45 and 57-61.

Further Embodiment 237 The method of further embodiment 234, wherein the guide RNA molecule targeting Exon 2 of the HPRT 1 gene has at least 97% sequence identity to any one of SEQ ID NOS: 45 and 57-61.

Further Embodiment 238 The method of further embodiment 234, wherein the guide RNA molecule targeting Exon 2 of the HPRT 1 gene has at least 99% sequence identity to any one of SEQ ID NOS: 45 and 57-61.

Further Embodiment 239 The method of further embodiment 234, wherein the guide RNA molecule targeting Exon 2 of the HPRT 1 gene comprises any one of SEQ ID NOS: 45 and 57-61

Further Embodiment 240 A method of treating a patient with HPRT deficient lymphocytes including the steps of: (a) isolating lymphocytes from a donor subject; (b) contacting the isolated lymphocytes with (i) an endonuclease, and (ii) a guide RNA molecule targeting a sequence within Exon 2 of the HPRT 1 gene; (c) exposing the population of HPRT deficient lymphocytes to an agent which positively selects for HPRT deficient lymphocytes to provide a preparation of modified lymphocytes; (d) administering a therapeutically effective amount of the preparation of the modified lymphocytes to the patient following hematopoietic stem-cell transplantation; and (e) optionally administering a dihydrofolate reductase inhibitor following the development of graft-versus-host disease (GvHD) in the patient.

Further Embodiment 241 The method of further embodiment 240, wherein the guide RNA molecule targeting Exon 2 of the HPRT 1 gene has at least 90% sequence identity to any one of SEQ ID NOS: 45 and 57-61.

Further Embodiment 242 The method of further embodiment 240, wherein the guide RNA molecule targeting Exon 2 of the HPRT 1 gene has at least 95% sequence identity to any one of SEQ ID NOS: 45 and 57-61.

Further Embodiment 243 The method of further embodiment 240, wherein the guide RNA molecule targeting Exon 2 of the HPRT 1 gene has at least 97% sequence identity to any one of SEQ ID NOS: 45 and 57-61.

Further Embodiment 244 The method of further embodiment 240, wherein the guide RNA molecule targeting Exon 2 of the HPRT 1 gene has at least 99% sequence identity to any one of SEQ ID NOS: 45 and 57-61.

Further Embodiment 245 The method of further embodiment 240, wherein the guide RNA molecule targeting Exon 2 of the HPRT 1 gene comprises any one of SEQ ID NOS: 45 and 57-61.

Further Embodiment 246 Use of a preparation of modified lymphocytes for providing the benefits of a lymphocyte infusion to a subject in need of treatment thereof following hematopoietic stem-cell transplantation, wherein the preparation of the modified lymphocytes are generated by: (a) isolating lymphocytes from a donor subject; (b) contacting the isolated lymphocytes (i) an endonuclease, and (ii) a guide RNA molecule targeting a sequence within Exon 2 of the HPRT 1 gene to provide a population of substantially HPRT deficient lymphocytes; and (c) exposing the population of HPRT deficient lymphocytes to an agent which positively selects for HPRT deficient lymphocytes to provide a preparation of modified lymphocytes.

Further Embodiment 247 The use of further embodiment 246, wherein the guide RNA molecule targeting Exon 2 of the HPRT 1 gene has at least 90% sequence identity to any one of SEQ ID NOS: 45 and 57-61.

Further Embodiment 248 The use of further embodiment 246, wherein the guide RNA molecule targeting Exon 2 of the HPRT 1 gene has at least 95% sequence identity to any one of SEQ ID NOS: 45 and 57-61.

Further Embodiment 249 The use of further embodiment 246, wherein the guide RNA molecule targeting Exon 2 of the HPRT 1 gene has at least 97% sequence identity to any one of SEQ ID NOS: 45 and 57-61.

Further Embodiment 250 The use of further embodiment 246, wherein the guide RNA molecule targeting Exon 2 of the HPRT 1 gene has at least 99% sequence identity to any one of SEQ ID NOS: 45 and 57-61.

Further Embodiment 251 The use of further embodiment 246, wherein the guide RNA molecule targeting Exon 2 of the HPRT 1 gene comprises any one of SEQ ID NOS: 45 and 57-61.

Further Embodiment 252 A method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA molecule targeting a sequence within Chromosome X located between about 134473409 to about 134473460 based on genome build GRCh38 or the equivalent positions in a genome build other than GRCh38; (b) positively selecting for the population of substantially HPRT deficient lymphocytes ex vivo to provide a population of modified lymphocytes; (c) administering a therapeutically effective amount of the population of modified lymphocytes to the patient.

Further Embodiment 253 The method of further embodiment 252, further comprising administering an HSC graft to the patient.

Further Embodiment 254 The method of further embodiment 252, wherein the HSC graft is administered prior to, contemporaneously with, or following the administration of the population of modified lymphocytes.

Further Embodiment 255 The method of any one of further embodiments 252 to 254, wherein the guide RNA molecule is at least about 85% complementary to the sequence within Chromosome X located between about 134473409 to about 134473460 based on genome build GRCh38 or the equivalent position in a genome build other than GRCh38.

Further Embodiment 256 The method of further embodiment 255, wherein the sequence targeted has a length ranging from between about 14 nucleotides to about 30 nucleotides.

Further Embodiment 257 The method of further embodiment 255, wherein the sequence targeted has a length ranging from between about 18 nucleotides to about 26 nucleotides.

Further Embodiment 258 The method of further embodiment 255, wherein the sequence targeted has a length ranging from between about 21 nucleotides to about 25 nucleotides.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1. A method of providing benefits of a lymphocyte infusion to a patient in need of treatment thereof while mitigating side effects comprising: (a) generating a population of substantially HPRT deficient lymphocytes by transfecting or transducing lymphocytes obtained from a donor sample with (i) an endonuclease, and (ii) a guide RNA molecule targeting a sequence within one of Exon 2, Exon 3 or Exon 8 of the HPRT 1 gene; (b) positively selecting for the population of substantially HPRT deficient lymphocytes ex vivo to provide a population of modified lymphocytes; and (c) administering a therapeutically effective amount of the population of modified lymphocytes to the patient.

2. The method of claim 1, further comprising administering an HSC graft to the patient prior to, contemporaneously with, or following the administration of the population of modified lymphocytes.

3. The method of claim 1, wherein the guide RNA molecule targeting Exon 2 of the HPRT 1 gene has at least 95% sequence identity to any one of SEQ ID NOS: 45 and 57-61.

4. The method of claim 1, wherein the guide RNA molecule targeting Exon 2 of the HPRT 1 gene comprises any one of SEQ ID NOS: 45 and 57-61.

5. The method of claim 1, wherein the guide RNA molecule targeting the sequence within the one of Exon 3 of the HPRT 1 gene has at least 95% sequence identity to any one of SEQ ID NOS: 41-44, 46 and 50-51.

6. The method of claim 1, wherein the guide RNA molecule targeting the sequence within the one of Exon 3 of the HPRT 1 gene comprises any one of SEQ IDS: 41-44, 46 and 50-51.

7. The method of claim 1, wherein the guide RNA molecule targeting the sequence within the one of Exon 8 of the HPRT 1 gene has at least 95% sequence identity to any one of SEQ ID NOS: 47-49, 46, 55 and 56.

8. The method of claim 1, wherein the guide RNA molecule targeting the sequence within the one of Exon 8 of the HPRT 1 gene comprises any one of SEQ IDS: 47-49, 46, 55 and 56.

9. The method of claim 1, wherein the endonuclease comprises a Cas protein.

10. The method of claim 1, wherein the Cas protein comprises a Cas9 protein or a Cas12a protein.

11. The method of claim 1, wherein the lymphocytes obtained from the donor sample are transfected or transduced with a viral delivery vehicle, a non-viral delivery vehicle, or through a physical method.

12. The method of claim 11, wherein the physical method is selected from microinjection and electroporation.

13. The method of claim 11, wherein the non-viral delivery vehicle is a nanocapsule, optionally wherein the nanocapsule comprises at least one targeting moiety.

14. The method of claim 11, wherein the viral delivery vehicle is an expression vector, and wherein the expression vector includes a first nucleic acid sequence encoding for the endonuclease and a second nucleic acid encoding for the guide RNA molecule.

15. The method of claim 14, wherein the expression vector is a lentiviral expression vector.

16. The method of claim 1, wherein a level of HPRT1 gene expression within the population of substantially HPRT deficient lymphocytes is reduced by at least about 70%, preferably reduced by at least about 80%, more preferably reduced by at least about 90% as compared with the donor lymphocytes which have not been transfected.

17. The method of claim 1, wherein the positive selection comprises contacting the generated population of substantially HPRT deficient lymphocytes with a purine analog, preferably wherein the purine analog is selected from the group consisting of 6-TG and 6-MP.

18. The method of claim 1, wherein the positive selection comprises contacting the generated population of substantially HPRT deficient lymphocytes with both a purine analog and allopurinol.

19. The method of claim 1, further comprising administering to the patient one or more doses of a dihydrofolate reductase inhibitor if the side effects arise.

20. The method of claim 19, wherein the side effects are graft-versus-host disease (GvHD).

21. The method of claim 19, wherein the dihydrofolate reductase inhibitor is selected from the group consisting of MTX or MPA.

22. A composition prepared according to a process comprising (a) isolating lymphocytes from a donor subject; (b) contacting the isolated lymphocytes with comprising (i) an endonuclease, and (ii) a guide RNA molecule targeting a sequence within one of Exon 2, Exon 3 or Exon 8 of the HPRT 1 gene to provide a population of substantially HPRT deficient lymphocytes; and (c) exposing the population of HPRT deficient lymphocytes to an agent which positively selects for HPRT deficient lymphocytes to provide a preparation of modified lymphocytes.

23. A kit comprising: (i) a guide RNA molecule having at least 95% sequence identity to any one of SEQ ID NOS: 40-61; and (ii) a Cas protein.

24. The kit of claim 23, wherein the Cas protein is selected from the group consisting of a Cas9 protein and a Cas12 protein.


SEQUENCE LISTING (61 sequences)

SEQ ID NO: 1 - DNA, 52 bp, synthetic construct
  misc_feature 1..52: note = Synthetic: shHPRT 734
  source 1..52: mol_type = other DNA
aggatatgcccttgactatttgtccgacatagtcaagggcatatcctttttt

SEQ ID NO: 2 - DNA, 51 bp, synthetic construct
  misc_feature 1..51: note = Synthetic: Modified sh734
  source 1..51: mol_type = other DNA
aggatatgcccttgactatgccctgacccagcatagtcaagggcatatcct

SEQ ID NO: 3 - DNA, 46 bp, synthetic construct
  misc_feature 1..46: note = Synthetic: shRNA targeting an HPRT gene
  source 1..46: mol_type = other DNA
aggatatgcccttgactatttgtccgacatagtcaagggcatatcc

SEQ ID NO: 4 - DNA, 46 bp, synthetic construct
  misc_feature 1..46: note = Synthetic: shRNA targeting an HPRT gene
  source 1..46: mol_type = other DNA
tcctatacgggaactgataaacaggctgtatcagttcccgtatagg

SEQ ID NO: 5 - DNA, 56 bp, synthetic construct
  misc_feature 1..56: note = Synthetic: shHPRT 616
  source 1..56: mol_type = other DNA
gcaggcagtataatccaaatacctgacccatatttggattatactgcctgcttttt

SEQ ID NO: 6 - DNA, 55 bp, synthetic construct
  misc_feature 1..55: note = Synthetic: shHPRT 211
  source 1..55: mol_type = other DNA
ggttatgaccttgatttatacctgacccatattaaatcaaggtcataaccttttt

SEQ ID NO: 7 - DNA, 56 bp, synthetic construct
  misc_feature 1..56: note = Synthetic: shHPRT 734.1
  source 1..56: mol_type = other DNA
gggatatgcccttgactaatacctgacccatattagtcaagggcatatcccttttt

SEQ ID NO: 8 - DNA, 111 bp, synthetic construct
  misc_feature 1..111: note = Synthetic: miRNA734 de novo (DNA form)
  source 1..111: mol_type = other DNA
acccgtacatatttttgtgtagctctagtttatagtcaagggcatatccttgtgttttttttgaaggatatgcccttgactataaactagcgctacactttttcgtcttgt

SEQ ID NO: 9 - DNA, 111 bp, synthetic construct
  misc_feature 1..111: note = Synthetic: miRNA211 de novo (DNA form)
  source 1..111: mol_type = other DNA
acccgtacatatttttgtgtagctctagttataaatcaaggtcataaccttgtgttttttttgaaggttatgaccttgatttataactagcgctacactttttcgtcttgt

SEQ ID NO: 10 - DNA, 166 bp, synthetic construct
  misc_feature 1..166: note = Synthetic: miRNA211-3G
  source 1..166: mol_type = other DNA
ccggatcaacgccctaggtttatgtttggatgaactgacatacgcgtatccgtcttttaaatcaaggtcataaccgtagtgaaatatatattaaacaggttatgaccttgatttaaaatacggtaacgcggaattcgcaactattttatcaattttttgcgtcgac

SEQ ID NO: 11 - DNA, 166 bp, synthetic construct
  misc_feature 1..166: note = Synthetic: miRNA734-3G
  source 1..166: mol_type = other DNA
ccggatcaacgccctaggtttatgtttggatgaactgacatacgcgtatccgtcttatagtcaagggcatatcctgtagtgaaatatatattaaacaaggatatgcccttgactataatacggtaacgcggaattcgcaactattttatcaattttttgcgtcgac

SEQ ID NO: 12 - DNA, 1435 bp, Homo sapiens
  misc_feature 1..1435: note = Homo sapiens hypoxanthine phosphoribosyltransferase 1 (HPRT1), mRNA
  source 1..1435: mol_type = other DNA
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 13 - DNA, 299 bp, synthetic construct
  misc_feature 1..299: note = Synthetic: 7sk/sh734 expression cassette sequence
  source 1..299: mol_type = other DNA
accatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatcct

SEQ ID NO: 14 - DNA, 248 bp, Homo sapiens
  misc_feature 1..248: note = Homo sapiens cell-line HEK-293 7SK RNA promoter region
  source 1..248: mol_type = other DNA
tcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 15 - DNA, 248 bp, Homo sapiens
  misc_feature 1..248: note = Homo sapiens cell-line HEK-293 7SK RNA promoter region with mutation 1
  source 1..248: mol_type = other DNA
tcgacgtgcagtcgggctactgccccacccatagtaccggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 16 - DNA, 3901 bp, synthetic construct
  misc_feature 1..3901: note = Synthetic: TL20 viral Backbone
  source 1..3901: mol_type = other DNA
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagct

SEQ ID NO: 17 - DNA, 6565 bp, synthetic construct
  misc_feature 1..6565: note = Synthetic: pTL20c Plasmid Sequence
  source 1..6565: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 18 - DNA, 592 bp, synthetic construct
  misc_feature 1..592: note = Synthetic: WPRE Sequence
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 19 - DNA, 769 bp, synthetic construct
  misc_feature 1..769: note = Synthetic: REV response element
  source 1..769: mol_type = other DNA
aggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccg

SEQ ID NO: 20-21 - skipped (2 entries)

SEQ ID NO: 22 - RNA, 111 nt, synthetic construct
  misc_feature 1..111: note = Synthetic: miRNA734 de novo (RNA form)
  source 1..111: mol_type = other RNA
acccgtacatatttttgtgtagctctagtttatagtcaagggcatatccttgtgttttttttgaaggatatgcccttgactataaactagcgctacactttttcgtcttgt

SEQ ID NO: 23 - RNA, 111 nt, synthetic construct
  misc_feature 1..111: note = Synthetic: miRNA211 de novo (RNA form)
  source 1..111: mol_type = other RNA
acccgtacatatttttgtgtagctctagttataaatcaaggtcataaccttgtgttttttttgaaggttatgaccttgatttataactagcgctacactttttcgtcttgt

SEQ ID NO: 24 - DNA, 42 bp, synthetic construct
  misc_feature 1..42: note = Synthetic: miRNA 451 hairpin sequence
  source 1..42: mol_type = other DNA
aaaccgttaccattactgagtttagtaatggtaatggttctc

SEQ ID NO: 25 - DNA, 20 bp, synthetic construct
  misc_feature 1..20: note = Synthetic: Sequence with Chromosome X targeted by a gRNA
  source 1..20: mol_type = other DNA
gttatggcgacccgcagccc

SEQ ID NO: 26 - DNA, 20 bp, synthetic construct
  misc_feature 1..20: note = Synthetic: Sequence with Chromosome X targeted by a gRNA
  source 1..20: mol_type = other DNA
gcgggtcgccataacggagc

SEQ ID NO: 27 - DNA, 20 bp, synthetic construct
  misc_feature 1..20: note = Synthetic: Sequence with Chromosome X targeted by a gRNA
  source 1..20: mol_type = other DNA
cgtgacgtaaagccgaaccc

SEQ ID NO: 28 - DNA, 20 bp, synthetic construct
  misc_feature 1..20: note = Synthetic: Sequence with Chromosome X targeted by a gRNA
  source 1..20: mol_type = other DNA
ggcacggaaagcgaccacct

SEQ ID NO: 29 - DNA, 20 bp, synthetic construct
  misc_feature 1..20: note = Synthetic: Sequence with Chromosome X targeted by a gRNA
  source 1..20: mol_type = other DNA
catgcgtatttgacacacga

SEQ ID NO: 30 - DNA, 20 bp, synthetic construct
  misc_feature 1..20: note = Synthetic: Sequence with Chromosome X targeted by a gRNA
  source 1..20: mol_type = other DNA
gctaagtgctagagttacgg

SEQ ID NO: 31 - DNA, 20 bp, synthetic construct
  misc_feature 1..20: note = Synthetic: Sequence with Chromosome X targeted by a gRNA
  source 1..20: mol_type = other DNA
gaggcgcgggatccgcagtg

SEQ ID NO: 32 - DNA, 20 bp, synthetic construct
  misc_feature 1..20: note = Synthetic: Sequence with Chromosome X targeted by a gRNA
  source 1..20: mol_type = other DNA
ttattactgttccccgccag

SEQ ID NO: 33 - DNA, 20 bp, synthetic construct
  misc_feature 1..20: note = Synthetic: IN-1
  source 1..20: mol_type = other DNA
attatgctgaggatttggaa

SEQ ID NO: 34 - DNA, 22 bp, synthetic construct
  misc_feature 1..22: note = Synthetic: IDT-1
  source 1..22: mol_type = other DNA
gatgatctctctcaactttaac

SEQ ID NO: 35 - DNA, 20 bp, synthetic construct
  misc_feature 1..20: note = Synthetic: IDT-6
  source 1..20: mol_type = other DNA
catacctaatcattatgctg

SEQ ID NO: 36 - DNA, 20 bp, synthetic construct
  misc_feature 1..20: note = Synthetic: IDT-4
  source 1..20: mol_type = other DNA
ggttatgaccttgatttatt

SEQ ID NO: 37 - DNA, 20 bp, synthetic construct
  misc_feature 1..20: note = Synthetic: IDT-2
  source 1..20: mol_type = other DNA
catggactaattatggacag

SEQ ID NO: 38 - DNA, 20 bp, synthetic construct
  misc_feature 1..20: note = Synthetic: IDT-3
  source 1..20: mol_type = other DNA
tagccctctgtgtgctcaag

SEQ ID NO: 39 - DNA, 23 bp, synthetic construct
  misc_feature 1..23: note = Synthetic: Nat Paper
  source 1..23: mol_type = other DNA
gccctggccggttcaggcccacg

SEQ ID NO: 40 - DNA, 23 bp, synthetic construct
  misc_feature 1..23: note = Synthetic: gRNA 12
  source 1..23: mol_type = other DNA
gccccccttgagcacacagaggg

SEQ ID NO: 41 - DNA, 23 bp, synthetic construct
  misc_feature 1..23: note = Synthetic: gRNA 13
  source 1..23: mol_type = other DNA
agccccccttgagcacacagagg

SEQ ID NO: 42 - DNA, 23 bp, synthetic construct
  misc_feature 1..23: note = Synthetic: gRNA 15
  source 1..23: mol_type = other DNA
gatgtgatgaaggagatgggagg

SEQ ID NO: 43 - DNA, 23 bp, synthetic construct
  misc_feature 1..23: note = Synthetic: gRNA 16
  source 1..23: mol_type = other DNA
ctgataaaatctacagtcatagg

SEQ ID NO: 44 - DNA, 23 bp, synthetic construct
  misc_feature 1..23: note = Synthetic: gRNA 17
  source 1..23: mol_type = other DNA
gtagccctctgtgtgctcaaggg

SEQ ID NO: 45 - DNA, 23 bp, synthetic construct
  misc_feature 1..23: note = Synthetic: gRNA 18
  source 1..23: mol_type = other DNA
ttatgctgaggatttggaaaggg

SEQ ID NO: 46 - DNA, 23 bp, synthetic construct
  misc_feature 1..23: note = Synthetic: gRNA 19
  source 1..23: mol_type = other DNA
gtgctttgatgtaatccagcagg

SEQ ID NO: 47 - DNA, 23 bp, synthetic construct
  misc_feature 1..23: note = Synthetic: gRNA 20
  source 1..23: mol_type = other DNA
tgaagtattcattatagtcaagg

SEQ ID NO: 48 - DNA, 23 bp, synthetic construct
  misc_feature 1..23: note = Synthetic: gRNA 21
  source 1..23: mol_type = other DNA
tatcctacaacaaacttgtctgg

SEQ ID NO: 49 - DNA, 23 bp, synthetic construct
  misc_feature 1..23: note = Synthetic: gRNA 22
  source 1..23: mol_type = other DNA
gaagtattcattatagtcaaggg

SEQ ID NO: 50 - DNA, 20 bp, synthetic construct
  misc_feature 1..20: note = Synthetic
  source 1..20: mol_type = other DNA
tagccccccttgagcacaca

SEQ ID NO: 51 - DNA, 20 bp, synthetic construct
  misc_feature 1..20: note = Synthetic
  source 1..20: mol_type = other DNA
atgtaatccagcaggtcagc

SEQ ID NO: 52 - DNA, 20 bp, synthetic construct
  misc_feature 1..20: note = Synthetic
  source 1..20: mol_type = other DNA
tattcagtgctttgatgtaa

SEQ ID NO: 53 - DNA, 20 bp, synthetic construct
  misc_feature 1..20: note = Synthetic
  source 1..20: mol_type = other DNA
ctgacctgctggattacatc

SEQ ID NO: 54 - DNA, 20 bp, synthetic construct
  misc_feature 1..20: note = Synthetic
  source 1..20: mol_type = other DNA
tcagactgaagagctattgt

SEQ ID NO: 55 - DNA, 20 bp, synthetic construct
  misc_feature 1..20: note = Synthetic
  source 1..20: mol_type = other DNA
aaattccagacaagtttgtt

SEQ ID NO: 56 - DNA, 20 bp, synthetic construct
  misc_feature 1..20: note = Synthetic
  source 1..20: mol_type = other DNA
ttgtaggatatgcccttgac

SEQ ID NO: 57 - DNA, 20 bp, synthetic construct
  misc_feature 1..20: note = Synthetic
  source 1..20: mol_type = other DNA
caaatcctcagcataatgat

SEQ ID NO: 58 - DNA, 20 bp, synthetic construct
  misc_feature 1..20: note = Synthetic
  source 1..20: mol_type = other DNA
ttttgcatacctaatcatta

SEQ ID NO: 59 - DNA, 20 bp, synthetic construct
  misc_feature 1..20: note = Synthetic
  source 1..20: mol_type = other DNA
catacctaatcattatgctg

SEQ ID NO: 60 - DNA, 20 bp, synthetic construct
  misc_feature 1..20: note = Synthetic
  source 1..20: mol_type = other DNA
gaaagggtgtttattcctca

SEQ ID NO: 61 - DNA, 20 bp, synthetic construct
  misc_feature 1..20: note = Synthetic
  source 1..20: mol_type = other DNA
ttcctcatggactaattatg